(19) United States
(12) Patent Application Publication
(10) Pub. No.: US 2006/0053498 A1
(43) Pub. Date: Mar. 9, 2006

(54) Full-length human cdnas encoding potentially secreted proteins

(76) Inventors: Stephane Bejanin, Rochechouart (FR); Jean-Baptiste Dumas Milne Edwards, Paris (FR); Jean-Yves Giordano, Paris (FR); Severin Jobert, Paris (FR); Hiroaki Tanaka, Antony (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/475,075

(22) PCT Filed: Apr. 18, 2001
(86) PCT No.: PCT/IB01/00914

§ 371 Date: Jul. 12, 2005

(51) Int. Cl. A01K 67/00; C07K 14/47; C12Q 1/68; C07H 21/04; C12P 21/06

(52) U.S. Cl. 800/8; 435/6; 435/69.1; 435/320.1; 435/325; 530/350; 536/23.5

(57) ABSTRACT

The invention concerns GENSET polynucleotides and polypeptides. Such GENSET products may be used as reagents in forensic analyses, as chromosome markers, as tissue/cell/organelle-specific markers, in the production of expression vectors. In addition, they may be used in screening and diagnosis assays for abnormal GENSET expression and/or biological activity and for screening compounds that may be used in the treatment of GENSET-related disorders.

### CROSS REFERENCE TO RELATED APPLICATIONS

The present application is related to and claims priority from U.S. provisional Application Nos. 60/197,873, filed Apr. 18, 2000, 60/224,009, filed Aug. 7, 2000, 60/260,328, filed Jan. 8, 2001, and 60/224,006, filed Aug. 4, 2000, the entire disclosures of each of which is herein incorporated by reference.

### FIELD OF THE INVENTION

The present invention is directed to GENSET polypeptides, fragments thereof, and the regulatory regions located in the 5′- and 3′-ends of the genes encoding the polypeptides. The invention also concerns polypeptides encoded by GENSET genes and fragments thereof. The present invention also relates to recombinant vectors including the polynucleotides of the present invention, particularly recombinant vectors comprising a GENSET gene regulatory region or a sequence encoding a GENSET polypeptide, and to host cells containing the polynucleotides of the invention, as well as to methods of making such vectors and host cells. The present invention further relates to the use of these recombinant vectors and host cells in the production of the polypeptides of the invention. The invention further relates to antibodies that specifically bind to the polypeptides of the invention and to methods for producing such antibodies and fragments thereof. The invention also provides methods of detecting the presence of the polynucleotides and polypeptides of the present invention in a sample, methods of diagnosis and screening of abnormal GENSET polypeptide expression and/or biological activity, methods of screening compounds for their ability to modulate the activity or expression of the GENSET polypeptides, and uses of such compounds.

### BACKGROUND OF THE INVENTION

The estimated 30,000-12,000 genes scattered along the human chromosomes offer tremendous promise for the understanding, diagnosis, and treatment of human diseases. In addition, probes capable of specifically hybridizing to loci distributed throughout the human genome find application in the construction of high resolution chromosome maps and in the identification of individuals.

In the past, the characterization of even a single human gene was a painstaking process, requiring years of effort. Recent developments in the areas of cloning vectors, DNA sequencing, and computer technology have merged to greatly accelerate the rate at which human genes can be isolated, sequenced, mapped, and characterized.

Currently, two different approaches are being pursued for identifying and characterizing the genes distributed along the human genome. In one approach, large fragments of genomic DNA are isolated, cloned, and sequenced. Potential open reading frames in these genomic sequences are identified using bio-informatics software. However, this approach entails sequencing large stretches of human DNA which do not encode proteins in order to find the protein encoding sequences scattered throughout the genome. In addition to requiring extensive sequencing, the bio-informatics software may mischaracterize the genomic sequences obtained, i.e., labeling non-coding DNA as coding DNA and vice versa.

An alternative approach takes a more direct route to identifying and characterizing human genes. In this approach, complementary DNAs (cDNAs) are synthesized from isolated messenger RNAs (mRNAs) which encode human proteins. Using this approach, sequencing is only performed on DNA which is derived from protein coding fragments of the genome. Often, only short stretches of the cDNAs are sequenced to obtain sequences called expressed sequence tags (ESTs). The ESTs may then be used to isolate or purify cDNAs which include sequences adjacent to the EST sequences. The cDNAs may contain all of the sequence of the EST which was used to obtain them or only a fragment of the sequence of the EST which was used to obtain them. In addition, the cDNAs may contain the full coding sequence of the gene from which the EST was derived or, alternatively, the cDNAs may include fragments of the coding sequence of the gene from which the EST was derived. It will be appreciated that there may be several cDNAs which include the EST sequence as a result of alternate splicing or the activity of alternative promoters.

In the past, these short EST sequences were often obtained from oligo-dT primed cDNA libraries. Accordingly, they mainly corresponded to the 3′ untranslated region of the mRNA. In part, the prevalence of EST sequences derived from the 3′ end of the mRNA is a result of the fact that typical techniques for obtaining cDNAs are not well suited for isolating cDNA sequences derived from the 5′ ends of mRNAs (Adams et al, Nature 377:3-174, 1996, Hillier et al., Genome Res. 6:807-828, 1996). In addition, in those reported instances where longer cDNA sequences have been obtained, the reported sequences typically correspond to coding sequences and do not include the full 5′ untranslated region (5′UTR) of the mRNA from which the cDNA is derived. Indeed, 5′UTRs have been shown to affect either the stability or translation of mRNAs. Thus, regulation of gene expression may be achieved through the use of alternative 5′UTRs as shown, for instance, for the translation of the tissue inhibitor of metalloprotease mRNA in mitogenically activated cells (Waterhouse et al., J Biol Chem. 265:5585-9. 1990). Furthermore, modification of 5′UTR through mutation, insertion or translocation events may even be implied in pathogenesis. For instance, the Fragile X syndrome, the most common cause of inherited mental retardation, is partly due to an insertion of multiple CGG trinucleotides in the 5′UTR of the Fragile X mRNA resulting in the inhibition of protein synthesis via ribosome stalling (Feng et al., Science 268:731-4, 1995). An aberrant mutation in regions of the 5′UTR known to inhibit translation of the proto-oncogene c-myc was shown to result in upregulation of c-myc protein levels in cells derived from patients with multiple myelomas (Willis et al., Curr Top Microbiol Immunol 224:269-76, 1997). In addition, the use of oligo-dT primed cDNA libraries does not allow the isolation of complete 5′UTRs since such incomplete sequences obtained by this process may not include the first exon of the mRNA, particularly in situations where the first exon is short. Furthermore, they may not include some exons, often short ones, which are located upstream of splicing sites. Thus, there is a need to obtain sequences derived from the 5′ ends of mRNAs.

Moreover, despite the great amount of EST data that large-scale sequencing projects have yielded (Adams et al., Nature 377:174, 1996; Hillier et a., Genome Res. 6:807-828, 1996), information concerning the biological function of the mRNAs corresponding to such obtained cDNAs has revealed to be limited. Indeed, whereas the knowledge of the complete coding sequence is absolutely necessary to investigate the biological function of mRNAs, ESTs yield only partial coding sequences. So far, large-scale full-length cDNA cloning has been achieved only with limited success because of the poor efficiency of methods for constructing full-length cDNA libraries. Indeed, such methods require either a large amount of mRNA (Ederly et al., 1995), thus resulting in non representative full-length libraries when small amounts of tissue are available or require PCR amplification (Maruyama et al., 1994; CLONTECHniques, 1996) to obtain a reasonable number of clones, thus yielding strongly biased cDNA libraries where rare and long cDNAs are lost. Thus, there is a need to obtain full-length cDNAs, i.e. cDNAs containing the full coding sequence of their corresponding mRNAs.

While many sequences derived from human chromosomes have practical applications, approaches based on the identification and characterization of those chromosomal sequences which encode a protein product are particularly relevant to diagnostic and therapeutic uses. Of the 30,000-120,000 protein coding genes, those genes encoding proteins which are secreted from the cell in which they are synthesized, as well as the secreted proteins themselves, are particularly valuable as potential therapeutic agents. Such proteins are often involved in cell to cell communication and may be responsible for producing a clinically relevant response in their target cells. In fact, several secretory proteins, including tissue plasminogen activator, G-CSF, GM-CSF, erythropoietin, human growth hormone, insulin, interferon-α, interferon-β, interferon-γ, and interleukin-2, are currently in clinical use. These proteins are used to treat a wide range of conditions, including acute myocardial infarction, acute ischemic stroke, anemia, diabetes, growth hormone deficiency, hepatitis, kidney carcinoma, chemotherapy induced neutropenia and multiple sclerosis. For these reasons, cDNAs encoding secreted proteins or fragments thereof represent a particularly valuable source of therapeutic agents. Thus, there is a need for the identification and characterization of secreted proteins and the nucleic acids encoding them.

In addition to being therapeutically useful themselves, secretory proteins include short peptides, called signal peptides, at their amino termini which direct their secretion. These signal peptides are encoded by the signal sequences located at the 5′ ends of the coding sequences of genes encoding secreted proteins. Because these signal peptides will direct the extracellular secretion of any protein to which they are operably linked, the signal sequences may be exploited to direct the efficient secretion of any protein by operably linking the signal sequences to a gene encoding the protein for which secretion is desired. In addition, fragments of the signal peptides called membrane-translocating sequences may also be used to direct the intracellular import of a peptide or protein of interest. This may prove beneficial in gene therapy strategies in which it is desired to deliver a particular gene product to cells other than the cells in which it is produced. Signal sequences encoding signal peptides also find application in simplifying protein purification techniques. In such applications, the extracellular secretion of the desired protein greatly facilitates purification by reducing the number of undesired proteins from which the desired protein must be selected. Thus, there exists a need to identify and characterize the 5′ fragments of the genes for secretory proteins which encode signal peptides.

Sequences coding for secreted proteins may also find application as therapeutics or diagnostics. In particular, such sequences may be used to determine whether an individual is likely to express a detectable phenotype, such as a disease, as a consequence of a mutation in the coding sequence for a secreted protein. In instances where the individual is at risk of suffering from a disease or other undesirable phenotype as a result of a mutation in such a coding sequence, the undesirable phenotype may be corrected by introducing a normal coding sequence using gene therapy. Alternatively, if the undesirable phenotype results from overexpression of the protein encoded by the coding sequence, expression of the protein may be reduced using antisense or triple helix based strategies.

The secreted human polypeptides encoded by the coding sequences may also be used as therapeutics by administering them directly to an individual having a condition, such as a disease, resulting from a mutation in the sequence encoding the polypeptide. In such an instance, the condition can be cured or ameliorated by administering the polypeptide to the individual.

In addition, the secreted human polypeptides or fragments thereof may be used to generate antibodies useful in determining the tissue type or species of origin of a biological sample. The antibodies may also be used to determine the cellular localization of the secreted human polypeptides or the cellular localization of polypeptides which have been fused to the human polypeptides. In addition, the antibodies may also be used in immunoaffinity chromatography techniques to isolate, purify, or enrich the human polypeptide or a target polypeptide which has been fused to the human polypeptide.

Public information on the number of human genes for which the promoters and upstream regulatory regions have been identified and characterized is quite limited. In part, this may be due to the difficulty of isolating such regulatory sequences. Upstream regulatory sequences such as transcription factor binding sites are typically too short to be utilized as probes for isolating promoters from human genomic libraries. Recently, some approaches have been developed to isolate human promoters. One of them consists of making a CpG island library (Cross et al., Nature Genetics 6:236-244, 1994). The second consists of isolating human genomic DNA sequences containing SpeI binding sites by the use of SpeI binding protein (Mortlock et al., Genome Res. 6:327-335, 1996). Both of these approaches have their limits due to a lack of specificity and of comprehensiveness. Thus, there exists a need to identify and systematically characterize the 5′ fragments of the genes.

cDNAs including the 5′ ends of their corresponding mRNA may be used to efficiently identify and isolate 5′UTRs and upstream regulatory regions which control the location, developmental stage, rate, and quantity of protein synthesis, as well as the stability of the mRNA (Theil et al., BioFactors 4:87-93, (1993). Once identified and characterized, these regulatory regions may be utilized in gene therapy or protein purification schemes to obtain the desired amount and locations of protein synthesis or to inhibit, reduce, or prevent the synthesis of undesirable gene products.

In addition, cDNAs containing the 5′ ends of secretory protein genes may include sequences useful as probes for chromosome mapping and the identification of individuals. Thus, there is a need to identify and characterize the sequences upstream of the 5′ coding sequences of genes encoding secretory proteins.

### SUMMARY OF THE INVENTION

The present invention provides a purified or isolated polynucleotide comprising, consisting of, or consisting essentially of a nucleotide sequence selected from the group consisting of: (a) the sequences of SEQ ID NOs:1-169, 339-455, 561-784; (b) the sequences of clone inserts of the deposited clone pool; (c) the coding sequences of SEQ ID NOs:1-169, 339-455, 561-784; (d) the coding sequences of the clone inserts of the deposited clone pool; (e) the sequences encoding one of the polypeptides of SEQ ID NOs:170-338, 456-560, 785-918; (f) the sequences encoding one of the polypeptides encoded by the clone inserts of the deposited clone pool; (g) the genomic sequences coding for the GENSET polypeptides; (h) the 5′ transcriptional regulatory regions of GENSET genes; (i) the 3′ transcriptional regulatory regions of GENSET genes; (j) the polynucleotides comprising the nucleotide sequence of any combination of (g)-(i); (k) the variant polynucleotides of any of the polynucleotides of (a)-(j); (1) the polynucleotides comprising a nucleotide sequence of (a)-(k), wherein the polynucleotide is single stranded, double stranded, or a portion is single stranded and a portion is double stranded; (m) the polynucleotides comprising a nucleotide sequence complementary to any of the single stranded polynucleotides of (l). The invention further provides for fragments of the nucleic acid molecules of (a)-(m) described above.

Further embodiments of the invention include purified or isolated polynucleotides that comprise, consist of, or consist essentially of a nucleotide sequence at least 70% identical, more preferably at least 75%, and even more preferably at least 80%, 85%, 90%, 95%, 96%, 97%, 98%, or 99% identical, to any of the nucleotide sequences in (a)-(m) above, e.g. over a region of at least about 25, 50, 100, 150, 250, 500, 1000, or more contiguous nucleotides, or a polynucleotide which hybridizes under stringent hybridization conditions to a polynucleotide in (a)-(m) above.

The present invention also relates to recombinant vectors, which include the purified or isolated polynucleotides of the present invention, and to host cells recombinant for the polynucleotides of the present invention, as well as to methods of making such vectors and host cells. The present invention further relates to the use of these recombinant vectors and recombinant host cells in the production of GENSET polypeptides.

The invention further provides a purified or isolated polypeptide comprising, consisting of, or consisting essentially of an amino acid sequence selected from the group consisting of: (a) the polypeptides of SEQ ID NOs:170-338, 456-560, 785-918; (b) the polypeptides encoded by the clone inserts of the deposited clone pool; (c) the epitope-bearing fragments of the polypeptides of SEQ ID NOs:170-338, 456-560, 785-918; (d) the epitope-bearing fragments of the polypeptides encoded by the clone inserts contained in the deposited clone pool; (e) the domains of the polypeptides of SEQ ID NOs: 170-338, 456-560, 785-918; (f) the domains of the polypeptides encoded by the clone inserts contained in the deposited clone pool; and (g) the allelic variant polypeptides of any of the polypeptides of (a)-(f). The invention further provides for fragments of the polypeptides of (a)-(g) above, such as those having biological activity or comprising biologically functional domain(s).

The present invention further includes polypeptides with an amino acid sequence with at least 70% similarity, and more preferably at least 75%, 80%, 85%, 90%, 95%, 96%, 97%, 98%, or 99% similarity to those polypeptides described in (a)-(g), as well as polypeptides having an amino acid sequence at least 70% identical, more preferably at least 75% identical, and still more preferably 80%, 85%, 90%, 95%, 96%, 97%, 98%, or 99% identical to those polypeptides described in (a)-(g), e.g. over a region of at least about 25, 50, 100, 150, 250, 500, 1000, or more amino acids. The invention further relates to methods of making the polypeptides of the present invention.

The present invention further relates to transgenic plants or animals, wherein said transgenic plant or animal is transgenic for a polynucleotide of the present invention and expresses a polypeptide of the present invention.

The invention further relates to antibodies that specifically bind to GENSET polypeptides of the present invention and fragments thereof as well as to methods for producing such antibodies and fragments thereof.

The invention also provides kits, uses and methods for detecting GENSET gene expression and/or biological activity in a biological sample. One such method involves assaying for the expression of a GENSET polynucleotide in a biological sample using the polymerase chain reaction (PCR) to amplify and detect GENSET polynucleotides or Southern and Northern blot hybridization to detect GENSET genomic DNA, cDNA or mRNA. Alternatively, a method of detecting GENSET gene expression in a test sample can be accomplished using a compound which binds to a GENSET polypeptide of the present invention or a portion of a GENSET polypeptide.

The present invention also relates to diagnostic methods and uses of GENSET polynucleotides and polypeptides for identifying individuals or non-human animals having elevated or reduced levels of GENSET gene products, which individuals are likely to benefit from therapies to suppress or enhance GENSET gene expression, respectively, and to methods of identifying individuals or non-human animals at increased risk for developing, or at present having, certain diseases/disorders associated with GENSET polypeptide expression or biological activity.

The present invention also relates to kits, uses and methods of screening compounds for their ability to modulate (e.g. increase or inhibit) the activity or expression of GENSET polypeptides including compounds that interact with GENSET gene regulatory sequences and compounds that interact directly or indirectly with a GENSET polypeptide. Uses of such compounds are also within the scope of the present invention.

The present invention also relates to pharmaceutical or physiologically acceptable compositions comprising, an active agent, the polypeptides, polynucleotides or antibodies of the present invention, as well as, typically, a pharmaceutically acceptable carrier.

The present invention also relates to computer systems containing cDNA codes and polypeptide codes of sequences of the invention and to computer-related methods of comparing sequences, identifying homology or features using GENSET polypeptides or GENSET polynucleotide sequences of the invention.

In another aspect, the present invention provides an isolated polynucleotide, the polynucleotide comprising a nucleic acid sequence encoding: i) a polypeptide comprising an amino acid sequence having at least about 80% identity to any one of the sequences shown as SEQ ID NOs:170-338, 456-560, 785-918 or any one of the sequences of polypeptides encoded by the clone inserts of the deposited clone pool; or a biologically active fragment of the polypeptide.

In one embodiment, the polypeptide comprises any one of the sequences shown as SEQ ID NOs:170-338, 456-560, 785-918 or any one of the sequences of the polypeptides encoded by the clone inserts of the deposited clone pool. In another embodiment, the polypeptide comprises a signal peptide. In another embodiment, the polypeptide is a mature protein. In another embodiment, the nucleic acid sequence has at least about 80% identity over at least about 100 contiguous nucleotides to any one of the sequences shown as SEQ ID NOs:1-169, 339-455, 561-784 or any one of the sequences of the clone inserts of the deposited clone pool. In another embodiment, the polynucleotide hybridizes under stringent conditions to a polynucleotide comprising any one of the sequences shown as SEQ ID NOs:1-169, 339-455, 561-784 or any one of the sequences of the clone inserts of the deposited clone pool. In another embodiment, the nucleic acid sequence comprises any one of the sequences shown as SEQ ID NOs:1-169, 339-455, 561-784 or any one the sequences of the clone inserts of the deposited clone pool. In another embodiment, the polynucleotide is operably linked to a promoter.

In another aspect, the present invention provides an expression vector comprising any of the herein-described polynucleotides, operably linked to a promoter. In another aspect, the present invention provides a host cell recombinant for any of the herein-described polynucleotides. In another aspect, the present invention provides a non-human transgenic animal comprising the host cell.

In another aspect, the present invention provides a method of making a GENSET polypeptide, the method comprising a) providing a population of host cells comprising a herein-described polynucleotide and b) culturing the population of host cells under conditions conducive to the production of the polypeptide within said host cells.

In one embodiment, the method further comprises purifying the polypeptide from the population of host cells.

In another aspect, the present invention provides a method of making a GENSET polypeptide, the method comprising a) providing a population of cells comprising a polynucleotide encoding a herein-described polypeptide; b) culturing the population of cells under conditions conducive to the production of the polypeptide within the cells; and c) purifying the polypeptide from the population of cells.

In another aspect, the present invention provides an isolated polynucleotide, the polynucleotide comprising a nucleic acid sequence having at least about 80% identity over at least about 100 contiguous nucleotides to any one of the sequences shown as SEQ ID NOs:1-169, 339-455, 561-784 or any one of the sequences of the clone inserts of the deposited clone pool.

In one embodiment, the polynucleotide hybridizes under stringent conditions to a polynucleotide comprising any one of the sequences shown as SEQ ID NOs:1-169, 339-455, 561-784 or any one of the sequences of the clone inserts of the deposited clone pool. In another embodiment, the polynucleotide comprises any one of the sequences shown as SEQ ID NOs:1-169, 339-455, 561-784 or any one of the sequences of the clone inserts of the deposited clone pool.

In another aspect, the present invention provides a biologically active polypeptide encoded by any of the herein-described polynucleotides.

In another aspect, the present invention provides an isolated polypeptide or biologically active fragment thereof, the polypeptide comprising an amino acid sequence having at least about 80% sequence identity to any one of the sequences shown as SEQ ID NOs:170-338, 456-560, 785-918 or any one of the sequences of polypeptides encoded by the clone inserts of the deposited clone pool.

In one embodiment, the polypeptide is selectively recognized by an antibody raised against an antigenic polypeptide, or an antigenic fragment thereof, the antigenic polypeptide comprising any one of the sequences shown as SEQ ID NOs:170-338, 456-560, 785-918 or any one of the sequences of polypeptides encoded by the clone inserts of the deposited clone pool. In another embodiment, the polypeptide comprises any one of the sequences shown as SEQ ID NOs:170-338, 456-560, 785-918 or any one of the sequences of polypeptides encoded by the clone inserts of the deposited clone pool. In another embodiment, the polypeptide comprises a signal peptide. In another embodiment, the polypeptide is a mature protein.

In another aspect, the present invention provides an antibody that specifically binds to any of ther herein-described polypeptides.

In another aspect, the present invention provides a method of determining whether a GENSET gene is expressed within a mammal, the method comprising the steps of: a) providing a biological sample from said mammal; b) contacting said biological sample with either of: i) a polynucleotide that hybridizes under stringent conditions to any of the herein-described polynucleotides; or ii) a polypeptide that specifically binds to any of the herein-described polypeptides; and c) detecting the presence or absence of hybridization between the polynucleotide and an RNA species within the sample, or the presence or absence of binding of the polypeptide to a protein within the sample; wherein a detection of the hybridization or of the binding indicates that the GENSET gene is expressed within the mammal.

In one embodiment, the polynucleotide is a primer, and the hybridization is detected by detecting the presence of an amplification product comprising the sequence of the primer. In another embodiment, the polypeptide is an antibody.

In another aspect, the present invention provides a method of determining whether a mammal has an elevated or reduced level of GENSET gene expression, the method comprising the steps of: a) providing a biological sample from the mammal; and b) comparing the amount of any of the herein-described polypeptides, or of an RNA species encoding the polypeptide, within the biological sample with a level detected in or expected from a control sample; wherein an increased amount of the polypeptide or the RNA species within the biological sample compared to the level detected in or expected from the control sample indicates that the mammal has an elevated level of the GENSET gene expression, and wherein a decreased amount of the polypeptide or the RNA species within the biological sample compared to the level detected in or expected from the control sample indicates that the mammal has a reduced level of the GENSET gene expression.

In another aspect, the present invention provides a method of identifying a candidate modulator of a GENSET polypeptide, the method comprising: a) contacting any of the herein-described polypeptides with a test compound; and b) determining whether the compound specifically binds to the polypeptide; wherein a detection that the compound specifically binds to the polypeptide indicates that the compound is a candidate modulator of the GENSET polypeptide.

In one embodiment, the method further comprises testing the biological activity of the GENSET polypeptide in the presence of the candidate modulator, wherein an alteration in the biological activity of the GENSET polypeptide in the presence of the compound in comparison to the activity in the absence of said compound indicates that the compound is a modulator of the GENSET polypeptide.

In another aspect, the present invention provides a method for the production of a pharmaceutical composition, the method comprising a) identifying a modulator of a GENSET polypeptide using any of the herein-described methods; and b) combining the modulator with a pharmaceutically acceptable carrier.

### BRIEF DESCRIPTION OF DRAWINGS

FIG. 1 is a block diagram of an exemplary computer system.

FIG. 2 is a flow diagram illustrating one embodiment of a process 200 for comparing a new nucleotide or protein sequence with a database of sequences in order to determine the identity levels between the new sequence and the sequences in the database.

FIG. 3 is a flow diagram illustrating one embodiment of a process 250 in a computer for determining whether two sequences are homologous.

FIG. 4 is a flow diagram illustrating one embodiment of an identifier process 300 for detecting the presence of a feature in a sequence.

### BRIEF DESCRIPTION OF THE TABLES

Table I provides the SEQ ID Nos in the present application (with the SEQ ID Nos corresponding to nucleic acid sequences preceded by “NUC”, and the SEQ ID Nos corresponding to the encoded polypeptide sequences preceded by “PRT”) that correspond to a SEQ ID NO in priority application number 60/197,873. Applicants' internal designation number (Clone ID) corresponding to each sequence identification (SEQ ID) number is also provided.

Table II lists the putative chromosomal location of the polynucleotides of the present invention. The SEQ ID NO listed for each polynucleotide is that from the priority application 60/197,873; the corresponding SEQ ID NOs for the sequence in the present application can be determined by referring to Table I.

Table III lists the number of hits in Genset's cDNA libraries of tissues and cell types for polynucleotides of the invention. The following abbreviations are used to refer to each cell or tissue type: A=Brain; B=Fetal brain; C=Fetal kidney; D=Fetal liver; E=Pituitary gland; F=Liver; G=Placenta; H=Prostate; I=Salivary gland; J=Stomach/Intestine; and K=Testis. The SEQ ID NO listed for each polynucleotide is that from the priority application 60/197,873; the corresponding SEQ ID NOs for the sequence in the present application can be determined by referring to Table I.

Table IV lists the number of hits in publicly available library of tissues and cell types for polynucleotides of the invention. The SEQ ID NO listed for each polynucleotide is that from the priority application 60/197,873; the corresponding SEQ ID NOs for each sequence in the present application can be determined by referring to Table I.

Table V lists the tissues and cell types in which the polynucleotide sequences of the present invention are over- or under-represented. The SEQ ID NO listed for each polynucleotide is that from the priority application 60/197,873; the corresponding SEQ ID NOs for each sequence in the present application can be determined by referring to Table I.

### BRIEF DESCRIPTION OF THE SEQUENCE LISTING

SEQ ID NOs:1-169, 339-455, 561-784 are the nucleotide sequences of cDNAs, with open reading frames as indicated as features (CDS). When appropriate, the locations of the potential polyadenylation site and polyadenylation signal are also indicated.

SEQ ID NOs:170-338, 456-560, 785-918 are the amino acid sequences of proteins encoded by the cDNAs of SEQ ID NOs:1-169, 339-455, 561-784.

SEQ ID NOs:1-85, 339-400, 406-407, 413-415, 561-594, and 634-651 are the nucleotide sequences of cDNAs encoding a potentially secreted protein. The locations of the ORFs and sequences encoding signal peptides are listed in the accompanying Sequence Listing. In addition, the von Heijne score of the signal peptide computed as described below is listed as the “score” in the accompanying Sequence Listing. The sequence of the signal-peptide is listed as “seq” in the accompanying Sequence Listing. The “/” in the signal peptide sequence indicates the location where proteolytic cleavage of the signal peptide occurs to generate a mature protein. When appropriate, the locations of the first and last nucleotides of the coding sequences, eventually the locations of the first and last nucleotides of the polyA and the locations of the first and last nucleotides of the polyA sites are indicated.

SEQ ID NOs:86-169, 401-405, 408-412, 416-455, 595-633, 652-784 are the nucleotide sequences of cDNAs in which no sequence encoding a signal peptide has been identified to date. However, it remains possible that subsequent analysis will identify a sequence encoding a signal peptide in these nucleic acids. The locations of the ORFs are listed in the accompanying Sequence Listing. When appropriate, the locations of the first and last nucleotides of the coding sequences, eventually the locations of the first and last nucleotides of the polyA and the locations of the first and last nucleotides of the polyA sites are indicated.

SEQ ID NOs:170-254, 456-517, 520-521, 527-529, 785-818, and 858-875 are the amino acid sequences of polypeptides which contain a signal peptide. These polypeptides are encoded by the cDNAs of SEQ ID NOs: 1-85, 339-400, 406-407, 413-415, 561-594, and 634-651. The location of the signal peptide is listed in the accompanying Sequence Listing.

SEQ ID NOs:255-338, 517-519, 522-526, 530-560, 819-857, 876-918 are the amino acid sequences of polypeptides in which no signal peptide has been identified to date. However, it remains possible that subsequent analysis will identify a signal peptide in these polypeptides. These polypeptides are encoded by the nucleic acids of SEQ ID NOs: 86-169, 401-405, 408-412, 416-455, 595-633, 652-784.

In accordance with the regulations relating to Sequence Listings, the following codes have been used in the Sequence Listing to describes nucleotide sequences. The code “r” in the sequences indicates that the nucleotide may be a guanine or an adenine. The code “y” in the sequences indicates that the nucleotide may be a thymine or a cytosine. The code “m” in the sequences indicates that the nucleotide may be an adenine or a cytosine. The code “k” in the sequences indicates that the nucleotide may be a guanine or a thymine. The code “s” in the sequences indicates that the nucleotide may be a guanine or a cytosine. The code “w” in the sequences indicates that the nucleotide may be an adenine or an thymine. In addition, all instances of the symbol “n” in the nucleic acid sequences mean that the nucleotide can be adenine, guanine, cytosine or thymine.

In some instances, the polypeptide sequences in the Sequence Listing contain the symbol “Xaa.” These “Xaa” symbols indicate either (1) a residue which cannot be identified because of nucleotide sequence ambiguity or (2) a stop codon in the determined sequence where applicants believe one should not exist (if the sequence were determined more accurately). In some instances, several possible identities of the unknown amino acids may be suggested by the genetic code.

In the case of secreted proteins, it should be noted that, in accordance with the regulations governing Sequence Listings, in the appended Sequence Listing the encoded protein (i.e. the protein containing the signal peptide and the mature protein or part thereof) extends from an amino acid residue having a negative number through a positively numbered amino acid residue. Thus, the first amino acid of the mature protein resulting from cleavage of the signal peptide is designated as amino acid number 1, and the first amino acid of the signal peptide is designated with the appropriate negative number.

### DETAILED DESCRIPTION OF THE INVENTION AND PREFERRED EMBODIMENTS

### Definitions

Before describing the invention in greater detail, the following definitions are set forth to illustrate and define the meaning and scope of the terms used to describe the invention herein.

The term “GENSET gene,” when used herein, encompasses genomic, mRNA and cDNA sequences encoding a GENSET polypeptide, including the 5′ and 3′ untranslated regions of said sequences.

The term “GENSET polypeptide biological activity” or “GENSET biological activity” is intended for polypeptides exhibiting any activity similar, but not necessarily identical, to an activity of a GENSET polypeptide of the invention. The GENSET polypeptide biological activity of a given polypeptide may be assessed using any suitable biological assay, a number of which are known to those skilled in the art. In contrast, the term “biological activity” refers to any activity that any polypeptide may have.

The term “corresponding mRNA” refers to mRNA which was or can be a template for cDNA synthesis for producing a cDNA of the present invention.

The term “corresponding genomic DNA” refers to genomic DNA which encodes an mRNA of interest, e.g. corresponding to a cDNA of the invention, which genomic DNA includes the sequence of one of the strands of the mRNA, in which thymidine residues in the sequence of the genomic DNA (or cDNA) are replaced by uracil residues in the mRNA.

The term “deposited clone pool” is used herein to refer to the pool of clones entitled cDNA-8-2000, deposited with the ATCC on Sep. 27, 2000, or the pool of clones entitled cDNA-11-2000, deposited with the ATCC on Nov. 27, 2000, or any other deposited clone pool containing a clone corresponding to any of the herein-described sequences.

The term “heterologous”, when used herein, is intended to designate any polynucleotide or polypeptide other than a GENSET polynucleotide or GENSET polypeptide of the invention, respectively.

“Providing” with respect to, e.g. a biological sample, population of cells, etc. indicates that the sample, population of cells, etc. is somehow used in a method or procedure. Significantly, “providing” a biological sample or population of cells does not require that the sample or cells are specifically isolated or obtained for the purposes of the invention, but can instead refer, for example, to the use of a biological sample obtained by another individual, for another purpose.

An “amplification product” refers to a product of any amplification reaction, e.g. PCR, RT-PCR, LCR, etc.

A “modulator” of a protein or other compound refers to any agent that has a functional effect on the protein, including physical binding to the protein, alterations of the quantity or quality of expression of the protein, altering any measurable or detectable activity, property, or behavior of the protein, or in any way interacts with the protein or compound.

“A test compound” can be any molecule that is evaluated for its ability to modulate a protein or other compound.

An antibody or other compound that specifically binds to a polypeptide or polynucleotide of the invention is also said to “selectively recognize” the polypeptide or polynucleotide.

The term “isolated” with respect to a molecule requires that the molecule be removed from its original environment (e. g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment. For example, a naturally-occurring polynucleotide present in a living animal is not isolated, but the same polynucleotide, separated from some or all of the coexisting materials in the natural system, is isolated. Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified polynucleotide makes up less than 5% of the number of nucleic acid inserts in the vector molecules. Further specifically excluded are whole cell genomic DNA or whole cell RNA preparations (including said whole cell preparations which are mechanically sheared or enzymatically digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude. To illustrate, individual cDNA clones isolated from a cDNA library have been conventionally purified to electrophoretic homogeneity. The sequences obtained from these clones could not be obtained directly either from the library or from total human DNA. The cDNA clones are not naturally occurring as such, but rather are obtained via manipulation of a partially purified naturally occurring substance (messenger RNA). The conversion of mRNA into a cDNA library involves the creation of a synthetic substance (cDNA) and pure individual cDNA clones can be isolated from the synthetic library by clonal selection. Thus, creating a cDNA library from messenger RNA and subsequently isolating individual clones from that library results in an approximately 10⁴-10⁶ fold purification of the native message.

The term “purified” is further used herein to describe a polypeptide or polynucleotide of the invention which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero-dimers, trimers, etc. The term “purified” may also be used to specify the separation of covalently closed (i.e. circular) polynucleotides from linear polynucleotides. A polynucleotide is substantially pure when at least about 50%, preferably 60 to 75% of a sample exhibits a single polynucleotide sequence and conformation (linear versus covalently close). A substantially pure polypeptide or polynucleotide typically comprises about 50%, preferably 60 to 90% weight/weight of a polypeptide or polynucleotide sample, respectively, more usually about 95%, and preferably is over about 99% pure. Polypeptide and polynucleotide purity, or homogeneity, is indicated by a number of means well known in the art, such as agarose or polyacrylamide gel electrophoresis of a sample, followed by visualizing a single band upon staining the gel. For certain purposes higher resolution can be provided by using HPLC or other means well known in the art. As an alternative embodiment, purification of the polypeptides and polynucleotides of the present invention may be expressed as “at least” a percent purity relative to heterologous polypeptides and polynucleotides (DNA, RNA or both). As a preferred embodiment, the polypeptides and polynucleotides of the present invention are at least; 10%, 20%, 30%, 40%, 50%, 60%, 70%, 80%, 90%, 95%, 96%, 96%, 98%, 99%, or 100% pure relative to heterologous polypeptides and polynucleotides, respectively. As a further preferred embodiment the polypeptides and polynucleotides have a purity ranging from any number, to the thousandth position, between 90% and 100% (e.g., a polypeptide or polynucleotide at least 99.995% pure) relative to either heterologous polypeptides or polynucleotides, respectively, or as a weight/weight ratio relative to all compounds and molecules other than those existing in the carrier. Each number representing a percent purity, to the thousandth position, may be claimed as individual species of purity.

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (i.e. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modification such as (a) an alternative linking group, (b) an analogous form of purine, (c) an analogous form of pyrimidine, or (d) an analogous sugar. For examples of analogous linking groups, purine, pyrimidines, and sugars, see, for example, PCT publication No. WO 95/04064, which disclosure is hereby incorporated by reference in its entirety. Preferred modifications of the present invention include, but are not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v) ybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. The polynucleotide sequences of the invention may be prepared by any known method, including synthetic, recombinant, ex vivo generation, or a combination thereof, as well as utilizing any purification methods known in the art. Methylenemethylimino linked oligonucleosides as well as mixed backbone compounds having, may be prepared as described in U.S. Pat. Nos. 5,378,825; 5,386,023; 5,489,677; 5,602,240; and 5,610,289, which disclosures are hereby incorporated by reference in their entireties. Formacetal and thioformacetal linked oligonucleosides may be prepared as described in U.S. Pat. Nos. 5,264,562 and 5,264,564, which disclosures are hereby incorporated by reference in their entireties. Ethylene oxide linked oligonucleosides may be prepared as described in U.S. Pat. No. 5,223,618, which disclosure is hereby incorporated by reference in its entirety. Phosphinate oligonucleotides may be prepared as described in U.S. Pat. No. 5,508,270, which disclosure is hereby incorporated by reference in its entirety. Alkyl phosphonate oligonucleotides may be prepared as described in U.S. Pat. No. 4,469,863, which disclosure is hereby incorporated by reference in its entirety. 3′-Deoxy-3′-methylene phosphonate oligonucleotides may be prepared as described in U.S. Pat. Nos. 5,610,289 or 5,625,050 which disclosures are hereby incorporated by reference in their entireties. Phosphoramidite oligonucleotides may be prepared as described in U.S. Pat. No. 5,256,775 or U.S. Pat. No. 5,366,878 which disclosures are hereby incorporated by reference in their entireties. Alkylphosphonothioate oligonucleotides may be prepared as described in published PCT applications WO 94/17093 and WO 94/02499 which disclosures are hereby incorporated by reference in their entireties. 3′-Deoxy-3′-amino phosphoramidite oligonucleotides may be prepared as described in U.S. Pat. No. 5,476,925, which disclosure is hereby incorporated by reference in its entirety. Phosphotriester oligonucleotides may be prepared as described in U.S. Pat. No. 5,023,243, which disclosure is hereby incorporated by reference in its entirety. Borano phosphate oligonucleotides may be prepared as described in U.S. Pat. Nos. 5,130,302 and 5,177,198 which disclosures are hereby incorporated by reference in their entireties.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another by virtue of their sequence identities in a manner like that found in double-helical DNA with thymine or uracil residues linked to adenine residues by two hydrogen bonds and cytosine and guanine residues linked by three hydrogen bonds (see Stryer, 1995, which disclosure is hereby incorporated by reference in its entirety).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance Creighton (1993); Seifter et al., (1990); Rattan et al., (1992)). Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

As used herein, the term “non-human animal” refers to any non-human animal, including insects, birds, rodents and more usually mammals. Preferred non-human animals include: primates; farm animals such as swine, goats, sheep, donkeys, cattle, horses, chickens, rabbits; and rodents, preferably rats or mice. As used herein, the term “animal” is used to refer to any species in the animal kingdom, preferably vertebrates, including birds and fish, and more preferable a mammal. Both the terms “animal” and “mammal” expressly embrace human subjects unless preceded with the term “non-human”.

The term “domain” refers to an amino acid fragment with specific biological properties. This term encompasses all known structural and linear biological motifs. Examples of such motifs include but are not limited to leucine zippers, helix-turn-helix motifs, glycosylation sites, ubiquitination sites, alpha helices, and beta sheets, signal peptides which direct the secretion of proteins, sites for post-translational modification, enzymatic active sites, substrate binding sites, and enzymatic cleavage sites.

Although each of these terms has a distinct meaning, the terms “comprising”, “consisting of” and “consisting essentially of” may be interchanged for one another throughout the instant application. The term “having” has the same meaning as “comprising” and may be replaced with either the term “consisting of” or “consisting essentially of”.

Unless otherwise specified in the application, nucleotides and amino acids of polynucleotides and polypeptides, respectively, of the present invention are contiguous and not interrupted by heterologous sequences.

Identity Between Nucleic Acids Or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, CLUSTALW, FASTDB (Pearson and Lipman, 1988; Altschul et al., 1990; Thompson et al., 1994; Higgins et al., 1996; Altschul et al., 1990; Altschul et al., 1993; Brutlag et al, 1990), the disclosures of which are incorporated by reference in their entireties.

In a particularly preferred embodiment, protein and nucleic acid sequence homologies are evaluated using the Basic Local Alignment Search Tool (“BLAST”) which is well known in the art (see, e.g., Karlin and Altschul, 1990; Altschul et al., 1990, 1993, 1997), the disclosures of which are incorporated by reference in their entireties. In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992; Henikoff and Henikoff, 1993, the disclosures of which are incorporated by reference in their entireties). Less preferably, the PAM or PAM250 matrices may also be used (see, e.g., Schwartz and Dayhoff, eds., 1978, the disclosure of which is incorporated by reference in its entirety). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990), the disclosure of which is incorporated by reference in its entirety. The BLAST programs may be used with the default parameters or with modified parameters provided by the user.

Another preferred method for determining the best overall match between a query nucleotide sequence (a sequence of the present invention) and a subject sequence, also referred to as a global sequence alignment, can be determined using the FASTDB computer program based on the algorithm of Brutlag et al. (1990), the disclosure of which is incorporated by reference in its entirety. In a sequence alignment the query and subject sequences are both DNA sequences. An RNA sequence can be compared by first converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using 10, the specified parameters, to arrive at a final percent identity score. This corrected score is what is used for the purposes of the present invention. Only nucleotides outside the 5′ and 3′ nucleotides of the subject sequence, as displayed by the FASTDB alignment, which are not matched/aligned with the query sequence, are calculated for the purposes of manually adjusting the percent identity score. For example, a 90 nucleotide subject sequence is aligned to a 100 nucleotide query sequence to determine percent identity. The deletions occur at the 5′ end of the subject sequence and therefore, the FASTDB alignment does not show a matched/alignment of the first 10 nucleotides at 5′ end. The 10 unpaired nucleotides represent 10% of the sequence (number of nucleotides at the 5′ and 3′ ends not matched/total number of nucleotides in the query sequence) so 10% is subtracted from the percent identity score calculated by the FASTDB program. If the remaining 90 nucleotides were perfectly matched the final percent identity would be 90%. In another example, a 90 nucleotide subject sequence is compared with a 100 nucleotide query sequence. This time the deletions are internal deletions so that there are no nucleotides on the 5′ or 3′ of the subject sequence which are not matched/aligned with the query. In this case the percent identity calculated by FASTDB is not manually corrected. Once again, only nucleotides 5′ and 3′ of the subject sequence which are not matched/aligned with the query sequence are manually corrected. No other manual corrections are made for the purposes of the present invention.

Another preferred method for determining the best overall match between a query amino acid sequence (a sequence of the present invention) and a subject sequence, also referred to as a global sequence alignment, can be determined using the FASTDB computer program based on the algorithm of Brutlag et al. (1990). In a sequence alignment the query and subject sequences are both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. If the subject sequence is shorter than the query sequence due to N-or C-terminal deletions, not because of internal deletions, the results, in percent identity, must be manually corrected. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query amino acid residues outside the farthest N- and C-terminal residues of the subject sequence. For example, a 90 amino acid residue subject sequence is aligned with a 100-residue query sequence to determine percent identity. The deletion occurs at the N-terminus of the subject sequence and therefore, the FASTDB alignment does not match/align with the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90-residue subject sequence is compared with a 100-residue query sequence. This time the deletions are internal so there are no residues at the N- or C-termini of the subject sequence, which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other manual corrections are made for the purposes of the present invention.

The term “percentage of sequence similarity” refers to comparisons between polypeptide sequences and is determined by comparing two optimally aligned sequences over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which an identical or equivalent amino acid residue occurs in both sequences to yield the number of matched positions, dividing the number of matched positions by the total number of positions in the window of comparison and multiplying the result by 100 to yield the percentage of sequence similarity. Similarity is evaluated using any of the variety of sequence comparison algorithms and programs known in the art, including those described above in this section. Equivalent amino acid residues are defined herein in the “Mutated polypeptides” section.

### Polynucleotides of the Invention

The present invention concerns GENSET genomic and cDNA sequences. The present invention encompasses GENSET genes, polynucleotides comprising GENSET genomic and cDNA sequences, as well as fragments and variants thereof. These polynucleotides may be purified, isolated, or recombinant.

Also encompassed by the present invention are allelic variants, orthologs, splice variants, and/or species homologues of the GENSET genes. Procedures known in the art can be used to obtain full-length genes and cDNAs, allelic variants, splice variants, full-length coding portions, orthologs, and/or species homologues of genes and cDNAs corresponding to a nucleotide sequence selected from the group consisting of sequences of SEQ ID NOs:1-169, 339-455, 561-784 and sequences of clone inserts of the deposited clone pool, using information from the sequences disclosed herein or the clone pool deposited with the ATCC or other depositary authority. For example, allelic variants, orthologs and/or species homologues may be isolated and identified by making suitable probes or primers from the sequences provided herein and screening a suitable nucleic acid source for allelic variants and/or the desired homologue using any technique known to those skilled in the art including those described into the section entitled “To find similar sequences”.

In a specific embodiment, the polynucleotides of the invention are at least 15, 30, 50, 100, 125, 500, or 1000 continuous nucleotides. In another embodiment, the polynucleotides are less than or equal to 300 kb, 200 kb, 100 kb, 50 kb, 10 kb, 7.5 kb, 5 kb, 2.5 kb, 2 kb, 1.5 kb, or 1 kb in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 75, 50, 25, 20, 15, 10, 5, 4, 3, 2, or 1 naturally occurring genomic flanking gene(s).

Deposited Clone Pool of the Invention

Expression of GENSET genes has been shown to lead to the production of at least one mRNA species per GENSET gene, which cDNA sequence is set forth in the appended sequence listing as SEQ ID NOs:1-169, 339-455, 561-784. The cDNAs (SEQ ID NOs:1-169, 339-455, 561-784) corresponding to these GENSET mRNA species were cloned either in the vector pBluescriptII SK⁻ (Stratagene) or in a vector called pPT. Cells containing the cloned cDNAs of the present invention are maintained in permanent deposit by the inventors at Genset, S.A., 24 Rue Royale, 75008 Paris, France. Each cDNA can be removed from the Bluescript vector in which it was inserted by performing a NotI Pst I double digestion, or from the pPT vector by performing a MunI HindIII double digestion, to produce the appropriate fragment for each clone, provided the cDNA sequence does not contain any of the corresponding restriction sites within its sequence. Alternatively, other restriction enzymes of the multicloning site of the vector may be used to recover the desired insert as indicated by the manufacturer.

Pools of cells containing certain cDNAs of the invention, from which the cells containing a particular polynucleotide is obtainable, have also been deposited with the American Tissue Culture Collection (ATCC), 10801 University Boulevard, Manassas, Va. 20110-2209, United States. These cDNA clones have been transfected into separate bacterial cells (E-coli) for these composite deposits.

Bacterial cells containing a particular clone can be obtained from the composite deposit as follows:

An oligonucleotide probe or probes should be designed to the sequence that is known for that particular clone. This sequence can be derived from the sequences provided herein, or from a combination of those sequences. The design of the oligonucleotide probe should preferably follow these parameters:

(a) It should be designed to an area of the sequence which has the fewest ambiguous bases (“N's”), if any;

(b) Preferably, the probe is designed to have a Tm of approximately 80 degrees Celsius (assuming 2 degrees for each A or T and 4 degrees for each G or C). However, probes having melting temperatures between 40 degrees Celsius and 80 degrees Celsius may also be used provided that specificity is not lost.

The oligonucleotide should preferably be labeled with gamma[³²P]ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantified by measurement in a scintillation counter. Preferably, specific activity of the resulting probe should be approximately 4×10⁶ dpm/pmole.

The bacterial culture containing the pool of full-length clones should preferably be thawed and 100 ul of the stock used to inoculate a sterile culture flask containing 25 ml of sterile L-broth containing ampicillin at 100 ug/ml. The culture should preferably be grown to saturation at 37 degrees Celsius, and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 ug/ml and agar at 1.5% in a 150 mm petri dish when grown overnight at 37 degrees Celsius. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65 degrees Celsius for 1 hour with gentle agitation in 6×SSC (20× stock is 175.3 g NaCl/liter, 88.2 g Na citrate/liter, adjusted to pH 7.0 with NaOH) containing 0.5% SDS, 100 pg/ml of yeast RNA, and 10 mM EDTA (approximately 10 ml per 150 mm filter). Preferably, the probe is then added to the hybridization mix at a concentration greater than or equal to 1×10⁶ dpm/ml. The filter is then preferably incubated at 65 degrees Celsius with gentle agitation overnight. The filter is then preferably washed in 500 ml of 2×SSC/0.1% SDS at room temperature with gentle shaking for 15 minutes. A third wash with 0.1×SSC/0.5% SDS at 65 degrees Celsius for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing. The plasmid DNA obtained using these procedures may then be manipulated using standard cloning techniques familiar to those skilled in the art.

Alternatively, to recover cDNA inserts from the pool of bacteria, a PCR can be performed on plasmid DNA isolated using standard procedures and primers designed at both ends of the cDNA insertion, including primers designed in the multicloning site of the vector. If a specific cDNA of interest is to be recovered, primers may be designed in order to be specific for the 5′ end and the 3′ end of this cDNA using sequence information available from the appended sequence listing. The PCR product which corresponds to the cDNA of interest can then be manipulated using standard cloning techniques familiar to those skilled in the art.

Therefore, an object of the invention is an isolated, purified, or recombinant polynucleotide comprising a nucleotide sequence selected from the group consisting of cDNA inserts of the deposited clone pool. Moreover, preferred polynucleotides of the invention include purified, isolated, or recombinant GENSET cDNAs consisting of, consisting essentially of, or comprising a nucleotide sequence selected from the group consisting of cDNA inserts of the deposited clone pool.

cDNA Sequences of the Invention

Another object of the invention is a purified, isolated, or recombinant polynucleotide comprising a nucleotide sequence selected from the group consisting of sequences of SEQ ID NOs:1-169, 339-455, 561-784, complementary sequences thereto, and fragments thereof. Moreover, preferred polynucleotides of the invention include purified, isolated, or recombinant GENSET cDNAs consisting of, consisting essentially of, or comprising a sequence selected from the group consisting of SEQ ID NOs:1-169, 339-455, 561-784.

Accordingly, the coding sequence (CDS) or open reading frame (ORF) of each cDNA of the invention refers to the nucleotide sequence beginning with the first nucleotide of the start codon and ending with the last nucleotide of the stop codon. Similarly, the 5′ untranslated region (or 5′UTR) of each cDNA of the invention refers to the nucleotide sequence starting at nucleotide 1 and ending at the nucleotide immediately 5′ to the first nucleotide of the start codon. The 3′ untranslated region (or 3′UTR) of each cDNA of the invention refers to the nucleotide sequence starting at the nucleotide immediately 3′ to the last nucleotide of the stop codon and ending at the last nucleotide of the cDNA.

Untranslated Regions

In addition, the invention concerns a purified, isolated, and recombinant nucleic acid comprising a nucleotide sequence selected from the group consisting of the 5′UTRs of sequences of SEQ ID NOs:1-169, 339-455, 561-784 and sequences of clone inserts of the deposited clone pool, sequences complementary thereto, and allelic variants thereof. The invention also concerns a purified, isolated, and/or recombinant nucleic acid comprising a nucleotide sequence selected from the group consisting of the 3′UTRs of sequences of SEQ ID NOs:1-169, 339-455, 561-784 and sequences of clone inserts of the deposited clone pool, sequences complementary thereto, and allelic variants thereof.

These polynucleotides may be used to detect the presence of GENSET mRNA species in a biological sample using either hybridization or RT-PCR techniques well known to those skilled in the art.

In addition, these polynucleotides may be used as regulatory molecules able to affect the processing and maturation of any polynucleotide including them (either a GENSET polynucleotide or an heterologous polynucleotide), preferably the localization, stability and/or translation of said polynucleotide including them (for a review on UTRs see Decker and Parker, 1995, Derrigo et al., 2000). In particular, 3′UTRs may be used in order to control the stability of heterologous mRNAs in recombinant vectors using any methods known to those skilled in the art including Makrides ((1999) Protein Expr Purif November 1999; 17(2):183-202), U.S. Pat. Nos. 5,925,564; 5,807,707 and 5,756,264, which disclosures are hereby incorporated by reference in their entireties.

Coding Sequences

Another object of the invention is an isolated, purified or recombinant polynucleotide comprising the coding sequence of a sequence selected from the group consisting of sequences of SEQ ID NOs:1-169, 339-455, 561-784, clone inserts of the deposited clone pool, and variants thereof.

A further object of the invention is an isolated, purified or recombinant polynucleotide encoding a polypeptide comprising a sequence selected from the group consisting of sequences of SEQ ID NOs: 170-338, 456-560, 785-918 and allelic variants thereof. Another object of the invention is an isolated, purified or recombinant polynucleotide encoding a polypeptide comprising a sequence selected from the group consisting of polypeptides encoded by cDNA inserts of the deposited clone pool and allelic variants thereof.

It will be appreciated that should the extent of the coding sequence differ from that indicated in the appended sequence listing as a result of a sequencing error, reverse transcription or amplification error, mRNA splicing, post-translational modification of the encoded protein, enzymatic cleavage of the encoded protein, or other biological factors, one skilled in the art would be readily able to identify the extent of the coding sequences in the sequences of SEQ ID NOs:1-169, 339-455, 561-784. Accordingly, the scope of any claims herein relating to nucleic acids containing the coding sequence of one of SEQ ID NOs:1-169, 339-455, 561-784 is not to be construed as excluding any readily identifiable variations from or equivalents to the coding sequences described in the appended sequence listing. Equivalents includes any alterations in a nucleotide coding sequence that does not result in an amino acid change, or that results in a conservative amino acid substitution, as defined below, in the polypeptide encoded by the nucleotide sequence. Similarly, should the extent of the polypeptides differ from those indicated in the appended sequence listing as a result of any of the preceding factors, the scope of claims relating to polypeptides comprising the amino acid sequence of the polypeptides of SEQ ID NOs:170-338, 456-560, 785-918 is not to be construed as excluding any readily identifiable variations from or equivalents to the sequences described in the appended sequence listing.

The above-disclosed polynucleotides that contain the coding sequence of the GENSET genes may be expressed in a desired host cell or a desired host organism, when this polynucleotide is placed under the control of suitable expression signals. The expression signals may be either the expression signals contained in the regulatory regions in the GENSET genes of the invention or, in contrast, the signals may be exogenous regulatory nucleic sequences. Such a polynucleotide, when placed under the suitable expression signals, may also be inserted in a vector for its expression and/or amplification.

Further included in the present invention are polynucleotides encoding the polypeptides of the present invention that are fused in frame to the coding sequences for additional heterologous amino acid sequences. Also included in the present invention are nucleic acids encoding polypeptides of the present invention together with additional, non-coding sequences, including, but not limited to, non-coding 5′ and 3′ sequences, vector sequence, sequences used for purification, probing, or priming. For example, heterologous sequences include transcribed, untranslated sequences that may play a role in transcription and mRNA processing, such as ribosome binding and stability of mRNA. The heterologous sequences may alternatively comprise additional coding sequences that provide additional functionalities. Thus, a nucleotide sequence encoding a polypeptide may be fused to a tag sequence, such as a sequence encoding a peptide that facilitates purification or detection of the fused polypeptide. In certain preferred embodiments of this aspect of the invention, the tag amino acid sequence is a hexa-histidine peptide, such as the tag provided in a pQE vector (QIAGEN), or in any of a number of additional, commercially available vectors. For instance, hexa-histidine provides for the convenient purification of the fusion protein (see, Gentz et al., 1989, Proc Natl Acad Sci USA Feb;86(3):821-4, the disclosure of which is incorporated by reference in its entirety). The “HA” tag is another peptide useful for purification which corresponds to an epitope derived from the influenza hemagglutinin protein (see, Wilson et al., 1984, Cell Jul;37(3):767-78, the disclosure of which is incorporated by reference in its entirety). As discussed below, other such fusion proteins include a GENSET polypeptide fused to Fc at the N- or C-terminus.

Suitable recombinant vectors that contain a polynucleotide such as described herein are disclosed elsewhere in the specification. Expression vectors encoding GENSET polypeptides or fragments thereof are described in the section entitled “Preparation of the polypeptides”.

Regulatory Sequences of the Invention

As mentioned, the genomic sequence of GENSET genes contain regulatory sequences in the non-coding 5′-flanking region and possibly in the non-coding 3′-flanking region that border the GENSET polypeptide coding regions containing the exons of these genes.

Polynucleotides derived from GENSET polynucleotide 5′ and 3′ regulatory regions are useful in order to detect the presence of at least a copy of a genomic nucleotide sequence of the GENSET gene or a fragment thereof in a test sample.

Preferred Regulatory Sequences

Polynucleotides carrying the regulatory elements located at the 5′ end and at the 3′ end of GENSET polypeptide coding regions may be advantageously used to control, e.g., the transcriptional and translational activity of a heterologous polynucleotide of interest.

Thus, the present invention also concerns a purified or isolated nucleic acid comprising a polynucleotide which is selected from the group consisting of the 5′ and 3′ GENSET polynucleotide regulatory regions, sequences complementary thereto, regulatory active fragments and variants thereof. The invention also pertains to a purified or isolated nucleic acid comprising a polynucleotide having at least 95% nucleotide identity with a polynucleotide selected from the group consisting of GENSET polynucleotide 5′ and 3′ regulatory regions, advantageously 99% nucleotide identity, preferably 99.5% nucleotide identity and most preferably 99.8% nucleotide identity with a polynucleotide selected from the group consisting of GENSET polynucleotide 5′ and 3′ regulatory regions, sequences complementary thereto, variants and regulatory active fragments thereof.

Another object of the invention consists of purified, isolated or recombinant nucleic acids comprising a polynucleotide that hybridizes, under the stringent hybridization conditions defined herein, with a polynucleotide selected from the group consisting of the nucleotide sequences of GENSET polynucleotide 5′ and 3′ regulatory regions, sequences complementary thereto, variants and regulatory active fragments thereof.

Preferred fragments of 5′ regulatory regions have a length of about 1500 or 1000 nucleotides, preferably of about 500 nucleotides, more preferably about 400 nucleotides, even more preferably 300 nucleotides and most preferably about 200 nucleotides.

Preferred fragments of 3′ regulatory regions are at least 20, 50, 100, 150, 200, 300 or 400 bases in length. “Regulatory active” polynucleotide derivatives of the 5′ or 3′ regulatory region are polynucleotides comprising or alternatively consisting of a fragment of said polynucleotide which is functional as a regulatory region for expressing a recombinant polypeptide or a recombinant polynucleotide in a recombinant cell host. It could act either as an enhancer or as a repressor. For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide.

The regulatory polynucleotides of the invention may be prepared from the nucleotide sequence of GENSET genomic or cDNA sequence, for example, by cleavage using suitable restriction enzymes, or by PCR. The regulatory polynucleotides may also be prepared by digestion of a GENSET gene-containing genomic clone by an exonuclease enzyme, such as Bal31 (Wabiko et al., DNA 5(4):305-14 (1986), the disclosure of which is incorporated by reference in its entirety). These regulatory polynucleotides can also be prepared by nucleic acid chemical synthesis, as described elsewhere in the specification.

The regulatory polynucleotides according to the invention may be part of a recombinant expression vector that may be used to express a coding sequence in a desired host cell or host organism. The recombinant expression vectors according to the invention are described elsewhere in the specification.

Preferred 5′-regulatory polynucleotides of the invention include 5′-UTRs of GENSET cDNAs, or regulatory active fragments or variants thereof. More preferred 5′-regulatory polynucleotides of the invention include sequences selected from the group consisting of 5′-UTRs of sequences of SEQ ID NOs:1-169, 339-455, 561-784, 5′-UTRs of clone inserts of the deposited clone pool, regulatory active fragments and variants thereof.

Preferred 3′-regulatory polynucleotide of the invention include 3′-UTRs of GENSET cDNAs, or regulatory active fragments or variants thereof. More preferred 3′-regulatory polynucleotides of the invention include sequences selected from the group consisting of 3′-UTRs of sequences of SEQ ID NOs:1-169, 339-455, 561-784, 3′-UTRs of clone inserts of the deposited clone pool, regulatory active fragments and variants thereof.

A further object of the invention consists of a purified or isolated nucleic acid comprising:

a) a polynucleotide comprising a 5′ regulatory nucleotide sequence selected from the group consisting of:

(i) a nucleotide sequence comprising a polynucleotide of a GENSET polynucleotide 5′ regulatory region or a complementary sequence thereto;

(ii) a nucleotide sequence comprising a polynucleotide having at least 95% of nucleotide identity with the nucleotide sequence of a GENSET polynucleotide 5′ regulatory region or a complementary sequence thereto;

(iii) a nucleotide sequence comprising a polynucleotide that hybridizes under stringent hybridization conditions with the nucleotide sequence of a GENSET polynucleotide 5′ regulatory region or a complementary sequence thereto; and

(iv) a regulatory active fragment or variant of the polynucleotides in (i), (ii) and (iii);

b) a nucleic acid molecule encoding a desired polypeptide or a nucleic acid molecule of interest, wherein said nucleic acid molecule is operably linked to the polynucleotide defined in (a); and

c) optionally, a polynucleotide comprising a 3′-regulatory polynucleotide, preferably a 3′-regulatory polynucleotide of a GENSET gene.

In a specific embodiment, the nucleic acid defined above includes the 5′-UTR of a GENSET cDNA, or a regulatory active fragment or variant thereof.

In a second specific embodiment, the nucleic acid defined above includes the 3′-UTR of a GENSET cDNA, or a regulatory active fragment or variant thereof.

The regulatory polynucleotide of the 5′ regulatory region, or its regulatory active fragments or variants, is operably linked at the 5′-end of the nucleic acid molecule encoding the desired polypeptide or nucleic acid molecule of interest.

The regulatory polynucleotide of the 3′ regulatory region, or its regulatory active fragments or variants, is advantageously operably linked at the 3′-end of the nucleic acid molecule encoding the desired polypeptide or nucleic acid molecule of interest.

The desired polypeptide encoded by the above-described nucleic acid may be of various nature or origin, encompassing proteins of prokaryotic viral or eukaryotic origin. Among the polypeptides expressed under the control of a GENSET polynucleotide regulatory region include bacterial, fungal or viral antigens. Also encompassed are eukaryotic proteins such as intracellular proteins, such as “house-keeping” proteins, membrane-bound proteins, such as mitochondrial membrane-bound proteins and cell surface receptors, and secreted proteins such as endogenous mediators such as cytokines. The desired polypeptide may be an heterologous polypeptide or a GENSET polypeptide, especially a protein with an amino acid sequence selected from the group consisting of sequences of SEQ ID NOs:170-338, 456-560, 785-918, fragments and variants thereof.

The desired nucleic acids encoded by the above-described polynucleotides, usually an RNA molecule, may be complementary to a desired coding polynucleotide, for example to a GENSET coding sequence, and thus useful as an antisense polynucleotide. Such a polynucleotide may be included in a recombinant expression vector in order to express the desired polypeptide or the desired nucleic acid in host cell or in a host organism. Suitable recombinant vectors that contain a polynucleotide such as described herein are disclosed elsewhere in the specification.

Polynucleotide Variants

The invention also relates to variants of the polynucleotides described herein and fragments thereof. “Variants” of polynucleotides, as the term is used herein, are polynucleotides that differ from a reference polynucleotide. Generally, differences are limited so that the nucleotide sequences of the reference and the variant are closely similar overall and, in many regions, identical. The present invention encompasses both allelic variants and degenerate variants.

Examples of variant sequences of polynucleotides of the invention are given in the appended sequence listing. Specifically, Table I includes sequences for which a plurality of closely related sequences, e.g. variants, are provided.

Allelic Variants

A variant of a polynucleotide may be a naturally occurring variant such as a naturally occurring allelic variant, or it may be a variant that is not known to occur naturally. By an “allelic variant” is intended one of several alternate forms of a gene occupying a given locus on a chromosome of an organism (see Lewin, 1990), the disclosure of which is incorporated by reference in its entirety. Diploid organisms may be homozygous or heterozygous for an allelic form. Non-naturally occurring variants of the polynucleotide may be made by art-known mutagenesis techniques, including those applied to polynucleotides, cells or organisms. See, for example, Table I, which includes sequences for which a plurality of closely related sequences, e.g. allelic variants of a single gene, are provided.

Degenerate Variant

In addition to the isolated polynucleotides of the present invention, and fragments thereof, the invention further includes polynucleotides which comprise a sequence substantially different from those described above but which, due to the degeneracy of the genetic code, still encode a GENSET polypeptide of the present invention. These polynucleotide variants are referred to as “degenerate variants” throughout the instant application. That is, all possible polynucleotide sequences that encode the GENSET polypeptides of the present invention are contemplated. This includes the genetic code and species-specific codon preferences known in the art. Thus, it would be routine for one skilled in the art to generate the degenerate variants described above, for instance, to optimize codon expression for a particular host (e.g., change codons in the human mRNA to those preferred by other mammalian or bacterial host cells).

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions. In the context of the present invention, preferred embodiments are those in which the polynucleotide variants encode polypeptides which retain substantially the same biological properties or activities as the GENSET protein. More preferred polynucleotide variants are those containing conservative substitutions.

Similar Polynucleotides

Other embodiments of the present invention provide a purified, isolated or recombinant polynucleotide which is at least 80%, 85%, 90%, 95%, 96%, 97%, 98% or 99% identical to a polynucleotide selected from the group consisting of sequences of SEQ ID NOs:1-169, 339-455, 561-784 and the clone inserts of the deposited clone pool. The above polynucleotides are included regardless of whether they encode a polypeptide having a GENSET biological activity. This is because even where a particular nucleic acid molecule does not encode a polypeptide having activity, one of skill in the art would still know how to use the nucleic acid molecule, for instance, as a hybridization probe or primer. Uses of the nucleic acid molecules of the present invention that do not encode a polypeptide having GENSET activity include, inter alia, isolating a GENSET gene or allelic variants thereof from a DNA library, and detecting GENSET mRNA expression in biological samples suspected of containing GENSET mRNA or DNA, e.g., by Northern Blot or PCR analysis.

The present invention is further directed to polynucleotides having sequences at least 50%. 60%, 70%, 80%, 90%, 95%, 96%, 97%, 98% or 99% identity to a polynucleotide selected from the group consisting of sequences of SEQ ID NOs:1-169, 339-455, 561-784 and clone inserts of the deposited clone pool, where said polynucleotide do, in fact, encode a polypeptide having a GENSET biological activity. Of course, due to the degeneracy of the genetic code, one of ordinary skill in the art will immediately recognize that a large number of the polynucleotides at least 50%. 60%, 70%, 80%, 90%, 95%, 96%, 97%, 98%, or 99% identical to a polynucleotide selected from the group consisting of sequences of SEQ ID NOs:1-169, 339-455, 561-784 and clone inserts of the deposited clone pool will encode a polypeptide having biological activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having biological activity. This is because the skilled artisan is fully aware of amino acid substitutions that are either less likely or not likely to significantly affect protein function (e.g., replacing one aliphatic amino acid with a second aliphatic amino acid), as further described below. By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the GENSET polypeptide. In other words, to obtain a polynucleotide having a nucleotide sequence at least 95% identical to a reference nucleotide sequence, up to 5% of the nucleotides in the reference sequence may be deleted, inserted, or substituted with another nucleotide. The query sequence may be an entire sequence selected from the group consisting of sequences of SEQ ID NOs:1-169, 339-455, 561-784 and sequences of clone inserts of the deposited clone pool, or the ORF (open reading frame) of a polynucleotide sequence selected from said group, or any fragment specified as described herein.

Hybridizing Polynucleotides

In another aspect, the invention provides an isolated or purified nucleic acid molecule comprising a polynucleotide which hybridizes under stringent hybridization conditions to any polynucleotide of the present invention using any methods known to those skilled in the art including those disclosed herein and in particular in the “To find similar sequences” section. Also contemplated are nucleic acid molecules that hybridize to the polynucleotides of the present invention at lower stringency hybridization conditions, preferably at moderate or low stringency conditions as defined herein. Such hybridizing polynucleotides may be of at least 15, 18, 20, 23, 25, 28, 30, 35, 40, 50, 75, 100, 200, 300, 500 or 1000 nucleotides in length.

Of particular interest are polynucleotides hybridizing to any polynucleotide of the invention and encoding GENSET polypeptides, particularly GENSET polypeptides exhibiting a GENSET biological activity.

Of course, a polynucleotide which hybridizes only to polyA+ sequences (such as any 3′ terminal polyA+ tract of a cDNA shown in the sequence listing), or to a 5′ complementary stretch of T (or U) residues, would not be included in the definition of “polynucleotide,” since such a polynucleotide would hybridize to any nucleic acid molecule containing a poly(A) stretch or the complement thereof (e.g., practically any double-stranded cDNA clone generated using oligo dT as a primer).

Complementary Polynucleotides

The invention further provides isolated nucleic acid molecules having a nucleotide sequence fully complementary to any polynucleotide of the invention. The present invention encompasses a purified, isolated or recombinant polynucleotide having a nucleotide sequence complementary to a sequence selected from the group consisting of sequences of SEQ ID NOs:1-169, 339-455, 561-784, sequences of clone inserts of the deposited clone pool and fragments thereof. Such isolated molecules, particularly DNA molecules, are useful as probes for gene mapping and for identifying GENSET mRNA in a biological sample, for instance, by PCR or Northern blot analysis.

Polynucleotide Fragments

The present invention is further directed to polynucleotides encoding portions or fragments of the nucleotide -sequences described herein. Uses for the polynucleotide fragments of the present invention include probes, primers, molecular weight markers and for expressing the polypeptide fragments of the present invention. Fragments include portions of polynucleotides selected from the group consisting of a) the sequences of SEQ ID NOs:1-169, 339-455, 561-784, b) genomic GENSET sequences, c) the polynucleotides encoding a polypeptide selected from the group consisting of the sequences of SEQ ID NOs:170-338, 456-560, 785-918, d) the sequences of clone inserts of the deposited clone pool, and e) the polynucleotides encoding the polypeptides encoded by the clone inserts of the deposited clone pool. Particularly included in the present invention is a purified or isolated polynucleotide comprising at least 8 consecutive bases of a polynucleotide of the present invention. In one aspect of this embodiment, the polynucleotide comprises at least 10, 12, 15, 18, 20, 25, 28, 30, 35, 40, 50, 75, 100, 150, 200, 300, 400, 500, 800, 1000, 1500, or 2000 consecutive nucleotides of a polynucleotide of the present invention.

In addition to the above preferred polynucleotide sizes, further preferred sub-genuses of polynucleotides comprise at least 8 nucleotides, wherein “at least 8” is defined as any integer between 8 and the integer representing the 3′ most nucleotide position as set forth in the sequence listing or elsewhere herein. Further included as preferred polynucleotides of the present invention are polynucleotide fragments at least 8 nucleotides in length, as described above, that are further specified in terms of their 5′ and 3′ position. The 5′ and 3′ positions are represented by the position numbers set forth in the appended sequence listing. For allelic, degenerate and other variants, position 1 is defined as the 5′ most nucleotide of the ORF, i.e., the nucleotide “A” of the start codon with the remaining nucleotides numbered consecutively. Therefore, every combination of a 5′ and 3′ nucleotide position that a polynucleotide fragment of the present invention, at least 8 contiguous nucleotides in length, could occupy on a polynucleotide of the invention is included in the invention as an individual species. The polynucleotide fragments specified by 5′ and 3′ positions can be immediately envisaged and are therefore not individually listed solely for the purpose of not unnecessarily lengthening the specification.

It is noted that the above species of polynucleotide fragments of the present invention may alternatively be described by the formula “a to b”; where “a” equals the 5′ most nucleotide position and “b” equals the 3′ most nucleotide position of the polynucleotide; and further where “a” equals an integer between 1 and the number of nucleotides of the polynucleotide sequence of the present invention minus 8, and where “b” equals an integer between 9 and the number of nucleotides of the polynucleotide sequence of the present invention; and where “a” is an integer smaller then “b” by at least 8.

Therefore, the present invention encompasses isolated, purified, or recombinant polynucleotides which consist of, consist essentially of, or comprise a contiguous span of at least 8, 10, 12, 15, 18, 20, 25, 28, 30, 35, 40, 50, 75, 100, 150, 200, 300, 400, 500, 1000 or 2000 nucleotides of a sequence selected from the group consisting of the sequences of SEQ ID NOs:1-169, 339-455, 561-784 and sequences fully complementary thereto.

Other preferred fragments of the invention are polynucleotides comprising polynucleotides encoding domains of polypeptides. Such fragments may be used to obtain other polynucleotides encoding polypeptides having similar domains using hybridization or RT-PCR techniques. Alternatively, these fragments may be used to express a polypeptide domain which may have a specific biological property. Thus, another object of the invention is an isolated, purified or recombinant polynucleotide encoding a polypeptide consisting of, consisting essentially of, or comprising a contiguous span of at least 5, 6, 8, 10, 12, 15, 20, 25, 30, 35, 40, 50, 60, 75, 100, 150 or 200 consecutive amino acids of a sequence selected from the group consisting of the sequences of SEQ ID NOs: 170-338, 456-560, 785-918, to the extent that a contiguous span of these lengths is consistent with the lengths of said selected sequence, where said contiguous span comprises at least 1, 2, 3, 5, or 10 of the amino acid positions of a domain of said selected sequence. The present invention also encompasses isolated, purified or recombinant polynucleotides encoding a polypeptide comprising a contiguous span of at least 5, 6, 8, 10, 12, 15, 20, 25, 30, 35, 40, 50, 60, 75, 100, 150 or 200 consecutive amino acids of a sequence selected from the group consisting of sequences of SEQ ID NOs: 170-338, 456-560, 785-918, to the extent that a contiguous span of these lengths is consistent with the lengths of said selected sequence, where said contiguous span is a domain of said selected sequence. The present invention also encompasses isolated, purified or recombinant polynucleotides encoding a polypeptide comprising a domain of a sequence selected from the group consisting of the sequences of SEQ ID NOs:170-338, 456-560, 785-918.

The present invention further encompasses any combination of the polynucleotide fragments listed in this section.

Oligonucleotide Primers and Probes

The present invention also encompasses fragments of GENSET polynucleotides for use as primers and probes. Polynucleotides derived from the GENSET genomic and cDNA sequences are useful in order to detect the presence of at least a copy of a GENSET polynucleotide or fragment, complement, or variant thereof in a test sample.

Structural Definition

Any polynucleotide of the invention may be used as a primer or probe. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of a sequence selected from the group consisting of the GENSET genomic sequences, the cDNA sequences and the sequences fully complementary thereto. Another object of the invention is a purified, isolated, or recombinant polynucleotide comprising the nucleotide sequence of a sequence selected from the group consisting of the sequences of SEQ ID NOs:1-169, 339-455, 561-784, sequences of clone inserts of the deposited clone pool, sequences fully complementary thereto, allelic variants thereof, and fragments thereof. Moreover, preferred probes and primers of the invention include purified, isolated, or recombinant GENSET cDNAs consisting of, consisting essentially of, or comprising the sequences of SEQ ID NOs:1-169, 339-455, 561-784 and sequences of clone inserts of the deposited clone pool. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of a sequence selected from the group consisting of the sequences of SEQ ID NOs:1-169, 339-455, 561-784 and the sequences fully complementary thereto.

Design of Primers and Probes

A probe or a primer according to the invention has between 8 and 1000 nucleotides in length, or is specified to be at least 12, 15, 18, 20, 25, 35, 40, 50, 60, 70, 80, 100, 250, 500 or 1000 nucleotides in length. More particularly, the length of these probes and primers can range from 8, 10, 15, 20, or 30 to 100 nucleotides, preferably from 10 to 50, more preferably from 15 to 30 nucleotides. Shorter probes and primers tend to lack specificity for a target nucleic acid sequence and generally require cooler temperatures to form sufficiently stable hybrid complexes with the template. Longer probes and primers are expensive to produce and can sometimes self-hybridize to form hairpin structures. The appropriate length for primers and probes under a particular set of assay conditions may be empirically determined by one of skill in the art. The formation of stable hybrids depends on the melting temperature (Tm) of the DNA. The Tm depends on the length of the primer or probe, the ionic strength of the solution and the G+C content. The higher the G+C content of the primer or probe, the higher is the melting temperature because G:C pairs are held by three H bonds whereas A:T pairs have only two. The GC content in the probes of the invention usually ranges between 10 and 75%, preferably between 35 and 60%, and more preferably between 40 and 55%.

For amplification purposes, pairs of primers with approximately the same Tm are preferable. Primers may be designed using the OSP software (Hillier and Green, 1991), the disclosure of which is incorporated by reference in its entirety, based on GC content and melting temperatures of oligonucleotides, or using PC-Rare (http:// bioinformatics.weizmann.ac.il/software/PC-Rare/doc/manuel.html) based on the octamer frequency disparity method (Griffais et al., 1991), the disclosure of which is incorporated by reference in its entirety. DNA amplification techniques are well known to those skilled in the art. Amplification techniques that can be used in the context of the present invention include, but are not limited to, the ligase chain reaction (LCR) described in EP-A-320 308, WO 9320227 and EP-A-439 182, the polymerase chain reaction (PCR, RT-PCR) and techniques such as the nucleic acid sequence based amplification (NASBA) described in Guatelli et al.(1990) and in Compton (1991), Q-beta amplification as described in European Patent Application No 4544610, strand displacement amplification as described in Walker et al. (1996) and EP A 684 315 and, target mediated amplification as described in PCT Publication WO 9322461, the disclosures of which are incorporated by reference in their entireties.

LCR and Gap LCR are exponential amplification techniques, both depending on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227), the disclosure of which is incorporated by reference in its entirety. Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770 or, to use Asymmetric Gap LCR (RT-AGLCR) as described by Marshall et al.(1994), the disclosures of which are incorporated by reference in its entireties. AGLCR is a modification of GLCR that allows the amplification of RNA.

PCR technology is the preferred amplification technique used in the present invention. A variety of PCR techniques are familiar to those skilled in the art. For a review of PCR technology, see White (1997), Erlich (1992) and the publication entitled “PCR Methods and Applications” ((1991) Cold Spring Harbor Laboratory Press), the disclosures of which are incorporated by reference in their entireties. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, Tth polymerase or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188, the disclosures of which are incorporated herein by reference in their entireties.

Preparation of Primers and Probes

Primers and probes can be prepared by any suitable method, including, for example, cloning and restriction of appropriate sequences and direct chemical synthesis by a method such as the phosphodiester method of Narang et al.(1979), the phosphodiester method of Brown et al.(1979), the diethylphosphoramidite method of Beaucage et al.(1981) and the solid support method described in EP 0 707 592, which disclosures are hereby incorporated by reference in their entireties.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047, which disclosures are hereby incorporated by reference in their entireties. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993, which disclosure is hereby incorporated by reference in its entirety, describes modifications, which can be used to render a probe non-extendable. Labeling of Probes

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al (1988) or Sanchez-Pescador et al (1988), which disclosures are hereby incorporated by reference in their entireties. In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron), which disclosures are hereby incorporated by reference in their entireties.

The detectable probe may be single stranded or double stranded and may be made using techniques known in the art, including in vitro transcription, nick translation, or kinase reactions. A nucleic acid sample containing a sequence capable of hybridizing to the labeled probe is contacted with the labeled probe. If the nucleic acid in the sample is double stranded, it may be denatured prior to contacting the probe. In some applications, the nucleic acid sample may be immobilized on a surface such as a nitrocellulose or nylon membrane. The nucleic acid sample may comprise nucleic acids obtained from a variety of sources, including genomic DNA, cDNA libraries, RNA, or tissue samples.

Procedures used to detect the presence of nucleic acids capable of hybridizing to the detectable probe include well known techniques such as Southern blotting, Northern blotting, dot blotting, colony hybridization, and plaque hybridization. In some applications, the nucleic acid capable of hybridizing to the labeled probe may be cloned into vectors such as expression vectors, sequencing vectors, or in vitro transcription vectors to facilitate the characterization and expression of the hybridizing nucleic acids in the sample. For example, such techniques may be used to isolate and clone sequences in a genomic library or cDNA library which are capable of hybridizing to the detectable probe as described herein.

Immobilization of Probes

A label can also be used to capture the primer, so as to facilitate the immobilization of either the primer or a primer extension product, such as amplified DNA, on a solid support. A capture label is attached to the primers or probes and can be a specific binding member which forms a binding pair with the solid phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can notably be used in Southern hybridization to genomic DNA. The probes can also be used to detect PCR amplification products. They may also be used to detect mismatches in the GENSET gene or mRNA using other techniques. They may also be used for in situ hybridization.

Any of the polynucleotides, primers and probes of the present invention can be conveniently immobilized on a solid support. The solid support is not critical and can be selected by one skilled in the art. Thus, latex particles, microparticles, magnetic beads, non-magnetic beads (including polystyrene beads), membranes (including nitrocellulose strips), plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Oligonucleotide Array

A substrate comprising a plurality of oligonucleotide primers or probes of the invention may be used either for detecting or amplifying targeted sequences in GENSET genes, may be used for detecting mutations in the coding or in the non-coding sequences of GENSET genes, and may also be used to determine GENSET gene expression in different contexts such as in different tissues, at different stages of a process (embryo development, disease treatment), and in patients versus healthy individuals as described elsewhere in the application.

As used herein, the term “array” means a one dimensional, two dimensional, or multidimensional arrangement of nucleic acids of sufficient length to permit specific detection of gene expression. For example, the array may contain a plurality of nucleic acids derived from genes whose expression levels are to be assessed. The array may include a GENSET genomic DNA, a GENSET cDNA, sequences complementary thereto or fragments thereof. Preferably, the fragments are at least 12, 15, 18, 20, 25, 30, 35, 40 or 50 nucleotides in length. More preferably, the fragments are at least 100 nucleotides in length. Even more preferably, the fragments are more than 100 nucleotides in length. In some embodiments the fragments may be more than 500 nucleotides in length.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854; PCT publications WO 90/15070 and 92/10092, which disclosures are hereby incorporated by reference in their entireties. These arrays may generally be produced using mechanical synthesis methods or light directed synthesis methods which incorporate a combination of photolithographic methods and solid phase oligonucleotide synthesis (Fodor et al., 1991), which disclosure is hereby incorporated by reference in its entirety. The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which disclosures are hereby incorporated by reference in their entireties, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

Consequently, the invention concerns an array of nucleic acid molecules comprising at least one polynucleotide of the invention, particularly a probe or primer as described herein. Preferably, the invention concerns an array of nucleic acids comprising at least two polynucleotides of the invention, particularly probes or primers as described herein. Preferably, the invention concerns an array of nucleic acids comprising at least five polynucleotides of the invention, particularly probes or primers as described herein.

A preferred embodiment of the present invention is an array of polynucleotides of at least 12, 15, 18, 20, 25, 30, 35, 40, 50, 100 or 500 nucleotides in length which includes at least 1, 2, 5, 10, 15, 20, 35, 50 or 100 sequences selected from the group consisting of the sequences of SEQ ID NOs:1-169, 339-455, 561-784 and sequences of clone inserts of the deposited clone pool, sequences fully complementary thereto, and fragments thereof.

Methods of Making the Polynucleotides of the Invention

The present invention also comprises methods of making the polynucleotides of the invention, including the polynucleotides of SEQ ID NOs:1-169, 339-455, 561-784, genomic DNA obtainable therefrom, or fragments thereof. These methods comprise sequentially linking together nucleotides to produce the nucleic acids having the preceding sequences. Polynucleotides of the invention may be synthesized either enzymatically using techniques well known to those skilled in the art including amplification or hybridization-based methods as described herein, or chemically.

A variety of chemical methods of synthesizing nucleic acids are known to those skilled in the art. In many of these methods, synthesis is conducted on a solid support. These included the 3′ phosphoramidite methods in which the 3′ terminal base of the desired oligonucleotide is immobilized on an insoluble carrier. The nucleotide base to be added is blocked at the 5′ hydroxyl and activated at the 3′ hydroxyl so as to cause coupling with the immobilized nucleotide base. Deblocking of the new immobilized nucleotide compound and repetition of the cycle will produce the desired polynucleotide. Alternatively, polynucleotides may be prepared as described in U.S. Pat. No. 5,049,656, which disclosure is hereby incorporated by reference in its entirety. In some embodiments, several polynucleotides prepared as described above are ligated together to generate longer polynucleotides having a desired sequence.

### Polypeptides of the Invention

The term “GENSET polypeptides” is used herein to embrace all of the proteins and polypeptides of the present invention. The present invention encompasses GENSET polypeptides, including recombinant, isolated or purified GENSET polypeptides consisting of, consisting essentially of, or comprising a sequence selected from the group consisting of SEQ ID NOs:170-338, 456-560, 785-918 and the polypeptides encoded by human cDNAs contained in the deposited clones. Other objects of the invention are polypeptides encoded by the polynucleotides of the invention as well as fusion polypeptides comprising such polypeptides.

Polypeptide Variants

The present invention further provides for GENSET polypeptides encoded by allelic and splice variants, orthologs, and/or species homologues. Procedures known in the art can be used to obtain, allelic variants, splice variants, orthologs, and/or species homologues of polynucleotides encoding by polypeptides of the group consisting of SEQ ID NOs:170-338, 456-560, 785-918 and polypeptides encoded by the clone inserts of the deposited clone pool, using information from the sequences disclosed herein or the clones deposited with the ATCC.

The polypeptides of the present invention also include polypeptides having an amino acid sequence at least 50% identical, more preferably at least 60% identical, and still more preferably 70%, 80%, 90%, 95%, 96%, 97%, 98% or 99% identical to a polypeptide selected from the group consisting of the sequences of SEQ ID NOs:170-338, 456-560, 785-918 and those encoded by the clone inserts of the deposited clone pool. By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, (indels) or substituted with another amino acid.

Further polypeptides of the present invention include polypeptides which have at least 90% similarity, more preferably at least 95% similarity, and still more preferably at least 96%, 97%, 98% or 99% similarity to those described above. By a polypeptide having an amino acid sequence at least, for example, 95% “similar” to a query amino acid sequence of the present invention, it is intended that the amino acid sequence of the subject polypeptide is similar (i.e. contains identical or equivalent amino acid residues) to the query sequence except that the subject polypeptide sequence may include up to five amino acid alterations per each 100 amino acids of the query amino acid sequence. In other words, to obtain a polypeptide having an amino acid sequence at least 95% similar to a query amino acid sequence, up to 5% (5 of 100) of the amino acid residues in the subject sequence may be inserted, deleted, (indels) or substituted with another non-equivalent amino acid.

These alterations of the reference sequence may occur at the amino or carboxy terminal positions of the reference amino acid sequence or anywhere between those terminal positions, interspersed either individually among residues in the reference sequence or in one or more contiguous groups within the reference sequence. The query sequence may be an entire amino acid sequence selected from the group consisting of sequences of SEQ ID NOs:170-338, 456-560, 785-918 and those encoded by the clone inserts of the deposited clone pool or any fragment specified as described herein.

The variant polypeptides described herein are included in the present invention regardless of whether they have their normal biological activity. This is because even where a particular polypeptide molecule does not have biological activity, one of skill in the art would still know how to use the polypeptide, for instance, as a vaccine or to generate antibodies. Other uses of the polypeptides of the present invention that do not have GENSET biological activity include, inter alia, as epitope tags, in epitope mapping, and as molecular weight markers on SDS-PAGE gels or on molecular sieve gel filtration columns using methods known to those of skill in the art. As described below, the polypeptides of the present invention can also be used to raise polyclonal and monoclonal antibodies, which are useful in assays for detecting GENSET protein expression or as agonists and antagonists capable of enhancing or inhibiting GENSET protein function. Further, such polypeptides can be used in the yeast two-hybrid system to “capture” GENSET protein binding proteins, which are also candidate agonists and antagonists according to the present invention (see, e.g., Fields et al. 1989, which disclosure is hereby incorporated by reference in its entirety).

Preparation of the Polypeptides of the Invention

The polypeptides of the present invention can be prepared in any suitable manner. Such polypeptides include isolated naturally occurring polypeptides, recombinantly produced polypeptides, synthetically produced polypeptides, or polypeptides produced by a combination of these methods. The polypeptides of the present invention are preferably provided in an isolated form, and may be partially or preferably substantially purified.

Consequently, the present invention also comprises methods of making the polypeptides of the invention, particularly polypeptides encoded by the cDNAs of SEQ ID NOs:1-169, 339-455, 561-784 or by the clone inserts of the deposited clone pool, genomic DNA obtainable therefrom, or fragments thereof and methods of making the polypeptides of SEQ ID NOs:170-338, 456-560, 785-918 or fragments thereof. The methods comprise sequentially linking together amino acids to produce the nucleic polypeptides having the preceding sequences. In some embodiments, the polypeptides made by these methods are 150 amino acids or less in length. In other embodiments, the polypeptides made by these methods are 120 amino acids or less in length.

Isolation

From Natural Sources

The GENSET proteins of the invention may be isolated from natural sources, including bodily fluids, tissues and cells, whether directly isolated or cultured cells, of humans or non-human animals. Methods for extracting and purifying natural proteins are known in the art, and include the use of detergents or chaotropic agents to disrupt particles followed by differential extraction and separation of the polypeptides by ion exchange chromatography, affinity chromatography, sedimentation according to density, and gel electrophoresis. See, for example, “Methods in Enymology, Academic Press, 1993” for a variety of methods for purifying proteins, which disclosure is hereby incorporated by reference in its entirety. Polypeptides of the invention also can be purified from natural sources using antibodies directed against the polypeptides of the invention, such as those described herein, in methods which are well known in the art of protein purification.

From Recombinant Sources

Preferably, the GENSET polypeptides of the invention are recombinantly produced using routine expression methods known in the art. The polynucleotide encoding the desired polypeptide is operably linked to a promoter into an expression vector suitable for any convenient host. Both eukaryotic and prokaryotic host systems are used in forming recombinant polypeptides. The polypeptide is then isolated from lysed cells or from the culture medium and purified to the extent needed for its intended use.

Any GENSET polynucleotide, including those described in SEQ ID NOs:1-169, 339-455, 561-784, those of clone inserts of the deposited clone pool, and allelic variants thereof may be used to express GENSET polypeptides. The nucleic acid encoding the GENSET polypeptide to be expressed is operably linked to a promoter in an expression vector using conventional cloning technology. The GENSET insert in the expression vector may comprise the full coding sequence for the GENSET protein or a portion thereof. For example, the GENSET derived insert may encode a polypeptide comprising at least 6, 8, 10, 12, 15, 20, 25, 30, 35, 40, 50, 60, 75, 100, 150 or 200 consecutive amino acids of a GENSET protein selected from the group consisting of sequences of SEQ ID NOs:170-338, 456-560, 785-918 and polypeptides encoded by the clone inserts of the deposited clone pool.

Consequently, a further embodiment of the present invention is a method of making a polypeptide comprising a protein selected from the group consisting of sequences of SEQ ID NOs:170-338, 456-560, 785-918 and polypeptides encoded by the clone inserts of the deposited clone pool, said method comprising the steps of:

a) obtaining a cDNA comprising a sequence selected from the group consisting of i) the sequences SEQ ID NOs:1-169, 339-455, 561-784, ii) the sequences of clone inserts of the deposited clone pool one, iii) sequences encoding one of the polypeptide of SEQ ID NOs:170-338, 456-560, 785-918, and iv) sequences of polynucleotides encoding a polypeptide which is encoded by one of the clone insert of the deposited clone pool;

b) inserting said cDNA in an expression vector such that the cDNA is operably linked to a promoter; and

c) introducing said expression vector into a host cell whereby said host cell produces said polypeptide.

In one aspect of this embodiment, the method further comprises the step of isolating the polypeptide. Another embodiment of the present invention is a polypeptide obtainable by the method described in the preceding paragraph.

The expression vector is any of the mammalian, yeast, insect or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence is optimized for the particular expression organism in which the expression vector is introduced, as explained in U.S. Pat. No. 5,082,767, which disclosure is hereby incorporated by reference in its entirety.

In one embodiment, the entire coding sequence of a GENSET cDNA and the 3′UTR through the poly A signal of the cDNA is operably linked to a promoter in the expression vector. Alternatively, if the nucleic acid encoding a portion of the GENSET protein lacks a methionine to serve as the initiation site, an initiating methionine can be introduced next to the first codon of the nucleic acid using conventional techniques. Similarly, if the insert from the GENSET cDNA lacks a poly A signal, this sequence can be added to the construct by, for example, splicing out the Poly A signal from pSG5 (Stratagene) using BglI and SalI restriction endonuclease enzymes and incorporating it into the mammalian expression vector pXT1 (Stratagene). pXT1 contains the LTRs and a portion of the gag gene from Moloney Murine Leukemia Virus. The position of the LTRs in the construct allow efficient stable transfection. The vector includes the Herpes Simplex Thymidine Kinase promoter and the selectable neomycin gene. The nucleic acid encoding the GENSET protein or a portion thereof is obtained by PCR from a vector containing a GENSET cDNA selected from the group consisting of the sequences of SEQ ID NOs:1-169, 339-455, 561-784 and the clone inserts of the deposited clone pool using oligonucleotide primers complementary to the GENSET cDNA or portion thereof and containing restriction endonuclease sequences for Pst I incorporated into the 5′ primer and BglII at the 5′ end of the corresponding cDNA 3′ primer, taking care to ensure that the sequence encoding the GENSET protein or a portion thereof is positioned properly with respect to the poly A signal. The purified fragment obtained from the resulting PCR reaction is digested with PstI, blunt ended with an exonuclease, digested with Bgl II, purified and ligated to pXTl, now containing a poly A signal and digested with BglII.

In another embodiment, it is often advantageous to add to the recombinant polynucleotide additional nucleotide sequence which codes for secretory or leader sequences, pro-sequences, sequences which aid in purification, such as multiple histidine residues, or an additional sequence for stability during recombinant production.

As a control, the expression vector lacking a cDNA insert is introduced into host cells or organisms.

Transfection of a GENSET expression vector into mouse NTH 3T3 cells is but one embodiment of introducing polynucleotides into host cells. Introduction of a polynucleotide encoding a polypeptide into a host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al. (1986), which disclosure is hereby incorporated by reference in its entirety. It is specifically contemplated that the polypeptides of the present invention may in fact be expressed by a host cell lacking a recombinant vector.

Recombinant cell extracts, or proteins from the culture medium if the expressed polypeptide is secreted, are then prepared and proteins separated by gel electrophoresis. If desired, the proteins may be ammonium sulfate precipitated or separated based on size or charge prior to electrophoresis. The proteins present are detected using techniques such as Coomassie or silver staining or using antibodies against the protein encoded by the GENSET cDNA of interest. Coomassie and silver staining techniques are familiar to those skilled in the art.

Proteins from the host cells or organisms containing an expression vector which contains the GENSET cDNA or a fragment thereof are compared to those from the control cells or organism. The presence of a band from the cells containing the expression vector which is absent in control cells indicates that the GENSET cDNA is expressed. Generally, the band corresponding to the protein encoded by the GENSET cDNA will have a mobility near that expected based on the number of amino acids in the open reading frame of the cDNA. However, the band may have a mobility different than that expected as a result of modifications such as glycosylation, ubiquitination, or enzymatic cleavage.

Alternatively, the GENSET polypeptide to be expressed may also be a product of transgenic animals, i.e., as a component of the milk of transgenic cows, goats, pigs or sheep which are characterized by somatic or germ cells containing a nucleotide sequence encoding the protein of interest.

A polypeptide of this invention can be recovered and purified from recombinant cell cultures by well-known methods including differential extraction, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. See, for example, “Methods in Enzymology”, supra for a variety of methods for purifying proteins. Most preferably, high performance liquid chromatography (“HPLC”) is employed for purification. A recombinantly produced version of a GENSET polypeptide can be substantially purified using techniques described herein or otherwise known in the art, such as, for example, by the one-step method described in Smith and Johnson (1988), which disclosure is hereby incorporated by reference in its entirety. Polypeptides of the invention also can be purified from recombinant sources using antibodies directed against the polypeptides of the invention, such as those described herein, in methods which are well known in the art of protein purification.

Preferably, the recombinantly expressed GENSET polypeptide is purified using standard immunochromatography techniques such as the one described in the section entitled “Immunoaffinity Chromatography”. In such procedures, a solution containing the protein of interest, such as the culture medium or a cell extract, is applied to a column having antibodies against the protein attached to the chromatography matrix. The recombinant protein is allowed to bind the immunochromatography column. Thereafter, the column is washed to remove non-specifically bound proteins. The specifically bound secreted protein is then released from the column and recovered using standard techniques.

If antibody production is not possible, the GENSET cDNA sequence or fragment thereof may be incorporated into expression vectors designed for use in purification schemes employing chimeric polypeptides. In such strategies the coding sequence of the GENSET cDNA or fragment thereof is inserted in frame with the gene encoding the other half of the chimera. The other half of the chimera may be beta-globin or a nickel binding polypeptide encoding sequence. A chromatography matrix having antibody to beta-globin or nickel attached thereto is then used to purify the chimeric protein. Protease cleavage sites may be engineered between the beta-globin gene or the nickel binding polypeptide and the GENSET cDNA or fragment thereof. Thus, the two polypeptides of the chimera may be separated from one another by protease digestion.

One useful expression vector for generating beta-globin chimerics is pSG5 (Stratagene), which encodes rabbit beta-globin. Intron II of the rabbit beta-globin gene facilitates splicing of the expressed transcript, and the polyadenylation signal incorporated into the construct increases the level of expression. These techniques as described are well known to those skilled in the art of molecular biology. Standard methods are published in methods texts such as Davis et al., (1986) and many of the methods are available from Stratagene, Life Technologies, Inc., or Promega. Polypeptide may additionally be produced from the construct using in vitro translation systems such as the In vitro Express™ Translation Kit (Stratagene).

Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

From Chemical Synthesis

In addition, polypeptides of the invention, especially short protein fragments, can be chemically synthesized using techniques known in the art (See, e.g., Creighton, 1983; and Hunkapiller et al., 1984), which disclosures are hereby incorporated by reference in their entireties. For example, a polypeptide corresponding to a fragment of a polypeptide sequence of the invention can be synthesized by use of a peptide synthesizer. A variety of methods of making polypeptides are known to those skilled in the art, including methods in which the carboxyl terminal amino acid is bound to polyvinyl benzene or another suitable resin. The amino acid to be added possesses blocking groups on its amino moiety and any side chain reactive groups so that only its carboxyl moiety can react. The carboxyl group is activated with carbodiimide or another activating agent and allowed to couple to the immobilized amino acid. After removal of the blocking group, the cycle is repeated to generate a polypeptide having the desired sequence. Alternatively, the methods described in U.S. Pat. No. 5,049,656, which disclosure is hereby incorporated by reference in its entirety, may be used.

Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Modifications

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein.

Also provided by the invention are chemically modified derivatives of the polypeptides of the invention which may provide additional advantages such as increased solubility, stability and circulating time of the polypeptide, or decreased immunogenicity. See U.S. Pat. No: 4,179,337. The chemical moieties for derivatization may be selected See U.S. Pat. No: 4,179,337, which disclosure is hereby incorporated by reference in its entirety. The chemical moieties for derivatization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, (coupling PEG to G-CSF), and Malik et al. (1992) (reporting pegylation of GM-CSF using tresyl chloride), which disclosures are hereby incorporated by reference in their entireties. For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Multimerization

The polypeptides of the invention may be in monomers or multimers (i.e., dimers, trimers, tetramers and higher multimers). Accordingly, the present invention relates to monomers and multimers of the polypeptides of the invention, their preparation, and compositions containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term “homomer”, refers to a multimer containing only polypeptides corresponding to the amino acid sequences of SEQ ID NOs:170-338, 456-560, 785-918 or encoded by the clone inserts of the deposited clone pool (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomenc multimer of the invention is at least a homodimer, at least a homotrimer, or at least a homotetramer.

As used herein, the term “heteromer” refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the sequence listing, or contained in the polypeptide encoded by a deposited clone). In one instance, the covalent associations are cross-linking between cysteine residues located within the polypeptide sequences, which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925, which disclosure is hereby incorporated by reference in its entirety). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, oseteoprotegerin (see, e.g., International Publication No: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves the use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins, and have since been found in a variety of different proteins (Landschulz et al., 1988). Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (1994) and in U.S. patent application Ser. No. 08/446,922, which disclosure is hereby incorporated by reference in its entirety. Other peptides derived from naturally occurring trimeric proteins may be employed in preparing trimeric polypeptides of the invention. In another example, proteins of the invention are associated by interactions between Flag® polypeptide sequence contained in fusion proteins of the invention containing Flag® polypeptide sequence. In a further embodiment, associations proteins of the invention are associated by interactions between heterologous polypeptide sequence contained in Flag® fusion proteins of the invention and anti Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, other techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hydrophobic or signal peptide) and which can be incorporated by membrane reconstitution techniques into liposomes (see, e.g., U.S. Pat. No. 5,478,925, which is herein incorporated by reference in its entirety).

Mutated Polypeptides

To improve or alter the characteristics of GENSET polypeptides of the present invention, protein engineering may be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they may be purified in higher yields and show better solubility than the corresponding natural polypeptide, at least under certain purification and storage conditions. Further, the polypeptides of the present invention may be produced as multimers including dimers, trimers and tetramers. Multimerization may be facilitated by linkers or recombinantly though heterologous polypeptides such as Fc regions.

N- and C-terminal Deletions

It is known in the art that one or more amino acids may be deleted from the N-terminus or C-terminus without substantial loss of biological function. For instance, Ron et al. (1993), reported modified KGF proteins that had heparin binding activity even if 3, 8, or 27 N-terminal amino acid residues were missing. Accordingly, the present invention provides polypeptides having one or more residues deleted from the amino terminus of the polypeptides of SEQ ID NOs:170-338, 456-560, 785-918 or that encoded by the clone inserts of the deposited clone pool. Similarly, many examples of biologically functional C-terminal deletion mutants are known. For instance, Interferon gamma shows up to ten times higher activities by deleting 810 amino acid residues from the C-terminus of the protein (See, e.g., Dobeli, et al. 1988), which disclosure is hereby incorporated by reference in its entirety. Accordingly, the present invention provides polypeptides having one or more residues deleted from the carboxy terminus of the polypeptides shown of SEQ ID NOs:170-338, 456-560, 785-918 or encoded by the clone inserts of the deposited clone pool. The invention also provides polypeptides having one or more amino acids deleted from both the amino and the carboxyl termini as described below.

Other Mutations

Other mutants in addition to N- and C-terminal deletion forms of the protein discussed above are included in the present invention. It also will be recognized by one of ordinary skill in the art that some amino acid sequences of the GENSET polypeptides of the present invention can be varied without significant effect of the structure or function of the protein. If such differences in sequence are contemplated, it should be remembered that there will be critical areas on the protein which determine activity. Thus, the invention further includes variations of the GENSET polypeptides which show substantial GENSET polypeptide activity. Such mutants include deletions, insertions, inversions, repeats, and substitutions selected according to general rules known in the art so as to have little effect on activity. For example, guidance concerning how to make phenotypically silent amino acid substitutions is provided.

There are two main approaches for studying the tolerance of an amino acid sequence to change (see, Bowie et al. 1994, which disclosure is hereby incorporated by reference in its entirety). The first method relies on the process of evolution, in which mutations are either accepted or rejected by natural selection.

The second approach uses genetic engineering to introduce amino acid changes at specific positions of a cloned gene and selections or screens to identify sequences that maintain functionality. These studies have revealed that proteins are surprisingly tolerant of amino acid substitutions. The studies indicate which amino acid changes are likely to be permissive at a certain position of the protein. For example, most buried amino acid residues require nonpolar side chains, whereas few features of surface side chains are generally conserved. Other such phenotypically silent substitutions are described by Bowie et al. (supra) and the references cited therein.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the fragment, derivative, analog, or homologue of the polypeptide of the present invention may be, for example: (i) one in which one or more of the amino acid residues are substituted with a conserved or non-conserved amino acid residue (preferably a conserved amino acid residue) and such substituted amino acid residue may or may not be one encoded by the genetic code: or (ii) one in which one or more of the amino acid residues includes a substituent group: or (iii) one in which the GENSET polypeptide is fused with another compound, such as a compound to increase the half-life of the polypeptide (for example, polyethylene glycol): or (iv) one in which the additional amino acids are fused to the above form of the polypeptide, such as an IgG Fc fusion region peptide or leader or secretory sequence or a sequence which is employed for purification of the above form of the polypeptide or a pro-protein sequence. Such fragments, derivatives and analogs are deemed to be within the scope of those skilled in the art from the teachings herein.

Thus, the GENSET polypeptides of the present invention may include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids generally represent equivalent changes: (1) Ala, Pro, Gly, Glu, Asp, Gin, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

A specific embodiment of a modified GENSET peptide molecule of interest according to the present invention, includes, but is not limited to, a peptide molecule which is resistant to proteolysis, is a peptide in which the —CONH— peptide bond is modified and replaced by a (CH2NH) reduced bond, a (NHCO) retro inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond. The invention also encompasses a human GENSET polypeptide or a fragment or a variant thereof in which at least one peptide bond has been modified as described above.

Amino acids in the GENSET proteins of the present invention that are essential for function can be identified by methods known in the art, such as site-directed mutagenesis or alanine-scanning mutagenesis (see, e.g., Cunningham et al. 1989, which disclosure is hereby incorporated by reference in its entirety). The latter procedure introduces single alanine mutations at every residue in the molecule. The resulting mutant molecules are then tested for biological activity using assays appropriate for measuring the function of the particular protein. Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic, (see, e.g., Pinckard et al., 1967; Robbins, et al., 1987; and Cleland, et al., 1993).

A further embodiment of the invention relates to a polypeptide which comprises the amino acid sequence of a GENSET polypeptide having an amino acid sequence which contains at least one conservative amino acid substitution, but not more than 50 conservative amino acid substitutions, not more than 40 conservative amino acid substitutions, not more than 30 conservative amino acid substitutions, and not more than 20 conservative amino acid substitutions. Also provided are polypeptides which comprise the amino acid sequence of a GENSET polypeptide, having at least one, but not more than 10, 9, 8, 7, 6, 5, 4, 3, 2 or 1 conservative amino acid substitutions.

Polypeptide Fragments

Structural Definition

The present invention is further directed to fragments of the amino acid sequences described herein such as the polypeptides of SEQ ID NOs:170-338, 456-560, 785-918 or those encoded by the clone inserts of the deposited clone pool. More specifically, the present invention embodies purified, isolated, and recombinant polypeptides comprising at least 6, preferably at least 8 to 10, more preferably 12, 15, 20, 25, 30, 35, 40, 50, 60, 75, 100, 125, 150, 175, 200, 225, 250, 275, or 300 consecutive amino acids of a polypeptide selected from the group consisting of the sequences of SEQ ID NOs:170-338, 456-560, 785-918, the polypeptides encoded by the clone inserts of the deposited clone pool, and other polypeptides of the present invention.

In addition to the above polypeptide fragments, further preferred sub-genuses of polypeptides comprise at least 6 amino acids, wherein “at least 6” is defined as any integer between 6 and the integer representing the C-terminal amino acid of the polypeptide of the present invention including the polypeptide sequences of the sequence listing below. Further included are species of polypeptide fragments at least 6 amino acids in length, as described above, that are further specified in terms of their N-terminal and C-terminal positions. However, included in the present invention as individual species are all polypeptide fragments, at least 6 amino acids in length, as described above, and may be particularly specified by a N-terminal and C-terminal position. That is, every combination of a N-terminal and C-terminal position that a fragment at least 6 contiguous amino acid residues in length could occupy, on any given amino acid sequence of the sequence listing or of the present invention is included in the present invention

The present invention also provides for the exclusion of any fragment species specified by N-terminal and C-terminal positions or of any fragment sub-genus specified by size in amino acid residues as described above. Any number of fragments specified by N-terminal and C-terminal positions or by size in amino acid residues as described above may be excluded as individual species.

The above polypeptide fragments of the present invention can be immediately envisaged using the above description and are therefore not individually listed solely for the purpose of not unnecessarily lengthening the specification. Moreover, the above fragments need not have a GENSET biological activity, although polypeptides having these activities are preferred embodiments of the invention, since they would be useful, for example, in immunoassays, in epitope mapping, epitope tagging, as vaccines, and as molecular weight markers. The above fragments may also be used to generate antibodies to a particular portion of the polypeptide. These antibodies can then be used in immunoassays well known in the art to distinguish between human and non-human cells and tissues or to determine whether cells or tissues in a biological sample are or are not of the same type which express the polypeptides of the present invention.

It is noted that the above species of polypeptide fragments of the present invention may alternatively be described by the formula “a to b”; where “a” equals the N-terminal most amino acid position and “b” equals the C-terminal most amino acid position of the polynucleotide; and further where “a” equals an integer between 1 and the number of amino acids of the polypeptide sequence of the present invention minus 6, and where “b” equals an integer between 7 and the number of amino acids of the polypeptide sequence of the present invention; and where “a” is an integer smaller then “b” by at least 6.

The present invention also provides for the exclusion of any species of polypeptide fragments of the present invention specified by 5′ and 3′ positions or sub-genuses of polypeptides specified by size in amino acids as described above. Any number of fragments specified by 5′ and 3′ positions or by size in amino acids, as described above, may be excluded.

Functional Definition

Domains

Preferred polynucleotide fragments of the invention are domains of polypeptides of the invention. Such domains may eventually comprise linear or structural motifs and signatures including, but not limited to, leucine zippers, helix-turn-helix motifs, post-translational modification sites such as glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites. Such domains may present a particular biological activity such as DNA or RNA-binding, secretion of proteins, transcription regulation, enzymatic activity, substrate binding activity, etc.

A domain has a size generally comprised between 3 and 1000 amino acids. In a preferred embodiment, domains comprise a number of amino acids that is any integer between 6 and 200. Domains may be synthesized using any methods known to those skilled in the art, including those disclosed herein, particularly in the section entitled “Preparation of the polypeptides of the invention”. Methods for determining the amino acids which make up a domain with a particular biological activity include mutagenesis studies and assays to determine the biological activity to be tested.

Alternatively, the polypeptides of the invention may be scanned for motifs, domains and/or signatures in databases using any computer method known to those skilled in the art. Searchable databases include Prosite (Hofmann et al., 1999; Bucher and Bairoch 1994), Pfam (Sonnhammer et al., 1997; Henikoffet al., 2000; Bateman et al., 2000), Blocks (Henikoffet al., 2000), Print (Attwood et al., 1996), Prodom (Sonnhammer and Kahn, 1994; Corpet et al. 2000), Sbase (Pongor et al., 1993; Murvai et al., 2000), Smart (Schultz et al., 1998), Dali/FSSP (Holm and Sander, 1996, 1997 and 1999), HSSP (Sander and Schneider 1991), CATH (Orengo et al., 1997; Pearl et al., 2000), SCOP (Murzin et al., 1995; Lo Conte et al., 2000), COG (Tatusov et al., 1997 and 2000), specific family databases and derivatives thereof (Nevill-Manning et al., 1998; Yona et al., 1999; Attwood et al., 2000), each of which disclosures are hereby incorporated by reference in their entireties. For a review on available databases, see issue 1 of volume 28 of Nucleic Acid Research (2000), which disclosure is hereby incorporated by reference in its entirety.

The domains of the present invention preferably comprises 6 to 200 amino acids (i.e. any integer between 6 and 200, inclusive) of a polypeptide of the present invention. Also, included in the present invention are domain fragments between the integers of 6 and the full length GENSET sequence of the sequence listing. All combinations of sequences between the integers of 6 and the full-length sequence of a GENSET polypeptide are included. The domain fragments may be specified by either the number of contiguous amino acid residues (as a sub-genus) or by specific N-terminal and C-terminal positions (as species) as described above for the polypeptide fragments of the present invention. Any number of domain fragments of the present invention may also be excluded in the same manner.

Epitopes and Antibody Fusions:

A preferred embodiment of the present invention is directed to epitope-bearing polypeptides and epitope-bearing polypeptide fragments. These epitopes may be “antigenic epitopes” or both an “antigenic epitope” and an “immunogenic epitope”. An “immunogenic epitope” is defined as a part of a protein that elicits an antibody response in vivo when the polypeptide is the immunogen. On the other hand, a region of polypeptide to which an antibody binds is defined as an “antigenic determinant” or “antigenic epitope.” The number of immunogenic epitopes of a protein generally is less than the number of antigenic epitopes (See, e.g., Geysen, et al., 1984), which disclosure is hereby incorporated by reference in its entirety. It is particularly noted that although a particular epitope may not be immunogenic, it is nonetheless useful since antibodies can be made to both immunogenic and antigenic epitopes.

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes may be produced by any conventional means (See, e.g., Houghten, 1985), also further described in U.S. Pat. No. 4,631,21, which disclosures are hereby incorporated by reference in their entireties. Methods for determining the amino acids which make up an epitope include x-ray crystallography, 2-dimensional nuclear magnetic resonance, and epitope mapping, e.g., the Pepscan method described by Geysen et al. (1984); PCT Publication No. WO 84/03564; and PCT Publication No. WO 84/03506, which disclosures are hereby incorporated by reference in their entireties. Another example is the algorithm of Jameson and Wolf, (1988) (said reference incorporated by reference in its entirety). The Jameson-Wolf antigenic analysis, for example, may be performed using the computer program PROTEAN, using default parameters (Version 4.0 Windows, DNASTAR, Inc., 1228 South Park Street Madison, Wis.

The epitope-bearing fragments of the present invention preferably comprise 6 to 50 amino acids (i.e. any integer between 6 and 50, inclusive) of a polypeptide of the present invention. Also, included in the present invention are antigenic fragments between the integers of 6 and the full length GENSET sequence of the sequence listing. All combinations of sequences between the integers of 6 and the full-length sequence of a GENSET polypeptide are included. The epitope-bearing fragments may be specified by either the number of contiguous amino acid residues (as a sub-genus) or by specific N-terminal and C-terminal positions (as species) as described above for the polypeptide fragments of the present invention. Any number of epitope-bearing fragments of the present invention may also be excluded in the same manner.

Antigenic epitopes are useful, for example, to raise antibodies, including monoclonal antibodies that specifically bind the epitope (see, Wilson et al., 1984; and Sutcliffe, et al., 1983, which disclosures are hereby incorporated by reference in their entireties). The antibodies are then used in various techniques such as diagnostic and tissue/cell identification techniques, as described herein, and in purification methods such as immunoaffinity chromatography.

Similarly, immunogenic epitopes can be used to induce antibodies according to methods well known in the art (See, Sutcliffe et al., supra; Wilson et al., supra; Chow et al.(1985); and Bittle, et al., (1985), which disclosures are hereby incorporated by reference in their entireties). A preferred immunogenic epitope includes the natural GENSET protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.).

Epitope-bearing polypeptides of the present invention are used to induce antibodies according to methods well known in the art including, but not limited to, in vivo immunization, in vitro immunization, and phage display methods (See, e.g., Sutcliffe, et al., supra; Wilson, et al., supra, and Bittle, et al., supra). If in vivo immunization is used, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine residues may be coupled to a carrier using a linker such as -maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μgs of peptide or carrier protein and Freund's adjuvant. Several booster injections may be needed, for instance, at intervals of about two weeks, to provide a useful titer of anti-peptide antibody, which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and discussed above, the polypeptides of the present invention comprising an immunogenic or antigenic epitope can be fused to heterologous polypeptide sequences. For example, the polypeptides of the present invention may be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo. This has been shown, e.g., for chimeric proteins consisting of the first two domains of the human CD4-polypeptide and various domains of the constant regions of the heavy or light chains of mammalian immunoglobulins (See, e.g., EPA 0,394,827; and Traunecker et al., 1988, which disclosures are hereby incorporated by reference in their entireties). Fusion proteins that have a disulfide-linked dimeric structure due to the IgG portion can also be more efficient in binding and neutralizing other molecules than monomeric polypeptides or fragments thereof alone (See, e.g., Fountoulakis et al., 1995, which disclosure is hereby incorporated by reference in its entirety). Nucleic acids encoding the above epitopes can also be recombined with a gene of interest as an epitope tag to aid in detection and purification of the expressed polypeptide.

Additional fusion proteins of the invention may be generated through the techniques of gene-shuffling, motif-shuffling, exon-shuffling, or codon-shuffling (collectively referred to as “DNA shuffling”). DNA shuffling may be employed to modulate the activities of polypeptides of the present invention thereby effectively generating agonists and antagonists of the polypeptides. See, for example, U.S. Pat. Nos.: 5,605,793; 5,811,238; 5,834,252; 5,837,458; and Patten, et al., (1997); Harayama, (1998); Hansson, et al (1999); and Lorenzo and Blasco, (1998). (Each of these documents are hereby incorporated by reference). In one embodiment, one or more components, motifs, sections, parts, domains, fragments, etc., of coding polynucleotides of the invention, or the polypeptides encoded thereby may be recombined with one or more components, motifs, sections, parts, domains, fragments, etc. of one or more heterologous molecules.

The present invention further encompasses any combination of the polypeptide fragments listed in this section.

Antibodies

Definitions

The present invention further relates to antibodies and T-cell antigen receptors (TCR), which specifically bind the polypeptides, and more specifically, the epitopes of the polypeptides of the present invention. The antibodies of the present invention include IgG (including IgG1, IgG2, IgG3, and IgG4), IgA (including IgA1 and IgA2), IgD, IgE, or IgM, and IgY. The term “antibody” (Ab) refers to a polypeptide or group of polypeptides which are comprised of at least one binding domain, where a binding domain is formed from the folding of variable domains of an antibody molecule to form three-dimensional binding spaces with an internal surface shape and charge distribution complementary to the features of an antigenic determinant of an antigen, which allows an immunological reaction with the antigen. As used herein, the term “antibody” is meant to include whole antibodies, including single-chain whole antibodies, and antigen binding fragments thereof. In a preferred embodiment the antibodies are human antigen binding antibody fragments of the present invention include, but are not limited to, Fab, Fab′F(ab)2 and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies, which specifically bind the polypeptides of the present invention. The present invention further includes antibodies that are anti-idiotypic to the antibodies of the present invention.

The antibodies of the present invention may be monospecific, bispecific, and trispecific or have greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, et al. (1991); U.S. Pat. Nos. 5,573,920, 4,474,893, 5,601,819, 4,714,681, 4,925,648; Kostelny et al. (1992), which disclosures are hereby incorporated by reference in their entireties.

Antibodies of the present invention may be described or specified in terms of the epitope(s) or epitope-bearing portion(s) of a polypeptide of the present invention, which are recognized or specifically bound by the antibody. The antibodies may specifically bind a complete protein encoded by a nucleic acid of the present invention, or a fragment thereof. Therefore, the epitope(s) or epitope bearing polypeptide portion(s) may be specified as described herein, e.g., by N-terminal and C-terminal positions, by size in contiguous amino acid residues, or otherwise described herein (including the sequence listing). Antibodies which specifically bind any epitope or polypeptide of the present invention may also be excluded as individual species. Therefore, the present invention includes antibodies that specifically bind specified polypeptides of the present invention, and allows for the exclusion of the same.

Thus, another embodiment of the present invention is a purified or isolated antibody capable of specifically binding to a polypeptide comprising a sequence selected from the group consisting of the sequences of SEQ ID NOs:170-338, 456-560, 785-918 and the sequences of the clone inserts of the deposited clone pool. In one aspect of this embodiment, the antibody is capable of binding to an epitope-containing polypeptide comprising at least 6 consecutive amino acids, preferably at least 8 to 10 consecutive amino acids, more preferably at least 12, 15, 20, 25, 30, 40, 50, or 100 consecutive amino acids of a sequence selected from the group consisting of SEQ ID NOs:170-338, 456-560, 785-918 and sequences of the clone inserts of the deposited clone pool.

Antibodies of the present invention may also be described or specified in terms of their cross-reactivity. Antibodies that do not specifically bind any other analog, ortholog, or homologue of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein, e.g., using FASTDB and the parameters set forth herein) to a polypeptide of the present invention are also included in the present invention. Further included in the present invention are antibodies, which only bind polypeptides encoded by polynucleotides, which hybridize to a polynucleotide of the present invention under stringent hybridization conditions (as described herein). Antibodies of the present invention may also be described or specified in terms of their binding affinity. Preferred binding affinities include those with a issociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

The invention also concerns a purified or isolated antibody capable of specifically binding to a mutated GENSET protein or to a fragment or variant thereof comprising an epitope of the mutated GENSET protein.

Preparation of Antibodies

The antibodies of the present invention may be prepared by any suitable method known in the art. Some of these methods are described in more detail in the example entitled “Preparation of Antibody Compositions to the GENSET protein”. For example, a polypeptide of the present invention or an antigenic fragment thereof can be administered to an animal in order to induce the production of sera containing “polyclonal antibodies”. As used herein, the term “monoclonal antibody” is not limited to antibodies produced through hybridoma technology but it rather refers to an antibody that is derived from a single clone, including eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art (See, e.g., Harlow et al. 1988; Hammerling, et al, 1981). (Said references incorporated by reference in their entireties). Fab and F(ab′)2 fragments may be produced, for example, from hybridoma-produced antibodies by proteolytic cleavage, using enzymes such as papain (to produce Fab fragments) or pepsin (to produce F(ab′)2 fragments).

Alternatively, antibodies of the present invention can be produced through the application of recombinant DNA technology or through synthetic chemistry using methods known in the art. For example, the antibodies of the present invention can be prepared using various phage display methods known in the art. In phage display methods, functional antibody domains are displayed on the surface of a phage particle, which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman et al. (1995); Ames, et al. (1995); Kettleborough, et al. (1994); Persic, et al. (1997); Burton et al. (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ F(ab)2 and F(ab′)2 fragments can also be employed using methods known in the art such as those disclosed in WO 92/22324; Mullinax et al. (1992); and Sawai et al. (1995); and Better et al. (1988) (said references incorporated by reference in their entireties).

Examples of techniques which can be used to produce single-chain Fvs and antibodies include those described in U.S. Pat. Nos. 4,946,778 and 5,258,498; Huston et al. (1991); Shu et al. (1993); and Skerra et al. (1988), which disclosures are hereby incorporated by reference in their entireties. For some uses, including in vivo use of antibodies in humans and in vitro detection assays, it may be preferable to use chimeric, humanized, or human antibodies. Methods for producing chimeric antibodies are known in the art. See e.g., Morrison, (1985); Oi et al., (1986); Gillies et al. (1989); and U.S. Pat. No. 5,807,715, which disclosures are hereby incorporated by reference in their entireties. Antibodies can be humanized using a variety of techniques including CDR-grafting (EP 0 239 400; WO 91/09967; U.S. Pat. No. 5,530,101; and 5,585,089), veneering or resurfacing, (EP 0 592 106; EP 0 519 596; Padlan, 1991; Studnicka et al., 1994; Roguska et al., 1994), and chain shuffling (U.S. Pat. No. 5,565,332), which disclosures are hereby incorporated by reference in their entireties. Human antibodies can be made by a variety of methods known in the art including phage display methods described above. See also, U.S. Pat. Nos. 4,444,887, 4,716,111, 5,545,806, and 5,814,318; WO 98/46645; WO 98/50433; WO 98/24893; WO 96/34096; WO 96/33735; and WO 91/10741 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387, which disclosures are hereby incorporated by reference in their entireties. Fused antibodies may also be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in vitro immunoassays and purification methods using methods known in the art (See e.g., Harbor et al. supra; WO 93/21232; EP 0 439 095; Naramura, M. et al. 1994; U.S. Pat. No. 5,474,981; Gillies et al., 1992; Fell et al., 1991) (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half-life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi et al. (1991); Zheng et al. (1995); and Vil et al. (1992) (said references incorporated by reference in their entireties).

Non-human animals or mammals, whether wild-type or transgenic, which express a different species of GENSET than the one to which antibody binding is desired, and animals which do not express GENSET (i.e. a GENSET knock out animal as described herein) are particularly useful for preparing antibodies. GENSET knock out animals will recognize all or most of the exposed regions of a GENSET protein as foreign antigens, and therefore produce antibodies with a wider array of GENSET epitopes. Moreover, smaller polypeptides with only 10 to 30 amino acids may be useful in obtaining specific binding to any one of the GENSET proteins. In addition, the humoral immune system of animals which produce a species of GENSET that resembles the antigenic sequence will preferentially recognize the differences between the animal's native GENSET species and the antigen sequence, and produce antibodies to these unique sites in the antigen sequence. Such a technique will be particularly useful in obtaining antibodies that specifically bind to any one of the GENSET proteins.

The antibodies of the invention may be labeled by, e.g., any one of the radioactive, fluorescent or enzymatic labels known in the art.

### Uses of Polynucleotides

Uses of Polynucleotides as Reagents

The polynucleotides of the present invention, particularly those described in the “Oligonucleotide primers and probes” section, may be used as reagents in isolation procedures, diagnostic assays, and forensic procedures. For example, sequences from the GENSET polynucleotides of the invention may be detectably labeled and used as probes to isolate other sequences capable of hybridizing to them. In addition, sequences from the GENSET polynucleotides of the invention may be used to design PCR primers to be used in isolation, diagnostic, or forensic procedures.

In Forensic Analyses

PCR primers may be used in forensic analyses, such as the DNA fingerprinting techniques described below. Such analyses may utilize detectable probes or primers based on the sequences of the polynucleotides of the invention. Consequently, the present invention encompasses methods of identification of an individual using the polynucleotides of the invention in forensic analyses, wherein said method includes the steps of:

a) obtaining a biological sample containing nucleic acid material from an individual;

b) obtaining an identification pattern for this individual using the polynucleotides of the invention, particularly using GENSET primers and probes;

c) comparing said identification pattern with a reference identification pattern; and

d) determining whether said identification pattern is identical to said reference identification pattern.

In one embodiment of this method, the identification pattern consists in sequences of amplicons obtained using GENSET primers as explained in the sections entitled “Forensic Matching by DNA Sequencing” and “Positive Identification by DNA Sequencing”.

In another embodiment, the identification pattern consists in unique band or dot patterns obtained using any method described in the sections entitled “Southern Blot Forensic Identification”, “Dot Blot Identification Procedure” and “Alternative “Fingerprint” Identification Technique”.

Table I provides sets of related cDNAs of the invention, e.g. sequences that represent allelic variants of a single sequence. Such variants are especially useful for the herein-described forensic analyses, and are also useful as polymorphic markers to examine, e.g. associations between the herein-discussed GENSET genes and various diseases or conditions.

Forensic Matching by DNA Sequencing

In one exemplary method, DNA samples are isolated from forensic specimens of, for example, hair, semen, blood or skin cells by conventional methods. A panel of PCR primers designed from different polynucleotides of the invention using any technique known to those skilled in the art including those described herein, is then utilized to amplify DNA of approximately 100-200 bases in length from the forensic specimen. Corresponding sequences are obtained from a test subject. Each of these identification DNAs is then sequenced using standard techniques, and a simple database comparison determines the differences, if any, between the sequences from the subject and those from the sample. Statistically significant differences between the suspect's DNA sequences and those from the sample conclusively prove a lack of identity. This lack of identity can be proven, for example, with only one sequence. Identity, on the other hand, should be demonstrated with a large number of sequences, all matching. Preferably, a minimum of 50 statistically identical sequences of 100 bases in length are used to prove identity between the suspect and the sample.

Positive Identification by DNA Sequencing

The “Forensic Matching by DNA Sequencing” technique described herein may also be used on a larger scale to provide a unique fingerprint-type identification of any individual. In this technique, primers are prepared from a large number of polynucleotides of the invention. Preferably, 20 to 50 different primers are used. These primers are used to obtain a corresponding number of PCR-generated DNA segments from the individual in question. Each of these DNA segments is sequenced. The database of sequences generated through this procedure uniquely identifies the individual from whom the sequences were obtained. The same panel of primers may then be used at any later time to absolutely correlate tissue or other biological specimen with that individual.

Southern Blot Forensic Identification

The “Positive Identification by DNA Sequencing” procedure described herein is repeated to obtain a panel of at least 10 amplified sequences from an individual and a specimen. Preferably, the panel contains at least 50 amplified sequences. More preferably, the panel contains 100 amplified sequences. In some embodiments, the panel contains 200 amplified sequences. This PCR-generated DNA is then digested with one or a combination of, preferably, four base specific restriction enzymes. Such enzymes are commercially available and known to those of skill in the art. After digestion, the resultant gene fragments are size separated in multiple duplicate wells on an agarose gel and transferred to nitrocellulose using Southern blotting techniques well known to those with skill in the art. For a review of Southern blotting see Davis et al. (1986), which disclosure is hereby incorporated by reference in its entirety.

A panel of probes based on the sequences of the polynucleotides of the invention, or fragments thereof of at least 10 bases, are radioactively or calorimetrically labeled using methods known in the art, such as nick translation or end labeling, and hybridized to the Southern blot using techniques known in the art. Preferably, the probe comprises at least 12, 15, or 17 consecutive nucleotides from the polynucleotide of the invention. More preferably, the probe comprises at least 20-30 consecutive nucleotides from the polynucleotide of the invention. In some embodiments, the probe comprises more than 30 nucleotides from the polynucleotide of the invention. In other embodiments, the probe comprises at least 40, at least 50, at least 75, at least 100, at least 150, or at least 200 consecutive nucleotides from the polynucleotide of the invention.

Preferably, at least 5 to 10 of these labeled probes are used, and more preferably at least about 20 or 30 are used to provide a unique pattern. The resultant bands appearing from the hybridization of a large sample of polynucleotide of the invention will be a unique identifier. Since the restriction enzyme cleavage will be different for every individual, the band pattern on the Southern blot will also be unique. Increasing the number of cDNA probes will provide a statistically higher level of confidence in the identification since there will be an increased number of sets of bands used for identification.

Dot Blot Identification Procedure

Another technique for identifying individuals using the polynucleotide sequences disclosed herein utilizes a dot blot hybridization technique.

Genomic DNA is isolated from nuclei of subject to be identified. Oligonucleotide probes of approximately 30 bp in length are synthesized that correspond to at least 10, preferably 50 sequences from the polynucleotide of the invention. The probes are used to hybridize to the genomic DNA through conditions known to those in the art. The oligonucleotides are end labeled with P³² using polynucleotide kinase (Pharmacia). Dot Blots are created by spotting the genomic DNA onto nitrocellulose or the like using a vacuum dot blot manifold (BioRad, Richmond Calif.). The nitrocellulose filter containing the genomic sequences is baked or UV linked to the filter, prehybridized and hybridized with labeled probe using techniques known in the art (Davis et al. 1986). The ³²P labeled DNA fragments are sequentially hybridized with successively stringent conditions to detect minimal differences between the 30 bp sequence and the DNA. Tetramethylammonium chloride is useful for identifying clones containing small numbers of nucleotide mismatches (Wood et al., 1985). A unique pattern of dots distinguishes one individual from another individual.

Alternative “Fingerprint” Identification Technique

In a representative alternative fingerprinting procedure, the probes are derived from cDNAs. Preferably, a plurality of probes having sequences from different genes are used as follows. Polynucleotides containing at least 10 consecutive bases from these sequences can be used as probes. Preferably, the probe comprises at least 12, 15, or 17 consecutive nucleotides from the polynucleotide of the invention. More preferably, the probe comprises at least 20-30 consecutive nucleotides from the polynucleotide of the invention. In some embodiments, the probe comprises more than 30 nucleotides from the polynucleotide of the invention. In other embodiments, the probe comprises at least 40, at least 50, at least 75, at least 100, at least 150, or at least 200 consecutive nucleotides from the polynucleotide of the invention.

Oligonucleotides, generally 20-mers, are prepared from a large number, e.g. 50, 100, or 200, of polynucleotides of the invention using commercially available oligonucleotide services such as Genset (Paris, France). Cell samples from the test subject are processed for DNA using techniques well known to those with skill in the art. The nucleic acid is digested with restriction enzymes such as EcoRI and XbaI. Following digestion, samples are applied to wells for electrophoresis. The procedure, as known in the art, may be modified to accommodate polyacrylamide electrophoresis, however in this example, samples containing 5 ug of DNA are loaded into wells and separated on 0.8% agarose gels. The gels are transferred onto nitrocellulose using standard Southern blotting techniques.

10 ng of each of the oligonucleotides are pooled and end-labeled with P³². The nitrocellulose is prehybridized with blocking solution and hybridized with the labeled probes. Following hybridization and washing, the nitrocellulose filter is exposed to X-Omat AR X-ray film. The resulting hybridization pattern will be unique for each individual.

It is additionally contemplated within this example that the number of probe sequences used can be varied for additional accuracy or clarity.

To Find Corresponding Genomic DNA Sequences

The GENSET cDNAs of the invention may also be used to clone sequences located upstream of the cDNAs of the invention on the corresponding genomic DNA. Such upstream sequences may be capable of regulating gene expression, including promoter sequences, enhancer sequences, and other upstream sequences which influence transcription or translation levels. Once identified and cloned, these upstream regulatory sequences may be used in expression vectors designed to direct the expression of an inserted gene in a desired spatial, temporal, developmental, or quantitative fashion.

Use of cDNAs or Fragments Thereof to Clone Upstream Sequences from Genomic DNA

Sequences derived from polynucleotides of the inventions may be used to isolate the promoters of the corresponding genes using chromosome walking techniques. In one chromosome walking technique, which utilizes the GenomeWalker™ kit available from Clontech, five complete genomic DNA samples are each digested with a different restriction enzyme which has a 6 base recognition site and leaves a blunt end. Following digestion, oligonucleotide adapters are ligated to each end of the resulting genomic DNA fragments.

For each of the five genomic DNA libraries, a first PCR reaction is performed according to the manufacturer's instructions (which are incorporated herein by reference) using an outer adaptor primer provided in the kit and an outer gene specific primer. The gene specific primer should be selected to be specific for the polynucleotide of the invention of interest and should have a melting temperature, length, and location in the polynucleotide of the invention which is consistent with its use in PCR reactions. Each first PCR reaction contains 5 ng of genomic DNA, 5 μl of 10×Tth reaction buffer, 0.2 mM of each dNTP, 0.2 μM each of outer adaptor primer and outer gene specific primer, 1.1 mM of Mg(OAc)₂, and 1 μl of the Tth polymerase 50× mix in a total volume of 50 μl. The reaction cycle for the first PCR reaction is as follows: 1 min at 94 degrees Celsius/2 sec at 94 degree Celsius, 3 min at 72 degrees Celsius (7 cycles)/2 sec at 94 degrees Celsius, 3 min at 67 degrees Celsius (32 cycles)/5 min at 67 degrees Celsius.

The product of the first PCR reaction is diluted and used as a template for a second PCR reaction according to the manufacturer's instructions using a pair of nested primers which are located internally on the amplicon resulting from the first PCR reaction. For example, 5 μl of the reaction product of the first PCR reaction mixture may be diluted 180 times. Reactions are made in a 50 μl volume having a composition identical to that of the first PCR reaction except the nested primers are used. The first nested primer is specific for the adaptor, and is provided with the GenomeWalker™ kit. The second nested primer is specific for the particular polynucleotide of the invention for which the promoter is to be cloned and should have a melting temperature, length, and location in the polynucleotide of the invention which is consistent with its use in PCR reactions. The reaction parameters of the second PCR reaction are as follows: 1 min at 94 degrees Celsius/2 sec at 94 degrees Celsius, 3 min at 72 degrees Celsius (6 cycles)/2 sec at 94 degrees Celsius, 3 min at 67 degrees Celsius (25 cycles)/5 min at 67 degrees Celsius

The product of the second PCR reaction is purified, cloned, and sequenced using standard techniques. Alternatively, two or more human genomic DNA libraries can be constructed by using two or more restriction enzymes. The digested genomic DNA is cloned into vectors which can be converted into single stranded, circular, or linear DNA. A biotinylated oligonucleotide comprising at least 15 nucleotides from the polynucleotide of the invention sequence is hybridized to the single stranded DNA. Hybrids between the biotinylated oligonucleotide and the single stranded DNA containing the polynucleotide of the invention sequence are isolated as described herein. Thereafter, the single stranded DNA containing the polynucleotide of the invention sequence is released from the beads and converted into double stranded DNA using a primer specific for the polynucleotide of the invention sequence or a primer corresponding to a sequence included in the cloning vector. The resulting double stranded DNA is transformed into bacteria. DNAs containing the GENSET polynucleotide sequences are identified by colony PCR or colony hybridization.

Identification of Promoters in Cloned Upstream Sequences

Once the upstream genomic sequences have been cloned and sequenced as described above, prospective promoters and transcription start sites within the upstream sequences may be identified by comparing the sequences upstream of the polynucleotides of the inventions with databases containing known transcription start sites, transcription factor binding sites, or promoter sequences.

In addition, promoters in the upstream sequences may be identified using promoter reporter vectors as follows. The expression of the reporter gene will be detected when placed under the control of regulatory active polynucleotide fragments or variants of the GENSET promoter region located upstream of the first exon of the GENSET gene. Suitable promoter reporter vectors, into which the GENSET promoter sequences may be cloned include pSEAP-Basic, pSEAP-Enhancer, pβgal-Basic, pβgal-Enhancer, or pEGFP-1 Promoter Reporter vectors available from Clontech, or pGL2-basic or pGL3-basic promoterless luciferase reporter gene vector from Promega. Briefly, each of these promoter reporter vectors include multiple cloning sites positioned upstream of a reporter gene encoding a readily assayable protein such as secreted alkaline phosphatase, luciferase, beta-galactosidase, or green fluorescent protein. The sequences upstream the GENSET coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequence within the upstream genomic DNA may be further defined by site directed mutagenesis, linker scanning analysis, or other techniques familiar to those skilled in the art. For example, the boundaries of promoters may be further investigated by constructing nested 5′ and/or 3′ deletions in the upstream DNA using conventional techniques such as Exonuclease III or appropriate restriction endonuclease digestion. The resulting deletion fragments can be inserted into the promoter reporter vector to determine whether the deletion has increased, reduced or illuminated promoter activity, such as described, for example, by Coles et al. (1998), the disclosure of which is incorporated herein by reference in its entirety. In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. This type of assay is well known to those skilled in the art and is described in WO 97/17359, U.S. Pat. No. 5,374,544; EP 582 796; U.S. Pat. Nos. 5,698,389; 5,643,746; 5,502,176; and 5,266,488; the disclosures of which are incorporated by reference herein in their entirety.

The strength and the specificity of the promoter of each GENSET gene can be assessed through the expression levels of a detectable polynucleotide operably linked to the GENSET promoter in different types of cells and tissues. The detectable polynucleotide may be either a polynucleotide that specifically hybridizes with a predefined oligonucleotide probe, or a polynucleotide encoding a detectable protein, including a GENSET polypeptide or a fragment or a variant thereof. This type of assay is well known to those skilled in the art and is described in U.S. Pat. Nos. 5,502,176; and 5,266,488; the disclosures of which are incorporated by reference herein in their entirety. Some of the methods are discussed in more detail elsewhere in the application.

The promoters and other regulatory sequences located upstream of the polynucleotides of the inventions may be used to design expression vectors capable of directing the expression of an inserted gene in a desired spatial, temporal, developmental, or quantitative manner. A promoter capable of directing the desired spatial, temporal, developmental, and quantitative patterns may be selected using the results of the expression analysis described herein. For example, if a promoter which confers a high level of expression in muscle is desired, the promoter sequence upstream of a polynucleotide of the invention derived from an mRNA which is expressed at a high level in muscle may be used in the expression vector. Such vectors are described in more detail elsewhere in the application.

Preferably, the desired promoter is placed near multiple restriction sites to facilitate the cloning of the desired insert downstream of the promoter, such that the promoter is able to drive expression of the inserted gene. The promoter may be inserted in conventional nucleic acid backbones designed for extrachromosomal replication, integration into the host chromosomes or transient expression. Suitable backbones for the present expression vectors include retroviral backbones, backbones from eukaryotic episomes such as SV40 or Bovine Papilloma Virus, backbones from bacterial episomes, or artificial chromosomes.

Preferably, the expression vectors also include a polyA signal downstream of the multiple restriction sites for directing the polyadenylation of mRNA transcribed from the gene inserted into the expression vector.

To Find Similar Sequences

Polynucleotides of the invention may be used to isolate and/or purify nucleic acids similar thereto using any methods well known to those skilled in the art including the techniques based on hybridization or on amplification described in this section. These methods may be used to obtain the genomic DNAs which encode the mRNAs from which the GENSET cDNAs are derived, mRNAs corresponding to GENSET cDNAs, or nucleic acids which are homologous to GENSET cDNAs or fragments thereof, such as variants, species homologues or orthologs. Thus, a plurality of cDNAs similar to GENSET polynucleotides may be provided as cDNA libraries for subsequent evaluation of the encoded proteins or used in diagnostic assays as described herein. cDNAs prepared by any method described therein may be subsequently engineered to obtain nucleic acids which include desired fragments of the cDNA using conventional techniques such as subcloning, PCR, or in vitro oligonucleotide synthesis. For example, nucleic acids which include only the coding sequences may be obtained using techniques known to those skilled in the art. Similarly, nucleic acids containing any other desired fragment of the coding sequences for the encoded protein may be obtained.

Indeed, cDNAs of the present invention or fragments thereof may be used to isolate nucleic acids similar to cDNAs from a cDNA library or a genomic DNA library. Such cDNA libraries or genomic DNA libraries may be obtained from a commercial source or made using techniques familiar to those skilled in the art such as those described in PCT publication WO 00/37491, which disclosure is hereby incorporated by reference in its entirety. Examples of methods for obtaining nucleic acids similar to GENSET polynucleotides are described below.

Hybridization-Based Methods

Techniques for identifying cDNA clones in a cDNA library which hybridize to a given probe sequence are disclosed in Sambrook et al., (1989) and in Hames and Higgins (1985), the disclosures of which are incorporated herein by reference in their entireties. The same techniques may be used to isolate genomic DNAs.

Briefly, cDNA or genomic DNA clones which hybridize to the detectable probe are identified and isolated for further manipulation as follows. Any polynucleotide fragment of the invention may be used as a probe, in particular those defined in the “Oligonucleotide primers and probes” section. A probe comprising at least 10 consecutive nucleotides from a GENSET cDNA or fragment thereof is labeled with a detectable label such as a radioisotope or a fluorescent molecule. Preferably, the probe comprises at least 12, 15, or 17 consecutive nucleotides from the cDNA or fragment thereof. More preferably, the probe comprises 20 to 30 consecutive nucleotides from the cDNA or fragment thereof. In some embodiments, the probe comprises more than 30 nucleotides from the cDNA or fragment thereof.

Techniques for labeling the probe are well known and include phosphorylation with polynucleotide kinase, nick translation, in vitro transcription, and non radioactive techniques. The cDNAs or genomic DNAs in the library are transferred to a nitrocellulose or nylon filter and denatured. After blocking of non specific sites, the filter is incubated with the labeled probe for an amount of time sufficient to allow binding of the probe to cDNAs or genomic DNAs containing a sequence capable of hybridizing thereto.

By varying the stringency of the hybridization conditions used to identify cDNAs or genomic DNAs which hybridize to the detectable probe, cDNAs or genomic DNAs having different levels of identity to the probe can be identified and isolated as described below.

Stringent Conditions

“Stringent hybridization conditions” are defined as conditions in which only nucleic acids having a high level of identity to the probe are able to hybridize to said probe. These conditions may be calculated as follows:

For probes between 14 and 70 nucleotides in length the melting temperature (Tm) is calculated using the formula: Tm=81.5+16.6(log (Na+))+0.41(fraction G+C)−600/N) where N is the length of the probe.

If the hybridization is carried out in a solution containing formamide, the melting temperature may be calculated using the equation: Tm=81.5+16.6(log (Na+))+0.41(fraction G+C)−(0.63% formamide)−(600/N) where N is the length of the probe.

Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% formamide. The formulas for SSC and Denhardt's solutions are listed in Sambrook et al., 1986.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to nucleic acids containing sequences complementary thereto or homologous thereto. For probes over 200 nucleotides in length, the hybridization may be carried out at 15-25° C. below the Tm. For shorter probes, such as oligonucleotide probes, the hybridization may be conducted at 15-25° C. below the Tm. Preferably, for hybridizations in 6×SSC, the hybridization is conducted at approximately 68° C. Preferably, for hybridizations in 50% formamide containing solutions, the hybridization is conducted at approximately 42° C.

Following hybridization, the filter is washed in 2×SSC, 0.1% SDS at room temperature for 15 minutes. The filter is then washed with 0.1×SSC, 0.5% SDS at room temperature for 30 minutes to 1 hour. Thereafter, the solution is washed at the hybridization temperature in 0.1×SSC, 0.5% SDS. A final wash is conducted in 0.1×SSC at room temperature.

Nucleic acids which have hybridized to the probe are identified by autoradiography or other conventional techniques.

Low and Moderate Conditions

Changes in the stringency of hybridization and signal detection are primarily accomplished through the manipulation of formamide concentration (lower percentages of formamide result in lowered stringency); salt conditions, or temperature. The above procedure may thus be modified to identify nucleic acids having decreasing levels of identity to the probe sequence. For example, the hybridization temperature may be decreased in increments of 5° C. from 68° C. to 42° C. in a hybridization buffer having a sodium concentration of approximately 1M. Following hybridization, the filter may be washed with 2×SSC, 0.5% SDS at the temperature of hybridization. These conditions are considered to be “moderate” conditions above 50° C. and “low” conditions below 50° C. Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of identity to the probe. Following hybridization, the filter may be washed with 6×SSC, 0.5% SDS at 50° C. These conditions are considered to be “moderate” conditions above 25% formamide and “low” conditions below 25% formamide. cDNAs or genomic DNAs which have hybridized to the probe are identified by autoradiography or other conventional techniques.

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Consequently, the present invention encompasses methods of isolating nucleic acids similar to the polynucleotides of the invention, comprising the steps of:

a) contacting a collection of cDNA or genomic DNA molecules with a detectable probe comprising at least 12, 15, 18, 20, 23, 25, 28, 30, 35, 40 or 50 consecutive nucleotides of a sequence selected from the group consisting of the sequences of SEQ ID NOs: 1-169, 339-455, 561-784, the sequences of clones inserts of the deposited clone pool and sequences complementary thereto under stringent, moderate or low conditions which permit said probe to hybridize to at least a cDNA or genomic DNA molecule in said collection;

b) identifying said cDNA or genomic DNA molecule which hybridizes to said detectable probe; and

c) isolating said cDNA or genomic DNA molecule which hybridized to said probe.

PCR-Based Methods

In addition to the above described methods, other protocols are available to obtain homologous cDNAs using GENSET cDNA of the present invention or fragment thereof as outlined in the following paragraphs.

cDNAs may be prepared by obtaining mRNA from the tissue, cell, or organism of interest using mRNA preparation procedures utilizing polyA selection procedures or other techniques known to those skilled in the art. A first primer capable of hybridizing to the polyA tail of the mRNA is hybridized to the mRNA and a reverse transcription reaction is performed to generate a first cDNA strand.

The term “capable of hybridizing to the polyA tail of said mRNA” refers to and embraces all primers containing stretches of thymidine residues, so-called oligo(dT) primers, that hybridize to the 3′ end of eukaryotic poly(A)+ mRNAs to prime the synthesis of a first cDNA strand. Techniques for generating said oligo (dT) primers and hybridizing them to mRNA to subsequently prime the reverse transcription of said hybridized mRNA to generate a first cDNA strand are well known to those skilled in the art and are described in Current Protocols in Molecular Biology, John Wiley and Sons, Inc. 1997 and Sambrook et al., 1989. Preferably, said oligo (dT) primers are present in a large excess in order to allow the hybridization of all mRNA 3′ ends to at least one oligo (dT) molecule. The priming and reverse transcription steps are preferably performed between 37° C. and 55° C. depending on the type of reverse transcriptase used. Preferred oligo(dT) primers for priming reverse transcription of mRNAs are oligonucleotides containing a stretch of thymidine residues of sufficient length to hybridize specifically to the polyA tail of mRNAs, preferably of 12 to 18 thymidine residues in length. More preferably, such oligo(T) primers comprise an additional sequence upstream of the poly(dT) stretch in order to allow the addition of a given sequence to the 5′ end of all first cDNA strands which may then be used to facilitate subsequent manipulation of the cDNA. Preferably, this added sequence is 8 to 60 residues in length. For instance, the addition of a restriction site in 5′ of cDNAs facilitates subcloning of the obtained cDNA. Alternatively, such an added 5′ end may also be used to design primers of PCR to specifically amplify cDNA clones of interest.

The first cDNA strand is then hybridized to a second primer. Any polynucleotide fragment of the invention may be used, and in particular those described in the “Oligonucleotide primers and probes” section. This second primer contains at least 10 consecutive nucleotides of a polynucleotide of the invention. Preferably, the primer comprises at least 10, 12, 15, 17, 18, 20, 23, 25, or 28 consecutive nucleotides of a polynucleotide of the invention. In some embodiments, the primer comprises more than 30 nucleotides of a polynucleotide of the invention. If it is desired to obtain cDNAs containing the full protein coding sequence, including the authentic translation initiation site, the second primer used contains sequences located upstream of the translation initiation site. The second primer is extended to generate a second cDNA strand complementary to the first cDNA strand. Alternatively, RT-PCR may be performed as described above using primers from both ends of the cDNA to be obtained.

The double stranded cDNAs made using the methods described above are isolated and cloned. The cDNAs may be cloned into vectors such as plasmids or viral vectors capable of replicating in an appropriate host cell. For example, the host cell may be a bacterial, mammalian, avian, or insect cell.

Techniques for isolating mRNA, reverse transcribing a primer hybridized to mRNA to generate a first cDNA strand, extending a primer to make a second cDNA strand complementary to the first cDNA strand, isolating the double stranded cDNA and cloning the double stranded cDNA are well known to those skilled in the art and are described in Current Protocols in Molecular Biology, John Wiley & Sons, Inc. 1997 and Sambrook et al., 1989.

Consequently, the present invention encompasses methods of making cDNAs. In a first embodiment, the method of making a cDNA comprises the steps of

a) contacting a collection of mRNA molecules from human cells with a primer comprising at least 12, 15, 18, 20, 23, 25, 28, 30, 35, 40, or 50 consecutive nucleotides of a sequence selected from the group consisting of the sequences complementary to SEQ ID NOs:1-169, 339-455, 561-784 and sequences complementary to a clone insert of the deposited clone pool;

b) hybridizing said primer to an mRNA in said collection;

c) reverse transcribing said hybridized primer to make a first cDNA strand from said mRNA;

d) making a second cDNA strand complementary to said first cDNA strand; and

e) isolating the resulting cDNA comprising said first cDNA strand and said second cDNA strand.

Another embodiment of the present invention is a purified cDNA obtainable by the method of the preceding paragraph. In one aspect of this embodiment, the cDNA encodes at least a portion of a human polypeptide.

In a second embodiment, the method of making a cDNA comprises the steps of

a) contacting a collection of mRNA molecules from human cells with a first primer capable of hybridizing to the polyA tail of said mRNA;

b) hybridizing said first primer to said polyA tail;

c) reverse transcribing said mRNA to make a first cDNA strand;

d) making a second cDNA strand complementary to said first cDNA strand using at least one primer comprising at least 12, 15, 18, 20, 23, 25, 28, 30, 35, 40, or 50 consecutive nucleotides of a sequence selected from the group consisting of SEQ ID NOs:1-169, 339-455, 561-784 and sequences of clone inserts of the deposited clone pool; and

e) isolating the resulting cDNA comprising said first cDNA strand and said second cDNA strand.

In another aspect of this method the second cDNA strand is made by

a) contacting said first cDNA strand with a second primer comprising at least 12, 15, 18, 20, 23, 25, 28, 30, 35, 40, or 50 consecutive nucleotides of a sequence selected from the group consisting of SEQ ID NOs:1-169, 339-455, 561-784 and sequences of clone inserts of the deposited clone pool, and a third primer which sequence is fully included within the sequence of said first primer;

b) performing a first polymerase chain reaction with said second and third primers to generate a first PCR product;

c) contacting said first PCR product with a fourth primer, comprising at least 12, 15, 18, 20, 23, 25, 28,30, 35, 40, or 50 consecutive nucleotides of said sequence selected from the group consisting of SEQ ID NOs:1-169, 339-455, 561-784 and sequences of clone inserts of the deposited clone pool, and a fifth primer, which sequence is fully included within the sequence of said third primer, wherein said fourth and fifth hybridize to sequences within said first PCR product; and

d) performing a second polymerase chain reaction, thereby generating a second PCR product.

Alternatively, the second cDNA strand may be made by contacting said first cDNA strand with a second primer comprising at least 12, 15, 18, 20, 23, 25, 28, 30, 35, 40, or 50 consecutive nucleotides of a sequence selected from the group consisting of SEQ ID NOs:1-169, 339-455, 561-784 and sequences of clone inserts of the deposited clone pool, and a third primer which sequence is fully included within the sequence of said first primer and performing a polymerase chain reaction with said second and third primers to generate said second cDNA strand.

Alternatively, the second cDNA strand may be made by:

a) contacting said first cDNA strand with a second primer comprising at least 12, 15, 18, 20, 23, 25, 28, 30, 35, 40, or 50 consecutive nucleotides of a sequence selected from the group consisting of SEQ ID NOs:1-169, 339-455, 561-784 and sequences of clone inserts of the deposited clone pool;

b) hybridizing said second primer to said first strand cDNA; and

c) extending said hybridized second primer to generate said second cDNA strand.

Another embodiment of the present invention is a purified cDNA obtainable by a method of making a cDNA of the invention. In one aspect of this embodiment, said cDNA encodes at least a portion of a human polypeptide.

Other Protocols

Alternatively, other procedures may be used for obtaining homologous cDNAs. In one approach, cDNAs are prepared from mRNA and cloned into double stranded phagemids as follows. The cDNA library in the double stranded phagemids is then rendered single stranded by treatment with an endonuclease, such as the Gene II product of the phage F1 and an exonuclease (Chang et al., 1993, which disclosure is hereby incorporated by reference in its entirety). A biotinylated oligonucleotide comprising the sequence of a fragment of a known GENSET cDNA, genomic DNA or fragment thereof is hybridized to the single stranded phagemids. Preferably, the fragment comprises at least 10, 12, 15, 17, 18, 20, 23, 25, or 28 consecutive nucleotides of a sequence selected from the group consisting of the sequences of SEQ ID NOs:1-169, 339-455, 561-784 and sequences of clone inserts of the deposited clone pool.

Hybrids between the biotinylated oligonucleotide and phagemids are isolated by incubating the hybrids with streptavidin coated paramagnetic beads and retrieving the beads with a magnet (Fry et al, 1992, which disclosure is hereby incorporated by reference in its entirety). Thereafter, the resulting phagemids are released from the beads and converted into double stranded DNA using a primer specific for the GENSET cDNA or fragment used to design the biotinylated oligonucleotide. Alternatively, protocols such as the Gene Trapper kit (Gibco BRL), which disclosure is which disclosure is hereby incorporated by reference in its entirety, may be used. The resulting double stranded DNA is transformed into bacteria. Homologous cDNAs to the GENSET cDNA or fragment thereof sequence are identified by colony PCR or colony hybridization.

As a Chromosome Marker

Chromosomal localization of the cDNA of the present invention were determined using information from public and proprietary databases. Table II lists the putative chromosomal location of the polynucleotides of the present invention. Column one lists the sequence identification number with the corresponding chromosomal location listed in column two. Thus, the present invention also relates to methods and compositions using the chromosomal location of the polynucleotides of the invention to construct a human high resolution map or to identify a given chromosome in a sample using any techniques known to those skilled in the art including those disclosed below.

GENSET polynucleotides may also be mapped to their chromosomal locations using any methods or techniques known to those skilled in the art including radiation hybrid (RH) mapping, PCR-based mapping and Fluorescence in situ hybridization (FISH) mapping described below.

Radiation Hybrid Mapping

Radiation hybrid (RH) mapping is a somatic cell genetic approach that can be used for high resolution mapping of the human genome. In this approach, cell lines containing one or more human chromosomes are lethally irradiated, breaking each chromosome into fragments whose size depends on the radiation dose. These fragments are rescued by fusion with cultured rodent cells, yielding subclones containing different fragments of the human genome. This technique is described by Benham et al. (1989) and Cox et al., (1990), which disclosures are hereby incorporated by reference in their entireties. The random and independent nature of the subclones permits efficient mapping of any human genome marker. Human DNA isolated from a panel of 80-100 cell lines provides a mapping reagent for ordering GENSET cDNAs or genomic DNAs. In this approach, the frequency of breakage between markers is used to measure distance, allowing construction of fine resolution maps as has been done using conventional ESTs (Schuler et al., 1996), which disclosure is hereby incorporated by reference in its entirety.

RH mapping has been used to generate a high-resolution whole genome radiation hybrid map of human chromosome 17q22-q25.3 across the genes for growth hormone (GH) and thymidine kinase (TK) (Foster et al., 1996), the region surrounding the Gorlin syndrome gene (Obermayr et al., 1996), 60 loci covering the entire short arm of chromosome 12 (Raeymaekers et al., 1995), the region of human chromosome 22 containing the neurofibromatosis type 2 locus (Frazer et al., 1992) and 13 loci on the long arm of chromosome 5 (Warrington et al., 1991), which disclosures are hereby incorporated by reference in their entireties.

Mapping of cDNAs to Human Chromosomes using PCR Techniques

GENSET cDNAs and genomic DNAs may be assigned to human chromosomes using PCR based methodologies. In such approaches, oligonucleotide primer pairs are designed from the cDNA sequence to minimize the chance of amplifying through an intron. Preferably, the oligonucleotide primers are 18-23 bp in length and are designed for PCR amplification. The creation of PCR primers from known sequences is well known to those with skill in the art. For a review of PCR technology see Erlich (1992), which disclosure is hereby incorporated by reference in its entirety.

The primers are used in polymerase chain reactions (PCR) to amplify templates from total human genomic DNA. PCR conditions are as follows: 60 ng of genomic DNA is used as a template for PCR with 80 ng of each oligonucleotide primer, 0.6 unit of Taq polymerase, and 1 uCu of a ³²P-labeled deoxycytidine triphosphate. The PCR is performed in a microplate thermocycler (Techne) under the following conditions: 30 cycles of 94 degrees Celsius, 1.4 min; 55 degrees Celsius, 2 min; and 72 degrees Celsius, 2 min; with a final extension at 72 degrees Celsius for 10 min. The amplified products are analyzed on a 6% polyacrylamide sequencing gel and visualized by autoradiography. If the length of the resulting PCR product is identical to the distance between the ends of the primer sequences in the cDNA from which the primers are derived, then the PCR reaction is repeated with DNA templates from two panels of human-rodent somatic cell hybrids, BIOS PCRable DNA (BIOS Corporation) and NIGMS Human-Rodent Somatic Cell Hybrid Mapping Panel Number 1 (NIGMS, Camden, N.J.).

PCR is used to screen a series of somatic cell hybrid cell lines containing defined sets of human chromosomes for the presence of a given cDNA or genomic DNA. DNA is isolated from the somatic hybrids and used as starting templates for PCR reactions using the primer pairs from the GENSET cDNAs or genomic DNAs. Only those somatic cell hybrids with chromosomes containing the human gene corresponding to the GENSET cDNA or genomic DNA will yield an amplified fragment. The GENSET cDNAs or genomic DNAs are assigned to a chromosome by analysis of the segregation pattern of PCR products from the somatic hybrid DNA templates. The single human chromosome present in all cell hybrids that give rise to an amplified fragment is the chromosome containing that GENSET cDNA or genomic DNA. For a review of techniques and analysis of results from somatic cell gene mapping experiments, see Ledbetter et al., (1990), which disclosure is hereby incorporated by reference in its entirety.

Mapping of cDNAs to Chromosomes Using Fluorescence in situ Hybridization

Fluorescence in situ hybridization (FISH) allows the GENSET cDNA or genomic DNA to be mapped to a particular location on a given chromosome. The chromosomes to be used for fluorescence in situ hybridization techniques may be obtained from a variety of sources including cell cultures, tissues, or whole blood.

In a preferred embodiment, chromosomal localization of a GENSET cDNA or genomic DNA is obtained by FISH as described by Cherif et al. (1990), which disclosure is hereby incorporated by reference in its entirety. Metaphase chromosomes are prepared from phytohemagglutinin (PHA)-stimulated blood cell donors. PHA-stimulated lymphocytes from healthy males are cultured for 72 h in RPMI-1640 medium. For synchronization, methotrexate (10 uM) is added for 17 h, followed by addition of 5-bromodeoxyuridine (5-BudR, 0.1 mM) for 6 h. Colcemid (1 ug/ml) is added for the last 15 min before harvesting the cells. Cells are collected, washed in RPMI, incubated with a hypotonic solution of KCl (75 mM) at 37 degrees Celsius for 15 min and fixed in three changes of methanol:acetic acid (3:1). The cell suspension is dropped onto a glass slide and air dried. The GENSET cDNA or genomic DNA is labeled with biotin-16 dUTP by nick translation according to the manufacturer's instructions (Bethesda Research Laboratories, Bethesda, Md.), purified using a Sephadex G-50 column (Pharmacia, Upssala, Sweden) and precipitated. Just prior to hybridization, the DNA pellet is dissolved in hybridization buffer (50% formamide, 2×SSC, 10% dextran sulfate, 1 mg/ml sonicated salmon sperm DNA, pH 7) and the probe is denatured at 70 degrees Celsius for 5-10 min.

Slides kept at −20 degrees Celsius are treated for 1 h at 37 degrees Celsius with RNase A (100 ug/ml), rinsed three times in 2×SSC and dehydrated in an ethanol series. Chromosome preparations are denatured in 70% formamide, 2×SSC for 2 min at 70 degrees Celsius, then dehydrated at 4 degrees Celsius. The slides are treated with proteinase K (10 ug/100 ml in 20 mM Tris-HCl, 2 mM CaCl₂) at 37 degrees Celsius for 8 min and dehydrated. The hybridization mixture containing the probe is placed on the slide, covered with a coverslip, sealed with rubber cement and incubated overnight in a humid chamber at 37 degrees Celsius. After hybridization and post-hybridization washes, the biotinylated probe is detected by avidin-FITC and amplified with additional layers of biotinylated goat anti-avidin and avidin-FITC. For chromosomal localization, fluorescent R-bands are obtained as previously described (Cherif et al., 1990). The slides are observed under a LEICA fluorescence microscope (DMRXA). Chromosomes are counterstained with propidium iodide and the fluorescent signal of the probe appears as two symmetrical yellow-green spots on both chromatids of the fluorescent R-band chromosome (red). Thus, a particular GENSET cDNA or genomic DNA may be localized to a particular cytogenetic R-band on a given chromosome.

Use of cDNAs to Construct or Expand Chromosome Maps

Once the GENSET cDNAs or genomic DNAs have been assigned to particular chromosomes using any technique known to those skilled in the art those skilled in the art, particularly those described herein, they may be utilized to construct a high resolution map of the chromosomes on which they are located or to identify the chromosomes in a sample.

Chromosome mapping involves assigning a given unique sequence to a particular chromosome as described above. Once the unique sequence has been mapped to a given chromosome, it is ordered relative to other unique sequences located on the same chromosome. One approach to chromosome mapping utilizes a series of yeast artificial chromosomes (YACs) bearing several thousand long inserts derived from the chromosomes of the organism from which the GENSET cDNAs or genomic DNAs are obtained. This approach is described in Nagaraja et al. (1997), which disclosure is hereby incorporated by reference in its entirety. Briefly, in this approach each chromosome is broken into overlapping pieces which are inserted into the YAC vector. The YAC inserts are screened using PCR or other methods to determine whether they include the GENSET cDNA or genomic DNA whose position is to be determined. Once an insert has been found which includes the GENSET cDNA or genomic DNA, the insert can be analyzed by PCR or other methods to determine whether the insert also contains other sequences known to be on the chromosome or in the region from which the GENSET cDNA or genomic DNA was derived. This process can be repeated for each insert in the YAC library to determine the location of each of the GENSET cDNA or genomic DNA relative to one another and to other known chromosomal markers. In this way, a high resolution map of the distribution of numerous unique markers along each of the organisms chromosomes may be obtained.

Identification of Genes Associated with Hereditary Diseases or Drug Response

This example illustrates an approach useful for the association of GENSET cDNAs or genomic DNAs with particular phenotypic characteristics. In this example, a particular GENSET cDNA or genomic DNA is used as a test probe to associate that GENSET cDNA or genomic DNA with a particular phenotypic characteristic.

GENSET cDNAs or genomic DNAs are mapped to a particular location on a human chromosome using techniques such as those described herein or other techniques known in the art. A search of Mendelian Inheritance in Man (V. McKusick, Mendelian Inheritance in Man; available on line through Johns Hopkins University Welch Medical Library) reveals the region of the human chromosome which contains the GENSET cDNA or genomic DNA to be a very gene rich region containing several known genes and several diseases or phenotypes for which genes have not been identified. The gene corresponding to this GENSET cDNA or genomic DNA thus becomes an immediate candidate for each of these genetic diseases.

Cells from patients with these diseases or phenotypes are isolated and expanded in culture. PCR primers from the GENSET cDNA or genomic DNA are used to screen genomic DNA, mRNA or cDNA obtained from the patients. GENSET cDNAs or genomic DNAs that are not amplified in he patients can be positively associated with a particular disease by further analysis. Alternatively, the PCR analysis may yield fragments of different lengths when the samples are derived from an individual having the phenotype associated with the disease than when the sample is derived from a healthy individual, indicating that the gene containing the cDNA may be responsible for the genetic disease.

Uses of Polynucleotides in Recombinant Vectors

The present invention also relates to recombinant vectors including the isolated polynucleotides of the present invention, and to host cells recombinant for a polynucleotide of the invention, such as the above vectors, as well as to methods of making such vectors and host cells and for using them for production of GENSET polypeptides by recombinant techniques.

Recombinant Vectors

The term “vector” is used herein to designate either a circular or a linear DNA or RNA molecule, which is either double-stranded or single-stranded, and which comprise at least one polynucleotide of interest that is sought to be transferred in a cell host or in a unicellular or multicellular host organism. The present invention encompasses a family of recombinant vectors that comprise a regulatory polynucleotide and/or a coding polynucleotide derived from either the GENSET genomic sequence or the cDNA sequence. Generally, a recombinant vector of the invention may comprise any of the polynucleotides described herein, including regulatory sequences, coding sequences and polynucleotide constructs, as well as any GENSET primer or probe as defined herein.

In a first preferred embodiment, a recombinant vector of the invention is used to amplify the inserted polynucleotide derived from a GENSET genomic sequence or a GENSET cDNA, for example any cDNA selected from the group consisting of sequences of SEQ ID NOs:1-169, 339-455, 561-784, sequences of clone inserts of the deposited clone pool, variants and fragments thereof in a suitable cell host, this polynucleotide being amplified at every time that the recombinant vector replicates.

A second preferred embodiment of the recombinant vectors according to the invention comprises expression vectors comprising either a regulatory polynucleotide or a coding nucleic acid of the invention, or both. Within certain embodiments, expression vectors are employed to express a GENSET polypeptide which can be then purified and, for example be used in ligand screening assays or as an immunogen in order to raise specific antibodies directed against the GENSET protein. In other embodiments, the expression vectors are used for constructing transgenic animals and also for gene therapy. Expression requires that appropriate signals are provided in the vectors, said signals including various regulatory elements, such as enhancers/promoters from both viral and mammalian sources that drive expression of the genes of interest in host cells. Dominant drug selection markers for establishing permanent, stable cell clones expressing the products are generally included in the expression vectors of the invention, as they are elements that link expression of the drug selection markers to expression of the polypeptide.

More particularly, the present invention relates to expression vectors which include nucleic acids encoding a GENSET protein, preferably a GENSET protein with an amino acid sequence selected from the group consisting of sequences of SEQ ID NOs:170-338, 456-560, 785-918, sequences of polypeptides encoded by the clone inserts of the deposited clone pool, variants and fragments thereof. The polynucleotides of the present invention may be used to express an encoded protein in a host organism to produce a beneficial effect. In such procedures, the encoded protein may be transiently expressed in the host organism or stably expressed in the host organism. The encoded protein may have any of the activities described herein. The encoded protein may be a protein which the host organism lacks or, alternatively, the encoded protein may augment the existing levels of the protein in the host organism.

Some of the elements which can be found in the vectors of the present invention are described in further detail in the following sections.

General Features of the Expression Vectors of the Invention

A recombinant vector according to the invention comprises, but is not limited to, a YAC (Yeast Artificial Chromosome), a BAC (Bacterial Artificial Chromosome), a phage, a phagemid, a cosmid, a plasmid or even a linear DNA molecule which may comprise a chromosomal, non-chromosomal, semi-synthetic and synthetic DNA. Such a recombinant vector can comprise a transcriptional unit comprising an assembly of:

(1) a genetic element or elements having a regulatory role in gene expression, for example promoters or enhancers. Enhancers are cis-acting elements of DNA, usually from about 10 to 300 bp in length that act on the promoter to increase the transcription.

(2) a structural or coding sequence which is transcribed into mRNA and eventually translated into a polypeptide, said structural or coding sequence being operably linked to the regulatory elements described in (1); and

(3) appropriate transcription initiation and termination sequences. Structural units intended for use in yeast or eukaryotic expression systems preferably include a leader sequence enabling extracellular secretion of translated protein by a host cell. Alternatively, when a recombinant protein is expressed without a leader or transport sequence, it may include a N-terminal residue. This residue may or may not be subsequently cleaved from the expressed recombinant protein to provide a final product.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium. In a specific embodiment wherein the vector is adapted for transfecting and expressing desired sequences in mammalian host cells, preferred vectors will comprise an origin of replication in the desired host, a suitable promoter and enhancer, and also any necessary ribosome binding sites, polyadenylation signals, splice donor and acceptor sites, transcriptional termination sequences, and 5′-flanking non-transcribed sequences. DNA sequences derived from the SV40 viral genome, for example SV40 origin, early promoter, enhancer, splice and polyadenylation signals may be used to provide the required non-transcribed genetic elements.

The in vivo expression of a GENSET polypeptide of the present invention may be useful in order to correct a genetic defect related to the expression of the native gene in a host organism, for the treatment or prevention of any disease or condition that can be treated or prevented by increasing the level of GENSET polypeptide expression, or to the production of a biologically inactive GENSET protein. Consequently, the present invention also comprises recombinant expression vectors mainly designed for the in vivo production of a GENSET polypeptide the present invention by the introduction of the appropriate genetic material in the organism or the patient to be treated. This genetic material may be introduced in vitro in a cell that has been previously extracted from the organism, the modified cell being subsequently reintroduced in the said organism, directly in vivo into the appropriate tissue.

Regulatory Elements

The suitable promoter regions used in the expression vectors according to the present invention are chosen taking into account the cell host in which the heterologous gene has to be expressed. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell, such as, for example, a human or a viral promoter.

A suitable promoter may be heterologous with respect to the nucleic acid for which it controls the expression or alternatively can be endogenous to the native polynucleotide containing the coding sequence to be expressed. Additionally, the promoter is generally heterologous with respect to the recombinant vector sequences within which the construct promoter/coding sequence has been inserted.

Promoter regions can be selected from any desired gene using, for example, CAT (chloramphenicol transferase) vectors and more preferably pKK232-8 and pCM7 vectors.

Preferred bacterial promoters are the LacI, LacZ, the T3 or T7 bacteriophage RNA polymerase promoters, the gpt, lambda PR, PL and trp promoters (EP 0036776), the polyhedrin promoter, or the p10 protein promoter from baculovirus (Kit Novagen), (Smith et al., 1983; O'Reilly et al., 1992; which disclosures are hereby incorporated by reference in their entireties), the lambda PR promoter or also the trc promoter.

Eukaryotic promoters include CMV immediate early, HSV thymidine kinase, early and late SV40, LTRs from retrovirus, and mouse metallothionein-L. Selection of a convenient vector and promoter is well within the level of ordinary skill in the art. The choice of a promoter is well within the ability of a person skilled in the field of genetic engineering. For example, one may refer to the book of Sambrook et al., (1989) or also to the procedures described by Fuller et al., (1996), which disclosures are hereby incorporated by reference in their entireties.

Other Regulatory Elements

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

Selectable Markers

Selectable markers confer an identifiable change to the cell permitting easy identification of cells containing the expression construct. The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. Coli, or levan saccharase for mycobacteria, this latter marker being a negative selection marker.

Preferred Vectors

Bacterial Vectors

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), and pGEM1 (Promega Biotec, Madison, Wis., USA).

Large numbers of other suitable vectors are known to those of skill in the art, and commercially available, such as the following bacterial vectors: pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIAexpress).

Bacteriophage Vectors

The P1 bacteriophage vector may contain large inserts ranging from about 80 to about 100 kb. The construction of P1 bacteriophage vectors such as p158 or p158/neo8 are notably described by Sternberg (1992, 1994), which disclosure is hereby incorporated by reference in its entirety. Recombinant P1 clones comprising GENSET nucleotide sequences may be designed for inserting large polynucleotides of more than 40 kb (See Linton et al., 1993), which disclosure is hereby incorporated by reference in its entirety. To generate P1 DNA for transgenic experiments, a preferred protocol is the protocol described by McCormick et al. (1994), which disclosure is hereby incorporated by reference in its entirety. Briefly, E. coli (preferably strain NS3529) harboring the P1 plasmid are grown overnight in a suitable broth medium containing 25 μg/ml of kanamycin. The P1 DNA is prepared from the E. coli by alkaline lysis using the Qiagen Plasmid Maxi kit (Qiagen, Chatsworth, Calif., USA), according to the manufacturer's instructions. The P1 DNA is purified from the bacterial lysate on two Qiagen-tip 500 columns, using the washing and elution buffers contained in the kit. A phenol/chloroform extraction is then performed before precipitating the DNA with 70% ethanol. After solubilizing the DNA in TE (10 mM Tris-HCl, pH 7.4, 1 mM EDTA), the concentration of the DNA is assessed by spectrophotometry.

When the goal is to express a P1 clone comprising GENSET polypeptide-encoding nucleotide sequences in a transgenic animal, typically in transgenic mice, it is desirable to remove vector sequences from the P1 DNA fragment, for example by cleaving the P1 DNA at rare-cutting sites within the P1 polylinker (SfiI, NotI or SalI). The P1 insert is then purified from vector sequences on a pulsed-field agarose gel, using methods similar to those originally reported for the isolation of DNA from YACs (See e. g., Schedl et al., 1993a; Peterson et al., 1993), which disclosures are hereby incorporated by reference in their entireties. At this stage, the resulting purified insert DNA can be concentrated, if necessary, on a Millipore Ultrafree-MC Filter Unit (Millipore, Bedford, Mass., USA—30,000 molecular weight limit) and then dialyzed against microinjection buffer (10 mM Tris-HCl, pH 7.4; 250 μM EDTA) containing 100 mM NaCl, 30 μM spermine, 70 μM spermidine on a microdyalisis membrane (type VS, 0.025 μM from Millipore). The intactness of the purified P1 DNA insert is assessed by electrophoresis on 1% agarose (Sea Kem GTG; FMC Bio-products) pulse-field gel and staining with ethidium bromide.

Viral Vectors

In one specific embodiment, the vector is derived from an adenovirus. Preferred adenovirus vectors according to the invention are those described by Feldman and Steg (1996), or Ohno et al., (1994), which disclosures are hereby incorporated by reference in their entireties. Another preferred recombinant adenovirus according to this specific embodiment of the present invention is the human adenovirus type 2 or 5 (Ad 2 or Ad 5) or an adenovirus of animal origin (French patent application No. FR-93.05954), which disclosure is hereby incorporated by reference in its entirety.

Retrovirus vectors and adeno-associated virus vectors are generally understood to be the recombinant gene delivery systems of choice for the transfer of exogenous polynucleotides in vivo, particularly to mammals, including humans. These vectors provide efficient delivery of genes into cells, and the transferred nucleic acids are stably integrated into the chromosomal DNA of the host. Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-190; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al. (1996), PCT Application No WO 93/25234, PCT Application No WO 94/06920, Roux et al., (1989), Julan et al., (1992), and Neda et al., (1991), which disclosures are hereby incorporated by reference in their entireties.

Yet another viral vector system that is contemplated by the invention comprises the adeno-associated virus (AAV). The adeno-associated virus is a naturally occurring defective virus that requires another virus, such as an adenovirus or a herpes virus, as a helper virus for efficient replication and a productive life cycle (Muzyczka et al., 1992), which disclosure is hereby incorporated by reference in its entirety. It is also one of the few viruses that may integrate its DNA into non-dividing cells, and exhibits a high frequency of stable integration (Flotte et al. 1992; Samulski et al., 1989; McLaughlin et al., 1989), which disclosures are hereby incorporated by reference in their entireties. One advantageous feature of AAV derives from its reduced efficacy for transducing primary cells relative to transformed cells.

BAC Vectors

The bacterial artificial chromosome (BAC) cloning system (Shizuya et al., 1992), which disclosure is hereby incorporated by reference in its entirety, has been developed to stably maintain large fragments of genomic DNA (100-300 kb) in E. coli. A preferred BAC vector comprises a pBeloBACll vector that has been described by Kim et al. (1996), which disclosure is hereby incorporated by reference in its entirety. BAC libraries are prepared with this vector using size-selected genomic DNA that has been partially digested using enzymes that permit ligation into either the Bam HI or HindIII sites in the vector. Flanking these cloning sites are T7 and SP6 RNA polymerase transcription initiation sites that can be used to generate end probes by either RNA transcription or PCR methods. After the construction of a BAC library in E. coli, BAC DNA is purified from the host cell as a supercoiled circle. Converting these circular molecules into a linear form precedes both size determination and introduction of the BACs into recipient cells. The cloning site is flanked by two Not I sites, permitting cloned segments to be excised from the vector by Not I digestion. Alternatively, the DNA insert contained in the pBeloBACll vector may be linearized by treatment of the BAC vector with the commercially available enzyme lambda terminase that leads to the cleavage at the unique cosN site, but this cleavage method results in a full length BAC clone containing both the insert DNA and the BAC sequences.

Baculovirus

Another specific suitable host vector system is the pVL1392/1393 baculovirus transfer vector (Pharmingen) that is used to transfect the SF9 cell line (ATCC No. CRL 1711) which is derived from Spodoptera frugiperda. Other suitable vectors for the expression of the GENSET polypeptide of the present invention in a baculovirus expression system include those described by Chai et al., (1993), Vlasak et al., (1983), and Lenhard et al., (1996), which disclosures are hereby incorporated by reference in their entireties.

Delivery of the Recombinant Vectors

To effect expression of the polynucleotides and polynucleotide constructs of the invention, the constructs must be delivered into a cell. This delivery may be accomplished in vitro, as in laboratory procedures for transforming cell lines, or in vivo or ex vivo, as in the treatment of certain diseases states. One mechanism is viral infection where the expression construct is encapsulated in an infectious viral particle.

Several non-viral methods for the transfer of polynucleotides into cultured mammalian cells are also contemplated by the present invention, and include, without being limited to, calcium phosphate precipitation (Graham et al., 1973; Chen et al., 1987); DEAE-dextran (Gopal, 1985); electroporation (Tur-Kaspa et al., 1986; Potter et al., 1984); direct microinjection (Harland et al., 1985); DNA-loaded liposomes (Nicolau et al., 1982; Fraley et al., 1979); and receptor-mediated transfection. (Wu and Wu, 1987, 1988), which disclosures are hereby incorporated by reference in their entireties. Some of these techniques may be successfully adapted for in vivo or ex vivo use.

Once the expression polynucleotide has been delivered into the cell, it may be stably integrated into the genome of the recipient cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle.

One specific embodiment for a method for delivering a protein or peptide to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect. This is particularly applicable for transfer in vitro but it may be applied to in vivo as well.

Compositions for use in vitro and in vivo comprising a “naked” polynucleotide are described in PCT application No. WO 90/11092 (Vical Inc.) and also in PCT application No. WO 95/11307 (Institut Pasteur, INSERM, Université d'Ottawa) as well as in the articles of Tascon et al. (1996) and of Huygen et al., (1996), which disclosures are hereby incorporated by reference in their entireties.

In still another embodiment of the invention, the transfer of a naked polynucleotide of the invention, including a polynucleotide construct of the invention, into cells may be accomplished with particle bombardment (biolistic), said particles being DNA-coated microprojectiles accelerated to a high velocity allowing them to pierce cell membranes and enter cells without killing them, such as described by Klein et al., (1987), which disclosure is hereby incorporated by reference in its entirety.

In a further embodiment, the polynucleotide of the invention may be entrapped in a liposome (Ghosh and Bacchawat, 1991; Wong et al., 1980; Nicolau et al., 1987, which disclosures are hereby incorporated by reference in their entireties).

In a specific embodiment, the invention provides a composition for the in vivo production of the GENSET polypeptides described herein. It comprises a naked polynucleotide operatively coding for this polypeptide, in solution in a physiologically acceptable carrier, and suitable for introduction into a tissue to cause cells of the tissue to express the said protein or polypeptide.

The amount of vector to be injected to the desired host organism varies according to the site of injection. As an indicative dose, it will be injected between 0.1 and 100 μg of the vector in an animal body, preferably a mammal body, for example a mouse body.

In another embodiment of the vector according to the invention, it may be introduced in vitro in a host cell, preferably in a host cell previously harvested from the animal to be treated and more preferably a somatic cell such as a muscle cell. In a subsequent step, the cell that has been transformed with the vector coding for the desired GENSET polypeptide or the desired fragment thereof is reintroduced into the animal body in order to deliver the recombinant protein within the body either locally or systemically.

Secretion Vectors

Some of the GENSET cDNAs or genomic DNAs of the invention may also be used to construct secretion vectors capable of directing the secretion of the proteins encoded by genes inserted in the vectors. Such secretion vectors may facilitate the purification or enrichment of the proteins encoded by genes inserted therein by reducing the number of background proteins from which the desired protein must be purified or enriched. Exemplary secretion vectors are described below.

The secretion vectors of the present invention include a promoter capable of directing gene expression in the host cell, tissue, or organism of interest. Such promoters include the Rous Sarcoma Virus promoter, the SV40 promoter, the human cytomegalovirus promoter, and other promoters familiar to those skilled in the art.

A signal sequence from a polynucleotide of the invention, preferably a signal sequences selected from the group of signal sequences of SEQ ID NOs: 1-85, 339-400, 406-407, 413-415, 561-594, and 634-651 and signal sequences of clone inserts of the deposited clone pool is operably linked to the promoter such that the mRNA transcribed from the promoter will direct the translation of the signal peptide. The host cell, tissue, or organism may be any cell, tissue, or organism which recognizes the signal peptide encoded by the signal sequence in the GENSET cDNA or genomic DNA. Suitable hosts include mammalian cells, tissues or organisms, avian cells, tissues, or organisms, insect cells, tissues or organisms, or yeast.

In addition, the secretion vector contains cloning sites for inserting genes encoding the proteins which are to be secreted. The cloning sites facilitate the cloning of the insert gene in frame with the signal sequence such that a fusion protein in which the signal peptide is fused to the protein encoded by the inserted gene is expressed from the mRNA transcribed from the promoter. The signal peptide directs the extracellular secretion of the fusion protein.

The secretion vector may be DNA or RNA and may integrate into the chromosome of the host, be stably maintained as an extrachromosomal replicon in the host, be an artificial chromosome, or be transiently present in the host. Preferably, the secretion vector is maintained in multiple copies in each host cell. As used herein, multiple copies means at least 2, 5, 10, 20, 25, 50 or more than 50 copies per cell. In some embodiments, the multiple copies are maintained extrachromosomally. In other embodiments, the multiple copies result from amplification of a chromosomal sequence.

Many nucleic acid backbones suitable for use as secretion vectors are known to those skilled in the art, including retroviral vectors, SV40 vectors, Bovine Papilloma Virus vectors, yeast integrating plasmids, yeast episomal plasmids, yeast artificial chromosomes, human artificial chromosomes, P element vectors, baculovirus vectors, or bacterial plasmids capable of being transiently introduced into the host.

The secretion vector may also contain a polyA signal such that the polyA signal is located downstream of the gene inserted into the secretion vector.

After the gene encoding the protein for which secretion is desired is inserted into the secretion vector, the secretion vector is introduced into the host cell, tissue, or organism using calcium phosphate precipitation, DEAE-Dextran, electroporation, liposome-mediated transfection, viral particles or as naked DNA. The protein encoded by the inserted gene is then purified or enriched from the supernatant using conventional techniques such as ammonium sulfate precipitation, immunoprecipitation, immunochromatography, size exclusion chromatography, ion exchange chromatography, and hplc. Alternatively, the secreted protein may be in a sufficiently enriched or pure state in the supernatant or growth media of the host to permit it to be used for its intended purpose without further enrichment.

The signal sequences may also be inserted into vectors designed for gene therapy. In such vectors, the signal sequence is operably linked to a promoter such that mRNA transcribed from the promoter encodes the signal peptide. A cloning site is located downstream of the signal sequence such that a gene encoding a protein whose secretion is desired may readily be inserted into the vector and fused to the signal sequence. The vector is introduced into an appropriate host cell. The protein expressed from the promoter is secreted extracellularly, thereby producing a therapeutic effect.

Cell Hosts

Another object of the invention comprises a host cell that has been transformed or transfected with one of the polynucleotides described herein, and in particular a polynucleotide either comprising a GENSET polypeptide-encoding polynucleotide regulatory sequence or the polynucleotide coding for a GENSET polypeptide. Also included are host cells that are transformed (prokaryotic cells) or that are transfected (eukaryotic cells) with a recombinant vector such as one of those described above. However, the cell hosts of the present invention can comprise any of the polynucleotides of the present invention. In a preferred embodiment, host cells contain a polynucleotide sequence comprising a sequence selected from the group consisting of sequences of SEQ ID NOs:1-169, 339-455, 561-784, sequences of clone inserts of the deposited clone pool, variants and fragments thereof. Preferred host cells used as recipients for the expression vectors of the invention are the following:

a) Prokaryotic host cells: Escherichia coli strains (I.E.DH5-α strain), Bacillus subtilis, Salmonella typhimurium, and strains from species like Pseudomonas, Streptomyces and Staphylococcus.

b) Eukaryotic host cells: HeLa cells (ATCC No.CCL2; No.CCL2.1; No.CCL2.2), Cv 1 cells (ATCC No.CCL70), COS cells (ATCC No.CRL1650; No.CRL1651), Sf-9 cells (ATCC No.CRL1711), C127 cells (ATCC No. CRL-1804), 3T3 (ATCC No. CRL-6361), CHO (ATCC No. CCL-61), human kidney 293. (ATCC No. 45504; No. CRL-1573) and BHK (ECACC No. 84100501; No. 84111301).

### c) Other Mammalian Host Cells

The present invention also encompasses primary, secondary, and immortalized homologously recombinant host cells of vertebrate origin, preferably mammalian origin and particularly human origin, that have been engineered to: a) insert exogenous (heterologous) polynucleotides into the endogenous chromosomal DNA of a targeted gene, b) delete endogenous chromosomal DNA, and/or c) replace endogenous chromosomal DNA with exogenous polynucleotides. Insertions, deletions, and/or replacements of polynucleotide sequences may be to the coding sequences of the targeted gene and/or to regulatory regions, such as promoter and enhancer sequences, operably associated with the targeted gene.

In addition to encompassing host cells containing-the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., (1989); and Zijlstra et al. (1989) (the disclosures of each of which are incorporated by reference in their entireties).

The present invention further relates to a method of making a homologously recombinant host cell in vitro or in vivo, wherein the expression of a targeted gene not normally expressed in the cell is altered. Preferably the alteration causes expression of the targeted gene under normal growth conditions or under conditions suitable for producing the polypeptide encoded by the targeted gene. The method comprises the steps of: (a) transfecting the cell in vitro or in vivo with a polynucleotide construct, said polynucleotide construct comprising; (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination.

The present invention further relates to a method of altering the expression of a targeted gene in a cell in vitro or in vivo wherein the gene is not normally expressed in the cell, comprising the steps of: (a) transfecting the cell in vitro or in vivo with a polynucleotide construct, said polynucleotide construct comprising: (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination, thereby producing a homologously recombinant cell; and (c) maintaining the homologously recombinant cell in vitro or in vivo under conditions appropriate for expression of the gene.

The present invention further relates to a method of making a polypeptide of the present invention by altering the expression of a targeted endogenous gene in a cell in vitro or in vivo wherein the gene is not normally expressed in the cell, comprising the steps of: a) transfecting the cell in vitro with a polynucleotide construct, said polynucleotide construct comprising: (i) a targeting sequence; (ii) a regulatory sequence and/or a coding sequence; and (iii) an unpaired splice donor site, if necessary, thereby producing a transfected cell; (b) maintaining the transfected cell in vitro or in vivo under conditions appropriate for homologous recombination, thereby producing a homologously recombinant cell; and c) maintaining the homologously recombinant cell in vitro or in vivo under conditions appropriate for expression of the gene thereby making the polypeptide.

The present invention further relates to a polynucleotide construct which alters the expression of a targeted gene in a cell type in which the gene is not normally expressed. This occurs when the polynucleotide construct is inserted into the chromosomal DNA of the target cell, wherein said polynucleotide construct comprises: a) a targeting sequence; b) a regulatory sequence and/or coding sequence; and c) an unpaired splice-donor site, if necessary. Further included are a polynucleotide construct, as described above, wherein said polynucleotide construct further comprises a polynucleotide which encodes a polypeptide and is in-frame with the targeted endogenous gene after homologous recombination with chromosomal DNA.

The compositions may be produced, and methods performed, by techniques known in the art, such as those described in U.S. Pat. NOs: 6,054,288; 6,048,729; 6,048,724; 6,048,524; 5,994,127; 5,968,502; 5,965,125; 5,869,239; 5,817,789; 5,783,385; 5,733,761; 5,641,670; 5,580,734; International Publication NOs:WO96/29411, WO 94/12650; and scientific articles described by Koller et al., (1994). (The disclosures of each of which are incorporated by reference in their entireties).

GENSET gene expression in mammalian cells, preferably human cells, may be rendered defective, or alternatively may be altered by replacing endogenous GENSET polypeptide-encoding genes in the genome of an animal cell by a GENSET polypeptide-encoding polynucleotide according to the invention. These genetic alterations may be generated by homologous recombination using previously described specific polynucleotide constructs.

Mammal zygotes, such as murine zygotes may be used as cell hosts. For example, murine zygotes may undergo microinjection with a purified DNA molecule of interest, for example a purified DNA molecule that has previously been adjusted to a concentration ranging from 1 ng/ml—for BAC inserts—to 3 ng/μl—for P1 bacteriophage inserts—in 10 mM Tris-HCl, pH 7.4, 250 μM EDTA containing 100 mM NaCl, 30 μM spermine, and70 μM spermidine. When the DNA to be microinjected has a large size, polyamines and high salt concentrations can be used in order to avoid mechanical breakage of this DNA, as described by Schedl et al (1993b), which disclosure is hereby incorporated by reference in its entirety.

Any one of the polynucleotides of the invention, including the polynucleotide constructs described herein, may be introduced in an embryonic stem (ES) cell line, preferably a mouse ES cell line. ES cell lines are derived from pluripotent, uncommitted cells of the inner cell mass of pre-implantation blastocysts. Preferred ES cell lines are the following: ES-E14TG2a (ATCC No.CRL-1821), ES-D3 (ATCC No.CRL1934 and No. CRL-11632), YS001 (ATCC No. CRL-11776), 36.5 (ATCC No. CRL-11116). ES cells are maintained in an uncommitted state by culture in the presence of growth-inhibited feeder cells which provide the appropriate signals to preserve this embryonic phenotype and serve as a matrix for ES cell adherence. Preferred feeder cells are primary embryonic fibroblasts that are established from tissue of day 13-day 14 embryos of virtually any mouse strain, that are maintained in culture, such as described by Abbondanzo et al. (1993) and are growth-inhibited by irradiation, such as described by Robertson (1987), or by the presence of an inhibitory concentration of LIF, such as described by Pease and Williams (1990), which disclosures are hereby incorporated by reference in their entireties.

The constructs in the host cells can be used in a conventional manner to produce the gene product encoded by the recombinant sequence.

Following transformation of a suitable host and growth of the host to an appropriate cell density, the selected promoter is induced by appropriate means, such as temperature shift or chemical induction, and cells are cultivated for an additional period. Cells are typically harvested by centrifugation, disrupted by physical or chemical means, and the resulting crude extract retained for further purification. Microbial cells employed in the expression of proteins can be disrupted by any convenient method, including freeze-thaw cycling, sonication, mechanical disruption, or use of cell lysing agents. Such methods are well known by the skilled artisan.

Transgenic Animals

The terms “transgenic animals” or “host animals” are used herein to designate animals that have their genome genetically and artificially manipulated so as to include one of the nucleic acids according to the invention. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention. In one embodiment, the invention encompasses non-human host mammals and animals comprising a recombinant vector of the invention or a GENSET gene disrupted by homologous recombination with a knock out vector.

The transgenic animals of the invention all include within a plurality of their cells a cloned recombinant or synthetic DNA sequence, more specifically one of the purified or isolated nucleic acids comprising a GENSET polypeptide coding sequence, a GENSET polynucleotide regulatory sequence, a polynucleotide construct, or a DNA sequence encoding an antisense polynucleotide such as described in the present specification.

Generally, a transgenic animal according the present invention comprises any of the polynucleotides, the recombinant vectors and the cell hosts described in the present invention. In a first preferred embodiment, these transgenic animals may be good experimental models in order to study the diverse pathologies related to the dysregulation of the expression of a given GENSET gene, in particular the transgenic animals containing within their genome one or several copies of an inserted polynucleotide encoding a native GENSET polypeptide, or alternatively a mutant GENSET polypeptide.

In a second preferred embodiment, these transgenic animals may express a desired polypeptide of interest under the control of the regulatory polynucleotides of the GENSET gene, leading to high yields in the synthesis of this protein of interest, and eventually to tissue specific expression of the protein of interest.

The design of the transgenic animals of the invention may be made according to the conventional techniques well known from the one skilled in the art. For more details regarding the production of transgenic animals, and specifically transgenic mice, it may be referred to U.S. Pat. No. 4,873,191, issued Oct. 10, 1989; U.S. Pat. No. 5,464,764 issued Nov. 7, 1995; and U.S. Pat. No. 5,789,215, issued Aug. 4, 1998; these documents being herein incorporated by reference to disclose methods producing transgenic mice.

Transgenic animals of the present invention are produced by the application of procedures which result in an animal with a genome that has incorporated exogenous genetic material. The procedure involves obtaining the genetic material which encodes either a GENSET polypeptide coding sequence, a GENSET polynucleotide regulatory sequence, or a DNA sequence encoding a GENSET polynucleotide antisense sequence, or a portion thereof, such as described in the present specification. A recombinant polynucleotide of the invention is inserted into an embryonic or ES stem cell line. The insertion is preferably made using electroporation, such as described by Thomas et al. (1987), which disclosure is hereby incorporated by reference in its entirety. The cells subjected to electroporation are screened (e.g. by selection via selectable markers, by PCR or by Southern blot analysis) to find positive cells which have integrated the exogenous recombinant polynucleotide into their genome, preferably via an homologous recombination event. An illustrative positive-negative selection procedure that may be used according to the invention is described by Mansour et al. (1988), which disclosure is hereby incorporated by reference in its entirety.

The positive cells are then isolated, cloned and injected into 3.5 days old blastocysts from mice, such as described by Bradley (1987), which disclosure is hereby incorporated by reference in its entirety. The blastocysts are then inserted into a female host animal and allowed to grow to term. Alternatively, the positive ES cells are brought into contact with embryos at the 2.5 days old 8-16 cell stage (morulae) such as described by Wood et al. (1993), or by Nagy et al. (1993), which disclosures are hereby incorporated by reference in their entireties, the ES cells being internalized to colonize extensively the blastocyst including the cells which will give rise to the germ line.

The offspring of the female host are tested to determine which animals are transgenic e.g. include the inserted exogenous DNA sequence and which ones are wild type.

Thus, the present invention also concerns a transgenic animal containing a nucleic acid, a recombinant expression vector or a recombinant host cell according to the invention.

In another embodiment, transgenic animals are produced by microinjecting polynucleotides ares microinjected into a fertilized oocyte. Typically, fertilized oocytes are microinjected using standard techniques, and then cultured in vitro until a “pre-implantation embryo” is obtained. Such pre-implantation embryos preferably contain approximately 16 to 150 cells. Methods for culturing fertilized oocytes to the pre-implantation stage are described, e.g., by Gordon et al. ((1984) Methods in Enzymology, 101, 414); Hogan et al. ((1986) in Manipulating the mouse embryo. A Laboratory Manual. Cold Spring Harbor Laboratory Press, Cold Spring Harbor, N.Y) (for the mouse embryo); Hammer et al. ((1985) Nature, 315, 680) (for rabbit and porcine embryos); Gandolfi et al. ((1987) J. Reprod. Fert. 81, 23-28); Rexroad et al. ((1988) J. Anim. Sci. 66, 947-953) (for ovine embryos); and Eyestone et al. ((1989) J. Reprod. Fert. 85, 715-720); Camous et al. ((1984) J. Reprod. Fert. 72, 779-785); and Heyman et al. ((1987) Theriogenology 27, 5968) (for bovine embryos); the disclosures of each of which are incorporated herein in their entireties. Pre-implantation embryos are then transferred to an appropriate female by standard methods to permit the birth of a transgenic or chimeric animal, depending upon the stage of development when the transgene is introduced.

As the frequency of transgene incorporation is often low, the detection of transgene integration in pre-implantation embryos is often desirable using any of the herein-described methods. Any of a number of methods can be used to detect the presence of a transgene in a pre-implantation embryo. For example, one or more cells may be removed from the pre-implantation embryo, and the presence or absence of the transgene in the removed cell or cells can be detected using any standard method e.g. PCR. Alternatively, the presence of a transgene can be detected in utero or post partum using standard methods.

In a particularly preferred embodiment of the present invention, transgenic mammals are generated that secrete recombinant GENSET polypeptides in their milk. As the mammary gland is a highly efficient protein-producing organ, such methods can be used to produce protein concentrations in the gram per liter range, and often significantly more. Preferably, expression in the mammary gland is accomplished by operably linking the polynucleotide encoding the GENSET polypeptide to a mammary gland specific promoter and, optionally, other regulatory elements. Suitable promoters and other elements include, but are not limited to, those derived from mammalian short and long WAP, alpha, beta, and kappa, casein, alpha and beta lactoglobulin, beta-CN 5′ genes, as well as the the mouse mammary tumor virus (MMTV) promoter. Such promoters and other elements may be derived from any mammal, including, but not limited to, cows, goats, sheep, pigs, mice, rabbits, and guinea pigs. Promoter and other regulatory sequences, vectors, and other relevant teachings are provided, e.g., by Clark (1998) J Mammary Gland Biol Neoplasia 3:337-50; Jost et al. (1999) Nat. Biotechnol 17:1604; U.S. Pat. Nos. 5,994,616; 6,140,552; 6,013,857; Sohn et al. (1999) DNA Cell Biol. 18:845-52; Kim et al. (1999) J. Biochem. (Japan) 126:320-5; Soulier et al. (1999) Euro. J. Biochem. 260:533-9; Zhang et al. (1997) Chin. J. Biotech. 13:271-6; Rijnkels et al. (1998) Transgen. Res. 7:5-14; Korhonen et al. (1997) Euro. J. Biochem. 245:482-9; Uusi-Oukari et al. (1997) Transgen. Res. 6:75-84; Hitchin et al. (1996) Prot. Expr. Purif. 7:247-52; Platenburg et al. (1994) Transgen. Res. 3:99-108; Heng-Cherl et al. (1993) Animal Biotech. 4:89-107; and Christa et al. (2000) Euro. J. Biochem. 267:1665-71; the entire disclosures of each of which is herein incorporated by reference.

In another embodiment, the polypeptides of the invention can be produced in milk by introducing polynucleotides encoding the polypeptides into somatic cells of the mammary gland in vivo, e.g. mammary secreting epithelial cells. For example, plasmid DNA can be infused through the nipple canal, e.g. in association with DEAE-dextran (see, e.g., Hens et al. (2000) Biochim. Biophys. Acta 1523:161-171), in association with a ligand that can lead to receptor-mediated endocytosis of the construct (see, e.g., Sobolev et al. (1998) 273:7928-33), or in a viral vector such as a retroviral vector, e.g. the Gibbon ape leukemia virus (see, e.g., Archer et al. (1994) PNAS 91:6840-6844). In any of these embodiments, the polynucleotide may be operably linked to a mammary gland specific promoter, as described above, or, alternatively, any strongly expressing promoter such as CMV or MoMLV LTR.

The suitability of any vector, promoter, regulatory element, etc. for use in the present invention can be assessed beforehand by transfecting cells such as mammary epithelial cells, e.g. MacT cells (bovine mammary epithelial cells) or GME cells (goat mammary epithelial cells), in vitro and assessing the efficiency of transfection and expression of the transgene in the cells.

For in vivo administration, the polynucleotides can be administered in any suitable formulation, at any of a range of concentrations (e.g. 1-500 μg/ml, preferably 50-100 μg/ml), at any volume (e.g. 1-100 ml, preferably 1 to 20 ml), and can be administered any number of times (e.g. 1, 2, 3, 5, or 10 times), at any frequency (e.g. every 1, 2, 3, 5, 10, or any number of days). Suitable concentrations, frequencies, modes of administration, etc. will depend upon the particular polynucleotide, vector, animal, etc., and can readily be determined by one of skill in the art.

In a preferred embodiment, a retroviral vector such as as Gibbon ape leukemia viral vector is used, as described in Archer et al. ((1994) PNAS 91:6840-6844). As retroviral infection typically requires cell division, cell division in the mammary glands can be stimulated in conjunction with the administration of the vector, e.g. using a factor such as estrodiol benzoate, progesterone, reserpine, or dexamethasone. Further, retroviral and other methods of infection can be facilitated using accessory compounds such as polybrene.

In any of the herein-described methods for obtaining GENSET polypeptides from milk, the quantity of milk obtained, and thus the quantity of GENSET polypeptides produced, can be enhanced using any standard method of lacation induction, e.g. using hexestrol, estrogen, and/or progesterone.

The polynucleotides used in such embodiments can either encode a full-length GENSET protein or a GENSET fragment. Typically, the encoded polypeptide will include a signal sequence to ensure the secretion of the protein into the milk.

Recombinant Cell Lines Derived From the Transgenic Animals of the Invention

A further object of the invention comprises recombinant host cells obtained from a transgenic animal described herein. In one embodiment the invention encompasses cells derived from non-human host mammals and animals comprising a recombinant vector of the invention or a GENSET gene disrupted by homologous recombination with a knock out vector.

Recombinant cell lines may be established in vitro from cells obtained from any tissue of a transgenic animal according to the invention, for example by transfection of primary cell cultures with vectors expressing onc-genes such as SV40 large T antigen, as described by Chou (1989), and Shay et al. (1991), which disclosures are hereby incorporated by reference in their entireties.

### Uses of Polypeptides of the Invention

The polypeptides and polynucleotides of the present invention can be used in any of a large number of ways, including numerous in vitro and in vivo uses. Specific uses for many of the herein-described polypeptides and polynucleotides are described in detail below.

Protein of SEQ ID NO:255 (internal designation 500762786_{—}255-24-5-0-A2-R_{—}104)

The cDNA of clone 500762786_{—}255-24-5-0-A2-R_{—}104 (SEQ ID NO:86) encodes the human EDR4 protein LFPAPAPPPAPAFAPPPKVPSPERSAPRVPLPSPQPSYPFRPAASGGTPPPACLPPAQPCQGSP AMNLFRFLGDLSHLLAIILLLLKIWKSRSCAAHPQLPLSFCLSVCLSVSLSLSXSLSLSFSVSK KKKK (SEQ ID NO:255). It will be appreciated that all characteristics and uses of the polynucleotides of SEQ ID NO:86 and polypeptides of SEQ ID NO:355, described throughout the present application also pertain to the human cDNA of clone 500762786_{—}255-24-5-0-A2-R_{—}104 and polypeptides encoded thereby. Polypeptide fragments having a biological activity described herein and polynucleotides encoding the same are included in the present invention. Related polynucleotide and polypeptide sequences included in the present invention are SEQ ID NOs:406 and 520.

The normal functioning of the eukaryotic cell requires that all newly synthesized proteins be correctly folded, modified, and delivered to specific inter and extracellular sites. Newly synthesized membrane and secretory proteins enter a cellular sorting and distribution network during or immediately after synthesis (cotranslationally or posttranslationally) and are routed to specific locations inside and outside of the cell. The initial compartment in this process is the endoplasmic reticulum (ER) where proteins undergo modifications such as glycosylation, disulfide bond formation, and assembly into oligomers. The proteins are then transported through an additional series of membrane-bound compartments which include the various cistemae of the Golgi complex, where further carbohydrate modifications occur. Transport between compartments occurs by means of vesicles that bud and fuse in a specific manner; once within the secretory pathway, proteins do not have to cross a membrane to reach the cell surface.

The complexity of this system has advantages for the cell because it allows proteins to fold and mature in closed compartments that contain the appropriate enzyme catalysts. It is, however, dependent on sorting mechanisms that position the enzymes correctly and maintain them in place.

The first organelle in this system, the ER, contains multiple enzymes involved in protein structure modifications. Among these are BiP (binding protein) which directs the correct folding of proteins and, PDI (protein disulfide isomerase) and a homologue of the 90 kDa heat-shock protein, both of which catalyze the formation and rearrangement of disulfide bonds (Gething, M. J. and Sambrook, J. (1992) Nature 355:33-45). These abundant soluble proteins must be retained in the ER and must be distinguished from the newly synthesized secretory proteins which are rapidly transported to the Golgi apparatus. The signal for retention in the ER in mammalian cells consists of the tetrapeptide sequence, KDEL, located at the carboxy terminus of proteins. This sequence was first identified when the sequences of rat BiP and PDI were compared and it was subsequently found at the carboxy terminus of other luminal ER proteins from a number of species (Munro, S. (1986) Cell 46:291-300; Pelham, H. R. (1989) Ann. Rev. Cell. Biol. 5:1-23). Proteins containing this sequence leave the ER but are quickly retrieved from the early Golgi compartment and returned to the ER, while proteins without this signal continue through the distribution pathway.

Two endoplasmic retrieval receptors were first identified in S. cerevesiae; two human endoplasmic retrieval receptors were subsequently isolated by the use of degenerate PCR primers based on the S. cerevesiae sequences (Hardwick, K. G. (1990) EMBO J. 9:623-630; Semenza, J. C. (1990) Cell 61:1349-1357; Lewis, M. J. and Pelham, H. R. (1990) Nature 348:162-163; Lewis, M. J. and Pelham, H. R. (1992) J. Mol. Biol. 226:913-916). Comparisons of these sequences shows that they consist of a conserved 7-transmembrane domain structure with only short loops in the cell cytoplasm and the ER lumen. Studies with these endoplasmic retrieval receptors show that ligand binding controls the movement of the receptor; when expressed in COS cells, the human receptor is normally concentrated in the Golgi, but moves to the ER when bound to a ligand such as KDEL-tagged hen lysozyme (Lewis, M. J. and Pelham, H. R. (1992) Cell 68:353-364).

The ER retrieval function of these molecules serves to maintain the pool of enzymes in the ER that are necessary to perform protein structure modifications, retains newly synthesized proteins in the ER until they have been correctly modified, and regulates the structure of the Golgi apparatus. Saccharomyces cerevisiae cells that lack an ER retrieval receptor (Erd2) have a defective Golgi apparatus and fail to grow. Analysis of yeast Erd2 mutants suggests that their growth requires both the retention of multiple proteins in the ER and the selective removal of specific proteins from the Golgi (Townsley, F. M. (1994) J. Cell Biol. 127:21-28). Overexpression of a human ER retrieval receptor in COS cells results in hyperactive retrograde traffic from the Golgi to the ER leading to a loss of the Golgi structure and the breakdown of the secretory pathway (Hsu V. W. (1992) Cell 69:625-635).

Disruptions in the cellular secretory pathway have been implicated in several human diseases. In familial hypercholesterolemia the low density lipoprotein receptors remain in the ER, rather than moving to the cell surface (Pathak, R. K. (1988) J. Cell Biol. 106:1831-1841). A form of congenital hypothyroidism is produced by a deficiency of thyroglobulin, the thyroid prohormone. In this disease the thyroglobulin is incorrectly folded and is therefore retained in the ER (Kim, P. S. (1996) J.Cell Biol. 133:517-527). Mutant forms of proteolipid protein (PLP) have been examined as they play a role in generating dysmyelinating or hypomyelinating diseases. In this case, the mutations that result in disease are mutations that arrest transport of PLP in the ER and the early Golgi; the subsequent accumulation of PLP in the ER results in rapid oligodendrocyte death (Gow, A. (1994) J. Neurosci. Res. 37:574-583).

The human ER retrieval receptor function is necessary for processing and presentation of specific antigens to T cells. Many antigens must be processed intracellularly before they can be presented, in association with major histocompatability complex (MHC) molecules at the cell surface, for recognition by the antigen-specific receptor of T cells. Disruption of the ER retrieval receptor function with an antibiotic, Brefeldin A, abolishes the ability of a cell to present these specific antigen complexes to T cells. These antigenic proteins must be retained in the ER for cleavage to smaller peptides which can then bind to MHC molecules and be released for presentation at the cell surface. (Kakiuchi, T. (1991) J. Immunol. 147:3289-3295).

The discovery of polynucleotides encoding a novel human KDEL receptor, and the molecules themselves, provides the means to further investigate the regulation of the cellular protein secretory pathway. Discovery of molecules related to a novel human KDEL receptor satisfies a need in the art by providing a means or a tool for the study of this pathway and the diseases that involve the dysfunction of this pathway.

In an embodiment of the present invention, ERD4 polypeptides of the present invention are used to purify KDEL containing proteins and other homologous proteins with similar signals for ER retention such as “HDEL”, “DDEL”, “ADEL”, “SDEL”, “RDEL”, “KEEL”, “QEDL”, “HIEL”, “HTEL” and “KQDL”. This may be carried out by covalently or non-covalently attached the EDR4 polypeptides of the present invention to a column or other solid support using techniques well known in the art (e.g., affinity chromatography, panning, etc.). Once bound to the ERD4 polypeptide, the complex is washed to remove contaminants. The target protein is released using increasing salt concentrations either in a gradient or step type purification. The bound target protein may also be released from the ERD4 polypeptide by a single step up in salt concentration.

In another embodiment of the present invention, the EDR4 polypeptides of the present invention are used to detect KDEL containing proteins and other homologous sequences as described above by methods comprising the steps of contacting KDEL or other homologous sequences with an EDR4 polypeptide under conditions that allow binding to said sequence, and detecting the presence of bound EDR4. The presence of bound EDR4 can be detected using methods known in the art, such as by labeling EDR4 directly or indirectly. Bound EDR4 can be detected, for example, by using an antibody that specifically binds to EDR4 or another EDR4-binding compound that is detectable directly or indirectly.

Preferred ERD4 polypeptides for binding KDEL containing proteins and other homologous sequences described above comprise the amino acid sequence -KIWK- or -MNLFRFLGDLSHLLAIILLLLKIWKSRSCA-.

The present invention is further directed to a transformant comprising the following expression units in a co-expressible state: an expression unit containing a gene coding for an ERD4 polypeptide which is capable of binding to a protein localizing in the endoplasmic reticulum and having a signal for staying therein; an expression unit containing a gene coding for said protein localizing in endoplasmic reticulum; and an expression unit containing a foreign gene coding for a polypeptide which is a subject of function of said protein localizing in endoplasmic reticulum, and to a transformant comprising, in a co-expressible state, a fusion gene which is composed of a DNA fragment coding for a human serum albumin prepro-sequence and a foreign gene coding for a useful polypeptide. The present invention is also directed to a process for producing said polypeptide by co-expressing said genes in said transformant such that the polypeptide is predominantly secreted out of the transformant cell. Consequently, the invention has an advantage of improving the productivity of said polypeptide.

More particularly, the invention relates to: A transformed yeast cell comprising the following expression units integrated on a yeast chromosome in a co-expressible state: a first expression unit containing a gene coding for a receptor for an endoplasmic reticulum retention signal, wherein the receptor is the receptor protein ERD4 or a fragment thereof which is capable of binding to a retention signal selected from the group consisting of “KDEL”, “HDEL”, “DDEL”, “ADEL”, “SDEL”, “RDEL”, “KEEL”, “QEDL”, “HIEL”, “HTEL” and “KQDL”. and a second expression unit containing a gene encoding a protein disulfide isomerase, wherein said isomerase comprises an endoplasmic reticulum retention signal, or a gene encoding a fusion protein comprising the amino acid sequence of said isomerase and a human serum albumin prepro-sequence. These methods can be carried out using methods known in the art or described in U.S. Pat. No. 5,578,466, incorporated herein by reference in its entirety.

Proteins of SEQ ID NO:193 and 194 (internal designation 585770_{—}215-16-5-0-E8-F and 123996_{—}140-002-5-0-B4-F)

The cDNA of clones 585770_{—}215-16-5-0-E8-F (SEQ ID NO:24) and 123996_{—}140-002-5-0 B4-F (SEQ ID NO:25) encode the human Smooth Muscle and Pain Effector (SMPE) proteins: MRGATRVSIMLLLVTVSDCAVITGACERDVQCGAGTCCAISLWLRGLRMCTPLGRXGEEC HPGSHKIPFFRKRKHHTCPCLPNLLCSRFPDGRYRCSMDLKNINF (SEQ ID NO: 193) and MRGATRVSIMLLLVTVSDCAVITGACERDVQCGAGTCCAISLWLRGLRMCTPLGREGEEC HPGSHKIPFFRKRKHHTCPCLPNLLCSRFPDGRYRCSMDLKNINF (SEQ ID NO: 194), respectively. It will be appreciated that all characteristics and uses of the polynucleotides of SEQ ID NOs:24 and 25 and polypeptides of SEQ ID NO: 193 and 194, described throughout the present application also pertain to the human cDNA of clones 585770_{—}215-16-5-0-E8-F and 123996_{—}140-002-5-0-B4-F, and the polypeptides encoded thereby. Polypeptide fragments having a biological 25 activity described herein and polynucleotides encoding the same are also included in the present invention. Related polynucleotide and polypeptide sequences included in the present invention are SEQ ID NOs:360 and 447.

SMPE contracts longitudinal ileal muscle and distal colon, and relaxes the proximal colon. SMPE binds with a high affinity to both ileum and brain membranes. Therefore, included as embodiments of the present invention is a method of causing gastrointestinal smooth muscle cells to contract, in vitro or in vivo, comprising the steps of contacting said cells with a contracting effective amount of an SMPE polypeptide. Preferrably, the gastrointestinal smooth muscle cells are those of the longitudinal ileal or distal colon. A further embodiments of the present invention is a method of causing gastrointestinal smooth muscle cells to relax comprising the steps of contacting said cells with a relaxing effective amount of an SMPE polypeptide. Preferrably, the gastrointestinal smooth muscle cells are proximal colon cells. SMPE can also be used in the same manner to contract uterine cells. Therefore, included in the present invention is a method of causing uterine smooth muscle cells to contract comprising contacting said cells with a contracting effective amount of an SMPE polypeptide. Further included in the present invention is a method of causing smooth muscle cells (e.g., bladder, vascular) to contract comprising contacting said cells with a contracting effective amount of an SMPE polypeptide. Further included in the present invention is a method of inhibiting angiogenesis comprising contacting vascular endothelial cells with an angiogenesis inhibiting effective amount of an SMPE polypeptide. The SMPE anti-angiogenic affect can be measured using assays known in the art. For example, the anti-angiogenic effect in vivo can be assayed by using the 10-day-old embryo chick chorioallantoic membrane model.

SMPE binds with a high affinity to both ileum and brain membranes. Thefore, as a further embodiment of the present invention is a method of binding an SMPE polypeptide to ileum or brain membranes. The method can be further used as a method of detecting ileum or brain membranes comprising the steps of contacting ileum or brain membranes with an SMPE polypeptide under conditions that allow binding to said membranes, and detecting the presence of SMPE. The presence of SMPE can be detected using methods known in the art, such as by labeling SMPE directly or indirectly. Bound SMPE can be detected, for example, by using an antibody that specifically binds to SMPE or another SMPE-binding compound that is detectable directly or indirectly.

SMPE is also expressed in spermatocytes. Therefore, a further embodiment of the present invention is a method of detecting testes or spermatocytes by detecting an SMPE polypeptide or nucleic acid. An SMPE polypeptide can be detected using anti-SMPE antibodies or other SMPE-binding compounds. SMPE polynucleotides, such as mRNA, can be detected using methods known in the art such as PCR (RT-PCR), hybridization (Northern blot analysis), etc.

SMPE elicits hyperalgesia when it contacts the CNS, e.g., the brain. Therefore, the present invention includes a method of causing hyperalgesia comprising contacting the CNS with a hyperalgesia effecting amount of an SMPE polypeptide. SMPE can be delivered to the CNS using methods well known in the art including those described in PCT application WO9906060, incorporated herein by reference in its entirety. Using the methods of WO9906060, the TGF-alpha or other polypeptide that binds the epidermal growth factor (EGF) receptor, is substituted with an SMPE polypeptide of the present invention.

Further included in the present invention are methods of inhibiting the above SMPE activities using an inhibitor of SMPE. A preferred inhibitor of SMPE is an anti-SMPE antibody. Thus, an embodiment of the present invention is a method of inhibiting smooth muscle contraction (bladder, gastrointestional cells, uterine) or pain comprising the step of contacting said cells with an effective contractive or pain inhibiting amount of an anti-SMPE antibody or other SMPE inhibitor.

The invention further relates to a method of screening for test compounds that bind and/or inhibit an SMPE activity above comprising the steps of contacting an SMPE polypeptide with said test compound and detecting or measuring whether said test compound binds said SMPE polypeptide. Alternatively, the method comprises the steps of contacting an SMPE polypeptide with a binding target (e.g., smooth muscle cells or brain cells) of said SMPE polypeptide in the presence of a test compound, and detecting or measuring the binding of the SMPE polypeptide to said binding target, wherein a difference in the amount of said binding in the presence of said test compound relative to the amount of binding in the absence of the test compound indicates that the test compound modulates, preferably inhibits, the binding of said polypeptide to said binding target. The method may alternatively comprise the steps of contacting an SMPE polypeptide with a binding target in the presence of a test compound, wherein the binding of said SMPE polypeptide with said binding target elicits or causes a biological activity (e.g., activities described above) which is detected or measured, and further wherein a difference in the level of said biological activity in the presence of the test compound relative to the amount of biological activity in the absence of the test compound indicates that the test compound modulates, preferably inhibits or activates, the biological activity of said SMPE polypeptide.

Preferred SMPE polypeptides for use in the methods described herein include the amino acid sequences -AVITGACERDVQCGAGTCCAISLWLRGLRMCTPLGREGEECHPGSHKIPFFRKRKHH- or -TGACERDVQCGAGTCCAISLWLRGLRMCT- of SEQ ID NO: 193 or 194.

Protein of SEQ ID NO:305 (internal designation 500691428_{—}255-2-5-0-D4-R_{—}104)

The human cDNA of clone 500691428_{—}255-2-5-0-D4-R_{—}104 (SEQ ID NO:136) encodes the human VESICLE-ASSOCIATED MEMBRANE PROTEIN 10 or VAMP-10 protein: MSATAATAPPAAPAGEGGPPAPPPNLTSNRRLQQTQAQVDEVVDIMRVNVDKVLERDQKL SELDDRADALQAGPSQFETSAAKLKRKYWWKNLKMMIILGVICAIILIIIIVYFST (SEQ ID NO:305). It will be appreciated that all characteristics and uses of the polynucleotides of SEQ ID NO:136 and polypeptides of SEQ ID NO:305 described throughout the present application also pertain to the human cDNA of clone 500691428_{—}255-2-5-0-D4-R_{—}104 and the polypeptides encoded thereby. Polypeptide fragments having a biological activity described herein and polynucleotides encoding the same are also included in the present invention. Related polynucleotide and polypeptide sequences included in the present invention are SEQ ID NOs:432 and 546.

VAMP-10 is an integral membrane protein involved in the movement of vesicles from the plasmalemma of one cell, across the synapse, to the plasma membrane of the receptive neuron. This regulated vesicle trafficking pathway and the endocytotic process may be blocked by the highly specific action of clostridial, tetanus toxin (TeTx) and botulinum toxin (BoNT) and other metalloendoprotease neurotoxins which prevents neurotransmitter release by cleaving VAMPs. VAMP-10 is important in membrane trafficking. It participates in axon extension via exocytosis during development, in the release of neurotransmitters and modulatory peptides, and in endocytosis. The tightly-regulated synaptic vesicle cycle at the nerve terminal consists of the formation of synaptic vesicles, the docking of vesicles comprising VAMP-10 to the presynaptic plasma membrane, the fusion of these membranes and consequent neurotransmitter release, endocytosis of the empty vesicles and the regeneration of fresh vesicles. Endocytotic vesicular transport includes such intracellular events as the fusions and fissions of the nuclear membrane, endoplasmic reticulum, Golgi apparatus, and various inclusion bodies such as peroxisomes or lysosomes.

VAMP-10, like other VAMPs, has a three domain organization. The domains include a variable proline-rich, N-terminal sequence, a highly conserved central hydrophilic core of amino acids, and a hydrophobic sequence of amino acids presumed to be the membrane anchor.

In one aspect, the invention includes a VAMP-10 polypeptide composition for use in delivering a second composition, preferably nucleic acids, polypeptides, or small molecules such as therapeutic drugs, to target biological cells either in vitro or in vivo. The composition comprises a VAMP-10 polypeptide as a first molecule and a second molecule. The second molecule may, if desirable, be covalently or non-covalently attached or fused to the VAMP-10 polypeptide. The VAMP-10 polypeptide composition may further comprise artificial lipids to facilitate delivery of the second molecule by lipisomes or lipid vesicles. Methods for using VAMP-10 polypeptides in these methods are known in the art and include U.S. Pat. Nos. 6,074,844, 6,203,794 and 6,099,857, incorporated by reference in their entireties. In a preferred embodiment, VAMP-10 polypeptides are used to faciliate delivery of a second composition, e.g., lipisome mediated DNA transfection, to cells in culture, preferably neuronal cells, and further preferably to the presynaptic membrane.

VAMP-10 polypeptides are also useful in methods of inhibiting the release of neurotransmitters by preventing the docking and/or fusing of a presynaptic vesicle to the presynaptic membrane. These polypeptides may be referred to as excitation-secretion uncoupling peptides (ESUPs). Fragments of VAMP-10 having this blocking activity can be identified using methods known in the art (See e.g., U.S. Pat. Nos. 6,090,631 and 6,169,074 incorporated by reference in their entireties). ESUPs of the present invention comprise synthetic and purified VAMP-10 peptide fragments which correspond in primary structure to peptides which serve as binding domains for the assembly of a ternary protein complex (“docking complex”) which is critical to neuronal vesicle docking with the cellular plasma membrane prior to neurotransmitter secretion. Preferably, the primary sequence of the ESUPs of the invention also includes amino acids which are identical in sequence to the VAMP-10 peptide products of BoTx and TeTx proteolytic cleavage in neuronal cells, or fragments thereof (“proteolytic products”). For optimal activity, ESUPs of the invention have a minimum length of about 20 amino acids and a maximal length of about 28 amino acids, although they may be larger or smaller. Preferably, the ESUPs correspond in primary structure to binding domains in the docking complex, most preferably the region of such binding domains that are involved in the formation of a coiled-coil structure in the native docking complex proteins. ESUPs may also be used as pharmaceutical carriers as part of fusion proteins to deliver substances of interest into neural cells in a targeted manner. Preferred VAMP-10, or ESUP, polypeptides for use in inhibiting the release of neurotransmitters include those comprising -NRRLQQTQAQVDEVVDIMRVNVDKVLERDQKLSELDDRADALQAGPSQFETSAAKLKRK- of SEQ ID NO:305. More preferred ESUP polypeptides comprise an amino acid sequence portion of SEQ ID NO:305 selected from the group consisting of: RVNVDKVLERDQKLSELDD; KVLERDQKLSELDDRA; VNVDKVLERDQKLSELDDRA; DIMRVNVDKVLERDQKLSELDDRADAL; DEVVDIMRVNVD; QAQVDEVVDIMRVNVD; LQQTQAQVDEVVDIMRVNVD; QQTQAQVDEVVD; NRRLQQTQAQVDEVVD; and NLTSNRRLQQTQAQVDEVVD.

The ESUPs above may be used to inhibit or treat pain according to U.S. Pat. Nos. 6,113,915 or 5,989,545 (incorporated by reference herein in their entireties) by substituting the polypeptides of the present invention for BoTx type A.

Because VAMP-10 is a component of vesicles, antibodies to VAMP-10 are useful in the detection of vesicles, preferably neuronal vesicles transporting neurotransmitters. VAMP-10 can be used during purification of vesicles as a marker for vesicles or vesicles can be detected using antibodies to VAMP-10 in assays such as immunohistochemistry. Following exocytosis of vesicles, a portion of the VAMP-10 inserted in the vesicle appears on the surface of the axon, thus making VAMP-10 useful for the detection and monitoring of exocytosis of synaptic vesicles.

Detection of VAMP-10 expression (mRNA or protein) levels or mutated forms of VAMP-10 is further useful in the determination or diagnosing of whether someone is at risk of developing or has a neurological disorder, such as mood disorders selected from depression, bipolar disorder, schizophrenia, etc.), wherein a decreased level in expression of VAMP-10, mRNA or protein, as compared to an individual without a neurological disorder indicates the individual has the disorder or is as risk of having the disorder in the future.

The present invention further includes a novel assay system for toxins, such as clostridial, tetanus toxin (TeTx) and botulinum toxin (BoNT), using novel reagents. Preferably, methods of U.S. Pat. No. 6,043,042, incorporated by reference in its entirety, are used to perform the assay, wherein a VAMP-10 polypeptide is the substrate cleaved by the test compound. More specifically, the assay comprises the steps of:

The invention relates to an assay for botulinum toxin or tetanus toxin comprising the steps of:

(a) combining a test compound with a substrate and with antibody, wherein the substrate has a cleavage site for the toxin and when cleaved by toxin forms a product, and wherein the antibody binds to the product but not to the substrate; and wherein the substrate is a VAMP-10 polypeptide; and

(b) testing for the presence of antibody bound to the product, which product is attached to a solid phase assay component.

Preferably, in the practice of this invention, the VAMP-10 polypeptide is cleaved by the toxin to generate new peptides having N- and C-terminal ends. In addition, the peptide substrate is attached to a solid phase component of the assay.

The assay according to the invention may utilize assay components (a) and (b):

(a) a peptide linked to a solid-phase, the peptide being cleavable by the toxin-to generate a cleavage product,

(b) an antibody that binds to the cleavage product but not to the uncleaved polypeptide or an antibody that binds a cleavage product that is either the N-terminal or C-terminal portion of the VAMP-10, and the assay may comprise the steps of:

(i) combining a test compound that may contain or consist of the toxin with the solid-phase peptide to form an assay mixture,

(ii) subsequently or simultaneously combining the assay mixture with the antibody, and

(iii) subsequently or simultaneously determining whether there has been formed any conjugate between the antibody and the cleavage product.

Preferably, the step (i) of the assay is carried out in the presence of a zinc compound and a VAMP-10 polypeptide.

In this embodiment, the assay comprises:

(i) combining the test compound with a solid phase comprising a VAMP-10 polypeptide,

(ii) washing the test compound from the solid phase,

(iii) combining the solid phase with an antibody adapted for binding selectively with peptide cleaved by toxin, and

(iv) detecting a conjugate of the antibody with cleaved peptide.

In another embodiment, the assay comprises:

(i) adding a test solution to an assay plate comprising immobilized peptide, the peptide being a VAMP-10 polypeptide;

(ii) incubating the assay plate,

(iii) washing the plate with a buffer,

(iv) adding to the plate an antibody solution, said solution comprising an antibody adapted selectively to bind to a peptide selected from the group consisting of (1) the 50 C-terminal amino acid residues SEQ ID NO:305, the 30 C-terminal amino acid residues SEQ ID NO:305, and the 20 C-terminal amino acid residues SEQ ID NO:305 (any other VAMP-10 polypeptide of the present invention may also be selected).

(2) a peptide the N-terminal end of which is selected from the group consisting of: (1) the 50 N-terminal amino acid residues SEQ ID NO:305, the 30 N-terminal amino acid residues SEQ ID NO:305, and the 25 N-terminal amino acid residues SEQ ID NO:305 (any other VAMP-10 polypeptide of the present invention may also be selected).

(v) incubating the assay plate,

(vi) washing the plate with a buffer, and

(vii) measuring the presence of antibody on the assay plate.

In this embodiment, the antibody may be linked to an enzyme and the presence of antibody on the plate is measured by adding an enzyme substrate and measuring the conversion of the substrate into detectable product. The detectable product may be colored and measured by absorbance at a selected wavelength.

In the practice of the invention, the inactive toxin present in the test compound may be converted to active toxin. This may be accomplished by adding a protease to the test compound.

The antibody-peptide conjugate may be detected using a further antibody specific to the first antibody and linked to an enzyme.

Proteins of SEQ ID NO:171 (Internal Designation Clone ID:589115) and Related Protein of SEQ ID NO:457.

The polynucleotides of SEQ ID NO:2 and SEQ ID NO:340 and polypeptides of SEQ ID NO: 171 and 457 encode a C-terminal variant of Apolipoprotein A1, herein referred to as ApoAI-CTV. An embodiment of the invention includes compositions of SEQ ID NO:2, 340, 171, and 457 which encode for this novel variant of the apolipoprotein family of lipid transporting proteins. Specifically, ApoAI-CTV is a component of high density lipoprotein which functions to remove cholesterol from circulation and thus providing protection against the development of atherosclerosis, coronary atherosclerotic lesions and subsequent microvascular and cardiovascular disease.

Preferred polynucleotides of the invention are compositions of the novel portion of the cDNA from bases 465 to 521 of SEQ ID NO:2 including the nucleic acids comprising the sequences -GCAGCTTTCTTAACTATCCTAACAAGCCTTGGACCAAATGGAAATAAAGCTTTTTGA-, -GAAGGCAGCTTTCTTAACTATCCTAACAAGCCTTGGACCAAATGGAAATAAAGCTTTGA-, or -AGCTCTACCGCCAGAAGGCAGCTTTCTTAACTATCCTAACAAGCCTFGGACCAAATGG AAATAAAGCTTTTTGATGAAAAAA- of SEQ ID NO:2 and 340.

Preferred polypeptides of the invention are compositions of the novel C-terminal portion comprising the amino acid sequence -AAFLTILTSLGPNGNKAF, -MELYRQKAAFLTILTSLGPNGNKAF, or -QKKWQEEMELYRQKAAFLTILTSLGPNGNKAF of SEQ ID NO: 171 and SEQ ID NO:457.

Further preferred polypeptides of the invention include the compositions comprising the apolipoprotein domain KAAVLTLAVLFLTGSQARHFWQQDEPPQSPWDRVKDLATVYVDVLKDSGRDYVS QFEGSALGKQLNLKLLDNWDSVTSAFSNLREQLGPVTQEXWDNLEKETEGLRQEMSKDLE EVKAKVQPYLDDFQKKWQEEMELYRQKAAFLTILTSLGPNGNKA of SEQ ID NO: 171 and 457, or the amino acid residue positions −17 to +141 of SEQ ID NO:171.

An embodiment of the invention includes a method for treatment of atherosclerosis or cardiovascular diseases, comprising administering to an individual a therapeutically effective amount of apoAI-CTV or variants or mixtures thereof to lower total plasma cholesterol at least 5% of pretreatment levels.

Further utility of the polypeptides of the present invention may be further confirmed by methods of production and use of other apolipoproteins by those skilled in the art or as described by Ageland et al in U.S. Pat. No. 5,990,081, which disclosure is hereby incorporated by reference in its entirety.

It will be appreciated that all characteristics and uses of the polynucleotides of SEQ ID NO:2 and SEQ ID NO:340 and polypeptides of SEQ ID NO:171 and 457 described throughout the present application also pertain to the human cDNA of clone 589115.

Proteins of SEQ ID NO:302 (Internal Designation Clone ID:1000853793) and Related Protein of SEQ ID NO:543.

MRLFLSLPVLVVVLSIVLEGPAPAQGTPDVSSALDKLKEFGNTLEDKARELISRIKQ SELSAKMREWFSETFQKVKDKLKIDS

The polynucleotides of SEQ ID NO:133 and SEQ ID NO:429 encode human apolipoprotein CI (ApoCI) polypeptide of SEQ ID NO:302 and SEQ ID NO:543, respectively. The ApoCI of the invention differs by 1 amino acid comprising the amino acid sequence FQKVKDKLKI, where aspartate (D at position 77 of SEQ ID NO:302) replaces a glutamate (E) of the ApoCI of GENPEP accession X00570, AF050154, and M20902. ApoCI is a member of the apolipoprotein family of lipid binding and transporting proteins specifically functioning to transport cholesterol esters.

It will be appreciated that all characteristics and uses of the polynucleotides of SEQ ID NO:133 and SEQ ID NO:429 and polypeptides of SEQ ID NO:302 and SEQ ID NO:543, described throughout the present application also pertain to the human cDNA of clone 1000853793, and the polypeptides encoded thereby.

Proteins of SEQ ID NO:295 (Internal Designation Clone ID:642948), SEQ ID NO:296 (Internal Designation Clone ID:638743), and SEQ ID NO:539.

MEASALTSSAVTSVAKWRVASGSAVVLPLARIATVVIGGWAMAAVPMVLSAMGFTAA GIASSSIAAKMMSAAAIANGGGVASGSLVATLQSLGATGLSGLTKFILGSIGSAIAAVIARFY

The polynucleotides of SEQ ID NO:126, 127, and 425 and the polypeptides of SEQ ID NO:295, 296 and 539 encode human transmembrane, alpha-interferon-inducible polypeptides, aINFIP-1, aINFIP-1, and aINFIP-3, respectively. Preferred polynucleotides and polypeptides of the invention comprise the nucleic acid sequences of SEQ ID NO:126, 127, and 425 and amino acid sequences of SEQ ID NO:295, 296 and 539.

Preferred polypeptides of SEQ ID NO:295 and SEQ ID NO:539 for use in the methods described herein include the amino acid sequences comprising -VLSAMGFTAAGIASSSIAAKMMSAAAIANGGGVASG-, -SSIAAKMMSAAAIANGGGVASGSLVATLQSLGAT-, or -VIGGVVAMAAVPMVLSAMGFTAAGIASSSIAAKMMSAAAIANGGGVASGSLVATLQSLG ATGLSGLTK-.

Preferred polypeptides of SEQ ID NO:296 for use in the methods described herein include the amino acid sequence -AAAIANXGGVASGSLVATLQSLGATGLSGLTKF- or -LSAMGFTAAGIASSSIAAKMMSAAAIANXGGVASGSLVATLQSLGATGLS-.

It will be appreciated that all characteristics and uses of the polynucleotides of SEQ ID NOs:126, 127, and 425 and the polypeptides of SEQ ID NOs:295, 296 and 539, described throughout the present application also pertain to the human cDNA of Clone ID:642948 and Clone ID:638743, and the polypeptides encoded thereby.

Sites of glycine myristylation within a polypeptide function to modulate the activity and compartmentalization of the protein (Resh, M. D. Biochim Biophys Acta 1451:1-16 (1999)).

Preferred polypeptides of the invention include fragments comprising the sites of N-myristylation. Preferred amino acids of said sites within SEQ ID NO:295 and SEQ ID NO:539 include GGVVAM (positions 39-44), GIASSS (positions 60-65), GGGVAS (positions 79-84), GSLVAT (positions 85-90), and GSIGSX (positions 108-113). Further preferred are amino acids within 6 residues preceding or 6 residues following said amino acid sequences. Further preferred amino acids include sequences comprising the sites of N-myristylation in the polypeptides of SEQ ID NO:296. Preferred amino acids of said sites within SEQ ID NO:296 include IATVVIGGVVAMAAVPMV, MGFTAAGIASSSIAAKMM, AAIANXGGVASGSLVATL, NXGGVASGSLVATLQSLGA, and LTKFILGSIGSAIAAVIAR.

Interferons (IFNs) are a part of the group of intercellular messenger proteins known as cytokines and are part of the body's natural defense to viruses and tumors. Type I IFNs (alpha and beta interferons) are produced in a variety of cells types and their biosynthesis is stimulated by viruses and other pathogens, and by various cytokines and growth factors. Both α- and γ-IFNs are immunomodulators and anti-inflammatory agents, activating macrophages, T-cells and natural killer cells (reviewed in Jonasch and Haluska, Oncologist 6(1):34 (2001)). As part of the body's natural defense to viruses and tumors, INFs affect the function of the immune system and have direct action on pathogens and tumor cells. IFNs mediate these multiple effects by inducing the synthesis of cellular proteins, including the polypeptides of the present invention, aINFIP-1, aINFIP-1, and aINFIP-3.

Antiviral activity of the aINFIP polypeptides are assayed according to conventional methods (Tovey et al, Proc. Soc. Exp. Biol. and Med., 1974 146: 809-815). Preferred polypeptides of SEQ ID NO:295, 296 and 539 and fragments thereof include those which possess antiviral function, where preferred antiviral activity is against herpes simplex virus and hepatitis virus C, alone or in combination with known antiviral treatments such as interferon alpha.

The antitumor activity the aINFIP polypeptides of the invention can be demonstrated by similar methods using tumor cell lines rather than treatment of cells with virus as used to test antiviral activity. Tumor cell lines examples include MCF-7 (human breast cancer derived), NOS-1 (human oral primary squamous cell carcinoma derived), and MedB-1 (human primary mediastinal large B-cell lymphoma derived).

Further utility of the polypeptides of the present invention may be further confirmed by methods of interferon inducible proteins in the inhibition of viral functions such as cell penetration, uncoating, RNA and protein synthesis, assembly and release described in Hardman et al., Pharmacological Basis of Therapeutics, McGraw-Hill, New York N.Y. pp 1211-1214, 25 (1996), disclosure of which is hereby incorporated by reference in its entirety.

Another embodiment of the present invention relates to the use of aINFIP polypeptides or fragments thereof to treat and/or prevent the ill-effect of bacterial infection. In a preferred embodiment, the protein of the invention may be used to counteract the effects of the bacterial endotoxin lipopolysaccharide (LPS). The methods for using such compositions is described in Dziegielewska and Andersen, Biol. Neonate, 74:372-5 (1998), the disclosure of which is incorporated herein by reference in its entirety.

Furthermore, the aINFIP polypeptides or fragments thereof may be used to identify specific molecules with which it binds such as agonists, antagonists or inhibitors. Another embodiment of the present invention relates to methods of using the aINFIP polypeptides or fragments thereof to identify and/or quantify cytokines of the interferon family as well as other cytokines such as IL 10 and tumor antigens, which may interact with the aINFIP polypeptides of the invention.

The aINFIP polypeptides of the invention or fragments thereof are included in pharmaceutical preparations for treatment, prevention or alleviation of cancers. In another embodiment of the present invention, the aINFIP polypeptides of the invention or fragments thereof are used included in pharmaceutical preparations for treatment, prevention or alleviation of viral or bacterial infections. In another embodiment of the present invention, the aINFIP polypeptides of the invention or fragments thereof are used to inhibit and/or modulate the effect of cytokines and related molecule such as Il-2, TNF alpha, CTLA4, CD28, and others, by preventing the binding of the endogenous cytokine to their natural receptors, thereby blocking cell proliferation or inhibitory signals generated by the ligand-receptor binding event.

In another embodiment of the present invention, the aINFIP polypeptides of the invention or fragments thereof are useful to correct defects in in vivo models of disease such as autoimmune, inflammation and tumor models, by injecting the protein either intra peritoneally intravenously, subcutaneously or directly in the diseased tissue.

The polynucleotides of SEQ ID NO:126, 127, and 425 or fragments thereof is useful in diagnostic assays for aINFIP-1, aINFIP-2, or aINFIP-3 gene expression in in vitro models or in conditions associated with expression of the aINFIP polypeptides of the invention. The diagnostic assay is useful to distinguish between absence, presence, and excess expression of the gene and to monitor regulation of levels of the gene of the invention during therapeutic intervention. The DNA may also be incorporated into effective eukaryotic expression vectors and directly targeted to a specific tissue, organ, or cell population for use in gene therapy to treat the above mentioned conditions, including tumors and/or to correct disease- or genetic-induced defects in any of the above mentioned proteins including the protein of the invention.

Protein of SEQ ID NO:170 (Internal Designation Clone ID:502084) and Related Protein of SEQ ID NO:456.

The polynucleotides of SEQ ID NO:339 and polypeptides of SEQ ID NO:456 encode neutrophil stimulating protein 2, previously described in WO 9006321 (GENPEP accession A01319) as a novel factor having neutrophil-stimulating activity. The polynucleotide of SEQ ID NO:1 encodes a novel polypeptide variant, neutrophil stimulating protein 2v, comprising the amino acid sequence of SEQ ID NO:170 in which an aspartate (D) residue is located at position +16 of SEQ ID NO:170 rather than a glutamate (E). Preferred compositions of the invention include the polypeptides of SEQ ID NO: 170. Further preferred amino acids of SEQ ID NO: 170 comprise the sequence LAKGKDESLDS, QXKRNLAKGKDESLDSDLYAE, or SSTKGQXKRNLAKGKDESLDSDLYAELRCMCIKTTSGIHPKNIQSLEVIGKGTHCNQVEVIA TLKDGRKICLDPDAPRIKKIVQKKLAGDESAD. It will be appreciated that all characteristics and uses of the polynucleotides of SEQ ID NO:1 and SEQ ID NO:339 and polypeptides of SEQ ID NO:170 and SEQ ID NO:456, described throughout the present application also pertain to the human cDNA of clone 502084, and the polypeptides encoded thereby.

A preferred embodiment of the invention includes use of the novel neutrophil stimulating protein 2v of SEQ ID NO:170 in a method to stimulate wound healing by contacting the wound area with effective amount of polypeptide of SEQ ID NO:170 or further use as described in U.S. Pat. No. 5,804,176, which disclosure is hereby incorporated by reference in its entirety. A further preferred includes use of neutrophil stimulating protein 2v in the enhancement of angiogenesis for revascularization after injury such following myocardial infarction, wherein site of injury is contacted with effective amount of polypeptide of SEQ ID NO:170 or use as further described in U.S. Pat. No. 5,871,723, which disclosure is hereby incorporated by reference in its entirety. Antibodies against neutrophil stimulating protein 2v, by preventing or blocking the deposition of connective tissue matrix, are useful in the treatment of fibrotic disorders by contacting the polypeptides of SEQ ID NO:170 with fibrotic tissue, such as in scleroderma, liver cirrhosis, and myelofibrosis.

An embodiment of the invention includes fragments of SEQ ID NO:170 which comprise domains which impart function to this cytokine. Preferred fragments include the amino acid sequence comprising the IL8 domain, DSDLYAELRCMCIKTTSGIHPKNIQSLEVIGKGTHCNQVEVIATLKDGRKICLDPDAPRIKKI VQKKL. Further preferred amino acids include the small cytokines (intercrine/chemokine) C-x-C subfamily signature of the amino acid sequence comprising CMCIKTTSGIHPKNIQSLEVIGKGTHCNQVEVIATLKDGRKICLD.

Further preferred polypeptides include portions comprising sites of Protein Kinase C phosphorylation including amino acid residues 2 to 4, residues 13 to 15, residues 36 to 38 and residues 97 to 99 of SEQ ID NO: or amino acids sequence comprising SLR, SAR, STK, and TLK. Further preferred polypeptides include portions of the amino acid sequence comprising sites of Casein kinase II phosphorylation including amino acid residues 97 to 100 or the amino acid sequence comprising TLKD.

Further preferred polypeptides include portions of the amino acid sequence comprising sites of N-myristylation or the amino acid residues comprising GTHCNQ.

Further preferred polypeptides include the small cytokines (intercrine/chemokine) C-x-C subfamily signature of the amino acid sequence comprising CMCIKTTSGIHPKNIQSLEVIGKGTHCNQVEVIATLKDGRKICLD.

Proteins of SEQ ID NO: 227 (Internal Designation Clone ID: 166601) and Related Protein of SEQ ID NO:502.

Polynucleotides of SEQ ID NO:58 and SEQ ID NO:385 encode the polypeptides of SEQ ID NO:227 and SEQ ID NO:502, respectively, with amino acid sequence MAAAAVPSLLLSLPPHQGLTFSNKIQPFGAQGVLHPEPGLRDWLLPTCSRQLRVALPEKGS EGSLCQTQLPATPCFLPSNTVRT. It will be appreciated that all characteristics and uses of the polynucleotides of SEQ ID NOs:58 and 385 and polypeptides of SEQ ID NO:227 and 502, described throughout the present application also pertain to the human cDNA of clone 166601, and the polypeptides encoded thereby.

The polynucleotides of SEQ ID NO:58 and 385 and polypeptides of SEQ ID NO:227 and 502 encode a transcriptional regulatory protein. Preferred polynucleotides of the invention include the nucleic acid sequences comprising Clone 166601, the polynucleotides comprising SEQ ID NO:58 and the polynucleotides comprising SEQ ID NO:385. Preferred polypeptides of the invention include the amino acid sequences derived from the nucleic acid sequence comprising Clone 166601, the polypeptides comprising the amino acid sequences of SEQ ID NO:227 and the polypeptides comprising the amino acid sequences of SEQ ID NO:502.

In an embodiment of the invention, preferred polypeptides include the portion comprising the site of protein kinase C phosphorylation or the amino acid sequences comprising SNK or TVR of SEQ ID NO:227 and 502.

In another embodiment, preferred polypeptides of the invention include the portion of the amino acid sequence comprising sites of myristylation or the amino acids comprising the sequence GLTFSN or GSEGSL of SEQ ID NO:227 or 502.

Proteins of SEQ ID NO:268 (Internal Designation Clone ID:211056) and Related Protein of SEQ ID NO:530.

The polynucleotides of SEQ ID NO:99 and SEQ ID NO:416 and polypeptides of SEQ ID 35 NO:268 and SEQ ID NO:530, respectively, encode a novel human tryptophan hydroxylase, including the amino acid sequence hereafter referred to as nhTOH. Tryptophan is taken up by active transport into the neurons where it is hydroxylated to 5-hydroxytryptophan (5HTP). The latter is then decarboxylated to serotonin, a neurotransmitter involved in central nervous disorders, especially mood disorders, sleep disorders, and eating disorders. Activity of the polypeptide of the invention increases production of serotonin levels and increase the metabolism of tryptophan. Thus polypeptides of the invention are useful in the in vitro production of the serotonin and metabolism f tryptophan. As example, an expression vector containing the polynucleotides of SEQ ID NO:99 or SEQ ID NO:416 can be introduced into a cell line by methods known in the art such as by calcium precipitation; tryptophan can be supplied in the media; and serotonin produced by the cells can be extracted by known methods.

The invention further relates to a method of screening for test compounds that bind hnTOH comprising the steps of contacting a hnTOH polypeptide with said test compound and detecting or measuring whether said test compound binds said hnTOH polypeptide. The invention further relates to a method of screening for test compounds that activate hnTOH comprising the steps of contacting a hnTOH polypeptide with said test compound and detecting or measuring whether said test compound activates said hnTOH polypeptide, for example by measuring serotonin production or tryptophan depletion.

Another embodiment includes physiologically acceptable compositions of test compounds found to increase serotonin production, referred to as activators, in a screen. Further embodiments include methods to use activators that have been identified in a screen or previously known in the art in the preparation of physiological acceptable formulations for use in in vivo. Further preferred are methods to use activators in a physiologically acceptable formulation in the treatment of CNS disorders in which tryptophan and serotonin levels are aberrant, particularly depression, anxiety disorder, bipolar disorder, and eating disorders.

It will be appreciated that all characteristics and uses of the polynucleotides of SEQ ID NO:99 and SEQ ID NO:416 and polypeptides of SEQ ID NO:268 and SEQ ID NO:530, described throughout the present application also pertain to the human cDNA of clone 211056, and the polypeptides encoded thereby.

Proteins of SEQ ID NO: 190 (Internal Designation Clone ID: 147648) and Related Protein of SEQ ID NO:474.

The polynucleotides of SEQ ID NO:21 and SEQ ID NO:357 and polypeptides of SEQ ID NO:190 and SEQ ID NO:474 encode a novel DNA binding polypeptide containing a leucine zipper pattern multimerization domain, thereafter referred to as LZP, also known as bZIP transcription factor basic domain signature (Hai et al., Genes Dev. 3:2083(1989)). An embodiment of the present invention includes the polynucleotides, polypeptides and fragments thereof comprising the sequences of SEQ ID NO:21, 357, 190, and 474 of the invention. Preferred polypeptides of the present invention are directed to the amino acid sequences which comprise the leucine zipper domain selected from the following amino acids of SEQ ID NO:190 and 474 including LAAGAVTLGIGFFALASALWFL; PKGFFNYLTYFLAAGAVTLGIG; or FFALASALWFLICKRREIFQNS. It will be appreciated that all characteristics and uses of-the polynucleotides of SEQ ID NO:21 and SEQ ID NO:357 and polypeptides of SEQ ID NO:190 and SEQ ID NO:474, described throughout the present application also pertain to the human cDNA of clone 147648, and the polypeptides encoded thereby.

Leucine-zippers permit dimerization of various cytoplasmic hormone receptors and enzymes (Forman, et al., Mol Endocrinol, 3, 1610-1626 (1989)). Leucine zippers are also a common feature of transcription factors, where they permit homo- or heterodimerization resulting in tight binding to DNA strands (for reviews, see Abel, et al., Nature 341, 24-25 (1989); Jones, et al., Cell 61, 9-11 (1990); Lamb, et al., Trends in Biochemical Sciences 16, 417-422 (1991)). Therefore, preferred polypeptides of the present invention are useful tools in several areas of biotechnology, especially in protein engineering, where their ability to mediate homodimerization or hetero-dimerization has found several applications, including but not limited to immunochemistry, antibody generation, preparation of soluble oligomeric proteins, complementation assasys. The utility of the present invention may be further confirmed by methods described, for example, by Bosslet et al (U.S. Pat. No. 5,643,731) in which use of a pair of leucine zippers for in vitro diagnosis, in particular for the immunochemical detection and determination of an analyte in a biological liquid; by Tso et al (U.S. Pat. No. 5,932,448) in which use of leucine zippers for producing bispecific antibody heterodimers; by Conrad et al (U.S. Pat. No. 5,965,712), Ciardelli et al (U.S. Pat. No. 5,837,816), and Spriggs et al (WO9410308) in which methods of preparing soluble oligomeric proteins using leucine zippers have been described; and by Pelletier et al (WO9834120) in which methods to use leucine zipper forming sequences in protein fragment complementation assays to detect biomolecular interactions has been described, all examples which disclosures are hereby incorporated by reference in their entireties.

The multimerization activity of the polypeptides of the present invention containing leucine zipper domains may be assayed using any of the assays known to those skilled in the art including circular dichroism spectrum and thermal melting analyses as described in U.S. Pat. No. 5,942,433, which disclosures are hereby incorporated by reference in their entirety. Alternatively, the leucine zipper motif in LZP could be used by those skilled in art as a “bait protein” in a well established yeast double hybridization system to identify its interacting protein partners in vivo from cDNA library derived from different tissues or cell types of a given organism. Alternatively, LZP or part thereof could be used by those skilled in art in mammalian cell transfection experiments. When fused to a suitable peptide tag such as [His]₆ tag in a protein expression vector and introduced into culture cells, this expressed fusion protein can be immunoprecipitated with its potential interacting proteins by using anti-tag peptide antibody. This method could be chosen either to identify the associated partner or to confirm the results obtained by other methods such as those just mentioned.

In a preferred embodiment, the invention relates to compositions and methods of using the LZP polynuceotides and polypeptides of SEQ ID NO: and SEQ ID NO: or fragment thereof for preparing soluble multimeric proteins, which consist in multimers of fusion proteins containing a leucine zipper fused to a protein of interest, using any technique known to those skilled in the art including those described in international patent WO9410308, which disclosure is hereby incorporated by reference in its entirety. In another preferred embodiment, LZP or derivative thereof is used to produce bispecific antibody heterodimers as described in U.S. Pat. No. 5,932,448, which disclosure is hereby incorporated by reference in its entirety. Briefly, leucine zippers capable of forming heterodimers are respectively linked to epitope binding components with different specificities. Bispecific antibodies are formed by pairwise association of the leucine zippers, forming an heterodimer which links two distinct epitope binding components. In still another preferred embodiment, LZP or part thereof or derivative thereof is used for detection and determination of an analyte in a biological liquid as described in U.S. Pat. No. 5,643,731, which disclosure is hereby incorporated by reference in its entirety. Briefly, a first leucine zipper is immobilized on a solid support and the second leucine zipper is coupled to a specific binding partner for an analyte in a biological fluid. The two peptides are then brought into contact thereby immobilizing the binding partner on the solid phase. The biological sample is then contacted with the immobilized binding partner and the amount of analyte in the sample bound to the binding partner determined. In still another preferred embodiment, the LZP or part thereof may be used to synthesize novel nucleic acid binding proteins which are able to multimerize with proteins of interest, for example to inhibit and/or control cellular growth using any genetic engineering technique known to those skilled in the art including the ones described in the U.S. Pat. No. 5,942,433, which disclosure is hereby incorporated by reference in its entirety.

In another embodiment, the invention relates to compositions and methods using the LZP or part thereof or derivative thereof in protein fragment complementation assays to detect biomolecular interactions in vivo and in vitro as described in international patent WO9834120, which disclosures is hereby incorporated by reference in its entirety. Such assays may be used to study the equilibrium and kinetic aspects of molecular interactions including protein-protein, protein-nucleic acid, protein-carbohydrate and protein-small molecule interactions, for screening cDNA libraries for binding to a target protein with unknown proteins or libraries of small organic molecules for biological activity.

Still, another object of the present invention relates to the use of the LZP or part thereof for identifying new leucine zipper domains using any techniques for detecting protein-protein interaction known to those skilled in the art. Among the traditional methods which may be employed are co-immunoprecipitation, crosslinking and co-purification through gradients or chromatographic columns of cell lysates. Once isolated as a protein interacting with the LZP, such an intracellular protein can be identified (e.g. its amino acid sequence determined) and can, in turn, be used, in conjunction with standard techniques, to identify other proteins with which it interacts. The amino acid sequence thus obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel el al., eds., Current Protocols in Molecular Biology, J.Wiley and Sons (New York, N.Y. 1993) and PR Protocols: A Guide to Methods and Applications, 1990, Innis, M. et al., eds. Academic Press, Inc., New York).

Alternatively, methods may be employed which result in the simultaneous identification of genes which encode the intracellular proteins that can dimerize with the LZP or part thereof using any technique known to those skilled in the art. These methods include, for example, probing cDNA expression libraries, in a manner similar to the well known technique of antibody probing of lambda.gt11 libraries, using as a probe a labeled version of the LZP or part thereof, or fusion protein, e.g., the LZP or part thereof fused to a marker (e.g., an enzyme, fluor, luminescent protein, or dye), or an Ig-Fc domain (for technical details on screening of cDNA expression libraries, see Ausubel et al, supra). Alternatively, another method for the detection of protein interaction in vivo, the two-hybrid system, may be used.

Proteins of SEQ ID NO:318 (Internal Designation Clone ID: 124608) and Related Protein of SEQ ID NO:556.

The polynucleotides of SEQ ID NO:149 and SEQ ID NO:442 and polypeptides of SEQ ID NO:318 and SEQ ID NO:556 encode an RNA-binding protein, hgRBP, which functions in RNA processing and protein expression. The preferred composition of SEQ ID NO:318 and 556 include MERPDKAALNALQPPEFRNESSLASTLKTLLFFTALMITVPIGLYFTTKSYIFEGALGMSNR DSYFYAAIVAVVAVHVVLALFVYVAWNEGSRQWREGKQD.

Further preferred polypeptides include those of SEQ ID NO:318 or SEQ ID NO:556 comprising an N-myristoylation site or the amino acid sequence at positions 43-48 or comprising the amino acid sequence GLYFTT which targets the protein to the membrane of the endoplasmic reticulum for function of hgRBP in translation of cellular mRNA into protein.

It will be appreciated that all characteristics and uses of the polynucleotides of SEQ ID NO: 149 and SEQ ID NO:442 and polypeptides of SEQ ID NO:318 and SEQ ID NO:556, described throughout the present application also pertain to the human cDNA 124608of clone , and the polypeptides encoded thereby.

Protein of SEQ ID NO:337 (Internal Designation Clone ID:113448)

The polynucleotides of SEQ ID NO:168 and related SEQ ID NO:454 and polypeptides of SEQ ID NO:337 encode a novel human RNA-binding protein involved in RNA processing and protein expression which is related to Clone ID:183902 and Clone ID:635993.

It will be appreciated that all characteristics and uses of the The polynucleotides of SEQ ID NO:168 and related SEQ ID NO:454 and polypeptides of SEQ ID NO:337, described throughout the present application also pertain to the human cDNA of clone 113448, and the polypeptides encoded thereby.

Protein of SEQ ID NO:328 (Internal Designation Clone ID: 183902)

Polynucleotides of SEQ ID NO: 159 and related SEQ ID NO:450 and polypeptides of SEQ ID NO:328 encode a novel human RNA-binding protein involved in RNA processing and protein expression which is related to Clone ID:113448 and Clone ID:635993.

It will be appreciated that all characteristics and uses of the The polynucleotides of SEQ ID NO:159 and related SEQ ID NO:450 and polypeptides of SEQ ID NO:328, described throughout the present application also pertain to the human cDNA of clone 183902, and the polypeptides encoded thereby.

Protein of SEQ ID NO:329 (Internal Designation Clone ID:635993)

Polynucleotides of SEQ ID NO:160 and related SEQ ID NO:451 and polypeptides of SEQ ID NO:329 encode a novel human RNA-binding protein involved in RNA processing and protein expression which is related to Clone ID:183902 and Clone ID:113448. It will be appreciated that all characteristics and uses of the The polynucleotides of SEQ ID NO:160 and related SEQ ID NO:451 and polypeptides of SEQ ID NO:329, described throughout the present application also pertain to the human cDNA of clone 635993, and the polypeptides encoded thereby.

Many eukaryotic proteins that bind single-stranded RNA contain one or more copies of a putative RNA-binding domain of about 90 amino acids. This is known as the eukaryotic RNA-binding region, RNP-1 signature or RNA recognition motif (RRM) (Bandziulis et al. Genes Dev. 3:431 (1989); Swanson et al. Trends Biochem. Sci. 13: 86-91 (1988)). RRMs are found in a variety of RNA binding proteins, including heterogeneous nuclear ribonucleoproteins (hnRNPs), proteins implicated in regulation of alternative splicing, and protein components of small nuclear ribonucleoproteins (snRNPs). The polypeptides of SEQ ID NO:337, 328, and 329 encode novel human RNA binding protein, hereafter referred to as ghRBP which contains one copy of an RRM. Further characteristic of a protein which binds to nucleic acids, ghRBP contains a zinc finger motif comprising the amino acid sequence. Preferred polynucleotides of the invention include polynucleotides comprising the nucleic acids of SEQ ID NO:159, 160, 168, 450,451,and 454. Preferred polypeptides of the invention are polypeptides comprising the amino acids of SEQ ID NO:337, 328 and 329. It will be appreciated that all characteristics and uses of the polynucleotides of SEQ ID NO:159, 160, 168, 450, 451 ,and 454 described throughout the present application also pertain to the human cDNA of Clone ID:183902, Clone ID:635993 and Clone ID:113448.

Preferred amino acids of the invention are residues which comprise the RNA-binding domain or portion thereof. Preferred amino acid sequences are selected from the following set of sequences including AFVRRXPWTAASSQLKEHFAQFGHVRRCILPFDKETGFHRGLGWVQFSSEEGLRNALQQE NHIIDGVKVQV; SINQPVAFVRRXPWTAASSQLKEHFAQFGHVRRCILPFDKETGFHRGLGWVQFSSEEGLRN ALQQENHIID; PWTAASSQLKEHFAQFGHVRRCILPFDKETGFHRGLGWVQFSSEEGLRNALQQENHIIDGV KVQVHTRRP. Further preferred are polypeptides of the invention include any fragment of SEQ ID NO: which binds to RNA.

An embodiment of the invention relates to methods of using the polypeptides of the invention to bind to RNA molecules in vitro by techniques that are known in the art. Preferred use of the polypeptides of the invention includes extraction of RNA from biological samples, chemical reagents, cell homogenates and tissue homogenates. Further utility of the polypeptides of the present invention or part thereof may be further confirmed by binding methods described in Trifillis, et al., RNA 5(8): 1071-82 (1999) and U.S. Pat. No. 6,107,029, which disclosures are hereby incorporated by reference in their entireties.

Proteins of SEQ ID NO:248 (Internal Designation Clone ID:199782). SEQ ID NO:249 (Internal Designation Clone ID:821212). SEQ ID NO:250 (Internal Designation Clone ID:202863) and Related Protein of SEQ ID NO:518.

The polynucleotides of SEQ ID NOs:79, 80, 81 and 401 and polypeptides of SEQ ID NOs:248, 249, 250 and 518 encode human RNA-associated polypeptides which act as splicing factors. It will be appreciated that all characteristics and uses of the polynucleotides of SEQ ID NOs:79, 80, 81 and 401 and polypeptides of SEQ ID NOs:248, 249, 250 and 518, described throughout the present application also pertain to the human cDNA of clones 199782, 821212, and 202863, and the polypeptides encoded thereby.

The translation of genetic information into protein depends on RNA and the first step in this process is the transcription of DNA into RNA while retaining all the genetic information encoded in DNA. The RNA transcript undergoes various processing steps which include splicing and polyadenylation. The mature RNA transcript is translated into protein by the ribosomal machinery. Nascent RNA transcripts are spliced in the nucleus by the spliceosomal complex which catalyzes the removal of introns and the rejoining of exons. At least 40 splicing factors have been identified and interaction of these factors are important in the conformational changes needed for the enzymatic removal of introns and religation of the exons. Both protein and RNA components are involved in the spliceosome assembly and the splicing reaction. There are 2 distinct catalytic steps involved in the RNA splicing reaction with distinct proteins and RNA species. Alternative splicing factors include developmentally regulated proteins that play key roles in developmental processes such as pattern formation and sex determination, respectively (Hodgkin, J. et al. (1994) Development 120:3681-3689). Alternate splicing is also involved in the tissue specific expression of isoforms of proteins, including structural proteins and enzymes.

An embodiment of the present invention relates to compositions of the polynucleotides of SEQ ID NO:79, 80, 81 and 401 and polypeptides of SEQ ID NO:248, 249, 250 and 518. Preferred amino acids of the invention comprise the zinc finger region or fragment thereof and are selected from the following sequences of amino acids from SEQ ID NO:248, 249, 250 and 518 including GACENCGAMTHKKKDCFE; NSIITKYRKGACENCGAM; or THKKKDCFERPRRVGAKF.

The polypeptides of SEQ ID NO:248, 249, 250 and 518 are involved in the splicesome complex and have function in the processing of RNA processing. Alternatively, the polypeptides of the present invention are involved in RNA processing and thus involved in protein expression. A preferred embodiment of the invention relates to a method of using the polynucleotides of polynucleotides of SEQ ID NO:79, 80, 81 and 401 in vitro. A preferred method of use relates to introduction of said polypeptides or fragments thereof into cells by techniques known in the art such as transfection or microinjection. Further preferred are methods to use the polynucleotides of the invention to alter protein expression in the given cell. Alternately, polypeptides of the present invention can be used in combination with reagents known in the art to alter protein expression in cell free expression systems, mammalian expression systems, insect expression systems, or bacterial expression systems. Furthermore, methods to use the polynucleotides or polypeptides the present invention to increase or decrease protein expression is preferred. The utility of the polypeptides of the invention or part thereof may be further confirmed using methods described in U.S. Pat. No. 6,020,164 and Chua and Reed, Gene Devel 13:841-850 (1999), which disclosures are hereby incorporated by reference in their entireties.

In another embodiment, methods to screen for inhibitors and activators of the polypeptides of the invention are preferred. In another embodiment, molecules or compounds which are identified in such a screen are further preferred. Further preferred are compounds which activate or inhibit activity of the polypeptides of the current invention. Activity of the the polypeptides of the invention is modified by phosphorylation at cAMP and cGMP dependent phosphorylation sites (including 3-6:55-58; 107-110) and casein kinase 11 phosphorylation sites (including 33-36:58-61:126-129). Preferred activators include but are not limited to compounds which promote accumulation of intracellular cAMP and cGMP. Further preferred activators include those compounds which activate casein kinase II. Preferred inhibitors are those compounds which inhibit intracellular cAMP and cGMP accumulation, or those compounds which promote cAMP and cGMP degradation. Further preferred inhibitors include compounds which promote the deactivation of casein kinase II. Furthermore, inhibitors and activators of the polypeptides of the present invention include compounds known in the art as well as compounds to be identified by the method of screening. Furthermore, compounds that inhibit or activate the activity of the polypeptides of the present invention by means other than phosphorylation or dephosphorylation are also preferred.

In another embodiment, a method for the use of the polynucleotides or polypeptides of the present invention in the treatment, prevention, attenuation or diagnosis of disorders of RNA processing or protein processing are preferred. Such disorders are selected from a group which includes but is not limited to cancers such as adenocarcinoma, leukemia, sarcoma, teratocarcinoma, and any disorder associated with cell growth and differentiation, embryogenesis, and morphogenesis involving any tissue, organ, or system, e.g., the brain, adrenal gland, or reproductive system.

Proteins of SEQ ID NO:256 (Internal Designation Clone ID:822794). SEQ ID NO:257 (Internal Designation Clone ID:337572) and Related Protein of SEQ ID NO:521.

The polynucleotides of SEQ ID NO:87, 88 and 407 and polypeptides of SEQ ID NO:256, 257 and 521 encode human nuclear polypeptides which interact with transcription factors of the Signal Transducers and Activators of Transcription (STAT) family of proteins involved in the regulation of cell division. It will be appreciated that all characteristics and uses of the polynucleotides of SEQ ID NO:87, 88 and 407 and polypeptides of SEQ ID NO:256, 257 and 521, described throughout the present application also pertain to the human cDNA of clones 822794 and 337572, and the polypeptides encoded thereby.

STATs are pleiotropic transcription factors which mediate cytokine-stimulated gene expression in multiple cell populations (Levy, Cytokine Growth Factor Rev., 8:81 (1997)). All STAT proteins contain a DNA binding domain, a Src homology 2 (SH2) domain, and a transactivation domain necessary for transcriptional activation of target gene expression. Janus kinases (JAK), including JAK1, JAK2, Tyk, and JAK3, are cytoplasmic protein tyrosine kinases (PTKs) which play pivotal roles in initiation of cytokine-triggered signaling events by activating the cytoplasmic latent forms of STAT proteins via tyrosine phosphorylation on a specific tyrosine residue near the SH2 domain (Ihle et al., Trends Genet., 11: 69 (1995); Darnell, Science 277(5332):1630 (1997); Johnston et al., Nature, 370: 1513 (1994)). Tyrosine phosphorylated STAT proteins dimerize through specific reciprocal SH2-phosphotyrosine interactions and translocate from the cytoplasm to the nucleus where they stimulate the transcription of specific target genes by binding to response elements in their promoters (Leonard, Nature Medicine, 2: 968 (1996); Zhong et al., PNAS USA, 91:4806 (1994) Darnell, Science, 277:1630 (1997)).

In an embodiment of the present invention, compositions of the polynucleotides and polypeptides or fragments thereof SEQ ID NO:87, 88 and 407 and SEQ ID NO:256, 257 and 521, respectively are included. Further preferred are polypeptides of the present invention which interact with activated STAT3, but may also interact with STAT1, STAT2 or other STAT homologues. Preferred polypeptides of the invention act to inhibit or decrease the activity of STATs. Further preferred amino acids of SEQ ID NO:256, 257 and 521 include the SAP domain VSSFRVSELQVLLGFAGRNKSGRKHDLLMRALHLL. Activation of cytokine receptors by their cognate ligands activate JAKs which in turn, activate STATS. Therefore cytokines and other hormones which signal through cytokine-like receptors may be modulated by polypeptides or polynucleotides of the present invention. Cytokines and other hormones which can thus be modulated by the present invention include but are not limited to interferons, interleukins, prolactin, and growth hormone. The utility of the polypeptides of the present invention or part thereof may be further confirmed using the methods described in WIPO Publication W09928465 which disclosure is hereby incorporated by reference in its entirety.

The polypeptides of this invention can be used in a method of inhibiting the activity of STAT proteins in a cell in vitro, the method comprising introducing a nucleic acid into the cell, wherein the nucleic acid comprises a nucleotide sequence encoding the amino acid sequence of SEQ ID NO:256, 257 and 521 or the amino acid sequence of SEQ ID NO:256, 257 and 521 with one or more conservative amino acid alterations, and wherein the nucleic acid expresses the amino acid sequence in an amount and for a time sufficient for the amino acid sequence to specifically bind to STAT proteins and to decrease STAT activity, thereby decreasing STAT activity in the cell.

Suitable compositions of polypeptides or polynucleotides of the present invention are useful as a method of treatment of pathologies such as diseases, syndromes, or other undesirable conditions resulting from defects in cell cycle progression. Such cell cycle defects may result from defects in the regulation of activated STAT or an upstream factor such as activated JNKs or activated cytokine receptors. Alternatively, polypeptides or polynucleotides of the present invention may be used in a method of treating pathologies resulting from defects in cell cycle progression due to defects in a step “downstream” of STAT regulation of cell cycle progression. In preferred embodiments, agonists of polypeptides or polynucleotides of the present invention are useful in the treatment of pathologies such as but not limited to hyperproliferative diseases such as cancer (e.g., leukemia, lymphoma, breast cancer, colon cancer, prostate cancer, Wilms' tumor), coronary artery disease, pulmonary vascular obstructive disease, either primary or as a feature of Eisenmenger's syndrome, and other disorders of abnormal cellular proliferation. Cells to be treated include but are not limited to hyperproliferative cells, cancer cells, vascular smooth muscle cells, endothelial cells, and gametes.

In some embodiments of the invention, antagonists of the polypeptides or polynucleotides of the present invention are used to stimulate, promote, or facilitate progression through the cell cycle, such as in the cellular regeneration of terminally differentiated cardiac myocytes or tissues, e.g., striated muscle myocytes. For example, this could allow restoration of damaged myocardium after cardiac injury, myocardial infarction, myocarditis, cardiomyopathy, trauma, as a consequence of cardiac surgery, etc., or repletion of striated muscle exhausted by muscular dystrophy.

In further embodiments, expression of the polypeptides encoded by the nucleic acids is expected to prevent, ameliorate, or lessen the cell cycle defect of the host cell, or to restore normal cell cycle progression of the host cell. Whether provided via nucleic acid or polypeptides delivered directly to cells, the therapeutic formulations of the invention can also be used as adjuncts to other forms of therapy, including but not limited to chemotherapy, and radiation therapy.

Protein of SEQ ID NO:330 (Internal Designation Clone ID:398703) and Related Protein of SEQ ID NO:330.

The polynucleotides of SEQ ID NO:161 and SEQ ID NO:452 and polypeptides of SEQ ID NO:330 encode a novel human deubiquitinating enzyme (GNP:AF017306). Deubiquitinating enzymes serve a number of functions (Hochstrasser Cur Opin Cell Biol 4:1024 (1992); Rose, In: Ubiguitin, Plenum Press, New York (1988)). First, ubiquitin must be cleaved from a set of biosyntheticprecursors, which occur either as a series of ubiquitin monomers in head-to-tail linkage or as fusions to certain ribosomal proteins (Finley & Chau, Annu Rev Cell Biol 7, 25-69 (1991)). Secondly, ubiquitin must be recycled from intracellular conjugates, both to maintain adequate pools of free ubiquitin and, in principle at least, to reverse the modification of inappropriately targeted proteins. Finally, deubiquitinating reactions may be integral to the degradation of ubiquitinated proteins by the 26S proteasome, a complex ATP-dependent enzyme whose exact composition and range of activities remain poorly characterized (Hershko & Ciechanover, Annu Rev Biochem 61, 761-807 (1992); Hadari et al., J Biol Chem 267, 719-727 (1992); Murakami et al., Nature 360, 597-9 (1992); Rechsteiner, J. Biol. Chem. 268, 6065-6068 (1993)).

An embodiment of the invention includes preferred polypeptides with ubiquitin-specific protease activity with a novel N-terminus of Clone ID:398703 comprising the amino acid sequence MCTTSLPCPIIMEPWGLATTKAAYVLFYQRRDDEFYKTPSLSSSGSSDGGTRPSSSQQGFGD DEACSMDTN encoded by ATGTGTACGACCTCATTGCCGTGTCCAATCATTATGGAGCCATGGGGGTTGGCCACTAC TAAAGCAGCTTATGTGCTATTTTACCAACGTCGAGATGATGAATTTTATAAGACACCTT CACTTAGCAGTTCTGGTTCCTCTGATGGAGGGACACGACCAAGCAGCTCTCAGCAGGG CTMTGGGGATGATGAGGCTTGCAGCATGGACACCAACTAA of SEQ ID NO: 161.

The preferred polypeptides of the invention are those which prevent or reverse ubiquitination of cellular proteins in vitro or in vivo. Further preferred are polypeptides of the invention which prevent or reverse ubiquitination of extracellular proteins in vitro or in vivo.

The polynucleotides of SEQ ID NO:161 and SEQ ID NO:452 encode polypeptides of SEQ ID NO:330 which contain protein domains or motifs including but not limited to a Protein kinase C phosphorylation site comprising the amino acid fragment SAR, and an N-myristylation site comprising amino acid fragment GLNMSE. Further preferred amino acids of SEQ ID NO: 330 include the Ubiquitin carboxyl-terminal hydrolases family 2 signature or amino acid sequence YDLIAVSNHYGAMGVGHY.

It will be appreciated that all characteristics and uses of the polynucleotides of SEQ ID NO:161 and SEQ ID NO:452 and polypeptides of SEQ ID NO:330, described throughout the present application also pertain to the human cDNA of clone 398703, and the polypeptides encoded thereby.

The utility of the polynucleotides and polypeptides of the present invention or part thereof may be further confirmed using methods which assess activity or function of deubiquitinating enzymes described in United States Patents 5391490 and 5565352 which disclosures are hereby incorportated by reference in their entireties.

Proteins of SEQ ID NO:277 (Internal Designation Clone ID:653966) and Related Protein of SEQ ID NO:535.

The polynucleotides of SEQ ID NO:108 and SEQ ID NO:421 and polypeptides of SEQ ID NO:277 and SEQ ID NO:535 encode human liver fatty acid binding protein (L-FABP) comprising the amino acid sequence of SEQ ID NO:277 and 535. The amino acid sequence of SEQ ID NO:277 and 535 are the same as human L-FABP (Genbank accession GNP:M10617; Lowe et al., JBC 260:3413-17 (1985)) and homologous to human FABP (Genbank accession GNP:M10050). The polypeptides of the present invention belong to the FABP/P2/CRBP/CRABP family of transporters and functionally binds to free fatty acids and derivatives thereof. L-FABP is normally expressed in the cytoplasm of hepatocytes, but preferred embodiments include use of the polypeptides of the present invention as extracellular polypeptides. Further preferred embodiments include use of the polypeptides of the present invention as serum or plasma polypeptides. Further preferred embodiments use polypeptides of the invention in vitro. Still further preferred embodiments include use of the polypeptides of the present invention in vivo.

Preferred amino acids of the invention include the lipocalin domain, from 2 to 127 or polypeptides comprising the amino acid sequence SFSGKYQLQSQENFEAFMKAIGLPEELIQKGKDIKGVSEIVQNGKHFKFTITAGSKVIQNEFT VGEECELETMTGEKVKTVVQLEGDNKLVTTFKNIKSVTELNGDIITNTMTLGDIVFKRISKR I. The polypeptides of SEQ ID NO:277 and SEQ ID NO:535 contain a cytosolic fatty-acid binding protein signature comprising the amino acid sequence GKYQLQSQENFEAFMKAI which functions in the polypeptides ability to bind small hydrophobic molecules, such as lipids, steroid hormones, and retinoids. Preferred amino acids of SEQ ID NO:277 and SEQ ID NO:535 include GKYQLQSQENFEAFMKAI, MSFSGKYQLQSQENFEAF, and LQSQENFEAFMKAIGLPE.

Phosphorylation status modulates the activity of L-FABP. Preferred polypeptides of the invention include the amino acids sequence comprising the sites of cAMP- and cGMP-dependent protein kinase phosphorylation including residues of SEQ ID NO:277 and 535 comprising the sequence KRIS.

Further preferred polypeptides of the invention include the amino acid sequence comprising the sites of Protein kinase C phosphorylation including residues at positions 4 to 6, 94 to 96, and 124 to 126 of SEQ ID NO:277 and 535. Still further preferred polypeptides of SEQ ID NO:277 and 535 include the amino acid sequence comprising SGK, TFK, and SKR.

Further preferred are polypeptides of SEQ ID NO:277 and 535 include the amino acid sequence comprising a Casein kinase II phosphorylation sites. Preferred amino acids of SEQ ID NO:277 and 535 include positions 64 to 67, 100 to 103, and 114 to 117. Further preferred amino acids comprise the sequences TVGE, SVTE, and TLGD.

A preferred polypeptide of SEQ ID NO:277 and 535 is one in which the amino acid asparagine (Asn) is located at residue 105, further referred to as the N-isoform. Further preferred is the polypeptide of SEQ ID NO:277 and 535 in which the amino acid aspartate (Asp) is located at residue 105 further referred to as the D-isoform. The rat homologue of the human D-isoform of the present invention was shown to have a greater affinity to lysophospholipids, prostaglandins, retinoids, bilirubin and bile salts compared to the rat homologue of the human N-isoform of the present invention by methods described by DiPietro and Santome, Biochim Biophys Acta 1478 :186-200 (2000) which disclosure is hereby incorporated by reference in its entirety. The rat homologues share only 82% identity with the of the human D- and N-isoforns, therefore it is not predictable to find that the human D-isoform has equal or greater affinity to lysophospholipids, prostaglandins, retinoids, bilirubin, bile salts and fatty acid compared to the human N-isoform.

Further preferred polypeptides of the present invention include the D-isoform polypeptide and fragments thereof which have an equal or at least 10%, 20%, 30%, 40%, 50%, 60% or 75% greater affinity for fatty acids, and lipophilic compounds selected from a group including but not limited to lysophospholipids, prostaglandins, retinoids, bilirubin, bile salts, steroid hormones (such as testosterone and estradiol), and cholesterol compared to the N-isoform.

Another embodiment of the invention includes polynucleotides or polypeptides of the invention or fragments thereof which bind lipophilic compounds selected from a group including but not limited to free fatty acids, lysophospholipids, prostaglandins, retinoids, bilirubin, bile salts, steroid hormones (such as testosterone and estradiol), and cholesterol in serum or plasma. Further preferred are polypeptides of the invention which bind lipophilic compounds in serum or plasma separated from whole blood in a process of purifying serum or plasma for use in vitro or in vivo. Further preferred are polypeptides of the invention which bind lipophilic compounds in serum or plasma in vivo.

In a further embodiment, polynucleotides or polypeptides of the invention or fragments thereof, in physiological appropriate formulations, are useful in the prevention, treatment or attenuation of conditions in which lipophilic compounds are elevated in the serum of mammals, preferably humans. Such conditions are selected from a group which include but are not limited to obesity, hyperlipidemia, hypercholesterolemia, hypertriglyceridemia, diabetes type I (IDDM) diabetes type II (NIDDM), atherosclerosis, and hypertension.

Mammary-derived growth inhibitor (MDGI) and heart-fatty acid binding protein (FABP), which belong to the FABP family, specifically inhibit growth of normal mouse mammary epithelial cells (MEC) and promote morphological differentiation, stimulates its own expression and promotes milk protein synthesis (U.S. Pat. No. 5,977,309, 24 Mar. 1995). In further preferred embodiments, polypeptides of the invention include those which locally signal growth cessation and stimulate differentiation of the developing epithelium. Further preferred polypeptides of the invention suppress the mitogenic effects of EGF family members, and inhibit c-fos, c-myc and c-ras expression.

In a further aspect of the present invention, there is provided a method for producing such polypeptide by recombinant techniques comprising culturing recombinant prokaryotic and/or eukaryotic host cells, containing a human fatty acid binding polypeptides or polynucleotides of the invention acid under conditions promoting expression of said protein and subsequent recovery of said protein.

In a further embodiment of the present invention, there is provided a method for utilizing such polypeptides, or polynucleotides of the invention for therapeutic purposes, for example, as a cell growth inhibitor and as to cause differentiation stimulatory activity on various responsive types of tissues and cells in vitro. Further preferred are methods for use of polypeptides, or polynucleotides of the invention, in appropriate physiological form, for therapeutic purposes for to inhibit cell proliferation or to induce cell differentiation in mammals, preferably humans.

It will be appreciated that all characteristics and uses of the polynucleotides of SEQ ID NO:108 and SEQ ID NO:421 and polypeptides of SEQ ID NO:277 and SEQ ID NO:535, described throughout the present application also pertain to the human cDNA of clone 653966, and the polypeptides encoded thereby.

Proteins of SEQ ID NO:313 (Internal Designation Clone ID:633418). SEQ ID NO:314 (Internal Designation Clone ID:422878) and Related Protein of SEQ ID NO:552.

The polynucleotides of SEQ ID NO:144, 145 and 438 and polypeptides of SEQ ID NO:313, 314and 552 encode a cleavage stimulation factor important in mRNA processing and protein expression. Protein kinase C phosphorylation increases activity of said polypeptides and preferred amino acids include SEK and SGR. Further, sites of tyrosine kinase phosphorylation increase activity of said polypeptides and preferred amino acids of SEQ ID NO:314, 552 include KKLEENPY.

It will be appreciated that all characteristics and uses of the polynucleotides of SEQ ID NO:144, 145 and 438 and polypeptides of SEQ ID NO:313, 314and 552, described throughout the present application also pertain to the human cDNA of clones 633418 and 422878, and the polypeptides encoded thereby.

Proteins of SEQ ID NO:219 (Internal Designation Clone ID:589848), SEQ ID NO:220 (Internal Designation Clone ID:211883). SEQ ID NO:221 (Internal Designation Clone ID:642603). SEQ ID NO:222 (Internal Designation Clone ID: 193316), and Related Protein of SEQ ID NO:497.

Polynucleotides of SEQ ID NO:50, 51, 52, 53, 380 and polypeptides of SEQ ID NO:219, 220, 221 and 497 encode RNA associated proteins with a ribosomal L34 domain comprising the amino acid sequence NEYQPSNIKRKNKHGWVRRLXTPAGXXXILRRMLKGRKSLSH or NEYQPSNIKRKNKHGWVRRLXTPAGVQVILRRMLKGRKSLSH. It will be appreciated that all characteristics and uses of the polynucleotides of SEQ ID NOs:50, 51, 52, 53,380 and polypeptides of SEQ ID NOs:219, 220, 221 and 497, described throughout the present application also pertain to the human cDNA of clones 589848, 211883, 642603 and 193316, and the polypeptides encoded thereby.

Proteins of SEQ ID NO:302 (Internal Designation Clone ID:1000891255) and Related Protein of SEQ ID NO:543.

Polynucleotides of SEQ ID NO:133 and 429 and polypeptides of SEQ ID NO:302 and 543 encode human ribosomal protein, hRIBPRT. An embodiment of the invention includes the compositions of the polypeptides of SEQ ID NO:302 and 543, comprising the amino acid sequence MVAAKKTKKSLESIKSRLQLVMKSGKYVLGYKQTLKMIRQGKAKLVILANNCPALRKSEI EYYAMLAKTGVHHYSGNNIELGTACGKYYRVCTLAIIDPXDSXIIRSMPEQTGEK, and the polynucleotides of SEQ ID NO:133 and 429, respectively, which encode human ribosomal protein, hRIBPRT. The polypeptides of the invention contain the ribosomal protein L30e/L7Ae/S12e/Gadd4 signature (Koonin E V, J Mol Med 75:236-238 (1997) and Nakanishi et al., Gene 35:289-96 (1985)).

Preferred polypeptides of the invention include the amino acid sequence comprising KSLESIKSRLQLVMKSGKYVLGYKQTLKMIRQGKAKLVILANNCPALRKSEIEYYAMLAK TGVHHYSGNNIELGTACGKYYRVCTLAIIDPXDSXIIR; KSLESIKSRLQLVMKSGKYVLGYKQTLKMIRQGKAKLVILANNCPALRK; and SEIEYYAMLAKTGVHHYSGNNIELGTACGKYYRVCTLAIIDPXDSXIIR. Further preferred amino acids of the invention include sites of PKC phosphorylation, comprising the amino acid sequences of SEQ ID NO:302 and 543 including TKK (positions 7-13); SIK (positions 13-15); SGK (positions 2426); and TLK (positions 34-36). Further preferred amino acids of the invention include sites of Casein Kinase II phosphorylation, comprising the amino acid sequences SEIE (positions 58-6 1) and SMPE (positions 107-110).

In another embodiment, the proteins of SEQ ID NO:302 and 543 can be used to bind to nucleic acids, preferably RNA, alone or in combination with other substances. For example, the proteins of the invention or part thereof can be added to a sample containing RNAs in optimum conditions for binding, and allowed to bind to RNAs. In a preferred such embodiment, the proteins of the invention or part thereof may be used to purify mRNAs, for example to specifically isolate RNA, e.g. from a specific cell type or from cells grown under particular conditions. Such RNAs could then be reverse transcribed and cloned, could be analyzed for relative expression analyses, etc. In addition, such methods may be used to specifically remove RNA from a sample, for example during the purification of DNA. To carry out any of these methods, the proteins of the invention or part thereof may be bound to a chromatographic support, either alone or in combination with other RNA binding proteins, to form an affinity chromatography column. A sample containing a mixture of nucleic acids to purify is then run through the column. Immobilizing the proteins of the invention or part thereof on a support is particularly advantageous for embodiments in which the method is to be practiced on a commercial scale. This immobilization facilitates the removal of RNAs from the batch of resin-coupled protein after binding, and allows subsequent re-use of the protein. Immobilization of the proteins of the invention or part thereof can be accomplished, for example, by inserting any matrix binding domain in the protein according to methods known to those skilled in the art. The resulting fusion product including the proteins of the invention or part thereof is then covalently, or by any other means, bound to a protein, carbohydrate or matrix (such as gold, “Sephadex” particles, polymeric surfaces).

Another embodiment of the present invention relates to methods and compositions using the proteins of the invention, or part thereof, to associate specific mRNAs to the inner face of lipidic bilayers of liposomes in order to further introduce these mRNAs into the cytoplasm of eukaryotic cells. Preferably, specific mRNAs are first associated with the protein of the invention and the RNA/protein complex formed in that way is then mixed with liposomes according to methods known to those skilled in the art. These liposomes are added to an in vitro culture of eukaryotic cells. In vivo, such a method might treat and/or prevent disorders linked to dysregulation of gene transcription such as cancer and other disorders relating to abnormal cellular differentiation, proliferation, or degeneration.

A decrease in ribosome function results in a significant inhibition of cell growth. Therefore, in another embodiment, the present proteins and nucleic acids can be used to modulate the rate of cell growth in vitro or in vivo. Accordingly, compounds that inhibits the expression or function of the proteins of the invention can be used to inhibit the growth rate of cells, and can thus be used, e.g. in the treatment or prevention of diseases or conditions associated with excessive cell growth, such as cancer or inflammatory conditions. Such compounds include, but are not limited to, antibodies, antisense molecules, dominant negative forms of the proteins, and any heterologous compounds that inhibit the expression or the activity of the proteins.

It will be appreciated that all characteristics and uses of the polynucleotides of SEQ ID NO:133 and 429 and polypeptides of SEQ ID NO:302 and 543, described throughout the present application also pertain to the human cDNA of clone 1000891255, and the polypeptides encoded thereby.

Proteins of SEQ ID NO:271 (Internal Designation Clone ID:493328), Related Clones 153261, 152042, 599054, and 650872 and Related Protein of SEQ ID NO:533.

The polynucleotides of SEQ ID Nos:102, 103, 104, 105, and 106 and polypeptides of SEQ ID Nos:271, 272, 273, 274, 275 and 533 encode for the HUMAN GENSET BINDING PROTEIN or HGBP-1. Preferred polypeptides comprise the amino acid sequence MKVKIKCWNGVATWLWVANDENCGICRMAFNGCCPDCKVPGDDCPLVWGQCSHCFHM HCILKWLHAQQVQQHCPMCRQEWKFKE. It will be appreciated that all characteristics and uses of the polynucleotides of SEQ ID Nos:102, 103, 104, 105, and 106 and polypeptides of SEQ ID Nos:271, 272, 273, 274, 275 and 533, described throughout the present application also pertain to the human cDNA of clones 493328, 153261, 152042, 599054, and 650872, and the polypeptides encoded thereby.

The protein of SEQ ID NO: 271 encoded by the extended cDNA SEQ ID NO:102 is the same as a hepatocellular carcinoma associated ring finger protein (EMBL AF247565) and Genset protein in WO0100806 (Genpep accession AX061622) with homology to an anaphase-promoting complex (APC) subunit from Drosophila (Embi accession number AJ251510). In addition, HGBP-1 exhibits the pfam PHD zinc finger signature from positions 33 to 79.

Zinc binding domains which contain a C₃HC₄ sequence motif are known as RING domains (Lovering, R. et al. (1993) Proc. Natl. Acad. Sci. USA 90:2112-2116). Zinc finger domains are found in numerous zinc binding proteins which are involved in protein-protein and protein-nucleic acid interactions. They are independently folded zinc-containing mini-domains which are used in a modular repeating fashion to achieve sequence-specific recognition of DNA (Klug 1993 Gene 135, 83-92). Such zinc binding proteins are commonly involved in the regulation of gene expression, and usually serve as transcription factors, either by directly affecting transcription or recruiting co-activators or co-repressors (see U.S. Pat. Nos. 5,866,325; 6,013,453 and 5,861,495). PHD fingers are C₄HC₃ zinc fingers spanning approximately 50-80 residues and distinct from RING fingers or LIM domains. They are thought to be mostly DNA or RNA binding domain but may also be involved in protein-protein interactions (for a review see Aasland et al, Trends Biochem Sci 20:56-59 (1995)).

HGBP-1 or part thereof is a zinc binding protein, which is able to bind nucleic acids, more preferably a transcription factor. Preferred polypeptides of the invention are polypeptides comprising the amino acids of SEQ ID NO: 271 from positions 33 to 79. Other preferred polypeptides of the invention are fragments of SEQ ID NO: 271 having any of the biological activity described herein. The nucleic acid binding activity of the protein of the invention or part thereof may be assayed using any of the assays known to those skilled in the art including those described in U.S. Pat. No. 6,013,453.

The invention relates to methods and compositions using the protein of the invention or part thereof to bind to nucleic acids, preferably DNA, alone or in combination with other substances. For example, the protein of the invention or part thereof is added to a sample containing nucleic acid in conditions allowing binding, and allowed to bind to nucleic acids. In a preferred embodiment, the protein of the invention or part thereof may be used to purify nucleic acids such as restriction fragments.

In another preferred embodiment, HGBP polypeptides or parts thereof may be used to visualize nucleic acids when the polypeptide is linked to an appropriate fusion partner, or is detected by probing with an antibody. Thus, HGBP polypeptides can be used to diagnose.

Alternatively, the protein of the invention or part thereof may be bound to a chromatographic support, either alone or in combination with other DNA binding proteins, using techniques well known in the art, to form an affinity chromatography column. A sample containing nucleic acids to purify is run through the column. Immobilizing the protein of the invention or part thereof on a support advantageous is particularly for those embodiments in which the method is to be practiced on a commercial scale. This immobilization facilitates the removal of the protein from the batch of product and subsequent reuse of the protein. Immobilization of the protein of the invention or part thereof can be accomplished, for example, by inserting a cellulose-binding domain in the protein. One of skill in the art will understand that other methods of immobilization could also be used and are described in the available literature.

In another embodiment, the present invention relates to compositions and methods using the protein of the invention or part thereof, especially the zinc binding domain, to alter the expression of genes of interest in a target cells. Such genes of interest may be disease related genes, such as oncogenes or exogenous genes from pathogens, such as bacteria or viruses using any techniques known to those skilled in the art including those described in U.S. Pat. Nos. 5,861,495; 5,866,325 and 6,013,453.

In a further embodiment, the protein of the invention or part thereof may be used to diagnose, treat and/or prevent disorders linked to dysregulation of gene transcription such as cancer and other disorders relating to abnormal cellular differentiation, proliferation, or degeneration, including hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease. The invention relates to methods of diagnosing, treating and/or preventing disorders described herein, comprising delivering to a patient, or causing to be present therein, a zinc finger polypeptide which inhibits the expression of a gene enabling the cells to divide. The target could be, for example an oncogene or a normal gene, which is overexpressed in the cancer cells.

ISPG Iron-Sulfur Cluster Protein (Clone ID:1000872335)

The polynucleotides of SEQ ID NOs:43 and 374 encodes the amino acids sequence of SEQ ID NOs:212 and 491 respectively, an iron-sulfur protein which mediates electron transfer in metabolic reactions, also referred to as ISPG. It will be appreciated that all characteristics and uses of the polynucleotides of SEQ ID NOs 43 and 374 and polypeptides of SEQ ID NOs:212 and 491, described throughout the present application also pertain to the human cDNA of clone 1000872335, and the polypeptides encoded thereby.

ISPG is an iron-sulfur protein that belongs to the broad family of the 2Fe-2S-type ferredoxins. The 2Fe-2S-type ferredoxins are proteins or domains of around one hundred amino acid residues that bind a single 2Fe-2S iron-sulfur cluster and are found in plants, animals and bacteria. Iron-sulfur cluster proteins are well known classes of proteins and are recognized as ideal devices for accepting, donating, storing and shifting electrons. It will be appreciated by the skilled artisan that stuctural aspect of iron-sulfur proteins have been studied extensively (reviewed in Beinert et al, Nature 1 Aug. 1997, 277:653-659), allowing modifications of the ISPG proteins to fine tune its properties for desired uses while retaining its biological function in mediating electron transfer and possible protein stabilization and iron or sulfide storage functions. The ISPG protein of SEQ ID NO 491 comprisese a glycosaminoglycan attachment site at amino acid positios 34 (SGSG); protein kinase C phosphorylation sites at amino acid positions 14 (SAR), 44 (TTR), and 86 (SGR); N-myristoylation sites at amino acid positions 11 (GGVSAR), 24 (GTXWNR), 31 (GGTSGS), 39 (GVALGT), 106 (GACEAS), a cytochrome c family heme-binding site at amino acid positions 114-119 and an iron-sulfur binding region signature at amino acid positions 108-118.

In view of its role in electron transfer reactions, ISPG is thought to be involved in a wide variety of metabolic reactions and disorders, and may be useful in the treatment of disorders of metabolism such as obesity, in the detection of toxic compounds, in prediction, diagnosis or treatment of conditions or traits related to drug metabolism or in treatments related to the synthesis of eg. steroid hormones.

In a preferred example, overexpression or administration of the ISPG protein may be used as a therapeutic treatment for obesity by accelerating the metabolic rate of a subject in need of treatment. There is accumulating evidence to support the hypothesis that a low-energy-output phenotype is at high risk of weight gain and obesity, irrespective of whether this is owing to a low resting metabolic rate and/or physical inactivity. The low-energy-output phenotype is associated with impaired appetite control, which is improved if energy output is increased, serving as the background for pharmacologic stimulation of energy expenditure as a tool to improve the results of obesity management. The ISPG protein and agonists or stimulators thereof may serve as a means to increase electron transfer and hence the metabolic rate of an individual in a similar goal as commonly cited targets such as leptin receptors, the sympathetic nervous system and its peripheral beta-adrenoceptors, selective thyroid hormone derivatives, and stimulation of the mitochondrial uncoupling proteins.

In addition, iron-sulfur proteins such as ISPG are generally recognized as being capable of several functions that are not of an oxidoreductive nature such as the binding and activation of substrates at the unique iron site (in the catalytic function of aconitase and relates enzymes), and apparently stabilizing radicals in reactions occurring by a free-radical pathway. There is also evidence suggesting that such iron-sulfur clusters can function in coupling electron transfer to proton transport. By binding Cys ligands from different subunits, iron-sulfur clusters effect dimer formation, as in the Fe protein of nitrogenase. Further, by straddling protein structural elements, iron-sulfur clusters are able to stabilize structures that are required for specific functions (eg. endonuclease III of E. coli). Proteins of ISPG's class have also been shown to protect proteins from the attack of intracellular proteases. Finally, proteins of ISPG's class are thought to be capable of serving as storage devices for iron and possibly sulfide.

Thus, in a few examples, ISPG may be used advantageously as an iron or metal biosensor, for the treatment and/or diagnosis of iron overload disorders, or in applications involving stabilizing target proteins such as for protein production or for mediating protein interactions.

Additionally, structural aspects of ISPG suggest that it may be capable of mediating steroid hormone synthesis, either in human or animals, or in engineered cell culture systems for the large scale production of hormones. ISPG may be used to act as an adrenal ferredoxin (known as adrenodoxin (ADX)), a vertebrate mitochondrial protein which transfers electrons from adrenodoxin reductase to cytochrome P450scc, which is involved in cholesterol side chain cleavage. Its primary function as a soluble electron carrier between the NADPH-dependent adrenodoxin reductase and several cytochromes P450 makes it an irreplaceable component of the steroid hormones biosynthesis in the adrenal mitochondria of vertebrates.

### Drug Metabolism

Previous studies have revealed that cytochrome P-450 isozymes are responsible for drug metabolism, and oxidation by P-450 isozymes is a common aspect of the overall clearance of drugs. Further studies have revealed that genetic polymorphism of cytochrome P-450 isozymes underlies a wide spectrum of substrates specificity in drug oxidation. In certain cases, genetic mutation and/or deletion of one critical isozyme gene results in a significant alteration of a phenotype projected on substrate specificity. It has been reported that CYP2D6 oxidizes more than 30 drugs (for example, M. Eichelbaum et al., Pharmacol. Ther., Vol. 46, pp. 377-, 1990). Many anti-cancer drugs are known to be oxygenated by cytochrome P450 enzymes to yield metabolites that are cytotoxic or cytostatic toward tumor cells. These include several commonly used cancer chemotherapeutic drugs, such as cyclophosphamide (CPA), its isomer ifosfamide (IFA), dacarbazine, procarbazine, thio-TEPA, etoposide, 2-aminoanthracene, 4-ipomeanol, and tamoxifen (LeBlanc, G. A. and Waxman, D. J., Drug Metab. Rev. 20:395-439 (1989); Ng, S. F. and Waxman D. J., Intl. J. Oncology 2:731-738 (1993); Goeptar, A. R., et al., Cancer Res. 54:2411-2418 (1994); van Maanen, J. M., et al., Cancer Res. 47:4658-4662 (1987); Dehal, S. S., et al., Cancer Res. 57:3402-3406 (1997); Rainov, N. G., et al., Human Gene Therapy 9:1261-1273 (1998)). Bioreductive metabolism that results in drug activation is also catalyzed by cytochrome P450 enzymes for a variety of anti-cancer drugs. Examples of such drugs include Adriamycin, mitomycin C, and tetramethylbenzoquinone (Goeptar, A. R., et al., Crit. Rev. Toxicol. 25:25-65 (1995); Goeptar, A. R., et al., Mol. Pharmacol. 44:1267-1277 (1993)). Those who have homozygous alteration in this recessive gene, are so-called “poor metabolizers (PMs)” and may suffer from severe side effects due to poor metabolism of drugs (for example, see M. Eichelbaum et al., Pharmacol. Ther., Vol. 46, pp. 377-, 1990). Such genetic alterations occur at rates of from I to 30% in different ethnic populations (for example, L. M. Distlerath et al., J. Biol. Chem., Vol. 260, pp. 9057-,1985).

The ISPG protein of the invention is thought to be capable of functioning as a soluble electron carrier in the electron transport chain involving one or more of the several available cytochromes P450 enzymes. The ISPG protein may thus be useful in methods of killing neoplastic cells involving P450 (and ISPG) gene transfer and the use of bioreductive drugs that are activated by cytochrome P450, and in methods for evaluating the susceptibility of a sample compound to metabolism with respect to a specific cytochrome P450 isozyme system.

Thus, in a first aspect, a drug activation/gene therapy strategy has been developed based on a cytochrome P450 gene (“CYP” or “P450”) in combination with a cancer chemotherapeutic agent that is activated through a P450-catalyzed monoxygenase reaction (Chen, L. and Waxman, D. J., Cancer Research 55:581-589 (1995); Wei, M. X., et al., Hum. Gene Ther. 5:969-978 (1994); U.S. Pat. No. 5,688,773, issued Nov. 18, 1997). Presently known drug-enzyme combinations can utilize established chemotherapeutic drugs widely used in cancer therapy. Such methods to obtain enhanced chemosensitivity have been demonstrated both in vitro and in studies using a subcutaneous rodent solid tumor model and human breast tumor grown in nude mice in vivo, and is strikingly effective in spite of the presence of a substantial liver-associated capacity for drug activation in these animals (Chen, L., et al., Cancer Res. 55:581-589 (1995); Chen, L., et al., Cancer Res. 56:1331-1340 (1996)). The P450-based approach also shows significant utility for gene therapy applications in the treatment of brain tumors (Wei, M. X., et al., Human Gene Ther. 5:969-978 (1994); Manome, Y., et al., Gene Therapy 3:513-520 (1996); Chase, M., et al., Nature Biotechnol. 16:444-448 (1998)).

Although the P450/drug activation system has shown great promise against several tumor types, further enhancement of the activity of this system is needed to achieve clinically effective, durable responses in cancer patients. This requirement is necessitated by two characteristics that are inherent to the P450 enzyme system: (1) P450 enzymes metabolize drugs and other foreign chemicals, including cancer chemotherapeutic drugs, at low rates, with a typical P450 turnover number (moles of metabolite formed/mole P450 enzyme) of only 10-30 per minute; and (2) P450 enzymes metabolize many chemotherapeutic drugs with high Km values, typically in the millimolar range. This compares to plasma drug concentrations that are only in the micromolar range for many chemotherapeutic drugs, including drugs such as CPA and IFA. Thus, current approaches to P450 gene therapy may result in intratumoral drug activation at a low absolute rate and under conditions that are not saturating with respect to drug substrate. Furthermore, since P450 is expressed at a very high level in liver tissue, only a very small fraction of the administered chemotherapeutic drug is metabolized via the tumor cell P450 gene product using the currently available methods for P450 gene therapy (Chen, L. and Waxman, D. J., Cancer Res. 55:581-589 (1995)). As described in U.S. Pat. No. 6,207,648, one enhancement involves introducing a P450 reductase (RED) gene in combination with a cytochrome P450 gene (and thus a P450 gene product) into neoplastic cells, the enzymatic conversion of a P450-activated chemotherapeutic drug to its therapeutically active metabolites is greatly enhanced within the cellular and anatomic locale of the tumor, thereby increasing both the selectivity and efficiency with which neoplastic cells are killed.

In a preferred embodiment, further enhancements to known prodrug-enzyme strategies may be achieved by introducing an ISPG gene into neoplastic cells, either alone or in combination with a P450 gene and/or a P450 reductase gene. Suitable vectors for the introduction and expression of said ISTG and P450 genes are known to one of skill in the art. The introduction of the ISPG gene and subsequent expression of the ISPG gene product may increase the enzymatic conversion of a P450-activated chemotherapeutic drug to its therapeutically active metabolites. Thus, the invention comprises a method for killing neoplastic cells comprising: (a) infecting the neoplastic cells with a vector for gene delivery, the vector comprising an ISPG gene capable of mediating enzymatic conversion of a chemotherapeutic agent by a P450 enzyme; (b) optionally infecting the neoplastic cells with a vector for gene delivery, the vector comprising a cytochrome P450 gene and/or a gene encoding RED; (b) treating the neoplastic cells with a chemotherapeutic agent that is activated by the product of the cytochrome P450 gene; and (c) killing the neoplastic cells.

The present invention also provides a reagent composition for use in evaluating drug metabolism by a specific cytochrome P450 isozyme, which comprises a liver microsome lacking said specific P450, said specific P450 isozyme and a carrier material. The liver microsome may be of human source lacking CYP2D6, CYP2C 19, or CYP2A6. The CYP2D6 isozyme, CYP2C 19 isozyme, and CYP2A6 isozyme to be added may be a recombinant CYP2D6-expressing microsome, a recombinant CYP2C 19-expressing microsome, or a recombinant CYP2A6-expressing microsome. The reagent composition may comprise more than one kind of PM microsomes.

According to the present invention, there can be provided a reagent composition and a method for accurately quantitating the contribution of certain P450 isozymes such as CYP2D6, CYP2C 19, and CYP2A6 in drug metabolism. The present invention provides a method for evaluating the susceptibility of a sample compound to metabolism with respect to a specific cytochrome P-450 isozyme, which comprises contacting the sample compound with a reagent composition prepared by adding said specific cytochrome P-450 isozyme and an ISPG protein to liver microsomes lacking said specific cytochrome P-450 isozyme in a carrier material. ISPG would be useful in order to enhance efficiency of the P-450 isozyme in drug metabolism, thereby effectively amplifying the power of the assay to detect the contribution of a particular P-450 enzyme. In another embodiment the contribution to drug metabolism of a particular P-450 system can be assessed by focussing on a particular iron-sulfur protein associated with said specific P-450 system. In this aspect, the method comprises contacting the sample compound with a reagent composition prepared by adding said specific ISPG protein to liver microsomes lacking said ISPG protein in a carrier material. The method may further comprise (a) incubating a mixture of the sample compound and the reagent composition; (b) extraction of the reaction mixture obtained in Step (a); and (c) analyzing the reaction products isolated in Step (b). For the purposes of quantitating the assay, a plurality of the reagent compositions having different amount of the specific P-450 isozyme or ISPG protein may be subjected to Step (a) to (c), respectively. For example, the specific P-450 isozyme to be used in the method may be selected from CYP2D6, CYP2C19, CYP2A6, CYPIA1 and CYP2E1.

### Iron Biosensor

Iron-sulfur clusters have been found to serve as sensors of iron, dioxygen, superoxide ion and possibly nitric oxide. Two main mechanisms of sensing have been described. In one example, the oxidation [Fe²S²]¹⁺→[Fe²S²]²⁺ by dioxygen provides the signal for activation of a defense mechanism against superoxide, as observed with the SoxR protein of E. coli, and thus may serve a cytoprotective function (eg. useful for treatment of ischemia, etc.). In an alternative mechanism, oxidative disassembly or reassembly of a cluster provides the controlling signal as in the FNR protein of E. coli.

In a further detailed example, the ISPG protein of the invention maybe be used as an iron biosensor. An example of an iron biosensor is provided in U.S. Pat. No. 5,516,697 (Kruzel et al.). In summary, ISPG can be immobilized in the vicinity of a device to measure the change in pH, causing a detectable variation of the potential upon the binding of iron.

In the process of sequestering iron, the sensing element, the ISPG protein is expected to release a number of protons of hydrogen (H+) directly proportional to the atoms of iron bound. The release of protons during the binding of iron by ISPG becomes the operative feature which is measured by the biosensors. The release of protons causes a change in pH and is measured by an ion-selective field effect transistor or by pH sensitive paper. A sample containing iron is placed into a buffered solution, usually water. The sample may be diluted one or more times. In one embodiment of the sensors of the present invention, the release of protons is measured as the variation of the potential on the surface of an ion-selective field effect transistor (an ISFET) (Reviewed in: Biosensor Technology, edited by Buck et al. and published by Marcel Decker, Inc., 1990, entitled “Solid State Potentiometric Sensors” by Jiri Janata, pp 17-34). In another embodiment of the present invention, the protons released upon binding of iron by ISPG are detected by the change in pH using pH sensitive paper. Preferably, the iron selective element (ISPG) is incorporated in close proximity or integrated with the signal transducer, to give a reagentless sensing system for iron. Since the signal can be amplified, only small quantities of ISPG are needed for detection of iron. The ISFET is modified by immobilizing ISPG on the surface of the ISFET or by a disposable membrane with immobilized ISPG that is in close proximity to the ISFET by attaching the ISPG-modified membrane to the surface of the ISFET. A sample containing iron, for example a biological sample such as body fluid from a mammal, particularly a human, is then contacted with the ISPG -modified ISFET.

In order to produce an ISPG-modified ISFET as an independent sensor, an existing system which uses an ISFET designed to measure pH can be modified. Systems which presently use an ISFET to measure pH are the Sentron 2001 pH system, manufactured by Integrated Sensor Technology, Federal Way, Wash.; the Corning 360i pH system, manufactured by Corning Incorporated, Corning N.Y.; or Orion 610 pH system, manufactured by Orion Analytical Technology, Inc., Boston, Mass. The modification required to measure the amount of iron in a sample is either to place an immobilized layer of ISPG on the ISFET of such a system or, alternatively, to provide a ISPG-modified membrane, i.e. a membrane coated with ISPG, which will be in close proximity to the existing ISFET so as to detect the release of protons when the ISPG binds iron in a sample and records the change in potential.

Iron sensitive biosensors (as well as treatments for iron overload disorders) are extremely valuable It is estimated that 30,000,000 Americans suffer from different types of iron related disorders, including a substantial proportion with profound iron deficiency syndrome. Detection of bioaccessible iron is one of the most important measurements that doctors can use for early detection if iron deficiency, iron overload or other types of immunological disorders. To date iron is measured through a combination of blood tests that detect iron and iron binding capacity of transferrin, the protein that transports iron through the body. The current technology involves very sophisticated instrumentation which make this analysis prohibitively expensive and often requires qualified personnel to analyze the sample. Therefore, there is a need for the direct assay of iron that combines simplicity and economics. ISPG may therefore be advantageously used in development of a biosensor for detecting the amount of iron in a sample.

### Steroid Biosynthesis

As noted above, ISPG may be used as an adrenal ferredoxin (known as adrenodoxin (ADX)), a vertebrate mitochondrial protein which transfers electrons from adrenodoxin reductase to cytochrome P450scc, which is involved in cholesterol side chain cleavage and is an irreplaceable component of the steroid hormones biosynthesis in the adrenal mitochondria.

In therapeutic embodiments, ISPG may have particular importance in treatment of disorders where it is desired to increase the level of steroid hormone synthesis. As P450scc has a critical role in synthesis of the conversion of cholesterol into pregnenolone, ISPG may be used as a limiter or enhancer of steroid synthesis. In but one example, evidence has been shown that cytochrome P450scc activity in the human placenta is limited by the supply of electrons to the P450scc. Furthermore, Tuckey et al. Eur. J. Biochem. July 1999;263(2): 319-325 have shown that p450scc activity can be increased considerably by adding adrenodoxin reductase and adrenodoxin. Thus, ISPG may be useful in the treatment of reproductive disorders by augmenting the electron supply to P450scc, and thus increasing the level of progesterone synthesis. Accordingly, in another example, ISPG may be used to limit steroid synthesis, whether for therapeutic or for research uses.

ISPG may also be used in biological steroid synthesis processes for the production of steroid hormones. For example, Duport et al, Nat Biotechnol February 1998; 16(2): 186-9 report a system for self-sufficient biosynthesis of pregnenolone and progesterone in engineered yeast wherein the first two steps of the steroidogenic pathway were reproduced in Saccharomyces cerevisiae. Engineering of sterol biosynthesis by disruption of the delta 22-desaturase gene and introduction of the Arabidopsis thaliana delta 7-reductase activity and coexpression of bovine side chain cleavage cytochrome P450, adrenodoxin, and adrenodoxin reductase, lead to pregnenolone biosynthesis from simple carbon source. As ISPG is thought to be capable of functioning as an adrenodoxin protein, ISPG may be used as a function substitute in the system of Duport et al for adrenodoxin.

MTG (METALLOTHIONEIN) (Clone ID:654627)

SEQ ID NOS 96 and 413 and clone FL 11:654627_{—}182-5-3-0-F10-F encode the polypeptide of SEQ ID NOs:265 and 527 respectively, a metallothionein protein which binds heavy metal. Said polypeptide of the invention is also referred herein as MTG. It will be appreciated that all characteristics and uses of the polynucleotides of SEQ ID NOs:96 and 413 and polypeptides of SEQ ID NO:265 and 527, described throughout the present application also pertain to the human cDNA of clone 654627, and the polypeptides encoded thereby.

Metallothioneins (MT) [1,2,3] are small proteins which bind heavy metals such as zinc, copper, cadmium, nickel, etc., through clusters of thiolate bonds. MT's occur throughout the animal kingdom and are also found in higher plants, fungi and some prokaryotes and are thought to play a role in metal detoxification or in the metabolism and homeostasis of metals. On the basis of structural relationships MT's have been subdivided into three classes. Class I includes mammalian MT's as well as MT's from crustacean and molluscs, but with clearly related primary structure. Class II groups together MT's from various species such as sea urchins, fungi, insects and cyanobacteria which display none or only very distant correspondence to class I MT's. Class III MT's are atypical polypeptides containing gamma-glutamylcysteinyl units.

Vertebrate class I MT's such as the MTG protein of the invention are proteins of typically 60 to 68 amino acid residues, 20 of these residues are cysteines that bind to 7 bivalent metal ions. As a signature pattern we chose a region that spans 19 residues and which contains seven of the metal-binding cysteines, this region is located in the N-terminal section of class-I MT's. A consensus pattern for class I Mrs is as follows: C-x-C-[GSTAP]-x(2-C-x-C-x(2)-C-x-C-x(2)-C-x-K.

The MTG protein of SEQ ID NO 527 has a metallothionein domain (Prosite ref. PS00203) at amino acid positions 13-31; an N-glycosylation site at position 4 (NCSC), a protein kinase C phosphorylation site at amino acid positions 18 (SCK), 28 (SCK) and 55 (SQR); a casein kinase II phosphorylation site at amino acid position 41 (TLVD); an N-myristoylation site at amino acid positions 10 (GVSCTC); and a prokaryotic membrane lipoprotein lipid attachment site at amino acid position 3 (PNCSCAAGVSC).

### Therapeutics

Discovery of new proteins related to metallothioneins, and the polynucleotides that encode them, satisfies a need in the art by providing new diagnostic or therapeutic compositions useful in diagnosing and treating heavy metal toxicity, cancer, inflammatory disease and immune disorders.

Acute or chronic exposure to heavy metals such as lead, arsenic, mercury or cadmium leads to a variety of diseases and disorders involving neuromuscular, CNS, cardiovascular, and gastrointestinal effects. MTs may play a role in the prevention or alleviation of these conditions. In addition, MTs are transcriptionally regulated by glucocorticoids, which suggests that MTs have a direct role in the effects of glucocorticoids to treat inflammatory disease, immune disorders, and cancer. It is therefore thought that MTG may have important applications in the treatment of inflammatory disease, immune disorders, and cancer as well as in cytoprotection in a variety of therapeutic applications.

In one preferred example, the MTG nucleic acids and protein may be used for suppressing the production of sunburn cells which is applicable in various manners with minimal adverse side effects, a method of inducing metallothionein, a method of treating skin diseases and a method of screening ultraviolet rays, and further relates to cosmetic compositions and UV screening compositions.

Conventionally, steroids and zinc oxide formulations have been topically used as medicines for treating skin diseases such as dermatitis, sunburn, neurodermatitis, eczema and anogenital pruritus. Steroids, however, have been difficult to administer in large quantities for a prolonged period due to their strong adverse side effects. Zinc oxide formulations, which have local astringent action, involve problems with respect to the manufacture of pharmaceuticals, since they are insoluble in water and are not usually administered internally.

Zinc, one of the indispensable trace metals in the living body, is known to participate in the development of sexual organs, promotion of wound healing and is also known to be a component of a metalloenzyme, an accelerator for dehydrogenase, and to have various functions such as activating the immune system. Zinc is further known to be an inducing factor of metallothionein (MT), a metal-combining protein. It is reported that MT functions as a scavenger of free radicals which are generated at the onset of inflammations [“Dermatologica”, Hanada, k., et al., 179 (suppl. 1) 143 (1989)].

As proposed in U.S. Pat. No. 5,582,817 (Otsu et al), MTG may be useful in treatment of dermatological inflammations caused by external irritative stimulants, such as sunburn or the like, where MTG could act to quench the free radicals released from leukocytes, especially granulocytes which gather at the inflamed region, and thereby exhibit an anti-oxidation action to diminish cell damage, especially to normal lymphocytes, to activate the immune system and further to prevent the accelerated aging of the skin. Formation of sunburn cells (SBCs) could be suppressed by administering zinc for inducing MTG to be present, or to increase MTG in the epidermal keratinous layer. Anti-oxidation action of MTG can also be useful in the treatment of skin problems resulting from radiation therapy by X rays, alpha rays, beta rays, gamma rays, neutron rays and accelerated electron rays.

Various zinc compounds have been studied by Otsu et al (supra) with respect to their pharmacological activities, who reported that zinc salts or zinc complexes of a certain compound have an excellent action of inducing metallothionen (MT) and suppressing sunburn cell (SBC) production due to UV rays, and thereby useful as components of cosmetic compositions or medicines for purposes of ameliorating sunburn, preventing sunburn, ameliorating sufferings from skin diseases and ameliorating other radiation induced disorders, leading to completion of the invention.

There are two different types of dermatological reactions caused by sunlight, one is an acute inflammatory change in the skin called sunburn, and the other is a subsequent melanin pigmentation called suntan. The light having a wave length in the range of 320 nm or less, called UVB, induces sunburn and is responsible for erythematous change. The erythemic reaction caused by UV rays, as opposed to a burn injury, does not occur immediately after the exposure to the sunlight, but rather occurs after a latent period of several hours. When sunburned skin is histopathologically examined, various degrees of inflammatory changes are recognized in the epidermis and dermis depending on the dose of radiation. Among such changes, a notable one is the generation of so-called sunburn cells (SBC) in the epidermis. A histologically stained tissue sample presents strongly and acidophilically stained cells which have pyknotic nuclei. This phenomenon indicates the necrosis of epidermal cells (“Fragrance Journal”, 9, 15-20 (1991). In order to prevent sunburn, para-aminobenzoic acid derivatives, cinnamic acid derivatives or the like UV absorbers mentioned above are used, but their UV absorbing effects are not necessarily satisfactory. What is more, they raise problems of cumbersome handling upon use, poor stability, low compatibility with other components of the composition, and also involve unsolved problems in water-resistance and oil-resistance.

In the field of medicines for the treatment of skin diseases, development of medicines which have minimal adverse side effects, and which have novel functions obtainable by both external and internal administrations has been desired. Also, in the field of the therapy and prevention of radiation disorders, medicines which can suppress and cure the disorders caused by oxidative reactions have been desired. Lastly, in the field of the manufacture of cosmetics, cosmetics which overcome the above-mentioned problems such as handling upon use and stability of the composition have been desired. Accordingly, the present invention encompasses providing therapeutic agents for treating skin diseases having the above-mentioned characteristics, wherein said agents are capable of inducing MTG for suppressing the formation of sunburn cells, and for use in cosmetic compositions. Also encompassed are methods of screening for therapeutic agents for treating skin diseases comprising bringing a test compound into contact with a cell, tissue or animal model of disease, and detecting induction of MTG expression or function.

### Gene Expression Systems

The MTG nucleic acid and proteins of the invention may also be advantageously used in the production of recombinant proteins as biopharmaceutical products at commercial scale.

Previously, genes have been extensively expressed in mammalian cell lines, particularly in mutant Chinese Hamster Ovary (CHO) cells deficient in the dihydrofolate reductase gene (dhfr) as devised by the method of Urlaub et al, PNAS U.S.A. 77, 42164220, 1980. A variety of expression systems have been used. Many vectors for the expression of genes in such cells are therefore available. Typically, the selection procedures used to isolate cells transformed with the expression vectors rely on using methotrexate to select for transformants in which both the dhfr and the target genes are coamplified. The dhfr gene, which enables cells to withstand methotrexate, is usually incorporated in the vector with the gene whose expression is desired. Selection of cells under increasing concentrations of methotrexate is then performed. This leads to amplification of the number of dhfr genes present in each cell of the population, as cells with higher copy numbers withstand greater concentrations of methotrexate. As the dhfr gene is amplified, the copy number of the gene of interest increases concomitantly with the copy number of the dhfr gene, so that increased expression of the gene of interest is achieved. Unfortunately, these amplified genes have been reported to be variably unstable in the absence of continued selection (Schimke, J. Biol. Chem. 263, 5989-5992, 1988). This instability is inherent to the presently available expression systems of CHO dhfr.sup.- cells. For many years, several promoters have been used to drive the expression of the target genes such as the SV40 early promoter, the CMV early promoter and the SR.alpha. promoter. The CMV and SR.alpha. promoters are claimed to be the strongest (Wenger et al, Anal. Biochem. 221, 416-418, 1994).

In one report, the .beta.-interferon promoter has also been used to drive the expression of the .beta.-interferon gene in the mutant CHO dhfr.sup.- cells (U.S. Pat. No. 5,376,567). In this system, however, the selected CHO dhfr⁻ cells had to be superinduced by the method of Tan et al (Tan et al, PNAS U.S.A. 67, 464471, 1970; Tan et al, U.S. Pat. No. 3,773,924) to effect a higher level of .beta.-interferon production. In this system a significant percentage of the superinduced .beta.-interferon produced by the CHO dhfr- cells was not glycosylated. The mouse metallothionein gene (mMT1) promoter has also been used for the expression of beta-interferon genes in CHO cells, BHK and LTK.sup.- mouse cells (Reiser et al 1987 Drug Res. 37, 4, 482485). However, the expression of .beta.-interferon with this promoter was not as good as the SV40 early promoter in CHO cells. Further, .beta.-interferon expression from these cells mediated by the mMT1 promoter was inducible by heavy metals. Heavy metals are however extremely toxic to the cells and this system was therefore abandoned. Instead, Reiser et al used the CHO dhfr- expression system in conjunction with the SV40 early promoter (Reiser et al, Drug Res. 37,4, 482485 (1987) and EP-A-0529300) to produce .beta.-interferon in CHO dhfr- cells as derived by the method of Urlaub et al (1980).

As described in U.S. Pat. No. 6,207,146 (Tan et al) beta.-interferon was expressed in wild-type CHO cells using a metallothionein based system. MTG may thus be used in similar applications so as to provide a system for expression of recombinant proteins. Tan et al demonstrates wild-type CHO cells transfected with a vector comprising a .beta.-interferon gene under the control of a mouse sarcoma viral enhancer and mouse metallothionein promoter (MSV-mMT1), a neo gene under the control of promoter capable of driving expression of the neo gene in both E. coli and mammalian cells and a human metallothionein gene having its own promoter. Transfected cells capable of expressing .beta.-interferon were selected by first exposing cells to geneticin (antiobiotic G418) and thus eliminating cells lacking the neo gene and then exposing the surviving cells to increasing concentrations of a heavy metal ion.

The heavy metal ion enhanced the MSV-mMT1 promoter for the .beta.-interferon gene, thus increasing .beta.-interferon expression. The heavy metal ion also induced the human metallothionein gene promoter, causing expression of human metallothionein. The human metallothionein protected the cells against the toxic effect of the heavy metal ion. The presence of the heavy metal ion ensured that there was continual selection of cells which had the transfecting vector, or at least the .beta.-interferon gene and the human metallothionein gene and their respective promoters, integrated into their genome.

The selected cells that had been successfully transfected expressed .beta.-interferon. Expression was surprisingly improved when the cells were cultured in the presence of Zn²⁺. The .beta.-interferon had improved properties, in particular a higher bioavailability, than prior .beta.-interferons.

These findings have general applicability and suggest that the MTG gene of the present invention may be used accordingly in expression systems. Accordingly, the present invention provides a nucleic acid vector comprising:

(i) a coding sequence which encodes a protein of interest and which is operably linked to a promoter capable of directing expression of the coding sequence in a mammalian cell in the presence of a heavy metal ion; (ii) a first selectable marker sequence which comprises an MTG gene of the invention and which is operably linked to a promoter capable of directing expression of the MTG gene in a mammalian cell in the presence of a heavy metal ion; and optionally (iii) a second selectable marker sequence which comprises a neo gene and which is operably linked to a promoter capable of directing expression of the neo gene in a mammalian cell;

CDPG (Glycosyl Phosphatidylinositol-Linked Glycoprotein) (Clone ID:1000902917)

SEQ ID NOS 3 and 341 and clone FL11:1000902917_{—}223-524-0-G3-F encode the polypeptide of SEQ ID NOS 172 and 458 respectively, a glycosyl phosphatidylinositol-linked glycoprotein protein which is thought to be a signal transducing polypeptide expressed in lymphoid, myeloid, and erythroid cells. Said polypeptide comprises a CD24 signal transducing domain as well as a GPI-anchor of the invention is also referred herein as CDPG. CDPG is believed to be highly glycosylated, and it is expected that CDPG molecular weight will vary among cell types and cell developmental stage due to differences in glycosylation patterns, providing further specificity in its use as a therapeutic target. It will be appreciated that all characteristics and uses of the polynucleotides of SEQ ID NOs: 3 and 341 and polypeptides of SEQ ID NO: 172 and 458, described throughout the present application also pertain to the human cDNA of clone 1000902917, and the polypeptides encoded thereby.

It is suggested that CDPG may have a specific role to play in early thymocyte development. The CDPG protein is thought to be extensively o-glycosylated may be capable of modulating b-cell activation responses. As a signaling transducer, the CDPG polypeptide's signal transducing function may in some embodiments be triggered by the binding of a lectin-like ligand to the CD24 domain carbohydrates, and the release of second messengers allowing signaling. The CDPG polypeptide is thought to have important functions in regulating the differentiation and/or growth of lymphoid, myeloid, and erythroid cells, including specifically promoting antigen dependent proliferation of b-cells, and preventing their terminal differentiation into antibody-forming cells.

Additionally, based on a growing body of evidence characterizing the CD24 domain function, it is proposed that CDPG may have a role as a potent stimulator of neurite outgrowth, and thus may be useful in the treatment of central nervous system disorders.

Fragments of CDPG may also be useful, eg. GPI-anchor domain for example in the development of soluble T-cell receptors (U.S. Pat. No. 6,080,840) or any suitable application where a temporally controlled solubilization of a protein of interest is desired. For example, a fragment comprising the CDPG GPI-anchor domain can be used in the production of soluble molecules by replacing the transmembrane domains of the cDNA of a protein of interest with a sequence comprising the CDPG glycosylphosphatidyl inositol (GPI) linkage. These chimeric cDNAs are then transferred into an expression vector containing a strong promoter and a mutant (e.g. DHFR) gene allowing high levels of transcriptional expression and amplification of the gene. These chimeric genes are then cotransfected into a selected cell type, preferably lacking the endogenous protein of interest, and transfectants are selected and can also be screened with antibodies for the protein of interest. These GPI linked proteins of interest can then be solubilized by cleavage with the enzyme phosphatidyl inositol specific phospholipase C (PI-PLC) and purified/concentrated from the supernatant (e.g. by passage over a protein of interest-reactive antibody affinity column).

### Therapeutics

CDPG or inhibitors of CDPG may be used in the treatment of any disorder where it is desired to regulate B-cell proliferation or differentiation. CDPG or inhibitors thereof may be useful in the treatment of B-cell neoplasms, a heterogeneous group of diseases characterized by different maturation states of the B-cell, which are related to the aggressiveness of the disorder. Chronic lymphocytic leukemia (CLL) is characterized by proliferation and accumulation of B-lymphocytic leukemia (BLL) is characterized by proliferation and accumulation of B-lymphocytes that appear morphologically mature but are biologically immature. This disorder accounts for 30% of leukemias in Western countries. The disorder is characterized by proliferation of biologically immature lymphocytes, unable to produce immunoglobulins, which cause lymph node enlargement. As a regulator of B-cell proliferation and differentiation, CDPG and/or inhibitors of CDPG may be useful for inhibiting proliferation of leukemic B-cells in CLL patients.

CDPG may also be useful in the modulation of cell growth in the CNS. CD24 is known to be highly expressed in neurons and has been demonstrated as capable of inhibiting neurite outgrowth of dorsal root ganglion neurons while promoting neurite outgrowth of cerebellar neurons via interaction with an L1 protein.

### Selectable Cell Markers

In one aspect, the CDPG polypeptide may be used as a selectable cell marker and to a method of using the selectable marker to identify a cell. Viruses such as recombinant retroviruses have been used as a vehicle for gene transfer based on their potential for highly efficient infection and non-toxic integration of their genome into a wide range of cell types. The transfer of exogenous genes into mammalian cells may be used, for example in gene therapy to correct an inherited or acquired disorder through the synthesis of missing or defective gene products in vivo. The expression of exogenous genes in cells may be useful in somatic gene therapy, to correct hereditable disorders at the level of the gene. Hemopoietic stem cells are particularly suited to somatic gene therapy as regenerative bone marrow cells may be readily isolated, modified by gene transfer and transplanted into an immunocompromised host to reconstitute the host's hemopoietic system.

Gene therapy involving hone marrow transplant with recombinant primary hemopoietic stem cells requires efficient gene transfer into the stem cells. As a very small number of primary stem cells can reconstitute the entire host hemopoietic system it is important that the transferred gene be efficiently expressed in the recombinant stem cells transferred. The transfer of foreign genes into a reconstituted host hemopoietic system has been limited by the availability of a selectable marker which permits the rapid and non-toxic selection of cells which are efficiently expressing the transferred gene. Currently available selection markers may not be suitable for primary hemopoietic stem cells since they may alter the proliferative ability or biological characteristics of the cells. The transfer of foreign genes into a reconstituted host hemopoietic system has also been limited by the availability of a viral vector capable of expression in hemopoietic stem cells, especially where more than one transcriptional unit is present in the vector (Botrell, D. R. L. et al., 1987, Mol. Biol. Med. 4:229).

U.S. Pat. No. 5,804,177 (Humphries et al) has demonstrated that the cell surface protein CD24 (also M1/69-J11d heat stable antigen) can be used as a dominant marker in a recombinant viral vector. A nucleotide sequence encoding the cell surface protein CD24 in a recombinant viral vector was used to infect hematopoietic stem cells and cells infected with the recombinant viral vector were rapidly and non-toxically selected for in vitro using fluorescence activated cell sorting (FACS), demonstrating a good correlation between proviral copy number and expression of selectable marker.

CD24 is a signal transducing molecule found on the surface of most human B cells that can modulate their responses to activation signals, and is structurally closely related to CDPG. The CD24 cDNA (approximately 300 bps) has been cloned (Kay, R. et al, 1991, J. Immunol. 147:1412) and encodes a mature peptide of only 31 to 35 amino acids that is extensively glycosylated and attached to the outer surface of the plasma membrane by a glycosyl phosphatidylinositol lipid anchor. M1/69-J11d heat stable antigen is a genetically similar homologous murine peptide widely expressed on a variety of hemopoietic cell types (Kay, R. et al., 1990, J. Immunol. 145:1952).

It is thus proposed that a recombinant viral vector can be used to successfully transfer and express the CDPG gene in primitive hemopoietic stem cells such that they are able to repopulate lethally irradiated recipients. Preferably foreign CDPG antigen expression in repopulated animals persists post transplantation such that the biological function of the repopulated hemopoietic cells is not affected by the expression of the CDPG antigen. CDPG may subsequently be found to be expressed in any or all of hemopoietic lineages including granulocytes, macrophages, pro-erythrocytes, erythrocytes and T and B lymphocytes. Therefore, the cell surface protein CDPG may be particularly useful as a marker for hematopoietic stem cells capable of repopulation in vivo and as a selectable marker in gene therapy. The recombinant viral vectors also have the advantage that the nucleotide sequence encoding the marker is very small, leaving a large amount of space for the insertion of additional genes of interest such as those coding for exogenous genes.

In a preferred embodiment of the invention a recombinant viral vector is used to introduce the nucleotide sequence into the cell. Preferably, the CDPG nucleotide sequence is operatively linked to one or more regulatory elements. The recombinant viral vector of the invention may be used as a marker for an exogenous gene to be expressed in a host cell. The invention further provides a method of identifying a cell and progeny thereof comprising: providing a cell; infecting the cell with a recombinant viral vector of the invention under suitable conditions to allow expression of the cell surface protein CDPG on the cell; and, identifying the cell and progeny thereof by detecting expression of the cell surface protein CDPG on the cell or progeny thereof. Cells infected with a recombinant viral vector of the invention and expressing the cell surface protein may be transplanted into a host, and the cell and progeny thereof may be identified after transplantation by removing biological samples from the host, and assaying for cells expressing the cell surface protein. A recombinant viral vector of the invention may be directly introduced into a host.

### Enriching Stem Cell Compositions

As it is proposed that CDPG is involved in early thymocyte development and is found on the cell surface due to its GPI-anchor, CDPG nucleic acids and polypeptides of the present invention may also be used to obtain novel antibody compositions useful for preparing cell preparations containing human hematopoietic cells.

There is a continued interest in developing stem cell purification techniques. Pure populations of stem cells will facilitate studies of hematopoiesis. Transplantation of hematopoietic cells from peripheral blood and/or bone marrow is also increasingly used in combination with high-dose chemo- and/or radiotherapy for the treatment of a variety of disorders including malignant, nonmalignant and genetic disorders. Very few cells in such transplants are capable of long-term hematopoietic reconstitution, and thus there is a strong stimulus to develop techniques for purification of hematopoietic stem cells. Furthermore, serious complications and indeed the success of a transplant procedure is to a large degree dependent on the effectiveness of the procedures that are used for the removal of cells in the transplant that pose a risk to the transplant recipient. Such cells include T lymphocytes that are responsible for graft versus host disease (GVHD) in allogenic grafts, and tumour cells in autologous transplants that may cause recurrence of the malignant growth.

Hematopoietic cells have been separated on the basis of physical characteristics such as density and on the basis of susceptibility to certain pharmacological agents which kill cycling cells. The advent of monoclonal antibodies against cell surface antigens has greatly expanded the potential to distinguish and separate distinct cell types. There are two basic approaches to separating cell populations from bone marrow and peripheral blood using monoclonal antibodies. They differ in whether it is the desired or undesired cells which are distinguished/labeled with the antibody(s). In positive selection techniques the desired cells are labeled with antibodies and removed from the remaining unlabeled/unwanted cells. In negative selection, the unwanted cells are labeled and removed. Antibody/complement treatment and the use of immunotoxins are negative selection techniques, but FACS sorting and most batch wise immunoadsorption techniques can be adapted to both positive and negative selection. In immunoadsorption techniques cells are selected with monoclonal antibodies and preferentially bound to a surface which can be removed from the remainder of the cells e,g. column of beads, flasks, magnetic particles. Immunoadsorption techniques have won favor clinically and in research because they maintain the high specificity of targeting cells with monoclonal antibodies, but unlike FACSorting, they can be scaled up to deal directly with the large numbers of cells in a clinical harvest and they avoid the dangers of using cytotoxic reagents such as immunotoxins, and complement.

Current positive selection techniques for the purification of hematopoietic stem cells target and isolate cells which express CD34. However, positive selection procedures suffer from many disadvantages including the presence of materials such as antibodies and/or magnetic beads on the CD34⁺ cells, and damage to the cells resulting from the removal of these materials.

Negative selection has been used to remove minor populations of cells from clinical grafts. These cells are either T-cells or tumour cells that pose a risk to the transplant recipient. The efficiency of these purges varies with the technique and depends on the type and number of antibodies used. Typically, the end product is very similar to the start suspension, missing only the tumor cells or T-cells.

As described in U.S. Pat. No. 5,877,299, Thomas et al developed a negative selection technique that uses an antibody composition containing antibodies specific for glycophorin A, CD3, CD24, CD16, CD14 and optionally CD45RA, CD36, CD2, CD19, CD56, CD66a, and CD66b, which reportedly gave a cell preparation highly enriched for human hematopoietic and progenitor cells. Maximum enrichment of early progenitor and stem cells (CD34⁺, CD38⁻ cells) was observed when anti-CD45R and anti-CD36 were included in the antibody composition. However, as CDPG is proposed as acting in early thymocyte development, CDPG may be used advantageuously to develop more effective antibody compositions for selecting hematopoietic stem cells. Accordingly, the invention encompasses antibodies specific for CDPG polypeptides of the invention and antibody compositions comprising, consisting of or consisting essentially of antibodies specific for CDPG, glycophorin A, CD3, CD24, CD16, CD14 and optionally CD45RA, CD36, CD2, CD19, CD56, CD66a, and CD66b.

Use of the antibody composition comprising CDPG in a negative selection technique to prepare a cell preparation which is enriched for hematopoietic stem cells and progenitor cells may offer significant advantages over conventional techniques. The antibody composition is applied in one step to a sample of peripheral blood, bone marrow, cord blood or frozen bone marrow, preferably without additional enrichments steps which could result in loss of, or damage to, progenitor and stem cells.

PRSG (Proline-Rich Calcium-Binding Protein) and HSTG (Basic Histidine Rich Salivary Gland Peptide) (Clone ID:338112 and 1000839315 Respectively)

SEQ ID NOS 22 and 358 and clone FLI 1: 1000839315_{—}220-26-1-0-F3-F encode the polypeptide of SEQ ID NOS 191 and 475 respectively, a basic histidine rich salivary gland peptide referred to herein as HSTG and expected to have potent antimicrobial properties. Preferably, the amino acid sequence of HSTG (SEQ ID NO 475) comprises a tyrosine at amino acid position 40. The HSTG protein also comprises a Pattern-DE: Protein kinase C phosphorylation site at amino acid position 51 (SSK). It will be appreciated that all characteristics and uses of the polynucleotides of SEQ ID NOs: 22 and 358 and polypeptides of SEQ ID NO: 191 and 475, described throughout the present application also pertain to the human cDNA of clone 1000839315, and the polypeptides encoded thereby.

SEQ ID NOS 8 and 345 and clone FL11:338112_{—}174-1-1-0-A11-F encode the polypeptide of SEQ ID NOS 177 and 462 respectively, a proline-rich protein referred to herein as PRSG believed to be a component of saliva and a calcium binding protein also possessing potent antimicrobial properties. Preferably, the amino acid sequence of PRSG (SEQ ID NO 462) comprises a proline residue at amino acid position 96, an arginine residue at amino acid position 100, a glutamine residue at amino acid positoin 102, and/or a glycine residue at amino acid position 103. The PRSG protein also comprises a casein kinase II phosphorylation site at amino acid positions 15 (SAQD), 24 (SQED), and 59 (SAGD) and an N-myristoylation site at amino acid position 52 (GGQQSQ). It will be appreciated that all characteristics and uses of the polynucleotides of SEQ ID NOs:8 and 345 and polypeptides of SEQ ID NO: 177 and 462, described throughout the present application also pertain to the human cDNA of clone 338112, and the polypeptides encoded thereby.

A study of saliva and its tooth-protective components reveals at least four important functions of saliva: (1) buffering ability, (2) a cleansing effect, (3) antibacterial action, and (4) maintenance of a saliva supersaturated in calcium phosphate. Several salivary constituents serve one or more of these functions. Research has yielded important information about organic and inorganic secretory products. It is also clear that saliva as a unique biologic fluid has to be considered in its entirety to account fully for its effects on teeth. Saliva is greater then the sum of its parts. One reason for this is that salivary components display redundancy of function, each often having more than one function. This redundancy, however, does not imply that proteins that share functional roles all contribute to the same degree. For instance, when comparing proteins that inhibit calcium phosphate precipitation, statherin and acidic proline-rich proteins are most potent, whereas histatins, cystatins, and mucins appear to play lesser roles. The complex interaction between proteins is another major factor contributing to saliva's function. In this regard, heterotypic complexes of various proteins have been shown to form on hydroxyapatite. Mucin binding to other salivary proteins, including proline-rich proteins, histatins, cystatins, and statherin, is well documented. The complexes, whether adsorbed to the tooth surface or in saliva, have important implications for bacterial clearance, selective bacterial aggregation on the tooth surface, and control of mineralization and demineralization. Finally, proteolytic activity of saliva generates numerous products whose biologic activities are often different from their parent compounds. The ability of saliva to deliver fluoride to the tooth surface constantly makes salivary fluoride an important player in caries protection largely by promoting remineralization and reducing demineralization. Saliva is well adapted to protection against dental caries. Saliva's buffering capability; the ability of the saliva to wash the tooth surface, to clear bacteria, and to control demineralization and mineralization; saliva's antibacterial activities; and perhaps other mechanisms all contribute to its essential role in the health of teeth. The fact that the protective function of saliva can be overwhelmed by bacterial action indicates the importance of prevention and therapy as in other infectious diseases. With knowledge of salivary components and their interactions, the use of modified oral molecules as therapeutic agents may become a important contributor to oral health.

Proline-rich proteins are major components of parotid and submandibular saliva in humans as well as other animals. They can be divided into acidic, basic and glycosylated proteins. The proline-rich proteins are apparently synthesized the acinar cells of the salivary glands and their phenotypic expression is under complex genetic control. The acidic proline-rich proteins will bind calcium with a strength which indicates that they may be important in maintaining the concentration of ionic calcium in saliva. Moreover they can inhibit formation of hydroxyapatite, whereby growth of hydroxyapatite crystals on the tooth surface in vivo may be avoided. Both of these activities as well as the binding site for hydroxyapatite are located in the N-terminal proline-poor part of the protein.

Basic histidine rich salivary gland peptides such as the peptide of SEQ ID NO. 191 and 475, also referred to as histatins are a group of electrophoretically distinct histidine-rich polypeptides with microbicidal activity found in human parotid and submandibular gland secretions. Histatins 1, 3, and 5 are homologous proteins that consist of 38, 32, and 24 amino acid residues, respectively, that have been shown to kill the pathogenic yeast, Candida albicans. More recently histatins 2, 4, 6, and 7-12 were isolated and characterized Troxier RF et al, J Dent Res January 1990;69(1):2-6. Histatin 2 was found to be identical to the carboxyl terminal 26 residues of histatin histatin 4 was found to be identical to the carboxyl terminal 20 residues of histatin 3; and histatin 6 was found to be identical to histatin 5, but contained an additional carboxyl terminal arginine residue. The amino acid sequences of histatins 7-12 formally corresponded to residues 12-24, 13-24, 12-25, 13-25, 5-11, and 5-12, respectively, of histatin 3, but could also arise proteolytically f histatin 5 or 6. Troxler et al provides further guidance on the structural elements and relationship of histatins to one another in the context of their genetic origin, biosynthesis and secretion into the oral cavity, and potential as reagents in anti-candidal studies. The HSTG polypeptide and fragments thereof are therefore expected to have valuable properties and uses in antimicrobial applications, particularly in antifungal applications. Supporting such uses is a considerable body of evidence, including MacKay B J et al, Infect Immun. June 1984;44(3):695-701, Growth-inhibitory and bactericidal effects of human parotid salivary histidine-rich polypeptides on Streptococcus mutants; MacKay B J et al, Infect Immun. June 1984;44(3):688-94, Isolation of milligram quantities of a group of histidine-rich polypeptides from human parotid saliva; Pollock J J et al, Infect Immun. June 1984;44(3):702-7, Fungistatic and fungicidal activity of human parotid salivary histidine-rich polypeptides on Candida albicans; and Xu T et al, Infect Immun. August 1991;59(8):2549-54, Anticandidal activity of major human salivary histatins. Furthermore, tissue distribution of RNAs for cystatins, histatins, statherin, and proline-rich salivary proteins in humans and macaques is further discussed in Sabatini et al, J Dent Res July 1989;68(7):113845.

In further embodiments, the skilled artisan will appreciate that fragments and analogues of PRSG and HSTG may readily be generated and selected. Selection of preferred fragments and analogies may be carried out by assaying for a desired antimicrobial activity. For example, synthetic histatin analogues and methods for obtaining such analogies with broad-spectrum antimicrobial activity are described in Helmerhorst E J et al, Biochem J 1997 Aug. 15;326 (Pt 1):3945, where histatin analogies inhibited the growth of the second most common yeast found in clinical isolates, Torulopsis glabrata, of oral- and non-oral pathogens such as Prevotella intermedia and Streptococcus mutants, and of a methicillin-resistant Staphylococcus aureus.

Thus, in preferred embodiments, the PRSG and/or HSTG polypeptides or fragments thereof may be used in oral, injectable, topical or edible compositions for the treatment of infection PRSG and/or HSTG polypeptides may also be used as antimicrobiavantifungal compositions for disinfection of surfaces (e.g. in industrial settings).

In a preferred example further discussed below, the PRSG and/or HSTG polypeptides or fragments thereof are used in oral, topical (e.g. mouthwash) or edible compositions optionally containing additional salivary proteins to provide an anticaries effect. While there is an interest in developing and marketing products which reduce caries without reliance on a high level of fluoride ions (such as in fluoridated water and fluoride toothpastes), there have not been many reports of such approaches meeting with success. While certain cysteine-rich proteins have been proposed useful in the treatment of dental caries (U.S. Pat. No. 5,688,766, Revis et al), the present invention provides PRSG and HSTG polypeptides which may provide higher potency, efficacy and range of disinfection and protection.

PRSG and HSTG polypeptide compositions can be administered in a formulation comprising a carrier. A preferred carrier composition for the active(s) of this invention are oral compositions. Such compositions include toothpastes, mouthrinses, liquid dentifrices, lozenges, chewing gums or other vehicle suitable for use in the oral cavity. Toothpastes and mouthrinses are the preferred systems. The abrasive polishing material contemplated for use in the toothpaste compositions of the present invention can be any material which does not excessively abrade dentin. These include, for example, silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, and resinous abrasive materials such as particulate condensation products of urea and formaldehyde, and others such as disclosed by Cooley et al. in U.S. Pat. No. 3,070,510, Dec. 25, 1962, incorporated herein by reference. Mixtures of abrasives may also be used. Silica dental abrasives, of various types, can provide the unique benefits of exceptional dental cleaning and polishing performance without unduly abrading tooth enamel or dentin. For these reasons, they are preferred for use herein. Flavoring agents can also be added to toothpaste compositions. Suitable flavoring agents include oil of wintergreen, oil of peppermint, oil of spearmint, oil of sassafras, and oil of clove. Sweetening agents which can be used include aspartame, acesulfame, saccharin, dextrose, levulose and sodium cyclamate. Flavoring and sweetening agents are generally used in toothpastes at levels of from about 0.005% to about 2% by weight. Toothpaste compositions can also contain emulsifying agents. Suitable emulsifying agents are those which are reasonably stable and foam throughout a wide pH range, including non-soap anionic, nonionic, cationic, zwittefionic and amphoteric organic synthetic surfactants. Water is also present in the toothpastes of this invention. Water employed in the preparation of commercially suitable toothpastes should preferably be deionized and free of organic impurities. In preparing toothpastes, it is necessary to add some thickening material to provide a desirable consistency. Preferred thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents in ane amount from 0.2% to 5.0% by weight of the total composition can be used. It is also desirable to include some humectant material in a toothpaste to keep it from hardening. Suitable humectants include glycerin, sorbitol, and other edible polyhydric alcohols at a level of from about 15% to about 70%.

Another preferred embodiment of the present invention is a mouthwash composition. Conventional mouthwash composition components can comprise the carrier for the agents of the present invention. Mouthwashes generally comprise from about 20:1 to about 2:1 of a water/ethyl alcohol solution or be alcohol free and preferably other ingredients such as flavor, sweeteners, humectants and sudsing agents such as those mentioned above for dentifrices. The humectants, such as glycerin and sorbitol give a moist feel to the mouth. Generally, on a weight basis the mouthwashes of the invention comprise 0% to 60% (preferably 5% to 20%) ethyl alcohol, 0% to 20% (preferably 5% to 20%) of a humectant, 0% to 2% (preferably 0.01% to 1.0%) emulsifying agents, 0% to 0.5% (preferably 0.005% to 0.06%) sweetening agent such as saccharin or natural sweeteners such as stevroside 0% to 0.3% (preferably 0.03% to 0.3%) flavoring agent, and the balance water.

The pH of the present compositions and/or the pH in the mouth can be any pH which is safe for the mouth's hard and soft tissues. Such pH's are generally from about 3 to about 10, preferably from about 4 to about 8. Other acceptable oral carders include gums, lozenges, as well as other forms. Such suitable forms are disclosed in U.S. Pat. No. 4,083,955, Apr. 11, 1978 to Grabenstetter et al. incorporated herein in its entirety by reference. Edible compositions are also suitable for use as the carrier compositions herein. Edible compositions include many types of solid as well as liquid compositions. Such compositions include, for example, soft drinks, citrus drinks, cookies, cakes, breads among many others. Such compositions may contain sugar or another sweetener, water, flour, shortening, other fibers such as wheat, corn, barley, rye, oats, psyllium and mixtures thereof.

HSDG (Hydroxysteroid Dehydrogenase) (Clone ID:495917)

SEQ ID NOS 54 and 381 and clone FL11:495917_{—}160-22-4-0-D8-F encode the polypeptide of SEQ ID NOS 223 and 498 respectively, a hydroxysteroid dehydrogenase referred to herein as HSDG. As the HSDG polypeptide is implicated in steroid hormone regulation, preferably glucocorticoid metabolism, HSDG may be useful in any applications where steroid hormones levels are to be increased or inhibited. HSDG may be useful in the treatment of disease treatable by steroid hormones. HSDG inhibitors may also be useful in systems for self-sufficient biosynthesis of steroid hormones such as glucocorticoids such as in engineered cells comprising elements of the synthesis pathway. HSDG inhibitors of endogenous HSDG activity may allow the recovery of higher amounts of glucocorticoids and/or other synthesized steroid hormones from these cell systems.

It will be appreciated that all characteristics and uses of the polynucleotides of SEQ ID NOs: 54 and 381 and polypeptides of SEQ ID NO: 223 and 498, described throughout the present application also pertain to the human cDNA of clone 495917, and the polypeptides encoded thereby.

The HSDG polypeptides of the invention comprise leucine zipper pattern (Prosite ref. PS00029) at amino acid positions 58-79 as well as a N-myristoylation site at amino acid position 36 (GANAGV) of SEQ ID NO 498. In one example, HSDG polypeptides may be used in the production or therapeutic modulation of glucocorticoids. The skilled artisan will recognize that any suitable HSDG polypeptides or variants or fragments thereof capable of metabolizing glucocorticoids can readily be used.

In one embodiment HSDG activity can be determined by detecting levels of glucocorticoids or metabolites or glucocorticoids. Moreover, structural aspects of 11beta-hydroxysteroid dehydrogenase have been documented in the art and may serve as a guidelines for developing suitable HSDG variants and fragments. The HSDG polypeptides of the invention may allow modulation of glucocorticoid activity or identification (e.g. drug screening) of compounds capable of specifically modulating glucocorticoid levels or activity. HSDG may be useful in allowing the modulation of steroid hormone synthesis, or glucocorticoid synthesis to be carried in a tissue specific manner, thereby offering improved methods for treating disease with decreased risk of side effects.

Corticosteroids, also referred to as glucocorticoids are steroid hormones, the most common form of which is cortisol. Modulation of glucocorticoid activity is important in regulating physiological processes in a wide range of tissues and organs. Glucocorticoids act within the gonads to directly suppress testosterone production (Monder, C et al, (1994) Steroids 59, 69-73). High levels of glucocorticoids may also result in excessive salt and water retention by the kidneys, producing high blood pressure.

Glucocorticoid action is mediated via binding of the molecule to a receptor, defined hereinafter as either a mineralocorticoid receptor (MR) or a glucocorticoid receptor (GR). Krozowski, Z. S. et al, ((1983) Proc. Natl. Acad. Sci. USA 80, 6056-6060) and Beaumont, K. et al, ((1983) Endocrinology 113, 2043-2049) showed that MR of adrenalectomised rats have an equal affinity for the mineralocorticoid aldosterone and glucocorticoids, for example corticosterone and cortisol. Confirmatory evidence has been found for human MR (Arriza, J. L et al, (1988) Neuron I, 887-900). In patients suffering from the congenital syndrome of Apparent Mineralocorticoid Excess (AME), cortisol levels are reportedly elevated and bind to and activate MRs normally occupied by aldosterone, the steroid that regulates salt and water balance in the body. Salt and water are retained in AME patients causing severe hypertension.

Like HSDG, the enzyme 11β-hydroxysteroid dehydrogenase (11βHSD), also discussed in U.S. Pat. No. 5,965,372 (Funder et al) may be involved in converting glucocorticoids into metabolites that are unable to bind to MRs (Edwards et al, (1988) Lancet. 2: 986-9; Funder et al, (1988) Science 242, 583,585), present in mineralocorticoid target tissues, for example kidney, pancreas, small intestine, colon, as well as the hippocampus, placenta and gonads. For example, in aldosterone target tissues 11βHSD inactivates glucocorticoid molecules, allowing the much lower circulating levels of aldosterone to maintain renal homeostasis. When the 11βHSD enzyme is inactivated, for example in AME patients or following administration of glycyrrhetinic acid, a component of licorice, severe hypertension results. Further, placental 11βHSD activity may protect the foetus from high circulating levels of glucocorticoid which may predispose to hypertension in later life (Edwards et al., 1993). Biochemical characterisation of activity has indicated the presence of at least two 11βHSD isoenzymes (11βHSD1 and 11βHSD2) with different cofactor requirements and substrate affinities. The 11βHSD1 enzyme is a low affinity enzyme that prefers NADP+ as a cofactor (Agarwal et al., 1989). The 11βHSD2 enzyme is a high affinity enzyme (Km for glucocorticoid=10 nM), requiring NAD+, not NADP+ as the preferred cofactor, belonging to a class of glucocorticoid dehydrogenase enzymes hereinafter referred to as “NAD+ dependent glucocorticoid dehydrogenase” enzymes.

Inverse correlation between 11βHSD enzyme activity in human granulosa-lutein cells and the success of IVF has further been shown, suggesting that activity of this enzyme might be related to the success of embryo attachment and implantation following IVF. The measurement of ovarian 11βHSD enzyme activity as a prognostic indicator for the outcome of assisted conception in all species, is the subject of UK Patent Application No 9305984. However, the disclosure of Michael et al. ((1993) Lancet 342, 711-712), and corresponding UK Patent Application No 9305984 do not identify, or even suggest which isoenzyme in the ovary might be a predictive indicator of IVF embryo transfer, or a means of distinguishing isoenzymes of 11βHSD in the prediction of IVF embryo transfer outcomes. In fact, the enzyme assay procedure might detect all isoenzymes of 11βHSD activity in the cell, some of which may be hitherto uncharacterised.

Thus, the human HSDG hydroxysteroid dehydrogenase enzyme and the nucleic acids encoding it providing novel means for the development of gene therapies and identification of HSDG activators and inhibitors which alter the endogenous activity of this hydroxysteroid dehydrogenase enzyme in a cell. The present invention also permits the screening, through genetic or immunological means, levels of expression of genes encoding the NAD+ dependent glucocorticoid dehydrogenase enzyme in various tissue or organ types, including for example, skin, colon, kidney, placenta, and gonads, amongst others.

S100G Calcium Binding Protein (Clone ID:200895)

The polynucleotides of clone FL11:200895_{—}116-055-1-0-H11-F and SEQ ID NOS 132 and 428 encode for the S100 calcium binding protein referred to herein as S100G. SEQ ID NOS 301 and 542 provide the amino acid sequence corresponding to the nucleic acid sequences of SEQ ID NOS 132 and 428, respectively. It will be appreciated that all characteristics and uses of the polynucleotides of SEQ ID NOs: 132 and 428 and polypeptides of SEQ ID NO: 301 and 542, described throughout the present application also pertain to the human cDNA of clone200895, and the polypeptides encoded thereby.

### Background

In nearly all eukaryotic cells, calcium (Ca²⁺) functions as an intracellular signaling molecule in diverse cellular processes including cell proliferation and differentiation, neurotransmitter secretion, glycogen metabolism, and skeletal muscle contraction. Within a resting cell, the concentration of Ca²⁺ in the cytosol is extremely low, <10⁻⁷ M. However, when the cell is stimulated by an external signal, such as a neural impulse or a growth factor, the cytosolic concentration of Ca²⁺ increases by about 50-fold. This influx of Ca²⁺ is caused by the opening of plasma membrane Ca²⁺ channels and the release of Ca²⁺ from intracellular stores such as the endoplasmic reticulum. Ca²⁺ directly activates regulatory enzymes, such as protein kinase C, which trigger signal transduction pathways.

The protein of SEQ ID NOS 301 and 542 is a calcium binding S100 protein typically found in heart and muscle. S100 proteins are low-molecular weight calcium binding proteins that are believed to play an important role in various cellular processes such as cytoskeletavmembrane interactions, cell division and differentiation. The expression of S100 proteins has been evaluated in a variety of disorders. For example, S100 protein have been evaluated as markers of inflammatory disease, including ulcerative colitits, Crohn's disease, and as serum markers for subjects with infections diseases including AIDS and malaria and for subjects with hematological disease. Evidence has accumulated that indicates that S100 proteins can alter cellular invasion and metastatic spead of cancer. For example, S100 protein is expressed in dendritic cells in human transitional cell carcinoma of the bladder and the invasive potential of these tumor has been found to correlate with the presence of S100 protein expressing cells.

### Therapeutics and Diagnostics

The S100G protein of SEQ ID NOS 301 and 542 disclosed herein provides new calcium binding S100 protein compositions useful in the diagnosis, prevention, and treatment of cancer, reproductive disorders, immune disorders, neuronal disorders, vesicle trafficking disorders and developmental disorders.

CBPs are implicated in a variety of disorders and several CBPs have proven to be effective therapeutic targets for which small molecule inhibitors could be developed. However, while several CBPs are targets for widely-used therapeutic treatments, it would be advantageous to provide further CBPs allowing more selective therapeutic treatments for disease to be developed. In one example, calcineurin is found in the cells of all eukaryotes ranging from yeast to mammals. Calcineurin is a target for inhibition by the immunosuppressive agents cyclosporin A and FK506 emphasizing its importance in immune disorders (Kissinger, C. R. et al. (1995) Nature 378:641-644). Calcineurin also plays a critical role in transcriptional regulation and growth control in T-lymphocytes (Wang, M. G. et al. (1996) Cytogenet. Cell Genet. 72:236-241). However, inhibition of calcineurin phosphatase activity has been implicated both in the mechanism of immunosuppression and in the observed toxic side effects of FKS06 in nonlymphoid cells, suggesting that identification of a new (FK binding proteins (FKBPs) that can mediate calcineurin inhibition and are restricted in its expression to T cells could provide new immunosuppressive drugs may be identified that, by virtue of their specific interaction with the FKBP, would be targeted in their site of action (Baughman G, et al Mol Cell Biol August 1995;15(8):4395402). In another CBP example, levels of CaM are increased several-fold in tumors and tumor-derived cell lines for various types of cancer (Rasmussen, C. D. and Means, A. R. (1989) Trends in Neuroscience 12:433438). Calcium binding S100β is another example of a CBP involved in a variety of disorders. Like the S100G protein of the invention, S100β contains an EF-hand motif. S100β is abundantly expressed in the nervous system. S100β levels are increased in the blood and cerebrospinal fluid of patients with neurological injury resulting from cerebral infarction, transient ischemic attacks, hemorrhagia, head trauma, and Down's syndrome. Furthermore, S100β and other neural-specific CBPs may also protect against neurodegenerative disorders, such as Alzheimer's, Parkinson's, and Huntington's diseases. S100β is produced and secreted by glial cells in the central and peripheral nervous systems (Allore, R. J. et al. (1990) J. Biol. Chem. 265:15537-15543). The accumulation of S100β in mature glial cells is associated with the microtubule network. S100β promotes neuronal differentiation and survival but may be detrimental to cells if overexpressed. The selective overproduction has been implicated in the progression of the neuropathological changes in Alzheimer's disease which may involve mitotic protein kinases (Marshak, D. R. and Pena, L. A. (1992) Prog. Clin. Biol. Res. 379:289-307). Adult T-cell leukaemia (ATL) is a mature T-cell malignancy which is caused by human T lymphotrophic virus type-I. Diminished surface expression of the T-cell receptor alpha beta (TCRαβ+) complex is a specific feature of ATL cells. S100β is not detectable in CD4+, TCRαβ+ ATL cells, but is expressed in CD4-, CD8-, TCRαβ+ leukaemic cells from four ATL patients. This suggested that increased levels of S100β may be associated with the diminished surface expression of the TCRαβ+ complex in ATL (Suzushima, H. et al. (1994) Leuk. Lymphoma 13:257-262). Elevated serum levels of S100β are associated with disseminated malignant melanoma metastases, suggesting that serum S100β may be of value as a clinical marker for progression of metastatic melanoma (Henze, G. et al. (1997) Dermatology 194:208-212). In yet another example, messenger RNA levels encoding human calgizzarin (an S100-like protein), as well as those encoding phospholipase A₂, are elevated in colorectal cancers compared with those of normal colorectal mucosa (Tanaka, M. et al. (1995) Cancer Lett. 89:195-200). Finally, an intracellular S100 calcium-binding protein has been isolated from rat peritoneum. This protein, MRP14, is one of two migration inhibitory factor-related proteins that are expressed in peritoneal macrophages in the arthritis-susceptible Lewis/N rat (Imamichi, T. et al. (1993) Biochem. Biophys. Res. Comm. 194:819-825).

However, despite the many uses and therapeutics based on S100 proteins and other-CBPs, it would be advantageous to selectively target CBPs which are involved in a disorder and which are found specifically in the targeted cells or tissues. Thus, in a first embodiment, the S100G protein of the invention can be used for the development of selective inhibitors of calcium signaling, eg. preferably inhibitors of transcriptional regulation and cell growth control.

In one embodiment, an antagonist of S100G may be administered to a subject to prevent or treat a neuronal disorder. Such disorders may include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, neurofibromatosis, Parkinson's disease, paranoid psychoses, schizophrenia, and Tourette's disorder. In one aspect, an antibody which specifically binds S100G may be used directly as an antagonist or indirectly as a targeting or delivery mechanism for bringing a pharmaceutical agent to cells or tissue which express S100G.

In one embodiment, an antagonist of S100G may be administered to a subject to prevent or treat a vesicle trafficking disorder. Such disorders may include, but are not limited to, cystic fibrosis, glucose-galactose malabsorption syndrome, hypercholesterolemia, diabetes mellitus, diabetes insipidus, hyper- and hypoglycemia, Grave's disease, goiter, Cushing's disease, and Addison's disease; gastrointestinal disorders including ulcerative colitis, gastric and duodenal ulcers; other conditions associated with abnormal vesicle trafficking including AIDS; allergies including hay fever, asthma, and urticaria (hives); autoimmune hemolytic anemia; proliferative glomerulonephritis; inflammatory bowel disease; multiple sclerosis; myasthenia gravis; rheumatoid and osteoarthritis; scleroderma; Chediak-Higashi and Sjogren's syndromes; systemic lupus erythematosus; toxic shock syndrome; traumatic tissue damage; and viral, bacterial, fungal, helminth, and protozoal infections. In one aspect, an antibody which specifically binds S100G may be used directly as an antagonist or indirectly as a targeting or delivery mechanism for bringing a pharmaceutical agent to cells or tissue which express S100G.

In one embodiment, an antagonist of S100G may be administered to a subject to prevent or treat an immunological disorder. Such disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, Werner syndrome, and autoimmune thyroiditis; complications of cancer, hemodialysis, and extracorporeal circulation; viral, bacterial, fungal, parasitic, protozoal, and helminthic infections; and trauma. In one aspect, an antibody which specifically binds S100P may be used directly as an antagonist or indirectly as a targeting or delivery mechanism for bringing a pharmaceutical agent to cells or tissue which express S100G.

In one embodiment, an antagonist of S100G may be administered to a subject to prevent or treat a neoplastic disorder. Such disorders may include, but are not limited to, adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In one aspect, an antibody which specifically binds S100P may be used directly as an antagonist or indirectly as a targeting or delivery mechanism for bringing a pharmaceutical agent to cells or tissue which express S100G.

In addition, the S100G nucleic acids and polypeptides of the present invention can be used to identify compounds for the treatment of a subject experiencing negative side effects from the administration of other pharmaceuticals, such as those drugs that disrupt the body's calcium homeostasis. Co-administration of said compounds would be useful to counter-effect iatrogenically caused dysfunction of calcium metabolism.

An antagonist of S100G may be produced using methods which are generally known in the art. In particular, purified S100G may be used to produce antibodies or to screen libraries of pharmaceutical agents to identify those which specifically bind S100G. In one example, a positive screening for drugs that specifically inhibit the Ca2+-signaling activity was carried out on the basis of the growth promoting effect on a yeast mutant with a peculiar phenotype (Shitamukai A et al, Biosci Biotechnol Biochem September 2000;64(9):1942-6). An inappropriate activation of a signaling pathway in yeast often has a deleterious physiological effect and causes various defects, including growth defects. In a certain genetic background (deltazds1) of Saccharomyces cerevisiae, the cell-cycle progression in G2 is specifically blocked in the medium with CaCl2 by the hyperactivation of the Ca2+-signaling pathways. Shitamukai et al provide an example of a drug screening procedure designed to detect the active compounds that specifically attenuate the Ca2+-signaling activity on the basis of the ability to abrogate the growth defect of the cells suffering from the hyperactivated Ca2+signal. Screening conditions were established for the drugs that suppress the Ca2+-induced growth inhibition using known calcineurin inhibitors as model compounds, and an indicator strain with an increased drug sensitivity was constructed with a syr1/erg3 null mutation.

In another embodiment, a vector expressing the complement of the polynucleotide encoding S100P may be administered to a subject to treat or prevent a neuronal disorder, immunological disorder, neoplastic disorder or vesicle trafficking disorder including, but not limited to, those described above. In other embodiments, any of the proteins, antagonists, antibodies, agonists, complementary sequences or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Expression of S100 proteins have been evaluated as serum markers for melanoma (Henze et al, Dermatology 194:208-212; Buer et al, 1997 Brit. J. Cancer 75: 1373-1376; Sherbert et al. 1998; Anticancer Res. 18:2415-2422) and more recently as serum markers for cancer in general (particularly breast, colon and lung cancers) and that their detectability in serum can have prognostic and/or therapeutic significance in cancer. Furthermore auto-antibodies to S100 proteins were found in cancer patients. (International Patent Publication No. WO 00/26668). The S100G protein of the invention may thus be used in the diagnosis and prevention of cancer, for identification of subjects predisposed to cancer, for monitoring patients undergoing treatment for cancer based on the increased level of S100G protein(s) in biological fluid samples of subjects.

Methods for diagnosis and prognosis of cancer in a subject may comprise

a) detecting a S100G protein in a biological fluid sample obtained from a subject, and

b) comparing the level of protein detected in the subject's sample to the level of protein detected in a control sample,

wherein an increase in the level of S100G protein detected in the subject's sample as compared to control samples is an indicator of a subject with cancer of at increased risk for cancer.

The invention also comprises methods for diagnosis and prognosis of a subject with cancer comprising:

a) contacting a serum sample derived from a subject with a sample containing S100G protein antigens under conditions such that a specific antigen-antibody complex binding can occur; and

b) detecting the presence of immunospecific binding of autoantibodies present in the suject's serum samples to the S100G protein;

wherein the presence of immunospecific binding of autoantibodies indicates the presence of cancer.

Assays for detection of S100G protein in a sample can be accomplished by any suitable method, including immunoassays where in S100G proteins are detected by their interaction with an S100G specific antibody. In addition, reagents other than antibodies such as for example polypeptides that specifically bind S100G may be used.

In yet further embodiments, the S100G protein of the invention may be useful for the development of specific anti-S100 antibodies which are specific for S100 proteins other than S100G. As several S100 proteins have been implicated in disease, it may be advantageous to develop a panel of S 100 specific antibodies to characterize disease, eg. cancers. Thus, S100G proteins and antibodies thereto may be advantageous to distinguish cancer types. S100G proteins and antibodies may also be useful in the screening of S100 specific antibodies by determining the selectivity of a given anti-S100 antibody for its target, and eliminating antibodies which are cross-reactive with S100G proteins.

### Stuctural Aspects of S100 Proteins of the Invention

The S100 proteins are a group of low molecular mass (approximately 10-12 kDa) acidic Ca²⁺-binding proteins, so named after the solubility of the first isolated protein in 100% saturated ammonium sulfate. The most striking conserved feature of these proteins is the presence of an EF-hand. The S100 proteins have two Ca²⁺-binding domains. One of these domains is a basic helix-loop-helix domain, the other domain is an acidic helix-loop-helix EF-hand (Kligman, D. and Hilt, D. C. (1 988) Trends Biochem. Sci. 13:437442). The EF-hand domain also encompasses a part of a region within S100 proteins which specifically identifies members of the S100 family of proteins which have a low affinity for Ca²⁺ ions (S100/ICaBP; PROSITE PS00303, SWISSPROT, PFAM PF01023). The EF-hand is characterized by a twelve amino acid residue-containing loop, flanked by two alpha-helices, orientated approximately 90 degrees with respect to one another. Aspartate (D) and glutamate (E) residues are usually found bordering the twelve amino acid loop. In addition, a conserved glycine residue in the central portion of the loop is found in most Ca²⁺-binding EF-hand domains. Oxygen ligands within this domain coordinate the Ca²⁺ ion (Kretsinger, R. H. and Nockolds, C. E. (1973) J. Biol. Chem. 248:3313-3326). It will also be appreciated by the skilled artisan that modifications of the S100G polypeptide may readily be made based on extensive knowledge of CBP stucture and Ca(2+) binding mechanisms, such as Sastry M et al, (Structure 1998;6:223-23 1), describing the three-dimensional structure of Ca(2+)bound calcyclin and its implications for Ca(2+}signal transduction by S100.

Recently, a protein designated S100A 13 has been discovered and characterized which shared significant primary structure similarity with the S100G polypeptide of the invention. (Wicki et al, (1996) BBRC 227:594-599).

The binding to calcium induces a conformational change in the S100 proteins, and this may then affect the secondary effector proteins. This mode of protein-protein interaction and modulation of the activity of the secondary effector protein is similar to that seen with calmodulin, also containing the EF-hands. The S100G protein of the invention comprises an ICaBP type calcium binding domain domain at amino acid positions 9 to 52 and casein kinase II phosphorylation site patterns at amino acid position 7 (TELE); 34 (SVNE); and 55 (SLDE) of SEQ ID NO 542.

The S100G polypeptides of the invention may thus also be used in any situation in vivo or in vitro where it is desired to modulate, preferably decrease the level of free calcium, or in applications involving sensing a change in calcium concentration. In one aspect, S100G nucleic acids and polypeptides may be used to develop a calcium biosensor. Calcium biosensors may have particular utility in detecting abnormalities in calcium transport that result in uncompensated influx into, or efflux from, the extracellular fluid, will result in hypercalcaemia or hypocalcaemia, respectively. Such abnormalities in serum calcium concentration may have profound effects on neurological, gastrointestinal, and renal function (Bushinsky DA et al, Lancet 1998 Jul. 25;352 (9124):306-1 1). Calcium biosensors may be developed by using any suitable means known in the art to detect a conformational change induced by the binding of calcium to the EF-hand domains of the S100G polypeptides. Preferably, the conformational change is detected by detecting a change in the ability of the S100G protein to bind a selected secondary effector protein

As the distribution of particular S100 proteins is dependent on specific cell types, the S100 proteins may be involved in transducing the signal of an increase in intracellular calcium in a cell type-specific fashion (Wu, T. et al. (1997) J. Biol. Chem. 272:17145-17153).

CaMLP (Calcium Binding Protein) (Clone ID:500742698)

Calcium is one of the “second messengers” which relays chemical and electrical signals within a cell. This signal transduction and, hence the regulation of biological processes, involves interaction of calcium ion with high-affinity calcium-binding proteins (CBPs). Disclosed herein in SEQ ID NOS 184 and 469 is one such protein, encoded by the nucleic acid sequences of SEQ ID NOS 15 and 352, respectively, and the clone FLI 1:500742698_{—}204-614-0-B2-F, and further referred to herein as CAMLP, which is thought to act as a Ca²⁺ sensing and binding protein involved in diverse aspects of cell proliferation (such as for example of hepatocytes, melanoma cells, leukemic lymphocytes, and HUVEC (human umbilical vein endothelial cells)) and differentiation. It will be appreciated that all characteristics and uses of the polynucleotides of SEQ ID NOs:15 and 352 and polypeptides of SEQ ID NO:184 and 469, described throughout the present application also pertain to the human cDNA of clone 500742698, and the polypeptides encoded thereby. Notably, the CaMLP polypeptide contains EF hand calcium-binding domains (PROSITE PS00018) at amino acid positions 81-93 and at position 129-141 of SEQ ID NO 469. CaMLP

The cellular processes in which Ca²⁺ functions as an intracellular signaling molecule are diverse, including cell proliferation and differentiation, neurotransmitter secretion, glycogen metabolism, and skeletal muscle contraction. Within a resting cell, the concentration of Ca²⁺ in the cytosol is extremely low, <10.sup.-7 M. However, when the cell is stimulated by an external signal, such as a neural impulse or a growth factor, the cytosolic concentration of Ca²⁺ increases by about 50-fold. This influx of Ca²⁺ is caused by the opening of plasma membrane Ca²⁺ channels and the release of Ca²⁺ from intracellular stores such as the endoplasmic reticulum. Ca²⁺ directly activates regulatory enzymes, such as protein kinase C, which trigger signal transduction pathways. Ca²⁺ also binds to specific Ca²⁺-binding proteins (CBPs) such as calbindins, troponin C, calmodulin, and S-100 proteins which then activate multiple target proteins including enzymes, membrane transport pumps, and ion channels. Calmodulin (CaM) is the most widely distributed and the most common mediator of calcium effects and appears to be the primary sensor of Ca²⁺ changes in eukaryotic cells. The binding of Ca²⁺ to CaM induces marked conformational changes in the protein permitting interaction with, and regulation of over 100 different proteins. CBP interactions are involved in a multitude of cellular processes including, but not limited to, gene regulation, DNA 30 synthesis, cell cycle progression, mitosis, cytokinesis, cytoskeletal organization, muscle contraction, signal transduction, ion homeostasis, exocytosis, and metabolic regulation (Celio, M. R. et al. (1996) Guidebook to Calcium-binding Proteins, Oxford University Press, Oxford, UK, pp. 15-20).

### Therapeutics

The CaMLP protein of SEQ ID NOS 184 and 469 disclosed herein provides new calcium binding protein compositions useful in the diagnosis, prevention, and treatment of cancer, reproductive disorders, immune disorders, neuronal disorders and developmental disorders.

Calcium binding proteins (CBPs) are implicated in a variety of disorders and several CBPs have proven to be effective therapeutic targets for which small molecule inhibitors could be developed. However, while several CBPs are targets for widely-used therapeutic treatments, it would be advantageous to provide further CBPs allowing more selective therapeutic treatments for disease to be developed. Evidence has accumulated for a large number of CBPs suggesting involvement in cell proliferative disorders. It is proposed that CaMLP may be useful as a tissue specific calmodulin homologue allowing the development of specific inhibitors and activators having increased selectivity and safety (decreased side effect profile). To date, calmodulin antagonists are reportedly useful for the treatment of some malignant tumors, particularly those of the central nervous system, as well as lung tumors. The antitumor activity of calmodulin antagonists, as well as successful chemotherapy using the same, has been described, for example, in Sculler et al. Cancer Res., 50:1645-1649 (1990) and Hait et al. Cancer Res., 50:6636-6640 (1990). U.S. Pat. No. 5,340,565, additionally describes the use of calmodulin antagonists or inhibitors as agents which enhance the effectiveness of a chemotherapeutic agent or radiation treatment. Specifically, described therein is a method of inhibiting or killing a tumor or cancer cell in a human patient undergoing radiation therapy or chemotherapy, for example with such chemotherapeutic agents as cisplatin (Platinol®), by additionally administering a calmodulin binding agent which inhibits calmodulin activity.

Calmodulin is also believed to play a pathogenic role in the tissue damage caused by burns and frostbite (Beitner et al., Gen. Pharmac. 20: 641-646, 1989), as well as in dermatitis and other conditions involving keratinocyte hyperproliferation. The methods of the present invention may be applied to the treatment of these and other conditions wherein antagonism of calmodulin activity is desirable.

Thus, as discussed above, CaMLP protein of the invention shares structural similarity with the ubiquitous intracellular receptor protein calmodulin suggesting that CaMLP may be useful in the development of selective CaMLP inhibitors. The nucleic acids and polypeptides of the invention thus provide a novel therapeutic target in particular for cell proliferative disorders. In one aspect, said nucleic acids and protein may be used in drug screening processes to develop selective calmodulin and other calcium binding protein antagonists which do not inhibit the polypeptide of the invention. In another aspect, the nucleic acids and polypeptides of the invention may be used in drug screening processes to identify selective modulators of the CaMLP without inhibiting calmodulin, thereby identifying compounds less likely to cause unwanted side effects. In yet another aspect, the nucleic acids and polypeptides of the invention may be used in drug screening processes to identify selective modulators of both calmodulin and CaMLP, thereby identifying compounds having increased potency.

Upon calcium binding, CaMLP may interact with a number of protein targets in a calcium dependent manner, thereby altering a number of complex biochemical pathways that can affect the overall behavior of cells. The calcium-calmodulin complex for example controls the biological activity of more than thirty different proteins including several enzymes, ion transporters, receptors, motor proteins, transcription factors, and cytoskeletal components in eukaryotic cells.

As described in U.S. Pat. No. 5,840,697, Blondelle et al have peptide inhibitors of calmodulin. A number of other calmodulin targeted compounds are known and used for a variety of therapeutic applications. For instance, chlorpromazine (Thorazine.RTM.) and related phenothiazine derivatives, disclosed, for example, in U.S. Pat. No. 2,645,640, are calmodulin antagonists useful as tranquilizers and sedatives. Naphthalenen-sulfonamides, also calmodulin antagonists, are known to inhibit cell proliferation, as disclosed, for example, in Hidaka et al. ((1981), PNAS, 78:43544357) and are useful as antitumor agents. In addition, the cyclic peptide cyclosporin A (Sandimmune®), disclosed in U.S. Pat. No. 4,117,118, is as an immunosuppressive agent which is thought to work by inhibiting calmodulin mediated responses in lymphoid cells.

Many existing calmodulin inhibitors have undesirable biological effects when administered at concentrations sufficient to block calmodulin. These undesirable biological effects include non-specific binding to other proteins or receptors, as described, for example, in Polak et al, ((1991), J. Neurosci. 11:534-542.) In addition, negative side effects such as toxicity can occur. A specific example is the toxic side effects from cyclosporin A. Therefore, a need exists for calmodulin targeted agents, and in particular calmodulin antagonists which inhibit calmodulin without having additional, undesirable biological or side effects. In particular there is a need for inhibitors which are specific to calmodulin and which do not have toxic side effects.

In addition, the CaMLP nucleic acids and polypeptides of the present invention can be used to identify compounds for the treatment of a subject experiencing negative side effects from the administration of other pharmaceuticals, such as those drugs that disrupt the body's calcium homeostasis. Co-administration of said compounds would be useful to counter-effect iatrogenically caused dysfunction of calcium metabolism. Such disorders include, but are not limited to, organ damage, autoimmune disorders, psychotic disorders, tumors and drug induced dysfunction, such as negative side effects subsequent to administration of pharmaceuticals. For example, organ or tissue transplantation can result in autoimmune disorders, such as tissue graft (allograft) rejections.

It is well known that calmodulin-targeted compounds which are antagonists can be used as immunosuppressive agents. In addition, also as described above, such compounds are widely used as sedative or anti-psychotic agents. Furthermore, there is evidence that calmodulin (and hence CaMLP) antagonists are useful for the treatment of some malignant tumors, particularly those of the central nervous system, as well as lung tumors. The antitumor activity of calmodulin antagonists, as well as successful chemotherapy using the same, has been described, for example, in Sculler et al. Cancer Res., 50:1645-1649 (1990) and Hait et al. Cancer Res., 50:6636-6640 (1990), both of which are incorporated herein by reference. U.S. Pat. No. 5,340,565, which is incorporated herein by reference, additionally describes the use of calmodulin antagonists or inhibitors as agents which enhance the effectiveness of a chemotherapeutic agent or radiation treatment. Specifically, described therein is a method of inhibiting or killing a tumor or cancer cell in a human patient undergoing radiation therapy or chemotherapy, for example with such chemotherapeutic agents as cisplatin (Platinol), by additionally administering a calmodulin binding agent which inhibits calmodulin activity.

It has also been found that extracellular calmodulin inhibits TNF release and facilitates elastase release, providing further suggestion that CaMLP, CaMLP analogues and CaMLP receptor agonists are useful agents for regulating the inflammatory process. CaMLP antagonists, which include CaMLP receptor antagonists and CaMLP -binding molecules, may be used to block the interaction of CaMLP with a receptor, thus providing the opposite effect from CaMLP, its analogues and receptor agonists. CaMLP may serve as a potent modulator of self-directed inflammation by assisting in the recognition of self vs. non-self as prokaryotes (e.g., bacterial pathogens) do not contain CaMLP. In some situations such as in tumor necrosis, release of extracellular CaMLP may lead to an inappropriate host response and failure of the immune/inflammatory systems to eradicate tumor cells. Further, a diagnostic test has been developed which can discern patient variabilities in TNF inhibition by calmodulin and other substances. This test can be utilized in monitoring individual patients for determining effective therapies, and for predicting efficacy of therapy with extracellular CAMLP, CaMLP analogues or CaMLP receptor agonists on the one hand and CaMLP antagonists on the other. A diagnostic test for elastase has also been developed with similar utility.

Lysozyme C Protein of SEQ ID NO: 196 (Internal Designation 482181) and Related Protein of SEQ ID NO:479

The polypeptides of SEQ ID NO: 196 and SEQ ID NO:479encoded by the cDNA of SEQ ID NO:27 and 362, respectively, belong to the widely conserved family of lysozyme C precursors (Prager and Jollés, Lysozymes: model enzymes in biochemistry and biology, ed. Jolles, 9-321 (1996), Qasba and Kumar, Crit. Rev. Biochem. Mol. Biol. 32:255-306 (1997)), which disclosures are hereby incorporated by reference in their entireties. The protein of SEQ ID NOs: 196 and 479 or part thereof plays a role in glycoprotein and/or peptidoglycan metabolism, probably as a glycosyl hydrolase of family 22. Thus, the protein of the invention or part thereof is involved in immune and inflammatory responses and has antiviral, antibacterial, anti-inflammatory and/or anti-histaminic functions. Preferred polypeptides of the invention are polypeptides comprising the amino acids of SEQ ID NO:196 from positions 19 to 100, or from positions 1 to 100. Other preferred polypeptides of the invention are fragments of SEQ ID NO: 196 having any of the biological activities described herein. The glycolytic activity of the protein of the invention or part thereof may be assayed using any of the assays known to those skilled in the art including those described in Gold and Schweiger, M. Methods in Enzymology, Vol. XX, Part C pp. 537-542, Ed. Moldave, Academic Press,New York and London, 1971 and in the U.S. Pat. No. 4,255,517, which disclosures are hereby incorporated by reference in their entireties.

Lysozymes, which are ubiquitous proteins found in most body secretions, are defined as 1,4-beta-N-acetylmuramidase which cleave the glycoside bond between the C-1 of N-acetyl-muramic acid and the C-4 of N-acetylglucosamine in the peptidoglycan of bacteria. They have various therapeutic properties, such as antiviral, antibacterial, anti-inflammatory and antihistaminic effects. The activity of lysozymes as an anti-bacterial agent appears to be based on both its direct bacteriolytic activity and also on stimulatory effects in connection with phagocytosis of polymorphonuclear leucocytes and macrophages (Biggar and Sturgess, J. M. Infect Immunol. 16: 974-982 (1977); Thacore and Willet, Am. Rev. Resp. Dis. 93: 786-790 (1966); Klockars and Roberts, P. Acta Haematol 55: 289-292 (1976)), which disclosures are hereby incorporated by reference in their entireties. Lysozymes have proven to be not only a selective factor but also an effective factor against microorganisms of the mouth (Iacono et al, J. J. Infect. Immunol. 29: 623-632 (1980)), which disclosure is hereby incorporated by reference in its entirety. Lysozymes can also kill pathogens by acting synergistically with other proteins such as complement or antibody to lyse pathogenic cells. Lysozymes, also inhibit chemotaxis of polymorphonuclear leukocytes and limit the production of oxygen free radicals following an infection. This limits the degree of inflammation, while at the same time enhances phagocytosis by these cells. Other postulated functions of lysozymes include immune stimulation (Jolles, P. Biomedicine 25: 275-276 (1976) Ossermann, E. F. Adv. Pathobiol 4: 98-102 (1976)) and immunological and non-immunological monitoring of host membranes for any neoplastic transformation (Jolles, P. Biomedicine 25: 275-276 (1976); Ossermann, E. F. Adv. Pathobiol 4: 98-102 (1976)), which disclosures are hereby incorporated by reference in their entireties. Lysozymes may thus be used in a wide spectrum of applications (see U.S. Pat. No. 5,618,712, which disclosure is hereby incorporated by reference in its entirety). Determination of the lysozymes from serum and/or urine is used to diagnose various diseases or as an indicator for their development. In acute lymphoblastic leukaemia the lysozyme serum level is significantly reduced, whereas in chronic myelotic leukaemia and in acute monoblastic and myelomonocytic leukaemia the lysozyme concentration in the serum is greatly increased. The therapeutically effective use of lysozyme is possible in the treatment of various bacterial and virus infections (Zona, Herpes zoster), in colitis, various types of pain, in allergies, inflammation and in pediatrics (the conversion of cows milk into a form suitable for infants by the addition of lysozyme).

The invention relates to methods and compositions using the protein of the invention or part thereof to hydrolyze one or several substrates, alone or in combination with other substances, preferably antiviral, antifungal and/or antibacterial substances including but not limited to immunoglobulins, lactoferrin, betalysin, fibronectin, and complement components. Such substrates are glycosylated compounds, preferably containing beta-1-4-glycoside bonds, more preferably containing beta-1-4-glycoside bonds between n-acetylomuraminic acid and n-acetyloglucosamine. For example, the protein of the invention or part thereof is added to a sample containing the substrate(s) in conditions allowing hydrolysis, and allowed to catalyze the hydrolysis of the substrate(s). In a preferred embodiment, the hydrolysis is carried out using a standard assay such as those described by Gold and Schweiger, supra, and U.S. Pat. Nos. 5,871,477 and 4,255,517, which disclosures are hereby incorporated by reference in their entireties. In a preferred embodiment, the protein of the invention or part thereof may be used to lyze recombinant bacteria in order to recover the recombinant DNA, the recombinant protein of interest, or both using, for example, any of the assays described in Sambrook, et al., Molecular Cloning: A Laboratory Manual, Second Edition, Cold Spring Harbor Laboratory Press (1989), which disclosure is hereby incorporated by reference in its entirety.

In an embodiment, the protein of the invention or part thereof is used to hydrolyze contaminating substrates, preferably exogenous substrates from bacterial, fungal or viral origins, in an aqueous sample or onto a material, preferably glassware and plasticware. In particular, the protein of the invention or part thereof may be used as a disinfectant in dental rinse, in protection of aqueous systems or in preparing material for medical applications using any of the methods and compositions described in U.S. Pat. Nos. 5,069,717, 4,355,022 and 5,001,062, which disclosures are hereby incorporated by reference in their entireties. In a preferred embodiment, the protein of the invention is used as a host resistance factor in infants' formulas to convert cow's milk into a form more suitable for infants as described in U.S. Pat. No. 6,020,015, which disclosure is hereby incorporated by reference in its entirety. In another preferred embodiment, the protein of the invention or part thereof may be used as a food preservative (see Hayashi et al., Agric. Biol. Chem. (European Edition of Japanese Journal of Agriculture, Biochemistry and Chemistry), Vol. 53, pp. 3173-3177, 1989), which disclosure is hereby incorporated by reference in its entirety. In addition, the protein of the invention or part thereof may be used to clarify xanthan gum fermented broth for applications in food and in cosmetic industries using the method described in U.S. Pat. No. 5,994,107, which disclosure is hereby incorporated by reference in its entirety. In another preferred embodiment, compositions comprising the protein of the present invention or part thereof are added to samples or materials as a “cocktail” with other antimicrobial substances, preferably antibiotics or hydrolytic enzymes such as those described in U.S. Pat. Nos. 5,458,876 and 5,041,326, which disclosures are hereby incorporated by reference in their entireties, to decontaminate the samples. For example, the protein of the invention or part thereof may be used in place or in combination with antibiotics in cell cultures. The advantage of using a cocktail of hydrolytic enzymes is that one is able to hydrolyze a wide range of substrates without knowing the specificity of any of the enzymes. Using a cocktail of hydrolytic enzymes also protects a sample or material from a wide range of future unknown contaminants from a vast number of sources. For example, the protein of the invention or part thereof is added to samples where contaminating substrates, preferably exogenous substrates from bacterial, fungal or viral origins, is undesirable in an amount sufficient to promote hydrolysis of said substrates. Alternatively, the protein of the invention or part thereof may be bound to a chromatographic support, either alone or in combination with other hydrolytic enzymes, using techniques well known in the art, to form an affinity chromatography column. A sample containing the undesirable substrate is run through the column to remove the substrate. Immobilizing the protein of the invention or part thereof on a support advantageous is particularly for those embodiments in which the method is to be practiced on a commercial scale. This immobilization facilitates the removal of the enzyme from the batch of product and subsequent reuse of the enzyme. Immobilization of the protein of the invention or part thereof can be accomplished, for example, by inserting a cellulose-binding domain in the protein. One of skill in the art will understand that other methods of immobilization could also be used and are described in the available literature. Alternatively, the same methods may be used to identify new substrates.

In addition, the protein of the invention or part thereof may be useful to identify or quantify the amount of a given substrate in biological fluids, foods, water, air, solutions and the like. In a preferred embodiment, the protein of the invention or part thereof is used in assays and diagnostic kits for the identification and quantification of exogenous substrates in bodily fluids including blood, lymph, saliva or other tissue samples, in addition to bacterial, fungal, plant, yeast, viral or mammalian cell cultures. In a preferred embodiment, the protein of the invention or part thereof is used to detect, identify, and or quantify eubacteria using reagents and assays described in U.S. Pat. No. 5,935,804, which disclosure is hereby incorporated by reference in its entirety. Briefly, the protein of the invention of part thereof is catalytically inactived, i.e. capable of binding but not cleaving a peptidoglycan comprising NAc-muramic acid in the eubacteria, using any of the methods known to those skilled in the art including those which produce a mutant enzyme, a recombinant-enzyme, or a chemically inactivated enzyme. The catalytically inactive protein of the invention is then incubated with an aliquot of a biological sample under conditions suitable for binding of the inactive enzyme to the peptidoglycan substrate. Then, the bound enzyme is detected to assess the presence or amount of the eubacteria in the biological sample.

In another embodiment, the nucleic acid of the invention or part thereof may be used to increase disease resistance of plants to bacterial, fungal and/or viral infections. A polynucleotide containing the nucleic acid of the invention or part thereof is introduced into the plant genome in conditions allowing correct expression of the transgenic protein using any methods known to those skilled in the art including those disclosed in U.S. Pat. Nos. 5,349,122 and 5,850,025, which disclosures are hereby incorporated by reference in their entireties.

In another preferred embodiment, the protein of the invention or part thereof may be useful to treat and/or prevent bacterial, fungal and viral infections in humans or in animals caused by various agents including but not limited to Streptococcus, Veillonella alcalescens, Actinomyces, Herpes simplex, Candida albicans, Micrococcus lysodeikticus and HIV by hydrolyzing the glycosylated compounds contained in such micro-organisms. In still a preferred embodiment, the protein of the invention or part thereof is used to prevent and/or treat bacterial, fungal and viral infections in immunocompromised individuals who lack fully functional immune systems, such as neonates or geriatric patients or HIV-infected individuals, or who suffer from a disease affecting the respiratory tract such as cystic fibrosis or the gastrointestinal tract such as ulcerative colitis or sprue.

In still another embodiment, the protein of the invention or part thereof may be used as a growth factor for in vitro cell culture, preferably for T cells and T cell lines, using techniques and methods taught in U.S. Pat. No. 5,468,635, which disclosure is hereby incorporated by reference in its entirety.

In addition, the protein of the invention or part thereof may be used to identify inhibitors for mechanistic and clinical applications. Such inhibitors may then be used to identify or quantify the protein of the invention in a sample, and to diagnose, treat or prevent any of the disorders where the protein's hydrolytic, immunostimulatory and/or inflammatory activities is/are undesirable and/or deleterious including but not limited to amyloidosis, colitis, lysosomal diseases, inflammatory and immune disorders including allergies and leukaemia. The protein of the invention may also be used to monitor host cell membranes for neoplastic transformation.

It will be appreciated that all characteristics and uses of the polynucleotides of SEQ ID NOs:27 and 362 and polypeptides of SEQ ID NO:196 and 479, described throughout the present application also pertain to the human cDNA of clone 482181, and the polypeptides encoded thereby.

Angiogenin Protein of SEQ ID NO: 176 (Internal Designation 114180) and Related Protein of SEQ ID NO:461

The polypeptides of SEQ ID NO:176 and SEQ ID NO:461 encoded by the extended cDNA SEQ ID NO:7 and SEQ ID NO:344, respectively, are ribonucleases that belongs to the pancreatic ribonuclease family (see reviews from Beintema, (1998) Cell. Mol. Life Sci. 54:763-5; Beintema and Kleineidam, (1998) Cell. Mol. Life Sci. 54:825-32, which disclosures are hereby incorporated by reference in their entireties). It will be appreciated that all characteristics and uses of the polynucleotides of SEQ ID NO:7 and SEQ ID NO:344 and polypeptides of SEQ ID NO:176 and SEQ ID NO:461, described throughout the present application also pertain to the human cDNA of clone 114180, and the polypeptides encoded thereby. In addition, the protein of the invention plays a role in angiogenesis as an angiogenin variant protein (see review from Badet, (1999) Pathol. Biol. 74:345-51, which disclosure is hereby incorporated by reference in its entirety). Preferred polypeptides of the invention are polypeptides comprising the amino acids of SEQ ID NO: 176 from positions 19 to 75, from positions 26 to 75, or from positions 63 to 69. Other preferred polypeptides of the invention are fragments of SEQ ID NO: 176 having any of the biological activity described herein. The ribonuclease activity of the protein of the invention or part thereof may be assayed using any of the assays known to those skilled in the art including those described in U.S. Pat. No. 5,866,119, which disclosure is hereby incorporated by reference in its entirety. The angiogenic activity of the protein of the invention or part thereof may be assayed using any of the assays known to those skilled in the art including those described by Fett et al. (1985) Biochem. 24, 5480-5486, which disclosure is hereby incorporated by reference in its entirety.

Ribonucleases are proteins which catalyze the hydrolysis of phosphodiester bonds in RNA chains. Pancreatic ribonucleases are pyrimidic-specific ribonucleases present in high quantity in the pancreas of a number of mammalia taxa and of a few reptiles. In addition to their function in hydrolysis of RNA, ribonucleases have evolved to support a variety of other physiological activities. Such activities include anti-parasite, anti-bacterium, anti-virus, anti-neoplastic activities, neurotoxicity, and angiogenesis. For example, bovine seminal ribonuclease is anti-neoplastic (Laceetti, et al. (1992) Cancer Res. 52: 4582-4586, which disclosure is hereby incorporated by reference in its entirety). Some frog ribonucleases display both anti-viral and anti-neoplastic activity (Youle, et al. (1994) Proc. Natl. Acad. Sci. USA 91: 6012-6016; Mikulski, et al. (1990) J. Natl. Cancer Inst. 82: 151-152; and Wu, et al. (1993) J. Biol. Chem. 268: 10686-10693), which disclosures are hereby incorporated by reference in their entireties. Eosinophil-derived neurotoxin (EDN) and eosinophil cationic protein (ECP) are related ribonucleases which possess neurotoxicity (Beintema, et al. (1988) Biochemistry 27: 45304538; Ackerman, (1993) In Makino, and Fukuda, Eosinophils: Biological and Clinical Aspects. CRC Press, Boca Raton, Fla., pp 33-74), which disclosures are hereby incorporated by reference in their entireties. In addition, ECP exhibits cytotoxic, anti-parasitic, and anti-bacterial activities. A EDN-related ribonuclease, named RNase k6, is shown to express in normal human monocytes and neutrophils, suggesting a role for this ribonuclease in host defense (Rosenberg, and Dyer, (1996) Nuc. Acid. Res. 24: 3507-3513), which disclosure is hereby incorporated by reference in its entirety.

Angiogenin is a tRNA-specific ribonuclease which binds protein partners on the surface of endothelial cells for endocytosis. Potential partners of angiogenin include heparin, plasminogen, elastase, angiostatin, actin, and a 170 kDa receptor on the surface of endothelial cells [Strydom, (1998) Cell. Mol. Life Sci. 54, 811-824, which disclosure is hereby incorporated by reference in its entirety ]. Endocytosed angiogenin is translocated to the nucleus where it promotes endothelial invasiveness required for blood vessel formation (Moroianu, and Riordan, (1994) Proc. Natl. Acad. Sci. USA 91: 1217-1221, which disclosure is hereby incorporated by reference in its entirety).

Although originally isolated from medium conditioned by human colon cancer cells (Fett et al. (1985), supra), and subsequently shown to be produced by several other histological types of human tumors [Rybak, et al. (1987) Biochem. Biophys. Res, Commun. 146, 1240-1248; Olson, et al., (1995) Proc. Natl. Acad. Sci. U.S.A. 92, 442446, which disclosures are hereby incorporated by reference in their entireties], angiogenin also is a constituent of human plasma and normally circulates at a concentration of 250-360 ng/ml [Shimoyama, et al. (1996) Cancer Res. 56, 2703-2706; Blaser, et al. (1993) Eur. J. Clin. Chem. Clin. Biochem. 31, 513-516, which disclosures are hereby incorporated by reference in their entireties]. It has also been shown that recurrent gastric cancer patients had a much higher serum concentration of angiogenin than primary gastric cancer patients [Shimoyama, and Kaminishi, (2000) J. Cancer Res. Clin. Oncol. 126, 468474, which disclosure is hereby incorporated by reference in its entirety].

Angiogenin is a potent inducer of angiogenesis [Fett, et al. supra]. Angiogenesis is a complex process of blood vessel formation comprising of several separate but interconnected steps at the cellular and biochemical level including: (i) activation of endothelial cells by the action of an angiogenic stimulus, (ii) adhesion and invasion of activated endothelial cells into the surrounding tissues and migration toward the source of the angiogenic stimulus, and (iii) proliferation and differentiation of endothelial cells to form a new microvasculature [Folkman, and Shing, (1992) J. Biol. Chem. 267, 10931-10934; Moscatelli, and Rifkin, (1988) Biochim. Biophys. Acta 948, 67-85, which disclosures are hereby incorporated by reference in their entireties]. While angiogenesis is a tightly-controlled process under usual physiological conditions, abnormal angiogenesis can have devastating consequences in pathological conditions such as arthritis, diabetic retinopathy and tumor growth. It is now well-established that the growth of virtually all solid tumors is angiogenesis dependent [Folkman, (1989) J. Natl. Cancer Inst. 82, 4-6, which disclosure is hereby incorporated by reference in its entirety]. Angiogenesis is also a prerequisite for the development of metastasis, since it provides the means whereby tumor cells disseminate from the original primary tumor and establish at distant sites [Mahadevan, and Hart, (1 990) Rev. Oncol. 3, 97-103; Blood, and Zetter (1990) Biochim. Biophys. Acta 1032, 89-118, which disclosures are hereby incorporated by reference in their entireties]. Therefore, interference with the process of tumor-induced angiogenesis can be an effective therapy for both primary and metastatic cancers. Indeed, several anti-angiogenic agents have been produced and are currently in the clinical trial stage.

The invention relates to methods and compositions using the protein of the invention or part thereof to hydrolyze one or several substrates, preferably nucleic acids, more preferably RNA, alone or in combination with other substances. For example, the protein of the invention or part thereof is added to a sample containing the substrate(s) in conditions allowing hydrolysis, and allowed to catalyze the hydrolysis of the substrate(s). Hydrolysis conditions as described in the U.S. Pat. No. 5,866,119 may be used, which disclosure is hereby incorporated by reference in its entirety.

In a preferred embodiment, the protein of the invention or part thereof may be used to remove contaminating RNA in a biological sample, alone or in combination with other nucleases. In a more preferred embodiment, the protein of the invention or part thereof may be used to purify DNA preparations from contaminating RNA, to remove RNA templates prior to second strand synthesis and prior to analysis of in vitro translation products. Compositions comprising the protein of the present invention or part thereof are added to biological samples as a “cocktail” with other nucleases. The advantage of using a cocktail of hydrolytic enzymes is that one is able to hydrolyze a wide range of substrates without knowing the specificity of any of the enzymes. Such cocktails of nucleases are commonly used in molecular biology assays, for example to remove unbound RNA in RNAse protection assays. Using a cocktail of hydrolytic enzymes also protects a sample from a wide range of future unknown RNA contaminants from a vast number of sources. For example, the protein of the invention or part thereof is added to samples where contaminating substrates is undesirable. Alternatively, the protein of the invention or part thereof may be bound to a chromatographic support, either alone or in combination with other hydrolytic enzymes, using techniques well known in the art, to form an affinity chromatography column. A sample containing the undesirable substrate is run through the column to remove the substrate. Immobilizing the protein of the invention or part thereof on a support is particularly advantageous for those embodiments in which the method is to be practiced on a commercial scale. This immobilization facilitates the removal of the enzyme from the batch of product and subsequent reuse of the enzyme. Immobilization of the protein of the invention or part thereof can be accomplished, for example, by inserting a cellulose-binding domain in the protein. One of skill in the art will understand that other methods of immobilization could also be used and are described in the available literature. Alternatively, the same methods may be used to identify new substrates.

In another embodiment, the protein of the invention or part thereof may be used to decontaminate or disinfect samples infected by undesirable parasite, bacteria and/or viruses using any of the methods known to those skilled in the art including those described in Youle et al, (1994), supra; Mikulski et al (1990) supra, Wu et al (1993) supra.

In another embodiment, the present invention relates to compositions and methods using the protein of the invention or part thereof to selectively kill cells. The protein of the invention or part thereof is linked to a recognition moiety capable of binding to a chosen cell, such as lectins, receptors or antibodies thus generating cytotoxic reagents using methods and techniques described in U.S. Pat. No. 5,955,073, which disclosure is hereby incorporated by reference in its entirety.

In still another embodiment, the invention relates to compositions and methods using the protein of the invention or part thereof to stimulate cell proliferation both in vitro and in vivo, especially endothelial cell growth. For example, soluble forms of the protein of the invention or part thereof may be added to cell culture medium in an amount effective to stimulate cell proliferation.

In still another embodiment, the protein of the invention or part thereof may be used in the diagnosis, prevention and/or treatment of disorders associated with excessive angiogenesis such as tumor growth, arthritis or diabetic retinopathy.

In a preferred embodiment, the protein of the invention may be used as a diagnostic marker to evaluate the risk of a given individual to develop a tumor, to evaluate the risk of recurrence of tumors or to evaluate the degree of cancer aggressiveness based on the facts that the level of circulating angiogenin is lower in normal individuals than in patients bearing tumors, and is lower in patients with primary cancers compared to patients with reoccurent tumors, as stated above. Thus, quantitative immunoassays can be used for the detection of abnormal levels of either the protein of SEQ ID NO: 176 or the mRNA encoding such protein as the polynucleotide of SEQ ID No:7, thereby identifying those individuals at risk for the development of tumors or the recurrence of tumors. Detection of abnormal levels of the protein of the invention may be performed using any techniques known to those skilled in the art including those described elsewhere in the application. For example, antibodies binding specifically to the protein of the invention, or fragments thereof, may be used in routine immunoassays to screen for the presence or absence of the protein of the invention, or fragments thereof. Alternatively, the nucleic acids which encode the protein of the invention, or fragments thereof, may be used in hybridization assays to detect and/or quantity the expression of said protein.

Another aspect of the invention provides for molecules which inhibit, or reduce, the biological activity or expression of SEQ ID NO: 276. Such molecules may be be administered to patients to prevent vascularization, especially tumor vascularization, thereby limiting tumor growth. Such antagonists and/or inhibitors may be antibodies specific for the protein of the invention that can be used directly as an antagonist, or indirectly as a targeting or delivery mechanism for bringing a pharmaceutical agent to cells or tissue which express the protein of the invention. Neutralizing antibodies, (i.e., those which inhibit protein-protein interactions) are especially preferred for therapeutic use. Alternatively, such molecules may be mutated forms of the protein of the invention or truncated forms which will be able to bind to the partners of the protein of the invention and compete with if for partners but without eliciting any of its biological activities. Other methods to inhibit the expression of the protein of the invention include antisense and triple helix strategies as described herein. Other antagonists or inhibitors of the protein of the invention may be produced using methods which are generally known in the art, including the screening of libraries of pharmaceutical agents to identify those which specifically bind the protein of the invention. The protein of the invention, or part thereof, preferably its functional or immunogenic fragments, or oligopeptides related thereto, can be used for screening libraries of compounds in any of a variety of drug screening techniques including those described herein.

Protease Inhibitor Protein of SEQ ID NO: 181 (Internal Designation 1000771934) and Related Protein of SEQ ID NO:466

The protein of SEQ ID NOs:181 and 466 encoded by the extended cDNA SEQ ID NO:12 and 349, respectively, is a protease inhibitor belonging to the WAP-type disulfide core family. It will be appreciated that all characteristics and uses of the polynucleotides of SEQ ID NOs:12 and 349 and polypeptides of SEQ ID NOs:181 and 466, described throughout the present application also pertain to the human cDNA of clone 1000771934, and the polypeptides encoded thereby. Preferred polypeptides of the invention are polypeptides comprising the amino acid fragments of SEQ ID NO: 181 from positions 32 to 73, 49 to 62, 76 to 122, 97 to 110 or any combination thereof. Other preferred polypeptides of the invention are fragments of SEQ ID NO:181 having any of the biological activity described herein. The protease inhibitor activity of the protein of the invention or part thereof may be assessed using any techniques known to those skilled in the art. Possible substrates for the protein of the invention include are not limited to trypsin, chymotrypsin, leukoproteinase, elastase, subtilisin, type IV collagenase and other serine proteases.

Proteases, which cleave proteins, are largely used in industry including in food processing, brewing, and alcohol production. Proteases are important components of laundry detergents and other products. Within biological research, proteases are used in purification processes to degrade unwanted proteins. It is often desirable to employ proteases of low specificity or mixtures of more specific proteases to obtain the necessary degree of degradation.

Proteases are also key components of a broad range of biological pathways, including blood coagulation and digestion. For example, the absence or insufficiency of a protease can result in a pathological condition that can be treated by replacement or augmentation therapy. Such therapies include the treatment of hemophilia with clotting factors VIII, IX, and VIIa. In another application, the proteolytic enzyme tissue plasminogen activator (t-PA) is used to activate the body's clot lysing mechanism, thereby reducing morbitity resulting from myocardial infarction. The protease thrombin is used to initiate the clotting of fibrinogen-based tissue adhesives during surgery. Neutrophils produce several antibacterial serine proteases (Gabay, Ciba Found. Symp. 186:237-247, 1994; Scocchi et al., Eur. J. Biochem. 209:589-595, 1992, which disclosures are hereby incorporated by reference in their entireties). Proteases also regulate cellular processes through receptor-mediated pathways by proteolytic activation of the cognate receptor (Vu et al., Cell 64:1057-1068, 1991; Blackhart et al., J. Biol. Chem. 271:16466-16471, 1996, which disclosures are hereby incorporated by reference in their entireties).

Overproduction or lack of regulation of proteases can also have pathological consequences. Elastase, released within the lung in response to the presence of foreign particles, can damage lung tissue if its activity is not tightly regulated. Emphysema in smokers is believed to arise from an imbalance between elastase and its inhibitor, alpha-1-antitrypsin. This balance may be restored by administration of exogenous alpha-1-antitrypsin.

In addition, protease inhibitors have been shown to inhibit the growth of microorganisms including human pathogenic bacteria such as strains of group A streptococci, including antibiotic-resistant strains (Merigan, T. et al (1996) Ann Intern Med 124:1039-1050; Stoka, V. (1995) FEBS. Lett 370:101-104; Vonderfecht, S. et al (1988) J Clin Invest 82:2011-2016; Collins, A. et al (1991) Antimicrob Agents Chemother 35:2444-2446, which disclosures are hereby incorporated by reference in their entireties).

In view of the growing use of proteases in industry, research, and medicine, there is an ongoing need in the art for new enzymes and new enzyme inhibitors. The present invention addresses these needs.

The invention relates to compositions and methods using the protein of the invention or part thereof to inhibit proteases, both in vitro or in vivo. Since proteases play an important role in the regulation of many biological processes in virtually all living organisms as well as a major role in diseases, inhibitors of proteases are useful in a wide variety of applications.

In one embodiment, the protein of the invention or part thereof may be useful to quantify the amount of a given protease in a biological sample, and thus used in assays and diagnostic kits for the quantification of proteases in bodily fluids or other tissue samples, in addition to bacterial, fungal, plant, yeast, viral or mammalian cell cultures. In a preferred embodiment, the sample is assayed using a standard protease substrate. A known concentration of protease inhibitor is added, and allowed to bind to a particular protease present. The protease assay is then rerun, and the loss of activity is correlated to the protease inhibitor activity using techniques well known to those skilled in the art.

In addition, the protein of the invention or part thereof may be used to remove, identify or inhibit contaminating proteases in a sample. Compositions comprising the polypeptides of the present invention may be added to biological samples as a “cocktail” with other protease inhibitors to prevent degradation of protein samples. The advantage of using a cocktail of protease inhibitors is that one is able to inhibit a wide range of proteases without knowing the specificity of any of the proteases. Using a cocktail of protease inhibitors also protects a protein sample from a wide range of future unknown proteases which may contaminate a protein sample from a vast number of sources. For example, the protein of the invention or part thereof are added to samples where proteolytic degradation by contaminating proteases is undesirable. Such protease inhibitor cocktails (see for example the ready to use cocktails sold by Sigma) are widely used in research laboratory assays to inhibit proteases susceptible of degrading a protein of interest for which the assay is to be performed. Alternatively, the protein of the invention or part thereof may be bound to a chromatographic support, either alone or in combination with other protease inhibitor, using techniques well known in the art, to form an affinity chromatography column. A sample containing the undesirable protease is run through the column to remove the protease. Alternatively, the same methods may be used to identify new proteases.

In a preferred embodiment, the protein of the invention or part thereof may be used to inhibit proteases implicated in a number of diseases where cellular proteolysis occur such as diseases characterized by tissue degradation including but not limited to arthritis, muscular dystrophy, inflammation, tumor invasion, glomerulonephritis, parasite-borne infections, Alzheimer's disease, periodontal disease, and cancer metastasis.

In another preferred embodiment, the protein of the invention or part thereof may be useful to inhibit exogenous proteases, both in vivo and in vitro, implicated in a number of infectious diseases including but not limited to gingivitis, malaria, leishmaniasis, filariasis, osteoporosis and osteoarthritis, and other bacterial, and parasite-borne or viral infections. In particular, the protein of the invention or part thereof may offer applications in viral diseases where the proteolysis of primary polypeptide precursors is essential to the replication of the virus, as for HIV and HCV.

In another embodiment, the protease inhibitors of the present invention may be used as antibacterial agents to retard or inhibit the growth of certain bacteria either in vitro or in vivo. Particularly, the polypeptides of the present invention may be used to inhibit the growth of group A streptococci on non-living matter such as surgical instruments, laboratory glassware and plasticware, and in culture of living plant, fungi, and animal cells.

Furthermore, the protease inhibitors of the present invention find use in drug potentiation applications. For example, therapeutic agents such as antibiotics or antitumor drugs can be inactivated through proteolysis by endogenous proteases, thus rendering the administered drug less effective or inactive. Accordingly, the protease inhibitors of the invention may be administered to a patient in conjunction with a therapeutic agent in order to potentiate or increase the activity of the drug. This co-administration may be by simultaneous administration, such as a mixture of the protease inhibitor and the drug, or by separate simultaneous or sequential administration.

Serpin Protein of SEQ ID NO: 179 (Internal Designation 784093) and Related Protein of SEQ ID NO:464

The protein of SEQ ID NOs: 179 and 464 and encoded by the extended cDNA SEQ ID NOs:10 and 347, respectively, is a serine protease inhibitor. It will be appreciated that all characteristics and uses of the polynucleotides of SEQ ID NOs:10 and 347 and polypeptides of SEQ ID NOs:179 and 464, described throughout the present application also pertain to the human cDNA of clone 784093, and the polypeptides encoded thereby. Preferred polypeptides of the invention are polypeptides comprising the amino acid fragments of SEQ ID NO:179 from positions 47 to 139. Other preferred polypeptides of the invention are fragments of SEQ ID NO:179 having any of the biological activity described herein. The protease inhibitor activity of the protein of the invention or part thereof may be assessed using any techniques known to those skilled in the art including those disclosed in the U.S. Pat. No. 5,955,284. Possible substrates for the protein of the invention include are not limited to serine proteases such as elastase, trypsin, chymotrypsin, thrombin III, plasmin, heparin, complement II, plasminogen activator, protein C, interleukin-IB coverting enzyme, preferably trypsin, elastase, and chymotrypsin.

Proteases are key components of a broad range of biological pathways, including blood coagulation and digestion. For example, the absence or insufficiency of a protease can result in a pathological condition that can be treated by replacement or augmentation therapy. Such therapies include the treatment of hemophilia with clotting factors VIII, IX, and VIIa. In another application, the proteolytic enzyme tissue plasminogen activator (t-PA) is used to activate the body's clot lysing mechanism, thereby reducing morbitity resulting from myocardial infarction. The protease thrombin is used to initiate the clotting of fibrinogen-based tissue adhesives during surgery. Neutrophils produce several antibacterial serine proteases (Gabay, Ciba Found. Symp. 186:237-247, 1994; Scocchi et al., Eur. J. Biochem. 209:589-595, 1992, which disclosures are hereby incorporated by reference in their entireties). Proteases also regulate cellular processes through receptor-mediated pathways by proteolytic activation of the cognate receptor (Vu et al., Cell 64:1057-1068, 1991; Blackhart et al., J. Biol. Chem. 271:16466-16471, 1996, which disclosures are hereby incorporated by reference in their entireties).

Overproduction or lack of regulation of proteases can also have pathological consequences. Elastase, released within the lung in response to the presence of foreign particles, can damage lung tissue if its activity is not tightly regulated. Emphysema in smokers is believed to arise from an imbalance between elastase and its inhibitor, alpha-1-antitrypsin. This balance may be restored by administration of exogenous alpha-1-antitrypsin.

The serine proteases (SP) are a large family of proteolytic enzymes that include the digestive enzymes, trypsin and chymotrypsin, components of the complement cascade and of the blood-clotting cascade, and enzymes that control the degradation and turnover of macromolecules of the extracellular matrix. SP are so named because of the presence of a serine residue in the active catalytic site for protein cleavage. They are characterized by a catalytic triad of serine, histidine, and aspartic acid residues. SP have a wide range of substrate specificities and can be subdivided into subfamilies on the basis of these specificities. The main sub-families are trypases (cleavage after arginine or lysine), aspases (cleavage after aspartate), chymases (cleavage after phenylalanine or leucine), metases (cleavage after methionine), and serases (cleavage after serine).

Serine proteases are used for a variety of industrial purposes. For example, the serine protease subtilisin is used in laundry detergents to aid in the removal of proteinaceous stains (e.g., Crabb, ACS Symposium Series 460:82-94, 1991, which disclosure is hereby incorporated by reference in its entirety). In the food processing industry, serine proteases are used to produce protein-rich concentrates from fish and livestock, and in the preparation of dairy products (Kida et al., Journal of Fermentation and Bioengineering 80:478-484, 1995; Haard and Simpson, in Martin, A. M., ed., Fisheries Processing: Biotechnological Applications, Chapman and Hall, London, 1994, 132-154; Bos et al., European Patent Office Publication 494 149 A1, which disclosures are hereby incorporated by reference in their entireties).

Serpins are irreversible serine protease inhibitors which are principally located extracellularly. Proteins which have been assigned to the serpin family include the following: .alpha.-1 protease inhibitor,.alpha.-1-antichymotrypsin, antithrombin III, .alpha.-2-antiplasmin, heparin cofactor II, complement C1 inhibitor, plasminogen activator inhibitors 1 and 2, glia derived nexin, protein C inhibitor, rat hepatocyte inhibitors, crmA (a viral serpin which inhibits interleukin 1-.beta. cleavage enzyme), human squamous cell carcinoma antigen which may modulate the host immune response against tumor cells, human maspin which seems to function as a tumor suppressor, lepidopteran protease inhibitor, leukocyte elastase inhibitor (the only known intracellular serpin), and products from three orthopoxviruses (these products may be involved in the regulation of the blood clotting cascade and/or of the complement cascade in the mammalian host).

In view of the growing use of proteases in industry, research, and medicine, there is an ongoing need in the art for new enzymes and new enzyme inhibitors. The present invention addresses these needs.

In one embodiment, the protein of the invention or part thereof may be useful to quantify the amount of a given protease in a biological sample, and thus used in assays and diagnostic kits for the quantification of proteases in bodily fluids or other tissue samples, in addition to bacterial, fungal, plant, yeast, viral or mammalian cell cultures. In a preferred embodiment, the sample is assayed using a standard protease substrate. A known concentration of protease inhibitor is added, and allowed to bind to a particular protease present. The protease assay is then rerun, and the loss of activity is correlated to the protease inhibitor activity using techniques well known to those skilled in the art. Preferred proteases in this embodiment are serine protease, more preferably elastase, trypsin and chymotrypsin.

In addition, the protein of the invention or part thereof may be used to remove, identify or inhibit contaminating proteases in a sample. Compositions comprising the polypeptides of the present invention may be added to biological samples as a “cocktail” with other protease inhibitors to prevent degradation of protein samples. The advantage of using a cocktail of protease inhibitors is that one is able to inhibit a wide range of proteases without knowing the specificity of any of the proteases. Using a cocktail of protease inhibitors also protects a protein sample from a wide range of future unknown proteases which may contaminate a protein sample from a vast number of sources. For example, the protein of the invention or part thereof are added to samples where proteolytic degradation by contaminating proteases is undesirable. Such protease inhibitor cocktails (see for example the ready to use cocktails sold by Sigma) are widely used in research laboratory assays to inhibit proteases susceptible of degrading a protein of interest for which the assay is to be performed. Alternatively, the protein of the invention or part thereof may be bound to a chromatographic support, either alone or in combination with other protease inhibitor, using techniques well known in the art, to form an affinity chromatography column. A sample containing the undesirable protease is run through the column to remove the protease. Alternatively, the same methods may be used to identify new proteases.

In a preferred embodiment, the protein of the invention or part thereof may be used to inhibit proteases implicated in a number of diseases where cellular proteolysis occur such as diseases characterized by tissue degradation including but not limited to arthritis, muscular dystrophy, inflammation, tumor invasion, glomerulonephritis, parasite-borne infections, Alzheimer's disease, periodontal disease, and cancer metastasis. In a more preferred embodiment, the invention relates to compositions and methods to use the protein of the invention or part thereof in diseases characterized by an abnormally elevated levels of trypsin, chymotrypsin or elastase, including but not limited to chronic emphysema of the lungs, cirrhosis, liver failure, cystic fibrosis, alpha1-antitrypsin deficiency associated disorders such as aneurysm or toxic shock. For prevention and/or treatment purposes, the protein of the invention may be used using any of the gene therapy methods described herein or known to those skilled in the art.

In another preferred embodiment, the protein of the invention or part thereof may be useful to inhibit exogenous proteases, both in vivo and in vitro, implicated in a number of infectious diseases including but not limited to gingivitis, malaria, leishmaniasis, filariasis, osteoporosis and osteoarthritis, and other bacterial, and parasite-borne or viral infections. In particular, the protein of the invention or part thereof may offer applications in viral diseases where the proteolysis of primary polypeptide precursors is essential to the replication of the virus, as for HIV and HCV.

In another embodiment, the protease inhibitors of the present invention may be used as antibacterial agents to retard or inhibit the growth of certain bacteria either in vitro or in vivo. Particularly, an amount of the polypeptides of the present invention effective to inhibit proliferation may be used to inhibit the growth of group A streptococci on non-living matter such as surgical instruments, laboratory glassware and plasticware, and in culture of living plant, fungi, and animal cells.

Furthermore, the protease inhibitors of the present invention find use in drug potentiation applications. For example, therapeutic agents such as antibiotics or antitumor drugs can be inactivated through proteolysis by endogenous proteases, thus rendering the administered drug less effective or inactive. Accordingly, the protease inhibitors of the invention may be administered to a patient in conjunction with a therapeutic agent in order to potentiate or increase the activity of the drug. This co-administration may be by simultaneous administration, such as a mixture of the protease inhibitor and the drug, or by separate simultaneous or sequential administration.

ATPKf Protein Sequence of SEQ ID No.253 (Internal Designation 1000867870)

The protein of SEQ ID NO:253 encoded by the extended cDNA SEQ ID NO:84 and relate polynucleotides of SEQ ID NO:404 is a variant of the human mitochondrial ATP synthase f subunit or ATPK (E.C. 3.6.1.34) and, as such, plays a role in cellular respiration. Preferred polypeptides of the invention are are polypeptides comprising the amino acids of SEQ ID NO: 253 from positions 5 to 88. Other preferred polypeptides of the invention are fragments of SEQ ID NO: 253 having any of the biological activity described herein. It will be appreciated that all characteristics and uses of the polynucleotides of SEQ ID NOs:84 and 404 and polypeptides of SEQ ID NO: 253 described throughout the present application also pertain to the human cDNA of clone 1000867870, and the polypeptides encoded thereby.

The mitochondrial electron transport (or respiratory) chain is a series of enzyme complexes in the mitochondrial membrane that is responsible for the transport of electrons from NADH to oxygen and the coupling of this oxidation to the synthesis of ATP (oxidative phosphorylation). ATP then provides the primary source of energy for driving a cell's many energy-requiring reactions. ATP synthase (F0 F1 ATPase) is the enzyme complex at the terminus of this chain and serves as a reversible coupling device that interconverts the energies of an electrochemical proton gradient across the mitochondrial membrane into either the synthesis or hydrolysis of ATP. This gradient is produced by other enzymes of the respiratory chain in the course of electron transport from NADH to oxygen. When the cell's energy demands are high, electron transport from NADH to oxygen generates an electrochemical gradient across the mitochondrial membrane. Proton translocation from the outer to the inner side of the membrane drives the synthesis of ATP. Under conditions of low energy requirements and when there is an excess of ATP present, this electrochemical gradient is reversed and ATP synthase hydrolyzes ATP. The energy of hydrolysis is used to pump protons out of the mitochondrial matrix. ATP synthase is, therefore, a dual complex, the F0 portion of which is a transmembrane proton carrier or pump, and the F1 portion of which is catalytic and synthesizes or hydrolyzes ATP. Mammalian ATP synthase complex consists of sixteen different polypeptides (Walker, J. E. and Collinson, T. R. (1994) FEBS Lett.346: 39-43, which disclosure is hereby incorporated by reference in its entirety). Six of these polypeptides (subunits alpha, beta, gamma, delta, epsilon, and an ATPase inhibitor protein IF I) comprise the globular catalytic F1 ATPase portion of the complex, which lies outside of the mitochondrial membrane. The remaining ten polypeptides (subunits a, b, c, d, e, f, g, F6, OSCP, and A6L) comprise the proton-translocating, membrane spanning F0 portion of the complex. Like other members of the respiratory chain, all but two of the polypeptide subunits of ATP synthase are nuclear gene products that are imported into the mitochondria. Enzyme complexes similar to mammalian ATP synthase are found in all cell types and in chloroplast and bacterial membranes. This universality indicates the central importance of this enzyme to ATP metabolism. Transcriptional regulation of these nuclear encoded genes appears to be the predominant means for controlling the biogenesis of ATP synthase. Multiple mitochondrial pathologies exist because of the essential role of mitochondrial oxidative phosphorylation in cellular energy production, in the generation of reactive oxygen species and in the initation of apoptosis (Wallace, Science, 283:1482-1488, 1999, which disclosure is hereby incorporated by reference in its entirety). It is now clear that mitochondrial diseases encompass an assemblage of clinical problems commonly involving tissues that have high energy requirements such as heart, muscle and the renal and endocrine systems. Over the past 11 years, a considerable body of evidence has accumulated implicating defects in the mitochondrial energy-generating pathway, oxidative phosphorylation, in a wide variety of degenerative diseases including myopathy and cardiomyopathy. Most classes of pathogenic mitochondrial DNA mutations affect the heart, in association with a variety of other clinical manifestations that can include skeletal muscle, the central nervous system (including eye), the endocrine system, and the renal system. Nuclear mutations causing mitochondrial disorders have been described. They are often found in highly conserved subunits. Mitochondrial disorders with nuclear mutations include: myopathies (PEO, MNGIE, congenital muscular dystrophy, carnitine disorders), encephalopathies (Leigh, Infantile, Wilson's disease, Deafness-Dystonia syndrome), other systemic disorders and cardiomyopathies.

The discovery of a new ATP synthase subunit, and polynucleotides encoding it satisfy a need in the art by providing new compositions which are useful for the diagnosis, prevention, and treatment of cancer, myopathies, immune disorders, and neurological disorders.

An object of the present invention relates to compositions and methods of targeting heterologous compounds, either polypeptides or polynucleotides to mitochondria by recombinantly or chemically fusing a fragment of the protein of the invention to an heterologous polypeptide or polynucleotide. Preferred fragments are signal peptide, amphiphilic alpha helices and/or any other fragments of the protein of the invention, or part thereof, that may contain targeting signals for mitochondria including but not limited to matrix targeting signals as defined in Herrman and Neupert, Curr. Opinion Microbiol. 3:210-4 (2000); Bhagwat et al. J. Biol. Chem. 274:24014-22 (1999), Murphy Trends Biotechnol. 15:326-30 (1997); Glaser et al. Plant Mol Biol 38:311-38 (1998); Ciminale et al. Oncogene 18:4505-14 (1999), which disclosures are hereby incorporated by reference in their entireties. Such heterologous compounds may be used to modulate mitochondria's activities. For example, they may be used to induce and/or prevent mitochondrial-induced apoptosis or necrosis. In addition, heterologous polynucleotides may be used for mitochondrial gene therapy to replace a defective mitochondrial gene and/or to inhibit the deleterious expression of a mitochondrial gene.

The invention further relates to methods and compositions using the protein of the invention or part thereof to diagnose, prevent and/or treat several disorders in which mitochondrial respiratory electron transport chain is impaired, including but not limited to mitochondriocytopathies, necrosis, aging, myopathies, cancer and neurodegenerative diseases such as Alzheimer's disease, Huntington's disease, Parkinson's disease, epilepsy, Down's syndrome, dementia, multiple sclerosis, and amyotrophic lateral sclerosis. For diagnostic purposes, the expression of the protein of the invention could be investigated using any of the Northern blotting, RT-PCR or immunoblotting methods described herein and compared to the expression in control individuals. For prevention and/or treatment purposes, the protein of the invention may be used to enhance electron transport and increase energy delivery using any of the gene therapy methods described herein or known to those skilled in the art.

In another embodiment, the invention further relates to methods and compositions using the protein of the invention or part thereof to diagnose, prevent and/or treat several disorders in which mitochondrial respiratory electron transport chain needs to be impaired, including but not limited to Sjogren's syndrome, Addison's disease, bronchitis, dermatomyositis, polymyositis, glomerulonephritis, diabetes mellitus, emphysema, Graves' disease, atrophic gastritis, lupus erythematosus, myasthenia gravis, multiple sclerosis, autoimmune thyroiditis, ulcerative colitis, anemia, pancreatitis, scleroderma, rheumatoid and osteoarthritis, asthma, allergic rhinitis, atopic dermatitis, dermatomyositis, polymyositis, and gout, using any techniques known to those skilled in the art including the antisense or triple helices strategies described herein.

Moreover, antibodies to the protein of the invention or part thereof may be used for detection of mitochondria organelles and/or mitochondrial membranes using any techniques known to those skilled in the art.

Oligomerization Protein Sequence of SEQ ID No. 310 (Internal Designation D150568)

The protein of SEQ ID NO: 310 encoded by the cDNA of SEQ ID NO: 141 and 435, is able to form homo-oligomers. Preferred polypeptides of the invention are polypeptides comprising the amino acids of SEQ ID NO:310 from positions 1 to 109. Other preferred polypeptides of the invention are fragments of SEQ ID NO: 310 having any of the biological activities described herein.

Multivalency is a prerequisite for a variety of macromolecular interactions such as binding of antibodies or lectins to specific targets, ligand recognition, activation or inhibition of receptors and cell adhesion. Dimerization and oligomerization of proteins are thus general biological control mechanisms that contribute to the activation of cell membrane receptors, transcription factors, vesicle fusion proteins, and other classes of intra- and extracellular proteins.

Multimerization domains have been shown to be useful tools in several areas of biotechnology, especially in protein engineering. For example, Tso et al have used leucine zippers for producing bispecific antibody heterodimers (U.S. Pat. No. 5,932,448)/Methods of preparing soluble oligomeric proteins using leucine zippers have been described by Conrad et al (U.S. Pat. No. 5,965,712), Ciardelli et al (U.S. Pat. No. 5,837,816), Spriggs et al (WO9410308)/Leucine zipper forming sequences have been used by Pelletier et al in protein fragment complementation assays to detect biomolecular interactions (WO9834120), which disclosures are hereby incorporated by reference in their entireties. Because of their usefulness in biotechnology, it is thus highly interesting to isolate new multimerization domains.

The multimerization activity of the protein of the invention or part thereof may be assayed using any of the assays known to those skilled in the art including circular dichroism spectrum, gel filtration chromatography and thermal melting analyses.

In one embodiment, the invention relates to compositions and methods of using the protein of the invention or part thereof for preparing soluble multimeric proteins, which consist in multimers of fusion proteins containing a multimerization domain fused to a protein of interest, using any technique known to those skilled in the art including those described in international patent WO9410308, which disclosure is hereby incorporated by reference in its entirety.

In another embodiment, the protein of the invention or part thereof or derivative thereof is used for detection and determination of an analyte in a biological liquid using the teachings of U.S. Pat. No. 5,643,731, which disclosure is hereby incorporated by reference in its entirety. Briefly, a first multimerization domain is immobilized on a solid support and the second multimerization domain is coupled to a specific binding partner for an analyte in a biological fluid. The two peptides are then brought into contact thereby immobilizing the binding partner on the solid phase. The biological sample is then contacted with the immobilized binding partner and the amount of analyte in the sample bound to the binding partner determined.

In still another embodiment, the protein of the invention or part thereof may be used to construct multimerization devices comprising hybrid molecules with a functional domain fused to a multimerization domain in order to yield multimeric complexes with improved pharmacokinetic and pharmacological properties as described in WO0102440, which disclosure is hereby incorporated by reference in its entirety. In a preferred embodiment, the protein of the invention or part thereof may be used to construct different fusion proteins with different functional domains such as enzyme moieties or cytotoxic moieties. Vectors encoding these different proteins may then be transfected in the same host cell in conditions allowing for multimerization, thus yielding multimeric multifunctional complexes.

Chaperone Protein of SEQ ID NO: 303 (Internal Designation D637548)

The protein of SEQ ID NO: 303 encoded by the cDNA of SEQ ID NO:134 is a chaperonin. Accordingly, the protein of SEQ ID NO:303 plays a role in protein synthesis/folding, cellular trafficking, and the cellular stress response. In addition, the protein of SEQ ID No: 303 has immunosupressant and growth factor properties. It is able to depress delayed type hypersensitivity reactions. It is a product of primary and neoplastic cell proliferation and under these conditions acts as a growth factor. It is also a product of platelet activation and may play a part in wound healing and skin repair. Preferred polypeptides of the invention are polypeptides comprising the amino acids of SEQ ID NO:303 from positions 9 to 33, or from positions 7 to 101. Other preferred polypeptides of the invention are fragments of SEQ ID NO: 303 having any of the biological activities described herein. The different activities of the protein of the invention or part thereof may be assayed using any of the assays described in U.S. Pat. No. 6,117,421 or any of the assays referred into U.S. Pat. No. 6117,421, which disclosures are hereby incorporated by reference in their entireties.

Chaperonins belong to a wider class of molecular chaperones, molecules involved in post-translational folding, targeting and assembly of other proteins, but which do not themselves form part of the final assembled structure as discussed by Ellis et al., 1991, Annu. Rev. Biochem. 60 321-347, which disclosures are hereby incorporated by reference in their entireties. Most molecular chaperones are “heat shock” or “stress” proteins (hsp); i.e. their production is induced or increased by a variety of cellular insults (such as metabolic disruption, oxygen radicals, inflammation, infection and transformation), heat being only one of the better studies stresses as reviewed by Lindquist et al., 1988, Annu. Rev. Genet. 22 631-677, which disclosure is hereby incorporated by reference in its entirety. As well as these quantitative changes in specific protein levels, stress can induce the movement of constitutively produced stress proteins to different cellular compartments as referred to in the Lindquist reference mentioned above. The heat shock response is one of the most highly conserved genetic system known and the various heat shock protein families are among the most evolutionarily stable proteins in existence. The major stress proteins accumulate to very high levels in stressed cells but occur at low to moderate levels in cells that have not been stressed. As well as enabling cells to cope under adverse conditions, members of these families perform essential functions in normal cells.

Chaperones are also involved in a number of disorders, especially autoimmune diseases such as type 1 diabetes, rheumatoid arthritis, systemic lupus erythematosus, Sjogren syndrome, and mixed connective tissue disease (Feige et al. EXS 1996; 77:359-73; Feili-Hariri et al. J Autoimmun 2000; 14:133-42, which disclosures are hereby incorporated by reference in their entireties). Chaperones are also involved in various disorders including tuberculosis and leprosy (Zugel et al. Clin Microbiol Rev 1999; 12:19-39), neurogenerative disorders such as Alzheimer and Parkinson diseases (Yoo et al. J Neural Transm Suppl 1999; 57:315-22), and malignant disorders (Csermely et al. Pharmacol Ther 1998; 79:129-68), which disclosures are hereby incorporated by reference in their entireties.

In one embodiment, the protein of the invention or part thereof may be used to detect a potential pregnancy, preferably within 6-24 hours of fertilization using the teaching of Morton et al., 1976, Proc. R. Soc. B. 193 413-41 and U.S. Pat. No. 6,117,421, which disclosures are hereby incorporated by reference in their entireties. Detection of the expression or activity of the protein of the invention may be performed using any techniques known to those skilled in the art including those described elsewhere in the application.

In another embodiment, molecules able to block the expression or activity of the protein of the invention, such as antibodies, antisense or triple helix oligonucleotides, dominant negative forms of the protein, polypeptides or small molecule inhibitors of the expression or activity of the proteins, may be used to induce abortion as described in U.S. Pat. No. 6,117,421.

In still another embodiment, the protein of the invention or part thereof may be used to treat and/or prevent infertility and miscarriage using the simple administration of the protein of the invention or part thereof or using any of the gene therapy methods described elsewhere in the application and the teaching of the U.S. Pat. No. 6,117,421.

In another embodiment, the present invention provide methods of using the present proteins to identify specific cell types in vitro and in vivo. For example, as chaperone proteins are often upregulated in response to cellular stress, the detection of cells expressing elevated levels of the proteins provides a tool for detecting cells under stress. As cellular stress has been implicated in a number of disorders, such as cardiovascular disorders, neurodegenerative disorders, and cancer, the ability to detect such stress thus provides a diagnostic or screening tool for such conditions.

In addition, the present polypeptides and polynucleotides can be used to develop diagnostic and screening assays for diseases characterized by an abnormal level or activity of the protein of SEQ ID NO: 303 such as malignant disorders of various types, and autoimmune diseases including type I diabetes, rheumatoid arthritis, systemic lupus erythematosus, Sjogren syndrome, Graves disease, multiple sclerosis, and mixed connective tissue disease. Such assays can be performed using any biological sample, such as serum or plasma.

In another embodiment, various disorders can be treated, attenuated and/or prevented by a protein of SEQ ID NO: 303, or part thereof, or any other compound that can affect the level or activity of the proteins such as nucleic acids, antibodies, or chemical substances. In a preferred embodiment, proteins or other compounds directed to the proteins of the invention can be used to treat or prevent disorders in which the activity or level of the protein of SEQ ID NO: 303 is unbalanced. Such diseases include, but are not limited to, infectious diseases, neurogenerative disorders as Alzheimer and Parkinson diseases, schizophrenia, alopecia, aging, atherosclerosis, malignant disorders of various types, and autoimmune diseases including type I diabetes, rheumatoid arthritis, systemic lupus erythematosus, Sjogren syndrome, mixed connective tissue disease, malignant disorders, autoimmune and any other neurodegenerative disorder. In another embodiment, the proteins of SEQ ID NO: 303 or part thereof can be used as vaccines for various disorders including, but not limited, to cancer (Wang et al. Immunol Invest 2000;29:131-7), tuberculosis (Silva et al. Microbes Infect 1999;1 :429-35), diabetes (Int Immunol 1999;1 1:957-66), and atherosclerosis (Xu et al. Arterioscler Thromb 1992; 12:789-99), which disclosures are hereby incorporated by reference in their entireties.

One embodiment of the present invention relates to methods and compositions using the protein of SEQ ID NO:303 or fragments thereof as a stabilizing adjuvant to slow down protein degradation, boost the yields of recombinant proteins, prevent the aggregation of proteins or regenerate denatured proteins. In a preferred embodiment, the protein of SEQ ID NO:303 of fragment thereof is mixed with a composition comprising the protein for which it is desired to slow down degradation, boost yield, or regenerate denatured proteins under conditions which facilitate the desired result. For example, numerous commercial assay kits commonly used by those skilled in the arts of molecular biology and biochemistry depend on the biological properties of proteins (mostly enzymes) which can be very short-lived in vitro due to the low stability of those proteins. An example is described in Eur. Patent DE4124286, the disclosure of which is incorporated herein by reference in its entirety, wherein the low intrinsic stability of test solutions used in optical tests is increased by addition of chaperone proteins, thus making the test more sensitive. Another example is given in U.S. Pat. No. 6,013,488, which disclosure is hereby incorporated by reference in its entirety, wherein a heat-labile reverse transcriptase is able to perform cDNA synthesis at high temperature levels in the presence of a chaperone.

The protein of SEQ ID NO:303 may also be used to increase the yield or activity of recombinant proteins, preferably secreted proteins. In recombinant DNA technology, a major unsolved problem is the solubility and biological activity of the recombinantly overexpressed protein in a host, especially a bacterial or yeast host. Many eukaryotic proteins, especially the secreted ones, require for correct folding a specific cellular machinery which is lacking in bacterial hosts such as E. coli or becomes insufficient in mammalian/yeast cells due to high expression of the protein. The ability of the protein of SEQ ID NO:303 or fragments thereof to ensure proper folding of recombinant proteins may be utilized as follows. The protein of SEQ ID NO:303, or fragment thereof, may be coexpressed with the recombinant protein in bacterial or eukaryotic hosts to cause the hosts to express the heterologous proteins or polypeptides in a form having increased solubility and/or biological activity. For example, the protein of SEQ ID NO:303 or fragments thereof may be used in the methods described in U.S. Pat. No. 5,773,245, the disclosure of which is incorporated herein by reference in its entirety. Therefore the invention relates to a method for the correct folding, deaggregation or prevention of aggregation of a monomeric protein in vivo comprising: (a) constructing a host cell transformed with (i) a first DNA encoding a polypeptide having the amino acid sequence of a bioactive protein or a precursor thereof, wherein said polypeptide or precursor can aggregate within the cell to result in a multimeric, non-bioactive protein or precursor thereof and (ii) a second DNA which enable the cell to co-express the protein of the invention or part thereof with the said polypeptide or precursor, (b) growing said host cell for sufficient time under conditions wherein said first DNA and said second DNA express said bioactive protein and said protein of the invention, respectively; and (c) obtaining monomeric protein that is a bioactive protein. Alternatively the protein of SEQ ID NO:303 or fragments thereof may be exogeneously added to the cell cultures as described in PCT application WO 00/08135, the disclosure of which is incorporated herein by reference in its entirety.

The protein of SEQ ID NO:303 or fragments thereof may further be used to regenerate denatured proteins. Recombinantly expressed proteins with poor biological activity are routinely denatured with a potent denaturing agent, such as guanidine hydrochloride, followed by refolding by dilution with a large amount of a diluent to reduce the concentration of the denaturing agent. However, this method often results in a poor refolding rate which may be significantly increased by addition of a cocktail of chaperone proteins in a fashion similar to that described in Eur. Patent EP0650975, the disclosure of which is incorporated herein by reference in its entirety. The advantage of using a cocktail of chaperone proteins is to accommodate differences in binding specificity of the Hsp different families and the different members within each family.

In another embodiment of the present invention, the protein of SEQ ID NO:303 may be used to promote tissue repair and/or increase cell survival in stress conditions such as hypoxy, oxidative stress, genotoxic agents and more generally harmful conditions leading to programmed cell death. Those conditions include but are not limited to infarction, heart surgery, stroke, neurodegenerative diseases, epilepsy, trauma, atherosclerosis, restenosis after angioplasty, and nerve damage.

In addition, the invention relates to compositions and methods for promoting cell growth both in vitro and in vivo using any of the techniques known to those skilled in the art including those described in the U.S. Pat. No. 6,117,421. For example, soluble forms of the protein of the invention or part thereof may be added to cell culture medium in an amount effective to stimulate cell proliferation. Alternatively, any of the gene therapy methods described herein may be used to overexpress the protein of the invention or part thereof in vivo. Alternatively, the protein of the invention or part thereof may be directly administered in an amount effective to promoting cell growth in said subject. These applications are particularly important in individuals suffering from wounds or tissue damage to enhance tissue repair, in individuals to which organ or skin grafts have been applied, in individuals suffering from an inflammatory condition or an allergic disease.

In addition, the invention relates to compositions and methods for promoting immunosupression in a subject using any of the techniques known to those skilled in the art including those described in the U.S. Pat. No. 6,117,421. For example, the protein of the invention or part thereof may be directly administered in an amount effective to achieve immunosupression in said subject. Alternatively, any of the gene therapy methods described herein may be used to overexpress the protein of the invention or part thereof in vivo. These applications are particularly important in cases in which immunosupression is desired such as in individuals suffering from autoimmune disease including any of the diseases cited above and in individuals that have received an heterologous graft they could reject.

Ion Transport Protein of SEQ ID NO: 276 (Internal Designation D538694)

The protein of SEQ ID NO: 276 encoded by the cDNA of SEQ ID NO:107 belongs to the FXDY family of small ion transport regulators or channels (Sweadner and Rael (2000) Genomics 68:41-56, which disclosure is hereby incorporated by reference in its entirety). The protein of SEQ ID NO: 276 or part thereof plays a role in the control of ion transport. Preferred polypeptides of the invention are polypeptides comprising the amino acids of SEQ ID NO:276 from positions 9 to 63, or from positions 16 to 29. Other preferred polypeptides of the invention are fragments of SEQ ID NO: 276 having any of the biological activities described herein. The activity of the protein of the invention or part thereof may be assayed using any of the assays known to those skilled in the art including those described in .

Osmoregulation occurs in all organisms, though the mechanisms differ according to the organism's environment. Fresh water inhabitants need to retain salts, whereas ocean inhabitants need to retain water. Terrestrial inhabitants need to conserve both water and salts. Organisms must balance these needs with a requirement to eliminate metabolic waste, such as nitrogenous waste, and generate secreted body fluids, such as saliva for digestion and sweat for thermoregulation.

In mammals, sweat glands, salivary glands, and the kidney all produce a primary secretion that is essentially isosmotic with blood and extracellular fluids. Modification of this primary secretion then occurs as much of the sodium chloride and water are reabsorbed as they pass through the excretory ducts of the glands and kidney, whereas potassium and bicarbonate ions are secreted. This modification of the primary secretion is important in the sweat glands to conserve sodium chloride in hot environments, and in the salivary glands to conserve sodium chloride when excessive quantities of saliva are lost. This modification is critical in the kidney to maintain proper sodium and water balance in the extracellular fluids, a balance which also regulates arterial pressure. Loss of this modification activity by the duct cells causes a large loss of sodium and water, resulting in severe dehydration and low blood volume, and ultimately to circulatory collapse.

Sodium absorption by the intestines, especially in the colon, is necessary to prevent loss of sodium in the stools. The loss of sodium absorption produces a failure to absorb anions and water as well. The unabsorbed sodium chloride and water then lead to diarrhea, with further loss of sodium chloride from the body. Other body fluids may be under regulation similar to that seen in the systems described above. For example, cerebrospinal fluid is produced by active sodium ion transport from the capillaries across the epithelium of the choroid plexus, which in turn attracts chloride ions and water. A counter flow of potassium and bicarbonate ions move out of the cerebrospinal fluid into the capillaries. A dysfunction in osmoregulation is associated with several disease states, including hyponatremia, renal failure, and hypernatremia. (Strange, K. (1992) J Am. Soc. Nephrol. 3:12-27, which disclosure is hereby incorporated by reference in its entirety).

In one embodiment, the protein of the invention may be useful in the diagnosis, prevention and/or treatment of osmoregulatory disorders including but not limited to diabetes insipidus, diarrhea, peritonitis, chronic renal failure, Addison's disease, syndrome of inappropriate antidiuretic hormone (SIADH), hypoaldosteronism, hyponatremia, adrenal insufficiency, hypothyroidism, hypernatremia, hypokalemia, Barter's syndrome, Cushing's syndrome, metabolic acidosis, metabolic alkalosis, encephalopathy, edema, hypotension, and hypertension. For example, any of the gene therapy methods described herein may be used to overexpress the protein of the invention or part thereof in vivo. Alternatively, the protein of the invention or part thereof may be directly administered in an amount effective to promoting cell growth in said subject. For diagnostic purposes, the expression of the protein of the invention could be investigated using any of the Northern blotting, RT-PCR or immunoblotting methods described herein and compared to the expression in control individuals. For prevention and/or treatment purposes, the protein of the invention may be used to enhance ion transport and prevent or treat osmoregulatory disorders using any of the gene therapy methods described herein.

Uses of Antibodies

Antibodies of the present invention have uses that include, but are not limited to, methods known in the art to purify, detect, and target the polypeptides of the present invention including both in vitro and in vivo diagnostic and therapeutic methods. An example of such use using immunoaffinity chromatography is given below. The antibodies of the present invention may be used either alone or in combination with other compositions. For example, the antibodies have use in immunoassays for qualitatively and quantitatively measuring levels of antigen-bearing substances, including the polypeptides of the present invention, in biological samples (See, e.g., Harlow et al., 1988). (Incorporated by reference in the entirety). The antibodies may also be used in therapeutic compositions for killing cells expressing the protein or reducing the levels of the protein in the body.

The invention further relates to antibodies that act as agonists or antagonists of the polypeptides of the present invention. For example, the present invention includes antibodies that disrupt the receptor/ligand interactions with the polypeptides of the invention either partially or fully. Included are both receptor-specific antibodies and ligand-specific antibodies. Included are receptor-specific antibodies, which do not prevent ligand binding but prevent receptor activation. Receptor activation (i.e., signaling) may be determined by techniques described herein or otherwise known in the art. Also include are receptor-specific antibodies which both prevent ligand binding and receptor activation. Likewise, included are neutralizing antibodies that bind the ligand and prevent binding of the ligand to the receptor, as well as antibodies that bind the ligand, thereby preventing receptor activation, but do not prevent the ligand from binding the receptor. Further included are antibodies that activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng et al. (1998); Chen et al. (1998); Harrop et al. (1998); Zhu et al. (1998); Yoon et al. (1998); Prat et al. (1998); Pitard et al. (1997); Liautard et al. (1997); Carlson et al. (1997); Taryman et al. (1995); Muller et al. (1998); Bartunek et al. (1996) (said references incorporated by reference in their entireties).

As discussed above, antibodies of the polypeptides of the invention can, in turn, be utilized to generate anti-idiotypic antibodies that “mimic” polypeptides of the invention using techniques well known to those skilled in the art (See, e.g. Greenspan and Bona (1989) and Nissinoff (1991), which disclosures are hereby incorporated by reference in their entireties). For example, antibodies which bind to and competitively inhibit polypeptide multimerization or binding of a polypeptide of the invention to ligand can be used to generate anti-idiotypes that “mimic” the polypeptide multimerization or binding domain and, as a consequence, bind to and neutralize polypeptide or its ligand. Such neutralization anti-idiotypic antibodies can be used to bind a polypeptide of the invention or to bind its ligands/receptors, and thereby block its biological activity.

Immunoaffinity Chromatography

Antibodies prepared as described herein are coupled to a support. Preferably, the antibodies are monoclonal antibodies, but polyclonal antibodies may also be used. The support may be any of those typically employed in immunoaffinity chromatography, including Sepharose CL-4B (Pharmacia, Piscataway, N.J.), Sepharose CL-2B (Pharmacia, Piscataway, N.J.), Affi-gel 10 (Biorad, Richmond, Calif.), or glass beads.

The antibodies may be coupled to the support using any of the coupling reagents typically used in immunoaffinity chromatography, including cyanogen bromide. After coupling the antibody to the support, the support is contacted with a sample which contains a target polypeptide whose isolation, purification or enrichment is desired. The target polypeptide may be a polypeptide selected from the group consisting of sequences of SEQ ID NOs:170-338, 456-560, 785-918 and polypeptides encoded by the clone inserts of the deposited clone pool, variants and fragments thereof, or a fusion protein comprising said selected polypeptide or a fragment thereof.

Preferably, the sample is placed in contact with the support for a sufficient amount of time and under appropriate conditions to allow at least 50% of the target polypeptide to specifically bind to the antibody coupled to the support.

Thereafter, the support is washed with an appropriate wash solution to remove polypeptides which have non-specifically adhered to the support. The wash solution may be any of those typically employed in immunoaffinity chromatography, including PBS, Tris-lithium chloride buffer (0.1M lysine base and 0.5M lithium chloride, pH 8.0), Tris-hydrochloride buffer (0.05M Tris-hydrochloride, pH 8.0), or Tris/Triton/NaCl buffer (50 mM Tris.cl, pH 8.0 or 9.0, 0.1% Triton X-100, and 0.5 MNaCl).

After washing, the specifically bound target polypeptide is eluted from the support using the high pH or low pH elution solutions typically employed in immunoaffinity chromatography. In particular, the elution solutions may contain an eluant such as triethanolamine, diethylamine, calcium chloride, sodium thiocyanate, potasssium bromide, acetic acid, or glycine. In some embodiments, the elution solution may also contain a detergent such as Triton X-100 or octyl-beta-D-glucoside.

### Expression of Genset Gene Products

Spatial Expression of the GENSET Genes of the Invention

Tissue expression of the cDNAs of the present invention was examined. Tables III and IV lists the number of hits for the cDNAs in Genset's libraries of tissues and cell types as well as in public databases. The tissues and cell types examined for polynucleotide expression were, for Table III: Brain; Fetal brain; Fetal kidney; Fetal liver; Pituitary gland; Liver; Placenta; Prostate; Salivary gland; Stomach/Intestine; and Testis. For each cDNA referred to by its corresponding sequence identification number from the priority application (see Table I for corresponding SEQ ID NO in present application), the number of proprietary 5′ESTs (i.e. cDNA fragments) expressed in a particular tissue referred to by its name is indicated in parentheses (second column). In addition, the bias in the spatial distribution of the polynucleotide sequences of the present invention was examined by comparing the relative proportions of the biological polynucleotides of a given tissue using the following statistical analysis. The under- or over-representation of a polynucleotide of a given cluster in a given tissue was performed using the normal approximation of the binomial distribution. When the observed proportion of a polynucleotide of a given tissue in a given consensus had less than 1% chance to occur randomly according to the chi2 test, the frequency bias was reported as “preferred”. The results are given in Table V as follows. For each polynucleotide showing a bias in tissue distribution as referred to by its sequence identification number in the first column, the list of tissues where the polynucleotides are under-represented is given in the second column entitled “low expression” and the list of tissues where the polynucleotides are over-represented is given in the third column entitled “high expression”.

Evaluation of Expression Levels and Patterns of GENSET Polypeptide-Encoding mRNAs

The spatial and temporal expression patterns of GENSET polypeptide-encoding mRNAs, as well as their expression levels, may also be further determined as follows.

Expression levels and patterns of GENSET polypeptide-encoding mRNAs may be analyzed by solution hybridization with long probes as described in International Patent Application No. WO 97/05277, the entire contents of which are hereby incorporated by reference. Briefly, a GENSET polynucleotide, or fragment thereof, corresponding to the gene encoding the mRNA to be characterized is inserted at a cloning site immediately downstream of a bacteriophage (T3, T7 or SP6) RNA polymerase promoter to produce antisense RNA. Preferably, the GENSET polynucleotide is at least a 100 nucleotides in length. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridizations are performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

The GENSET polypeptide-encoding cDNAs, or fragments thereof, may also be tagged with nucleotide sequences for the serial analysis of gene expression (SAGE) as disclosed in UK Patent Application No. 2 305 241 A, the entire contents of which are incorporated by reference. In this method, cDNAs are prepared from a cell, tissue, organism or other source of nucleic acid for which it is desired to determine gene expression patterns. The resulting cDNAs are separated into two pools. The cDNAs in each pool are cleaved with a first restriction endonuclease, called an “anchoring enzyme,” having a recognition site which is likely to be present at least once in most cDNAs. The fragments which contain the 5′ or 3′ most region of the cleaved cDNA are isolated by binding to a capture medium such as streptavidin coated beads. A first oligonucleotide linker having a first sequence for hybridization of an amplification primer and an internal restriction site for a “tagging endonuclease” is ligated to the digested cDNAs in the first pool. Digestion with the second endonuclease produces short “tag” fragments from the cDNAs. A second oligonucleotide having a second sequence for hybridization of an amplification primer and an internal restriction site is ligated to the digested cDNAs in the second pool. The cDNA fragments in the second pool are also digested with the “tagging endonuclease” to generate short “tag” fragments derived from the cDNAs in the second pool. The “tags” resulting from digestion of the first and second pools with the anchoring enzyme and the tagging endonuclease are ligated to one another to produce “ditags.” In some embodiments, the ditags are concatamerized to produce ligation products containing from 2 to 200 ditags. The tag sequences are then determined and compared to the sequences of the GENSET polypeptide-encoding cDNAs to determine which genes are expressed in the cell, tissue, organism, or other source of nucleic acids from which the tags were derived. In this way, the expression pattern of a GENSET polypeptide-encoding gene in the cell, tissue, organism, or other source of nucleic acids is obtained.

Quantitative analysis of GENSET gene expression may also be performed using arrays. For example, quantitative analysis of gene expression may be performed with GENSET polynucleotides, or fragments thereof in a complementary DNA microarray as described by Schena et al. (1995 and 1996) which disclosures are hereby incorporated by reference in their entireties. GENSET polypeptide-encoding cDNAs or fragments thereof are amplified by PCR and arrayed from 96-well microtiter plates onto silylated microscope slides using high-speed robotics. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C. Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of the expression of genes may also be performed with GENSET polypeptide-encoding cDNAs or fragments thereof in complementary DNA arrays as described by Pietu et al. (1996), which disclosure is hereby incorporated by reference in its entirety. The GENSET polynucleotides of the invention or fragments thereof are PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis of GENSET genes can be done through high density nucleotide arrays as described by Lockhart et al. (1996) and Sosnowski et al. (1997), which disclosures are hereby incorporated by reference in their entireties. Oligonucleotides of 15-50 nucleotides corresponding to sequences of a GENSET polynucleotide or fragments thereof are synthesized directly on the chip (Lockhart et al., supra) or synthesized and then addressed to the chip (Sosnowski et al., supra). Preferably, the oligonucleotides are about 20 nucleotides in length. cDNA probes labeled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., (supra) and application of different electric fields (Sosnowsky et al., supra), the dyes or labeling compounds are detected and quantified. Duplicate hybridizations are performed. Comparative analysis of the intensity of the signal originating from cDNA probes on the same target oligonucleotide in different cDNA samples indicates a differential expression of the GENSET polypeptide-encoding mRNA.

Uses of GENSET Gene Expression Data

Once the expression levels and patterns of a GENSET polypeptide-encoding mRNA has been determined using any technique known to those skilled in the art, in particular those described in the section entitled “Evaluation of Expression Levels and Patterns of GENSET polypeptide-encoding mRNAs”, or using the instant disclosure, these information may be used to design GENSET gene specific markers for detection, identification, screening and diagnosis purposes as well as to design DNA constructs with an expression pattern similar to a GENSET gene expression pattern.

Detection of GENSET Polypeptide Expression and/or Biological Activity

The invention further relates to methods of detection of GENSET polypeptide expression and/or biological activity in a biological sample using the polynucleotide and polypeptide sequences described herein. Such method scan be used, for example, as a screen for normal or abnormal GENSET polypeptide expression and/or biological activity and, thus, can be used diagnostically. The biological sample for use in the methods of the present invention includes a suitable sample from, for example, a mammal, particularly a human. For example, the sample can be issued from tissues or cell lines having the same origin as tissues or cell lines in which the polypeptide is known to be expressed, e.g. using data from Tables III, IV, or V.

Detection of GENSET Polypeptides

The invention further relates to methods of detection of GENSET polypeptide or encoding polynucleotides in a sample using the sequences described herein and any techniques known to those skilled in the art. For example, a labeled polynucleotide probe having all or a functional portion of the nucleotide sequence of a GENSET polypeptide-encoding polynucleotide can be used in a method to detect a GENSET polypeptide-encoding polynucleotide in a sample. In one embodiment, the sample is treated to render the polynucleotides in the sample available for hybridization to a polynucleotide probe, which can be DNA or RNA. The resulting treated sample is combined with a labeled polynucleotide probe having all or a portion of the nucleotide sequence of the GENSET polypeptide-encoding cDNA or genomic sequence, under conditions appropriate for hybridization of complementary sequences to occur. Detection of hybridization of polynucleotides from the sample with the labeled nucleic probe indicates the presence of GENSET polypeptide-encoding polynucleotides in a sample. The presence of GENSET polypeptide-encoding mRNA is indicative of GENSET polypeptide-encoding gene expression.

Consequently, the invention comprises methods for detecting the presence of a polynucleotide comprising a nucleotide sequence selected from a group consisting of the sequences of SEQ ID NOs:1-169, 339-455, 561-784, the sequences of clone inserts of the deposited clone pool, sequences fully complementary thereto, fragments and variants thereof in a sample. In a first embodiment, said method comprises the following steps of:

a) bringing into contact said sample and a nucleic acid probe or a plurality of nucleic acid probes which hybridize to said selected nucleotide sequence; and

b) detecting the hybrid complex formed between said probe or said plurality of probes and said polynucleotide.

In a preferred embodiment of the above detection method, said nucleic acid probe or said plurality of nucleic acid probes is labeled with a detectable molecule. In another preferred embodiment of the above detection method, said nucleic acid probe or said plurality of nucleic acid probes has been immobilized on a substrate. In still another preferred embodiment, said nucleic acid probe or said plurality of nucleic acid probes has a sequence comprised in a sequence complementary to said selected sequence.

In a second embodiment, said method comprises the steps of:

a) contacting said sample with amplification reaction reagents comprising a pair of amplification primers located on either side of the region of said nucleotide sequence to be amplified;

b) performing an amplification reaction to synthesize amplification products containing said region of said selected nucleotide sequence; and

c) detecting said amplification products.

In a preferred embodiment of the above detection method, when the polynucleotide to be amplified is a RNA molecule, preliminary reverse transcription and synthesis of a second cDNA strand are necessary to provide a DNA template to be amplified. In another preferred embodiment of the above detection method, the amplification product is detected by hybridization with a labeled probe having a sequence which is complementary to the amplified region. In still another preferred embodiment, at least one of said amplification primer has a sequence comprised in said selected sequence or in the sequence complementary to said selected sequence.

Alternatively, a method of detecting GENSET polypeptide expression in a test sample can be accomplished using any product which binds to a GENSET olypeptide of the present invention or a portion of a GENSET polypeptide. Such products may be antibodies, binding fragments of antibodies, polypeptides able to bind specifically to GENSET polypeptides or fragments thereof, including GENSET polypeptide agonists and antagonists. Detection of specific binding to the antibody indicates the presence of a GENSET polypeptide in the sample (e.g., ELISA).

Consequently, the invention is also directed to a method for detecting specifically the presence of a GENSET polypeptide according to the invention in a biological sample, said method comprising the steps of:

a) bringing into contact said biological sample with a product able to bind to a polypeptide of the invention or fragments thereof;

b) allowing said product to bind to said polypeptide to form a complex; and

b) detecting said complex.

In a preferred embodiment of the above detection method, the product is an antibody. In a more preferred embodiment, said antibody is labeled with a detectable molecule. In another more preferred embodiment of the above detection method, said antibody has been immobilized on a substrate.

In addition, the invention also relates to methods of determining whether a GENSET gene product (e.g. a polynucleotide or polypeptide) is present or absent in a biological sample, said methods comprising the steps of:

a) obtaining said biological sample from a human or non-human animal, preferably a mammal;

b) contacting said biological sample with a product able to bind to a GENSET polypeptide or encoding polynucleotide of the invention; and

c) determining the presence or absence of said GENSET polypeptide-encoding gene product in said biological sample.

The present invention also relates to kits that can be used in the detection of GENSET polypeptide-encoding gene expression products. The kit can comprise a compound that specifically binds a GENSET polypeptide (e.g. binding proteins, antibodies or binding fragments thereof (e.g. F(ab′)2 fragments) or a GENSET polypeptide-encoding mRNA (e.g. a complementary probe or primer), for example, disposed within a container means. The kit can further comprise ancillary reagents, including buffers and the like.

Detection of GENSET Polypeptide Biological Activity

The invention further includes methods of detecting specifically a GENSET polypeptide biological activity, and to identify compounds capable of modulating the activity of a GENSET polypeptide. Assessing the GENSET polypeptide biological activity may be performed by the detection of a change in any cellular property associated with the GENSET polypeptide, using a variety of techniques, including those described herein. To identify modulators of the polypeptides, a control is preferably used. For example, a control sample includes all of the same reagents but lacks the compound or agent being assessed; it is treated in the same manner as the test sample. A number of potentially assayable biological activities for many of the herein-described proteins are described supra, under the heading, “Uses of polypeptides of the invention.”

The present invention also relates to kits that can be used in the detection of GENSET polypeptide biological activity. The kit can comprise, e.g. substrates for GENSET polypeptides, GENSET-binding compounds, antibodies to GENSET polypeptides, etc., for example, disposed within a container means. The kit can further comprise ancillary reagents, including buffers and the like.

Identification of a Specific Context of GENSET Polypeptide-Encoding Gene Expression

When the expression pattern of a GENSET polypeptide-encoding mRNA shows that a GENSET polypeptide-encoding gene is specifically expressed in a given context, probes and primers specific for this gene as well as antibodies binding to the GENSET polypeptide-encoding polynucleotide may then be used as markers for the specific context. Examples of specific contexts are: specific expression in a given tissue/cell or tissue/cell type (see, e.g., Tables III-V), expression at a given stage of development of a process such as embryo development or disease development, or specific expression in a given organelle. Such primers, probes, and antibodies are useful commercially to identify tissues/cells/organelles of unknown origin, for example, forensic samples, differentiated tumor tissue that has metastasized to foreign bodily sites, or to differentiate different tissue types in a tissue cross-section using any technique known to those skilled in the art including in situ PCR or immunochemistry for example.

For example, the cDNAs and proteins of the sequence listing and fragments thereof, may be used to distinguish human tissues/cells from non-human tissues/cells and to distinguish between human tissues/cells/organelles that do and do not express the polynucleotides comprising the cDNAs. By knowing the expression pattern of a given GENSET polypeptide, either through routine experimentation or by using the instant disclosure, the polynucleotides and polypeptides of the present invention may be used in methods of determining the identity of an unknown tissue/cell sample/organelle. As part of determining the identity of an unknown tissue/cell sample/organelle, the polynucleotides and polypeptides of the present invention may be used to determine what the unknown tissue/cell sample is and what the unknown sample is not. For example, if a cDNA is expressed in a particular tissue/cell type/organelle, and the unknown tissue/cell sample/organelle does not express the cDNA, it may be inferred that the unknown tissue/cells are either not human or not the same human tissue/cell type/organelle as that which expresses the cDNA. These methods of determining tissue/cell/organelle identity are based on methods which detect the presence or absence of the mRNA (or corresponding cDNA) in a tissue/cell sample using methods well know in the art (e.g., hybridization, PCR based methods, immunoassays, immunochemistry, ELISA). Examples of such techniques are described in more detail below. Therefore, the invention encompasses uses of the polynucleotides and polypeptides of the invention as tissue markers. In a preferred embodiment, polynucleotides preferentially expressed in given tissues as indicated in Tables III-V and polypeptides encoded by such polynucleotides are used for this purpose. The invention also encompasses uses of polypeptides of the invention as organelle markers.

Consequently, the present invention encompasses methods of identification of a tissue/cell type/subcellular compartment, wherein said method includes the steps of:

a) contacting a biological sample which identity is to be assayed with a product able to bind a GENSET gene product; and

b) determining whether a GENSET gene product is expressed in said biological sample.

Products that are able to bind specifically to a GENSET gene product, namely a GENSET polypeptide or a GENSET polypeptide-encoding mRNA, include GENSET polypeptide binding proteins, antibodies or binding fragments thereof (e.g. F(ab′)2 fragments), as well as GENSET polynucleotide complementary probes and primers.

Step b) may be performed using any detection method known to those skilled in the art including those disclosed herein, especially in the section entitled “Detection of GENSET polypeptide expression and/or biological activity”.

Identification of Tissue Types or Cell Species by Means of Labeled Tissue Specific Antibodies

Identification of specific tissues is accomplished by the visualization of tissue specific antigens by means of antibody preparations which are conjugated, directly (e.g., green fluorescent protein) or indirectly to a detectable marker. Selected labeled antibody species bind to their specific antigen binding partner in tissue sections, cell suspensions, or in extracts of soluble proteins from a tissue sample to provide a pattern for qualitative or semi-qualitative interpretation.

Antisera for these procedures must have a potency exceeding that of the native preparation, and for that reason, antibodies are concentrated to a mg/ml level by isolation of the gamma globulin fraction, for example, by ion-exchange chromatography or by ammonium sulfate fractionation. Also, to provide the most specific antisera, unwanted antibodies, for example to common proteins, must be removed from the gamma globulin fraction, for example by means of insoluble immunoabsorbents, before the antibodies are labeled with the marker. Either monoclonal or heterologous antisera is suitable for either procedure.

A. Immunohistochemical Techniques

Purified, high-titer antibodies, prepared as described above, are conjugated to a detectable marker, as described, for example, by Fudenberg, (1980) or Rose et al., (1980), which disclosures are hereby incorporated by reference in their entireties.

A fluorescent marker, either fluorescein or rhodamine, is preferred, but antibodies can also be labeled with an enzyme that supports a color producing reaction with a substrate, such as horseradish peroxidase. Markers can be added to tissue-bound antibody in a second step, as described below. Alternatively, the specific anti-tissue antibodies can be labeled with ferritin or other electron dense particles, and localization of the ferritin coupled antigen-antibody complexes achieved by means of an electron microscope. In yet another approach, the antibodies are radiolabeled, with, for example ¹²⁵I, and detected by overlaying the antibody treated preparation with photographic emulsion. Preparations to carry out the procedures can comprise monoclonal or polyclonal antibodies to a single protein or peptide identified as specific to a tissue type, for example, brain tissue, or antibody preparations to several antigenically distinct tissue specific antigens can be used in panels, independently or in mixtures, as required. Tissue sections and cell suspensions are prepared for immunohistochemical examination according to common histological techniques. Multiple cryostat sections (about 4 um, unfixed) of the unknown tissue and known control, are mounted and each slide covered with different dilutions of the antibody preparation. Sections of known and unknown tissues should also be treated with preparations to provide a positive control, a negative control, for example, pre-immune sera, and a control for non-specific staining, for example, buffer. Treated sections are incubated in a humid chamber for 30 min at room temperature, rinsed, then washed in buffer for 30-45 min. Excess fluid is blotted away, and the marker developed. If the tissue specific antibody was not labeled in the first incubation, it can be labeled at this time in a second antibody-antibody reaction, for example, by adding fluorescein- or enzyme-conjugated antibody against the immunoglobulin class of the antiserum-producing species, for example, fluorescein labeled antibody to mouse IgG. Such labeled sera are commercially available. The antigen found in the tissues by the above procedure can be quantified by measuring the intensity of color or fluorescence on the tissue section, and calibrating that signal using appropriate standards.

B. Identification of Tissue Specific Soluble Proteins

The visualization of tissue specific proteins and identification of unknown tissues from that procedure is carried out using the labeled antibody reagents and detection strategy as described for immunohistochemistry; however the sample is prepared according to an electrophoretic technique to distribute the proteins extracted from the tissue in an orderly array on the basis of molecular weight for detection. A tissue sample is homogenized using a Virtis apparatus; cell suspensions are disrupted by Dounce homogenization or osmotic lysis, using detergents in either case as required to disrupt cell membranes, as is the practice in the art. Insoluble cell components such as nuclei, microsomes, and membrane fragments are removed by ultracentrifugation, and the soluble protein-containing fraction concentrated if necessary and reserved for analysis. A sample of the soluble protein solution is resolved into individual protein species by conventional SDS polyacrylamide electrophoresis as described, for example, by Davis et al., Section 19-2 (1986), using a range of amounts of polyacrylamide in a set of gels to resolve the entire molecular weight range of proteins to be detected in the sample. A size marker is run in parallel for purposes of estimating molecular weights of the constituent proteins. Sample size for analysis is a convenient volume of from 5 to 55 ul, and containing from about 1 to 100 ug protein. An aliquot of each of the resolved proteins is transferred by blotting to a nitrocellulose filter paper, a process that maintains the pattern of resolution. Multiple copies are prepared. The procedure, known as Western Blot Analysis, is well described in Davis et al., (1986) Section 19-3. One set of nitrocellulose blots is stained with Coomassie Blue dye to visualize the entire set of proteins for comparison with the antibody bound proteins. The remaining nitrocellulose filters are then incubated with a solution of one or more specific antisera to tissue specific proteins prepared as described herein. In this procedure, as in procedure A above, appropriate positive and negative sample and reagent controls are run.

In either procedure A or B, a detectable label can be attached to the primary tissue antigen-primary antibody complex according to various strategies and permutations thereof. In a straightforward approach, the primary specific antibody can be labeled; alternatively, the unlabeled complex can be bound by a labeled secondary anti-IgG antibody. In other approaches, either the primary or secondary antibody is conjugated to a biotin molecule, which can, in a subsequent step, bind an avidin conjugated marker. According to yet another strategy, enzyme labeled or radioactive protein A, which has the property of binding to any IgG, is bound in a final step to either the primary or secondary antibody. The visualization of tissue specific antigen binding at levels above those seen in control tissues to one or more tissue specific antibodies, prepared from the gene sequences identified from cDNA sequences, can identify tissues of unknown origin, for example, forensic samples, or differentiated tumor tissue that has metastasized to foreign bodily sites.

Screening and Diagnosis of Abnormal GENSET Polypeptide Expression and/or Biological Activity

Moreover, antibodies and/or primers specific for GENSET polypeptide expression may also be used to identify abnormal GENSET polypeptide expression and/or biological activity, and subsequently to screen and/or diagnose disorders associated with abnormal GENSET polypeptide expression. For example, a particular disease may result from lack of expression, over expression, or under expression of a GENSET polypeptide-encoding mRNA. By comparing mRNA expression patterns and quantities in samples taken from healthy individuals with those from individuals suffering from a particular disorder, genes responsible for this disorder may be identified. Primers, probes and antibodies specific for this GENSET polypeptide may then be used to elaborate kits of screening and diagnosis for a disorder in which the gene of interest is specifically expressed or in which its expression is specifically dysregulated, i.e. underexpressed or overexpressed.

Screening for Specific Disorders

The present invention also relates to methods and uses of GENSET polypeptides for identifying individuals having elevated or reduced levels of GENSET polypeptides, which individuals are likely to benefit from therapies to suppress or enhance GENSET polypeptide-encoding gene expression, respectively. One example of such methods and uses comprises the steps of:

a) obtaining from a mammal a biological sample;

b) detecting the presence in said sample of a GENSET polypeptide-encoding gene product (mRNA or protein);

c) comparing the amount of said GENSET polypeptide-encoding gene product present in said sample with that of a control sample; and

d) determing whether said human or non-human mammal has a reduced or elevated level of GENSET gene expression compared to the control sample.

A biological sample from a subject affected by, or at risk of developing, any disease or condition associated with a GENSET polypeptide can be screened for the presence of increased or decreased levels of GENSET gene product, relative to a normal population (standard or control), with an increased or decreased level of the GENSET polypeptide relative to the normal population being indicative of predisposition to or a present indication of the disease or condition, or any sympton associated with the disease or condition. Such individuals would be candidates for therapies, e.g., treatment with pharmaceutical compositions comprising the GENSET polypeptide, a polynucleotide encoding the GENSET polypeptide, or any other compound that affects the expression or activity of the GENSET polypeptide. Generally, the identification of elevated levels of the GENSET polypeptide in a patient would be indicative of an individual that would benefit from treatment with agents that suppress GENSET polypeptide expression or activity, and the identification of low levels of the GENSET polypeptide in a patient would be indicative of an individual that would benefit from agents that induce GENSET expression or activity.

Biological samples suitable for use in this method include any biological fluids, including, but not limited to, blood, saliva, milk, and urine. Tissue samples (e.g. biopsies) can also be used in the method of the invention, including samples derived from any tissue associated with GENSET gene expression (see, e.g. Tables III-V). Cell cultures or cell extracts derived, for example, from tissue biopsies can also be used. The detection step of the present method can be performed using standard protocols for protein/mRNA detection. Examples of suitable protocols include Northern blot analysis, immunoassays (e.g. RIA, Western blots, immunohistochemical analyses), and PCR.

Thus, the present invention further relates to methods and uses of GENSET polypeptides for identifying individuals or non-human animals at increased risk for developing, or present state of having, certain diseases/disorders associated with abnormal GENSET polypeptide expression or biological activity. One example of such methods comprises the steps of:

a) obtaining from a human or non-human mammal a biological sample;

b) detecting the presence in said sample of a GENSET gene product (mRNA or protein);

c) comparing the amount of said GENSET gene product present in said sample with that of a control sample; and

d) determing whether said human or non-human mammal is at increased risk for developing, or present state of having, a diseases or disorder.

In preferred embodiments, the biological sample is taken from animals presenting any symptom associated with any disease or condition associated with a GENSET gene product. In accordance with this method, the presence in the sample of altered (e.g. increased or decreased) levels of the GENSET product indicates that the subject is predisposed to the disease or condition. Biological samples suitable for use in this method include biological fluids including, but not limited to, blood, saliva, milk, and urine. Tissue samples (e.g. biopsies) can also be used in the method of the invention, including samples derived from any of the tissues listed in Tables III-V. Cell cultures or cell extracts derived, for example, from tissue biopsies can also be used.

The diagnostic methodologies described herein are applicable to both humans and non-human mammals.

Detection of GENSET Gene Mutations

The invention also encompasses methods and uses of GENSET polynucleotides to detect mutations in GENSET polynucleotides of the invention. Such methods may advantageously be used to detect mutations occurring in GENSET genes and preferably in their regulatory regions. When the mutation was proven to be associated with a disease, the detection of such mutations may be used for screening and diagnosis purposes.

In one embodiment of the oligonucleotide arrays of the invention, an oligonucleotide probe matrix may advantageously be used to detect mutations occurring in GENSET genes and preferably in their regulatory regions. For this particular purpose, probes are specifically designed to have a nucleotide sequence allowing their hybridization to the genes that carry known mutations (either by deletion, insertion or substitution of one or several nucleotides). By known mutations, it is meant, mutations on the GENSET genes that have been identified according, for example to the technique used by Huang et al. (1996) or Samson et al. (1996), which disclosures are hereby incorporated by reference in their entireties.

Another technique that is used to detect mutations in GENSET genes is the use of a high-density DNA array. Each oligonucleotide probe constituting a unit element of the high density DNA array is designed to match a specific subsequence of a GENSET genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the GENSET gene. In one such design, termed 4 L tiled array, is implemented a set of four probes (A, C, G, T), preferably 15-nucleotide oligomers. In each set of four probes, the perfect complement will hybridize more strongly than mismatched probes. Consequently, a nucleic acid target of length L is scanned for mutations with a tiled array containing 4 L probes, the whole probe set containing all the possible mutations in the known wild reference sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. in 1996, which disclosure is hereby incorporated by reference in its entirety.

Construction of DNA Constructs with a GENSET Gene Expression Pattern

In addition, characterization of the spatial and temporal expression patterns and expression levels of GENSET polypeptide-encoding mRNAs is also useful for constructing expression vectors capable of producing a desired level of gene product in a desired spatial or temporal manner, as discussed below.

DNA Constructs that Direct Temporal and Spatial GENSET Gene Expression in Recombinant Cell Hosts and in Transgenic Animals.

In order to study the physiological and phenotypic consequences of a lack of synthesis of a GENSET polypeptide, both at the cellular level and at the multi cellular organism level, the invention also encompasses DNA constructs and recombinant vectors enabling a conditional expression of a specific allele of a GENSET polypeptide-encoding genomic sequence or cDNA and also of a copy of this genomic sequence or cDNA harboring substitutions, deletions, or additions of one or more bases as regards to a nucleotide sequence selected from the group consisting of sequences of SEQ ID NOs:1-169, 339455, 561-784 and sequences of clone inserts of the deposited clone pool, or a fragment thereof, these base substitutions, deletions or additions being located either in an exon, an intron or a regulatory sequence, but preferably in the 5′-regulatory sequence or in an exon of the GENSET polypeptide-encoding genomic sequence or within the GENSET polypeptide-encoding cDNA.

A first preferred DNA construct is based on the tetracycline resistance operon tet from E. coli transposon Tn10 for controlling the GENSET gene expression, such as described by Gossen et al. (1992, 1995) and Furth et al. (1994), which disclosures are hereby incorporated by reference in their entireties. Such a DNA construct contains seven tet operator sequences from Tn10 (tetop) that are fused to either a minimal promoter or a 5′-regulatory sequence of the GENSET gene, said minimal promoter or said GENSET polynucleotide regulatory sequence being operably linked to a polynucleotide of interest that codes either for a sense or an antisense oligonucleotide or for a polypeptide, including a GENSET polypeptide, or a peptide fragment thereof. This DNA construct is functional as a conditional expression system for the nucleotide sequence of interest when the same cell also comprises a nucleotide sequence coding for either the wild type (tTA) or the mutant (rTA) repressor fused to the activating domain of viral protein VP16 of herpes simplex virus, placed under the control of a promoter, such as the HCMVIE1 enhancer/promoter or the MMTV-LTR. Indeed, a preferred DNA construct of the invention comprise both the polynucleotide containing the tet operator sequences and the polynucleotide containing a sequence coding for the tTA or the rTA repressor. In a specific embodiment, the conditional expression DNA construct contains the sequence encoding the mutant tetracycline repressor rTA, the expression of the polynucleotide of interest is silent in the absence of tetracycline and induced in its presence.

DNA Constructs Allowing Homologous Recombination: Replacement Vectors

A second preferred DNA construct will comprise, from 5′-end to 3′-end: (a) a first nucleotide sequence that is found in the GENSET polypeptide-encoding genomic sequence; (b) a nucleotide sequence comprising a positive selection marker, such as the marker for neomycin resistance (neo); and (c) a second nucleotide sequence that is found in the GENSET polypeptide-encoding genomic sequence, and is located on the genome downstream the first GENSET polypeptide-encoding nucleotide sequence (a).

In a preferred embodiment, this DNA construct also comprises a negative selection marker located upstream of the nucleotide sequence (a) or downstream from the nucleotide sequence (c). Preferably, the negative selection marker comprises the thymidine kinase (tk) gene (Thomas et al., 1986), the hygromycine beta gene (Te Riele et al, 1990), the hprt gene ( Van der Lugt et al., 1991; Reid et al., 1990) or the Diphteria toxin A fragment (Dt-A) gene (Nada et al., 1993; Yagi et al. 1990), which disclosures are hereby incorporated by reference in their entireties. Preferably, the positive selection marker is located within a GENSET exon sequence so as to interrupt the sequence encoding a GENSET polypeptide. These replacement vectors are described, for example, by Thomas et al. (1986; 1987), Mansour et al. (1988) and Koller et al. (1992).

The first and second nucleotide sequences (a) and (c) may be indifferently located within a GENSET polypeptide-encoding regulatory sequence, an intronic sequence, an exon sequence or a sequence containing both regulatory and/or intronic and/or exon sequences. The size of the nucleotide sequences (a) and (c) ranges from 1 to 50 kb, preferably from 1 to 10 kb, more preferably from 2 to 6 kb and most preferably from 2 to 4 kb.

DNA Constructs Allowing Homologous Recombination: Cre-LoxP System.

These new DNA constructs make use of the site specific recombination system of the P1 phage. The P1 phage possesses a recombinase called Cre which interacts specifically with a 34 base pairs loxP site. The loxP site is composed of two palindromic sequences of 13 bp separated by a 8 bp conserved sequence (Hoess et al., 1986), which disclosure is hereby incorporated by reference in its entirety. The recombination by the Cre enzyme between two loxP sites having an identical orientation leads to the deletion of the DNA fragment.

The Cre-loxP system used in combination with a homologous recombination technique has been first described by Gu et al. (1993, 1994), which disclosures are hereby incorporated by reference in their entireties. Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host. The recombinase enzyme may be brought at the desired time either by (a) incubating the recombinant cell hosts in a culture medium containing this enzyme, by injecting the Cre enzyme directly into the desired cell, such as described by Araki et al. (1995), which disclosure is hereby incorporated by reference in its entirety, or by lipofection of the enzyme into the cells, such as described by Baubonis et al (1993), which disclosure is hereby incorporated by reference in its entirety; (b) transfecting the cell host with a vector comprising the Cre coding sequence operably linked to a promoter functional in the recombinant cell host, which promoter being optionally inducible, said vector being introduced in the recombinant cell host, such as described by Gu et al. (1993) and Sauer et al (1988), which disclosures are hereby incorporated by reference in their entireties; (c) introducing in the genome of the cell host a polynucleotide comprising the Cre coding sequence operably linked to a promoter functional in the recombinant cell host, which promoter is optionally inducible, and said polynucleotide being inserted in the genome of the cell host either by a random insertion event or an homologous recombination event, such as described by Gu et al. (1994).

In a specific embodiment, the vector containing the sequence to be inserted in the GENSET gene by homologous recombination is constructed in such a way that selectable markers are flanked by loxP sites of the same orientation, it is possible, by treatment by the Cre enzyme, to eliminate the selectable markers while leaving the GENSET sequences of interest that have been inserted by an homologous recombination event. Again, two selectable markers are needed: a positive selection marker to select for the recombination event and a negative selection marker to select for the homologous recombination event. Vectors and methods using the Cre-loxP system are described by Zou et al. (1994), which disclosure is hereby incorporated by reference in its entirety.

Thus, a third preferred DNA construct of the invention comprises, from 5′-end to 3′-end: (a) a first nucleotide sequence that is comprised in the GENSET genomic sequence; (b) a nucleotide sequence comprising a polynucleotide encoding a positive selection marker, said nucleotide sequence comprising additionally two sequences defining a site recognized by a recombinase, such as a loxP site, the two sites being placed in the same orientation; and (c) a second nucleotide sequence that is comprised in the GENSET genomic sequence, and is located on the genome downstream of the first GENSET nucleotide sequence (a).

The sequences defining a site recognized by a recombinase, such as a loxP site, are preferably located within the nucleotide sequence (b) at suitable locations bordering the nucleotide sequence for which the conditional excision is sought. In one specific embodiment, two loxP sites are located at each side of the positive selection marker sequence, in order to allow its excision at a desired time after the occurrence of the homologous recombination event.

In a preferred embodiment of a method using the third DNA construct described above, the excision of the polynucleotide fragment bordered by the two sites recognized by a recombinase, preferably two loxP sites, is performed at a desired time, due to the presence within the genome of the recombinant host cell of a sequence encoding the Cre enzyme operably linked to a promoter sequence, preferably an inducible promoter, more preferably a tissue-specific promoter sequence and most preferably a promoter sequence which is both inducible and tissue-specific, such as described by Gu et al. (1994).

The presence of the Cre enzyme within the genome of the recombinant cell host may result from the breeding of two transgenic animals, the first transgenic animal bearing the GENSET-derived sequence of interest containing the loxP sites as described above and the second transgenic animal bearing the Cre coding sequence operably linked to a suitable promoter sequence, such as described by Gu et al. (1994).

Spatio-temporal control of the Cre enzyme expression may also be achieved with an adenovirus based vector that contains the Cre gene thus allowing infection of cells, or in vivo infection of organs, for delivery of the Cre enzyme, such as described by Anton and Graham (1995) and Kanegae et al. (1995), which disclosures are hereby incorporated by reference in their entireties.

The DNA constructs described above may be used to introduce a desired nucleotide sequence of the invention, preferably a GENSET genomic sequence or a GENSET cDNA sequence, and most preferably an altered copy of a GENSET genomic or cDNA sequence, within a predetermined location of the targeted genome, leading either to the generation of an altered copy of a targeted gene (knock-out homologous recombination) or to the replacement of a copy of the targeted gene by another copy sufficiently homologous to allow an homologous recombination event to occur (knock-in homologous recombination).

### Modifying Genset Polypoptide Expression and/or Biological Activity

Modifying endogenous GENSET expression and/or biological activity is expressly contemplated by the present invention.

Screening for Compounds that Modulate GENSET Expression and/or Biological Activity

The present invention further relates to compounds able to modulate GENSET expression and/or biological activity and methods to use these compounds. Such compounds may interact with the regulatory sequences of GENSET genes or they may interact with GENSET polypeptides directly or indirectly.

Compounds Interacting with GENSET Regulatory Sequences

The present invention also concerns a method for screening substances or molecules that are able to interact with the regulatory sequences of a GENSET gene, such as for example promoter or enhancer sequences in untranscribed regions of the genomic DNA, as determined using any techniques known to those skilled in the art including those described in the section entitled “Identification of Promoters in Cloned Upstream Sequences, or such as regulatory sequences located in untranslated regions of GENSET mRNA.

Sequences within untranscribed or untranslated regions of polynucleotides of the invention may be identified by comparison to databases containing known regulatory sequence such as transcription start sites, transcription factor binding sites, promoter sequences, enhancer sequences, 5′UTR and 3′UTR elements (Pesole et al., 2000; http://igs-server.cnrs-mrs.fr/˜gauthere/UTR/index.html). Alternatively, the regulatory sequences of interest may-be identified through conventional mutagenesis or deletion analyses of reporter plasmids using, for instance, techniques described in the section entitled “Identification of Promoters in Cloned Upstream Sequences”.

Following the identification of potential GENSET regulatory sequences, proteins which interact with these regulatory sequences may be identified as described below.

Gel retardation assays may be performed independently in order to screen candidate molecules that are able to interact with the regulatory sequences of the GENSET gene, such as described by Fried and Crothers (1981), Garner and Revzin (1981) and Dent and Latchman (1993), the teachings of these publications being herein incorporated by reference. These techniques are based on the principle according to which a DNA or mRNA fragment which is bound to a protein migrates slower than the same unbound DNA or mRNA fragment. Briefly, the target nucleotide sequence is labeled. Then the labeled target nucleotide sequence is brought into contact with either a total nuclear extract from cells containing regulation factors, or with different candidate molecules to be tested. The interaction between the target regulatory sequence of the GENSET gene and the candidate molecule or the regulation factor is detected after gel or capillary electrophoresis through a retardation in the migration.

Nucleic acids encoding proteins which are able to interact with the promoter sequence of the GENSET gene, more particularly a nucleotide sequence selected from the group consisting of the polynucleotides of the 5′ and 3′ regulatory region or a fragment or variant thereof, may be identified by using a one-hybrid system, such as that described in the booklet enclosed in the Matchmaker One-Hybrid System kit from Clontech (Catalog Ref. no. K1603-1, the technical teachings of which are herein incorporated by reference). Briefly, the target nucleotide sequence is cloned upstream of a selectable reporter sequence and the resulting polynucleotide construct is integrated in the yeast genome (Saccharomyces cerevisiae). Preferably, multiple copies of the target sequences are inserted into the reporter plasmid in tandem. The yeast cells containing the reporter sequence in their genome are then transformed with a library comprising fusion molecules between cDNAs encoding candidate proteins for binding onto the regulatory sequences of the GENSET gene and sequences encoding the activator domain of a yeast transcription factor such as GAL4. The recombinant yeast cells are plated in a culture broth for selecting cells expressing the reporter sequence. The recombinant yeast cells thus selected contain a fusion protein that is able to bind onto the target regulatory sequence of the GENSET gene. Then, the cDNAs encoding the fusion proteins are sequenced and may be cloned into expression or transcription vectors in vitro. The binding of the encoded polypeptides to the target regulatory sequences of the GENSET gene may be confirmed by techniques familiar to the one skilled in the art, such as gel retardation assays or DNAse protection assays.

Ligands Interacting with GENSET Polypeptides

For the purpose of the present invention, a ligand means a molecule, such as a protein, a peptide, an antibody or any synthetic chemical compound capable of binding to a GENSET protein or one of its fragments or variants or to modulate the expression of the polynucleotide coding for GENSET or a fragment or variant thereof.

In the ligand screening method according to the present invention, a biological sample or a defined molecule to be tested as a putative ligand of a GENSET protein is brought into contact with the corresponding purified GENSET protein, for example the corresponding purified recombinant GENSET protein produced by a recombinant cell host as described herein, in order to form a complex between this protein and the putative ligand molecule to be tested.

As an illustrative example, to study the interaction of a GENSET protein, or a fragment comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs: 170-338, 456-560, 785-918 and polypeptides encoded by the clone inserts of the deposited clone pool, with drugs or small molecules, such as molecules generated through combinatorial chemistry approaches, the microdialysis coupled to HPLC method described by Wang et al. (1997) or the affinity capillary electrophoresis method described by Bush et al. (1997), the disclosures of which are incorporated by reference, can be used.

In further methods, peptides, drugs, fatty acids, lipoproteins, or small molecules which interact with a GENSET protein, or a fragment comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs:170-338, 456-560, 785-918 and polypeptides encoded by the clone inserts of the deposited clone pool may be identified using assays such as the following. The molecule to be tested for binding is labeled with a detectable label, such as a fluorescent, radioactive, or enzymatic tag and placed in contact with immobilized GENSET protein, or a fragment thereof under conditions which permit specific binding to occur. After removal of non-specifically bound molecules, bound molecules are detected using appropriate means.

Various candidate substances or molecules can be assayed for interaction with a GENSET polypeptide. These substances or molecules include, without being limited to, natural or synthetic organic compounds or molecules of biological origin such as polypeptides. When the candidate substance or molecule comprises a polypeptide, this polypeptide may be the resulting expression product of a phage clone belonging to a phage-based random peptide library, or alternatively the polypeptide may be the resulting expression product of a cDNA library cloned in a vector suitable for performing a two-hybrid screening assay.

A. Candidate Ligands Obtained from Random Peptide Libraries

In a particular embodiment of the screening method, the putative ligand is the expression product of a DNA insert contained in a phage vector (Parmley and Smith, 1988). Specifically, random peptide phages libraries are used. The random DNA inserts encode for peptides of 8 to 20 amino acids in length (Oldenburg et al., 1992; Valadon et al., 1996; Lucas, 1994; Westerink, 1995; Felici et al., 1991), which disclosures are hereby incorporated by reference in their entireties. According to this particular embodiment, the recombinant phages expressing a protein that binds to an immobilized GENSET protein is retained and the complex formed between the GENSET protein and the recombinant phage may be subsequently immunoprecipitated by a polyclonal or a monoclonal antibody directed against the GENSET protein.

Once the ligand library in recombinant phages has been constructed, the phage population is brought into contact with the immobilized GENSET protein. Then the preparation of complexes is washed in order to remove the non-specifically bound recombinant phages. The phages that bind specifically to the GENSET protein are then eluted by a buffer (acid pH) or immunoprecipitated by the monoclonal antibody produced by the hybridoma anti-GENSET, and this phage population is subsequently amplified by an over-infection of bacteria (for example E. coli). The selection step may be repeated several times, preferably 24 times, in order to select the more specific recombinant phage clones. The last step comprises characterizing the peptide produced by the selected recombinant phage clones either by expression in infected bacteria-and isolation, expressing the phage insert in another host-vector system, or sequencing the insert contained in the selected recombinant phages.

B. Candidate Ligands Obtained by Competition Experiments.

Alternatively, peptides, drugs or small molecules which bind to a GENSET protein or fragment thereof comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs:170-338, 456-560, 785-918 and polypeptides encoded by the clone inserts of the deposited clone pool, may be identified in competition experiments. In such assays, the GENSET protein, or a fragment thereof, is immobilized to a surface, such as a plastic plate. Increasing amounts of the peptides, drugs or small molecules are placed in contact with the immobilized GENSET protein, or a fragment thereof, in the presence of a detectable labeled known GENSET protein ligand. For example, the GENSET ligand may be detectably labeled with a fluorescent, radioactive, or enzymatic tag. The ability of the test molecule to bind the GENSET protein, or a fragment thereof, is determined by measuring the amount of detectably labeled known ligand bound in the presence of the test molecule. A decrease in the amount of known ligand bound to the GENSET protein, or a fragment thereof, when the test molecule is present indicated that the test molecule is able to bind to the GENSET protein, or a fragment thereof.

C. Candidate Ligands Obtained by Affinity Chromatography.

Proteins or other molecules interacting with a GENSET protein, or a fragment thereof comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs: 170-338, 456-560, 785-918 and polypeptides encoded by the clone inserts of the deposited clone pool, can also be found using affinity columns which contain the GENSET protein, or a fragment thereof. The GENSET protein, or a fragment thereof, may be attached to the column using conventional techniques including chemical coupling to a suitable column matrix such as agarose, Affi Gel®, or other matrices familiar to those of skill in art. In some embodiments of this method, the affinity column contains chimeric proteins in which the GENSET protein, or a fragment thereof, is fused to glutathion S transferase (GST). A mixture of cellular proteins or pool of expressed proteins as described above is applied to the affinity column. Proteins or other molecules interacting with the GENSET protein, or a fragment thereof, attached to the column can then be isolated and analyzed on 2-D electrophoresis gel as described in Ramunsen et al. (1997), the disclosure of which is incorporated by reference. Alternatively, the proteins retained on the affinity column can be purified by electrophoresis based methods and sequenced. The same method can be used to isolate antibodies, to screen phage display products, or to screen phage display human antibodies.

D. Candidate Ligands Obtained by Optical Biosensor Methods

Proteins interacting with a GENSET protein, or a fragment comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs:170-338, 456-560, 785-918 and polypeptides encoded by the clone inserts of the deposited clone pool, can also be screened by using an Optical Biosensor as described in Edwards and Leatherbarrow (1997) and also in Szabo et al. (1995), the disclosures of which are incorporated by reference. This technique permits the detection of interactions between molecules in real time, without the need of labeled molecules. This technique is based on the surface plasmon resonance (SPR) phenomenon. Briefly, the candidate ligand molecule to be tested is attached to a surface (such as a carboxymethyl dextran matrix). A light beam is directed towards the side of the surface that does not contain the sample to be tested and is reflected by said surface. The SPR phenomenon causes a decrease in the intensity of the reflected light with a specific association of angle and wavelength. The binding of candidate ligand molecules cause a change in the refraction index on the surface, which change is detected as a change in the SPR signal. For screening of candidate ligand molecules or substances that are able to interact with the GENSET protein, or a fragment thereof, the GENSET protein, or a fragment thereof, is immobilized onto a surface. This surface comprises one side of a cell through which flows the candidate molecule to be assayed. The binding of the candidate molecule on the GENSET protein, or a fragment thereof, is detected as a change of the SPR signal. The candidate molecules tested may be proteins, peptides, carbohydrates, lipids, or small molecules generated by combinatorial chemistry. This technique may also be performed by immobilizing eukaryotic or prokaryotic cells or lipid vesicles exhibiting an endogenous or a recombinantly expressed GENSET protein at their surface.

The main advantage of the method is that it allows the determination of the association rate between the GENSET protein and molecules interacting with the GENSET protein. It is thus possible to select specifically ligand molecules interacting with the GENSET protein, or a fragment thereof, through strong or conversely weak association constants.

E. Candidate Ligands Obtained Through a Two-Hybrid Screening Assay.

The yeast two-hybrid system is designed to study protein-protein interactions in vivo (Fields and Song, 1989), which disclosure is hereby incorporated by reference in its entirety, and relies upon the fusion of a bait protein to the DNA binding domain of the yeast Gal4 protein. This technique is also described in the U.S. Pat. No. 5,667,973 and the U.S. Pat. No. 5,283,173, the technical teachings of both patents being herein incorporated by reference.

The general procedure of library screening by the two-hybrid assay may be performed as described by Harper et al. (1993) or as described by Cho et al. (1998) or also Fromont-Racine et al. (1997), which disclosures are hereby incorporated by reference in their entireties.

The bait protein or polypeptide comprises, consists essentially of, or consists of a GENSET polypeptide or a fragment thereof comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs:170-338, 456-560, 785-918 and polypeptides encoded by the clone inserts of the deposited clone pool.

More precisely, the nucleotide sequence encoding the GENSET polypeptide or a fragment or variant thereof is fused to a polynucleotide encoding the DNA binding domain of the GAL4 protein, the fused nucleotide sequence being inserted in a suitable expression vector, for example pAS2 or pM3.

Then, a human cDNA library is constructed in a specially designed vector, such that the human cDNA insert is fused to a nucleotide sequence in the vector that encodes the transcriptional domain of the GAL4 protein. Preferably, the vector used is the pACT vector. The polypeptides encoded by the nucleotide inserts of the human cDNA library are termed “prey” polypeptides.

A third vector contains a detectable marker gene, such as beta galactosidase gene or CAT gene that is placed under the control of a regulation sequence that is responsive to the binding of a complete Gal4 protein containing both the transcriptional activation domain and the DNA binding domain. For example, the vector pG5EC may be used.

Two different yeast strains are also used. As an illustrative but non limiting example the two different yeast strains may be the followings:

Y190, the phenotype of which is (MATa, Leu2-3, 112 ura3-12, trp1-901, his3-D200, ade2-101, gal4Dga1180D URA3 GAL-LacZ, LYS GAL-HI S3, cyh^{r});

Y187, the phenotype of which is (MATa gal4 gal80 his3 trp1-901 ade2-101 ura3-52 leu2-3, -112 URA3 GAL-lacZmet⁻), which is the opposite mating type of Y190.

Briefly, 20 μg of pAS2/GENSET and 20 μg of pACT-cDNA library are co-transformed into yeast strain Y190. The transformants are selected for growth on minimal media lacking histidine, leucine and tryptophan, but containing the histidine synthesis inhibitor 3-AT (50 mM). Positive colonies are screened for beta galactosidase by filter lift assay. The double positive colonies (His⁺, beta-gal⁺) are then grown on plates lacking histidine, leucine, but containing tryptophan and cycloheximide (10 mg/ml) to select for loss of pAS2/GENSET plasmids but retention of pACT-cDNA library plasmids. The resulting Y190 strains are mated with Y187 strains expressing GENSET or non-related control proteins; such as cyclophilin B, lamin, or SNF1, as Gal4 fusions as described by Harper et al. (1993) and by Bram et al. (1993), which disclosures are hereby incorporated by reference in their entireties, and screened for beta galactosidase by filter lift assay. Yeast clones that are beta gal-after mating with the control Gal4 fusions are considered false positives.

In another embodiment of the two-hybrid method according to the invention, interaction between the GENSET or a fragment or variant thereof with cellular proteins may be assessed using the Matchmaker Two Hybrid System 2 (Catalog No. K1604-1, Clontech). As described in the manual accompanying the kit, the disclosure of which is incorporated herein by reference, nucleic acids encoding the GENSET protein or a portion thereof, are inserted into an expression vector such that they are in frame with DNA encoding the DNA binding domain of the yeast transcriptional activator GAL4. A desired cDNA, preferably human cDNA, is inserted into a second expression vector such that they are in frame with DNA encoding the activation domain of GAL4. The two expression plasmids are transformed into yeast and the yeast are plated on selection medium which selects for expression of selectable markers on each of the expression vectors as well as GALA dependent expression of the HIS3 gene. Transformants capable of growing on medium lacking histidine are screened for GAL4 dependent lacZ expression. Those cells which are positive in both the histidine selection and the lacZ assay contain interaction between GENSET and the protein or peptide encoded by the initially selected cDNA insert

Compounds Modulating GENSET Biological Activity

Another method of screening for compounds that modulate GENSET expression and/or biological activity is by measuring the effects of test compounds on specific biological activity, e.g. a GENSET biological activity in a host cell. In one embodiment, the present invention relates to a method of identifying an agent which alters GENSET biological activity, wherein a nucleic acid construct comprising a nucleic acid which encodes a mammalian GENSET polypeptide is introduced into a host cell. The host cells produced are maintained under conditions appropriate for expression of the encoded mammalian GENSET polypeptides, whereby the nucleic acid is expressed. The host cells are then contacted with a compound to be assessed (an “agent,” or “test agent”), and the properties of the cells is assessed. Detection of a change in any GENSET polypeptide-associated property in the presence of the agent indicates that the agent alters GENSET activity. In a particular embodiment, the invention relates to a method of identifying an agent which is an activator of GENSET activity, wherein detection of an increase of any GENSET polypeptide-associated property in the presence of the agent indicates that the agent activates GENSET activity. In another particular embodiment, the invention relates to a method of identifying an agent which is an inhibitor of GENSET activity, wherein detection of a decrease of any GENSET polypeptide-associated property in the presence of the agent indicates that the agent inhibits GENSET activity.

In a particular embodiment, a high throughput screen can be used to identify agents that activate (enhance) or inhibit GENSET activity (See e.g., PCT publication WO 98/45438, which disclosure is hereby incorporated by reference in its entirety). For example, the method of identifying an agent which alters GENSET activity can be performed as follows. A nucleic acid construct comprising a polynucleotide which encodes a mammalian GENSET polypeptide is introduced into a host cell to produce recombinant host cells. The recombinant host cells are then maintained under conditions appropriate for expression of the encoded mammalian GENSET polypeptide, whereby the nucleic acid is expressed. The compound to be assessed is added to the recombinant host cells; the resulting combination is referred to as a test sample. A detectable, GENSET polypeptide-associated property of the cells is detected. A control can be used in the methods of detecting agents which alter GENSET activity. For example, the control sample includes the same reagents but lacks the compound or agent being assessed; it is treated in the same manner as the test sample.

Methods of Screening for Compounds Modulating GENSET Expression and/or Activity

The present invention also relates to methods of screening compounds for their ability to modulate (e.g. increase or inhibit) the activity or expression of GENSET. More specifically, the present invention relates to methods of testing compounds for their ability either to increase or to decrease expression or activity of GENSET. The assays are performed in vitro or in vivo.

In Vitro Methods

In vitro, cells expressing GENSET polypeptides are incubated in the presence and absence of the test compound. By determining the level of GENSET expression in the presence of the test compound or the level of GENSET activity in the presence of the test compound, compounds can be identified that suppress or enhance GENSET expression or activity. Alternatively, constructs comprising a GENSET regulatory sequence operably linked to a reporter gene (e.g. luciferase, chloramphenicol acetyl transferase, LacZ, green fluorescent protein, etc.) can be introduced into host cells and the effect of the test compounds on expression of the reporter gene detected. Cells suitable for use in the foregoing assays include, but are not limited to, cells having the same origin as tissues or cell lines in which the polypeptide is known to be expressed using the data from Tables III, IV, or V.

Consequently, the present invention encompasses a method for screening molecules that modulate the expression of a GENSET gene, said screening method comprising the steps of:

a) cultivating a prokaryotic or an eukaryotic cell that has been transfected with a nucleotide sequence encoding a GENSET protein or a variant or a fragment thereof, placed under the control of its own promoter;

b) bringing into contact said cultivated cell with a molecule to be tested;

c) quantifying the expression of said GENSET protein or a variant or a fragment thereof in the presence of said molecule.

Using DNA recombination techniques well known by the one skill in the art, the GENSET protein encoding DNA sequence is inserted into an expression vector, downstream from its promoter sequence. As an illustrative example, the promoter sequence of the GENSET gene is contained in the 5′ untranscribed region of the GENSET genomic DNA.

The quantification of the expression of a GENSET protein may be realized either at the mRNA level (using for example Northen blots, RT-PCR, preferably quantitative RT-PCR with primers and probes specific for the GENSET mRNA of interest) or at the protein level (using polyclonal or monoclonal antibodies in immunoassays such as ELISA or RIA assays, Western blots, or immunochemistry).

The present invention also concerns a method for screening substances or molecules that are able to increase, or in contrast to decrease, the level of expression of a GENSET gene. Such a method may allow the one skilled in the art to select substances exerting a regulating effect on the expression level of a GENSET gene and which may be useful as active ingredients included in pharmaceutical compositions for treating patients suffering from disorders associated with abnormal levels of GENSET products.

Thus, another part of the present invention is a method for screening a candidate molecule that modulates the expression of a GENSET gene, this method comprises the following steps:

a) providing a recombinant cell host containing a nucleic acid, wherein said nucleic acid comprises a GENSET 5′ regulatory region or a regulatory active fragment or variant thereof, operably linked to a polynucleotide encoding a detectable protein;

b) obtaining a candidate molecule; and

c) determining the ability of said candidate molecule to modulate the expression levels of said polynucleotide encoding the detectable protein.

In a further embodiment, said nucleic acid comprising a GENSET 5′ regulatory region or a regulatory active fragment or variant thereof, includes the 5′UTR region of a GENSET cDNA selected from the group comprising of the 5′UTRs of the sequences of SEQ ID NOs:1-69, 339-455, 561-784, sequences of clones inserts of the deposited clone pool, regulatory active fragments and variants thereof. In a more preferred embodiment of the above screening method, said nucleic acid includes a promoter sequence which is endogenous with respect to the GENSET 5′UTR sequence. In another more preferred embodiment of the above screening method, said nucleic acid includes a promoter sequence which is exogenous with respect to the GENSET 5′UTR sequence defined therein.

Preferred polynucleotides encoding a detectable protein are polynucleotides encoding beta galactosidase, green fluorescent protein (GFP) and chloramphenicol acetyl transferase (CAT).

The invention further relates to a method for the production of a pharmaceutical composition comprising a method of screening a candidate molecule that modulates the expression of a GENSET gene and furthermore mixing the identified molecule with a pharmaceutically acceptable carrier.

The invention also pertains to kits for the screening of a candidate substance modulating the expression of a GENSET gene. Preferably, such kits comprise a recombinant vector that allows the expression of a GENSET 5′ regulatory region or a regulatory active fragment or a variant thereof, operably linked to a polynucleotide encoding a detectable protein or a GENSET protein or a fragment or a variant thereof. More preferably, such kits include a recombinant vector that comprises a nucleic acid including the 5′UTR region of a GENSET cDNA selected from the group comprising the 5′UTRs of the sequences of SEQ ID NOs:1-169, 339-455, 561-784, sequences of clone inserts of the deposited clone pool, regulatory active fragments and variants thereof, being operably linked to a polynucleotide encoding a detectable protein.

For the design of suitable recombinant vectors useful for performing the screening methods described above, it will be referred to the section of the present specification wherein the preferred recombinant vectors of the invention are detailed.

Another object of the present invention comprises methods and kits for the screening of candidate substances that interact with a GENSET polypeptide, fragments or variants thereof. By their capacity to bind covalently or non-covalently to a GENSET protein, fragments or variants thereof, these substances or molecules may be advantageously used both in vitro and in vivo.

In vitro, said interacting molecules may be used as detection means in order to identify the presence of a GENSET protein in a sample, preferably a biological sample.

A method for the screening of a candidate substance that interact with a GENSET polypeptide, fragments or variants thereof, said methods comprising the following steps:

a) providing a polypeptide comprising, consisting essentially of, or consisting of a GENSET protein or a fragment comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs:170-338, 456-560, 785-918 and polypeptides encoded by the clone inserts of the deposited clone pool;

b) obtaining a candidate substance;

c) bringing into contact said polypeptide with said candidate substance;

d) detecting the complexes formed between said polypeptide and said candidate substance.

The invention further relates to a method for the production of a pharmaceutical composition comprising a method for the screening of a candidate substance that interact with a GENSET polypeptide, fragments or variants thereof and furthermore mixing the identified substance with a pharmaceutically acceptable carrier.

The invention further concerns a kit for the screening of a candidate substance interacting with the GENSET polypeptide, wherein said kit comprises:

a) a polypeptide comprising, consisting essentially of, or consisting of a GENSET protein or a fragment comprising a contiguous span of at least 6 amino acids, preferably at least 8 to 10 amino acids, more preferably at least 12, 15, 20, 25, 30, 40, 50, or 100 amino acids of a polypeptide selected from the group consisting of sequences of SEQ ID NOs:170-338, 456-560, 785-918 and polypeptides encoded by the clone inserts of the deposited clone pool; and

b) optionally means useful to detect the complex formed between said polypeptide or a variant thereof and the candidate substance.

In a preferred embodiment of the kit described above, the detection means comprises a monoclonal or polyclonal antibody binding to said GENSET protein or fragment or variant thereof.

In Vivo Methods

Compounds that suppress or enhance GENSET expression can also be identified using in vivo screens. In these assays, the test compound is administered (e.g. IV, IP, IM, orally, or otherwise), to the animal, for example, at a variety of dose levels. The effect of the compound on GENSET expression is determined by comparing GENSET levels, for example in tissues known to express the gene of interest using, for example the data obtained in Tables III, IV, or V, and using Northern blots, immunoassays, PCR, etc., as described above. Suitable test animals include, but are not limited to, rodents (e.g., mice and rats), primates, and rabbits. Humanized mice can also be used as test animals, that is mice in which the endogenous mouse protein is ablated (knocked out) and the homologous human protein added back by standard transgenic approaches. Such mice express only the human form of a protein. Humanized mice expressing only the human GENSET can be used to study in vivo responses to potential agents regulating GENSET protein or mRNA levels. As an example, transgenic mice have been produced carrying the human apoE4 gene. They are then bred with a mouse line that lacks endogenous apoE, to produce an animal model carrying human proteins believed to be instrumental in development of Alzheimer's pathology. Such transgenic animals are useful for dissecting the biochemical and physiological steps of disease, and for development of therapies for disease intervention (Loring, et al, 1996) (incorporated herein by reference in its entirety).

Uses for Compounds Modulating GENSET Expression and/or Biological Activity

Using in vivo (or in vitro) systems, it may be possible to identify compounds that exert a tissue specific effect, for example, that increase GENSET expression or activity only in tissues of interest, such as the adrenal gland, bone marrow, brain, cerebellum, colon, fetal brain, fetal kidney, fetal liver, heart, hypertrophic prostate, kidney, liver, lung, lymph ganglia, lymphocytes, muscle, ovary, pancreas, pituitary gland, placenta, prostate, salivary gland, spinal cord, spleen, stomach, intestine, substantia nigra, testis, thyroid, umbilical cord, and uterus. Screening procedures such as those described above are also useful for identifying agents for their potential use in pharmacological intervention strategies. Agents that enhance GENSET gene expression or stimulate its activity may thus be used to induce any phenotype associated with a GENSET gene, or to treat disorders resulting from a deficiency of a GENSET polypeptide activity or expression. Compounds that suppress GENSET polypeptide expression or inhibit its activity can be used to treat any disease or condition associated with increased or deleterious GENSET polypeptide activity or expression.

Also encompassed by the present invention is an agent which interacts with a GENSET gene or polypeptide directly or indirectly, and inhibits or enhances GENSET polypeptide expression and/or function. In one embodiment, the agent is an inhibitor which interferes with a GENSET polypeptide directly (e.g., by binding the GENSET polypeptide) or indirectly (e.g., by blocking the ability of the GENSET polypeptide to have a GENSET biological activity). In a particular embodiment, an inhibitor of a GENSET protein is an antibody specific for the GENSET protein or a functional portion of the GENSET protein; that is, the antibody binds a GENSET polypeptide. For example, the antibody can be specific for a polypeptide encoded by one of the nucleic acid sequences of human GENSETs (SEQ D NOs:1-169, 339-455, 561-784), a mammalian GENSET nucleic acid, or portions thereof. Alternatively, the inhibitor can be an agent other than an antibody (e.g., small organic molecule, protein or peptide) which binds the GENSET polypeptide and blocks its activity. For example, the inhibitor can be an agent which mimics the GENSET polypeptide structurally, but lacks its function. Alternatively, it can be an agent which binds to or interacts with a molecule which the GENSET polypeptide normally binds to or interacts with, thus blocking the GENSET polypepetide from doing so and preventing it from exerting the effects it would normally exert.

In another embodiment, the agent is an enhancer (activator) of a GENSET polypeptide which increases the activity of the GENSET polypeptide (increases the effect of a given amount or level of GENSET), increases the length of time it is effective (by preventing its degradation or otherwise prolonging the time during which it is active) or both either directly or indirectly. For example, GENSET polynucleotides and polypeptides can be used to identify drugs which increase or decrease the ability of GENSET polypeptides to induce GENSET biological activity, which drugs are useful for the treatment or prevention of any disease or condition associated with a GENSET biological activity.

The GENSET sequences of the present invention can also be used to generate nonhuman gene knockout animals, such as mice, which lack a GENSET gene or transgenically overexpress a GENSET gene. For example, such GENSET gene knockout mice can be generated and used to obtain further insight into the function of the GENSET gene as well as assess the specificity of GENSET activators and inhibitors. Also, over expression of the GENSET gene (e.g., a human GENSET gene) in transgenic mice can be used as a means of creating a test system for GENSET activators and inhibitors (e.g., against a human GENSET polypeptide). In addition, the GENSET gene can be used to clone the GENSET promoter/enhancer in order to identify regulators of GENSET gene transcription. GENSET gene knockout animals include animals which completely or partially lack the GENSET gene and/or GENSET activity or function. Thus the present invention relates to a method of inhibiting (partially or completely) a GENSET biological activity in a mammal (e.g., a human), the method comprising administering to the mammal an effective amount of an inhibitor of a GENSET polypeptide or polynucleotide. The invention also relates to a method of enhancing a GENSET biological activity in a mammal, the method comprising administering to the mammal an effective amount of an enhancer of a GENSET polypeptide or polynucleotide.

Inhibiting GENSET Gene Expression

Therapeutic compositions according to the present invention may comprise advantageously one or several GENSET oligonucleotide fragments as an antisense tool or a triple helix tool that inhibits the expression of the corresponding GENSET gene.

Antisense Approach

In antisense approaches, nucleic acid sequences complementary to an mRNA are hybridized to the mRNA intracellularly, thereby blocking the expression of the protein encoded by the mRNA. The antisense nucleic acid molecules to be used in gene therapy may be either DNA or RNA sequences. Preferred methods using antisense polynucleotide according to the present invention are the procedures described by Sczakiel et al. (1995), which disclosure is hereby incorporated by reference in its entirety.

Preferably, the antisense tools are chosen among the polynucleotides (15-200 bp long) that are complementary to GENSET mRNA, more preferably to the 5′end of the GENSET mRNA. In another embodiment, a combination of different antisense polynucleotides complementary to different parts of the desired targeted gene are used.

Other preferred antisense polynucleotides according to the present invention are sequences complementary to either a sequence of GENSET mRNAs comprising the translation initiation codon ATG or a sequence of GENSET genomic DNA containing a splicing donor or acceptor site.

Preferably, the antisense polynucleotides of the invention have a 3′ polyadenylation signal that has been replaced with a self-cleaving ribozyme sequence, such that RNA polymerase II transcripts are produced without poly(A) at their 3′ ends, these antisense polynucleotides being incapable of export from the nucleus, such as described by Liu et al. (1994), which disclosure is hereby incorporated by reference in its entirety. In a preferred embodiment, these GENSET antisense polynucleotides also comprise, within the ribozyme cassette, a histone stem-loop structure to stabilize cleaved transcripts against 3′-5′ exonucleolytic degradation, such as the structure described by Eckner et al. (1991), which disclosure is hereby incorporated by reference in its entirety.

The antisense nucleic acids should have a length and melting temperature sufficient to permit formation of an intracellular duplex having sufficient stability to inhibit the expression of the GENSET mRNA in the duplex. Strategies for designing antisense nucleic acids suitable for use in gene therapy are disclosed in Green et al., (1986) and Izant and Weintraub, (1984), the disclosures of which are incorporated herein by reference.

In some strategies, antisense molecules are obtained by reversing the orientation of the GENSET coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of GENSET antisense nucleic acids in vivo by operably linking DNA containing the antisense sequence to a promoter in a suitable expression vector.

Alternatively, oligonucleotides which are complementary to the strand normally transcribed in the cell may be synthesized in vitro. Thus, the antisense nucleic acids are complementary to the corresponding mRNA and are capable of hybridizing to the mRNA to create a duplex. In some embodiments, the antisense sequences may contain modified sugar phosphate backbones to increase stability and make them less sensitive to RNase activity. Examples of modifications suitable for use in antisense strategies include 2′ O-methyl RNA oligonucleotides and Protein-nucleic acid (PNA) oligonucleotides. Further examples are described by Rossi et al., (1991), which disclosure is hereby incorporated by reference in its entirety.

Various types of antisense oligonucleotides complementary to the sequence of the GENSET cDNA or genomic DNA may be used. In one preferred embodiment, stable and semi-stable antisense oligonucleotides described in International Application No. PCT WO94/23026, hereby incorporated by reference, are used. In these molecules, the 3′ end or both the 3′ and 5′ ends are engaged in intramolecular hydrogen bonding between complementary base pairs. These molecules are better able to withstand exonuclease attacks and exhibit increased stability compared to conventional antisense oligonucleotides.

In another preferred embodiment, the antisense oligodeoxynucleotides against herpes simplex virus types 1 and 2 described in International Application No. WO 95/04141, hereby incorporated by reference, are used.

In yet another preferred embodiment, the covalently cross-linked antisense oligonucleotides described in International Application No. WO 96/31523, hereby incorporated by reference, are used. These double- or single-stranded oligonucleotides comprise one or more, respectively, inter- or intra-oligonucleotide covalent cross-linkages, wherein the linkage consists of an amide bond between a primary amine group of one strand and a carboxyl group of the other strand or of the same strand, respectively, the primary amine group being directly substituted in the 2′ position of the strand nucleotide monosaccharide ring, and the carboxyl group being carried by an aliphatic spacer group substituted on a nucleotide or nucleotide analog of the other strand or the same strand, respectively.

The antisense oligodeoxynucleotides and oligonucleotides disclosed in International Application No. WO 92/18522, incorporated by reference, may also be used. These molecules are stable to degradation and contain at least one transcription control recognition sequence which binds to control proteins and are effective as decoys therefor. These molecules may contain “hairpin” structures, “dumbbell” structures, “modified dumbbell” structures, “cross-linked” decoy structures and “loop” structures.

In another preferred embodiment, the cyclic double-stranded oligonucleotides described in European Patent Application No. 0 572 287 A2, hereby incorporated by reference are used. These ligated oligonucleotide “dumbbells” contain the binding site for a transcription factor and inhibit expression of the gene under control of the transcription factor by sequestering the factor.

Use of the closed antisense oligonucleotides disclosed in International Application No. WO 92/19732, hereby incorporated by reference, is also contemplated. Because these molecules have no free ends, they are more resistant to degradation by exonucleases than are conventional oligonucleotides. These oligonucleotides may be multifunctional, interacting with several regions which are not adjacent to the target mRNA.

The appropriate level of antisense nucleic acids required to inhibit gene expression may be determined using in vitro expression analysis. The antisense molecule may be introduced into the cells by diffusion, injection, infection or transfection using procedures known in the art. For example, the antisense nucleic acids can be introduced into the body as a bare or naked oligonucleotide, oligonucleotide encapsulated in lipid, oligonucleotide sequence encapsidated by viral protein, or as an oligonucleotide operably linked to a promoter contained in an expression vector. The expression vector may be any of a variety of expression vectors known in the art, including retroviral or viral vectors, vectors capable of extrachromosomal replication, or integrating vectors. The vectors may be DNA or RNA.

The antisense molecules are introduced onto cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg bodyweight. Levels of oligonucleotide approaching 100 mg/kg bodyweight or higher may be possible after testing the toxicity of the oligonucleotide in laboratory animals. It is additionally contemplated that cells from the vertebrate are removed, treated with the antisense oligonucleotide, and reintroduced into the vertebrate.

In a preferred application of this invention, the polypeptide encoded by the gene is first identified, so that the effectiveness of antisense inhibition on translation can be monitored using techniques that include but are not limited to antibody-mediated tests such as RIAs and ELISA, functional assays, or radiolabeling.

An alternative to the antisense technology that is used according to the present invention comprises using ribozymes that will bind to a target sequence via their complementary polynucleotide tail and that will cleave the corresponding RNA by hydrolyzing its target site (namely “hammerhead ribozymes”). Briefly, the simplified cycle of a hammerhead ribozyme comprises (1) sequence specific binding to the target RNA via complementary antisense sequences; (2) site-specific hydrolysis of the cleavable motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Rossi et al, (1991) and Sczakiel et al. (1995), the specific preparation procedures being referred to in said articles being herein incorporated by reference.

Triple Helix Approach

The GENSET genomic DNA may also be used to inhibit the expression of the GENSET gene based on intracellular triple helix formation.

Triple helix oligonucleotides are used to inhibit transcription from a genome. They are particularly useful for studying alterations in cell activity when it is associated with a particular gene. The GENSET cDNAs or genomic DNAs of the present invention or, more preferably, a fragment of those sequences, can be used to inhibit gene expression in individuals having diseases associated with expression of a particular gene. Similarly, a portion of the GENSET genomic DNA can be used to study the effect of inhibiting GENSET gene transcription within a cell. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences from the GENSET genomic DNA are contemplated within the scope of this invention.

To carry out gene therapy strategies using the triple helix approach, the sequences of the GENSET genomic DNA are first scanned to identify 10-mer to 20-mer homopyrimidine or homopurine stretches which could be used in triple-helix based strategies for inhibiting GENSET expression. Following identification of candidate homopyrimidine or homopurine stretches, their efficiency in inhibiting GENSET expression is assessed by introducing varying amounts of oligonucleotides containing the candidate sequences into tissue culture cells which express the GENSET gene.

The oligonucleotides can be introduced into the cells using a variety of methods known to those skilled in the art, including but not limited to calcium phosphate precipitation, DEAE-Dextran, electroporation, liposome-mediated transfection or native uptake.

Treated cells are monitored for altered cell function or reduced GENSET expression using techniques such as Northern blotting, RNase protection assays, or PCR based strategies to monitor the transcription levels of the GENSET gene in cells which have been treated with the oligonucleotide. The cell functions to be monitored are predicted based upon the homologies of the target gene corresponding to the cDNA from which the oligonucleotide was derived with known gene sequences that have been associated with a particular function. The cell functions can also be predicted based on the presence of abnormal physiology within cells derived from individuals with a particular inherited disease, particularly when the cDNA is associated with the disease using techniques described in the section entitled “Identification of genes associated with hereditary diseases or drug response”.

The oligonucleotides which are effective in inhibiting gene expression in tissue culture cells may then be introduced in vivo using the techniques and at a dosage calculated based on the in vitro results, as described in the section entitled “Antisense Approach”.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al. (1989), which is hereby incorporated by this reference.

Treating GENSET Gene-Related Disorders

The present invention further relates to methods, uses of GENSET polypeptides and polynucleotides, and uses of modulators of GENSET polypeptides and polynucleotides, for treating diseases/disorders associated with GENSET genes by increasing or decreasing GENSET gene activity and/or expression. These methodologies can be effected using compounds selected using screening protocols such as those described herein and/or by using the gene therapy and antisense approaches described in the art and herein. Gene therapy can be used to effect targeted expression of GENSET genes in any tissue, e.g. a tissue associated with the disease or condition to be treated. The GENSET coding sequence can be cloned into an appropriate expression vector and targeted to a particular cell type(s) to achieve efficient, high level expression. Introduction of the GENSET coding sequence into target cells can be achieved, for example, using particle mediated DNA delivery, (Haynes, 1996 and Maurer, 1999), direct injection of naked DNA, (Levy et al., 1996; and Feigner, 1996), or viral vector mediated transport (Smith et al., 1996, Stone et al, 2000; Wu and Atai, 2000), each of which disclosures are hereby incorporated by reference in their entireties. Tissue specific effects can be achieved, for example, in the case of virus mediated transport by using viral vectors that are tissue specific, or by the use of promoters that are tissue specific. For instance, any tissue-specific promoter may be used to achieve specific expression, for example albumin promoters (liver specific; Pinkert et al., 1987 Genes Dev. 1:268-277), lymphoid specific promoters (Calame et al., 1988 Adv. Immunol. 43:235-275), promoters of T-cell receptors (Winoto et al., 1989 EMBO J. 8:729-733) and immunoglobulins (Banerji et al., 1983 Cell 33:729-740; Queen and Baltimore 1983 Cell 33:741-748), neuron-specific promoters (e.g. the neurofilament promoter; Byrne et al., 1989 Proc. Natl. Acad. Sci. USA 86:5473-5477), pancreas-specific promoters (Edlunch et al., 1985 Science 230:912-916) or mammary gland-specific promoters (milk whey promoter, U.S. Pat. No. 4,873,316 and European Application Publication No. 264, 166). Developmentally-regulated promoters can also be used, such as the murine homeobox promoters (Kessel et al., 1990 Science 249:374-379) or the alpha-fetoprotein promoter (Campes et al., 1989 Genes Dev. 3:537-546).

Combinatorial approaches can also be used to ensure that the GENSET coding sequence is activated in the target tissue (Butt and Karathanasis, 1995; Miller and Whelan, 1997), which disclosures are hereby incorporated by reference in their entireties. Antisense oligonucleotides complementary to GENSET mRNA can be used to selectively diminish or ablate the expression of the protein, for example, at sites of inflammation. More specifically, antisense constructs or antisense oligonucleotides can be used to inhibit the production of GENSET in high expressing cells such as those cited in the third column of Table V. Antisense mRNA can be produced by transfecting into target cells an expression vector with the GENSET gene sequence, or a portion thereof, oriented in an antisense direction relative to the direction of transcription. Appropriate vectors include viral vectors, including retroviral, adenoviral, and adeno-associated viral vectors, as well as nonviral vectors. Tissue specific promoters can be used, as described supra. Alternatively, antisense oligonucleotides can be introduced directly into target cells to achieve the same goal. (See also other delivery methodologies described herein in connection with gene therapy.). Oligonucleotides can be selected/designed to achieve a high level of specificity (Wagner et al., 1996), which disclosure is hereby incorporated by reference in its entirety. The therapeutic methodologies described herein are applicable to both human and non-human mammals (including cats and dogs).

### Pharmaceutical and Physiologically Acceptable Compositions

The present invention also relates to pharmaceutical or physiologically acceptable compositions comprising, as active agent, the polypeptides, nucleic acids or antibodies of the invention. The invention also relates to compositions comprising, as active agent, compounds selected using the above-described screening protocols. Such compositions include the active agent in combination with a pharmaceutical or physiologically acceptable carriers. In the case of naked DNA, the “carrier” may be gold particles. The amount of active agent in the composition can vary with the agent, the patient and the effect sought. Likewise, the dosing regimen can vary depending on the composition and the disease/disorder to be treated.

Therefore, the invention related to methods for the production of pharmaceutical composition comprising a method for selecting an active agent, compound, substance or molecule using any of the screening method described herein and furthermore mixing the identified active agent, compound, substance or molecule with a pharmaceutically acceptable carrier.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co. Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through a combination of active compounds with solid excipient, sulting mixture is optionally grinding, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titaniumdioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquidpolyethylene glycol with or without stabilizers.

Pharmaceutical formulations suitable for parenteral administration may be formulated in aqueous solutions, preferably in physiologically compatible buffers such as Hanks solution, Ringer's solution, or physiologically buffered saline. Aqueous injection suspensions may contain substances which increase the viscosity of the suspension, such as sodium carboxymethylcellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of a GENSET polypeptide, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example a GENSET polypeptide or fragments thereof, antibodies specific to GENSET polypeptides, agonists, antagonists or inhibitors of GENSET polypeptides, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions maybe administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### Use of Genset Sequences: Computer-Related Embodiments

As used herein the term “cDNA codes of SEQ ID NOs:1-169, 339455, 561-784” encompasses the nucleotide sequences of SEQ ID NOs:1-169, 339-455, 561-784 and of clones inserts of the deposited clone pool, fragments thereof, nucleotide sequences homologous thereto, and sequences complementary to all of the preceding sequences. The fragments include fragments of SEQ ID NOs:1-169, 339-455, 561-784 comprising at least 8, 10, 12, 15, 18, 20, 25, 28, 30, 35, 40, 50, 75, 100, 150, 200, 300, 400, 500, 1000 or 2000 consecutive nucleotides of SEQ ID NOs:1-169, 339-455, 561-784. Preferably the fragments include polynucleotides described herein as encoding polypeptides having a biological activity. Homologous sequences and fragments of SEQ ID NOs:1-169, 339-455, 561-784 refer to a sequence having at least 99%, 98%, 97%, 96%, 95%, 90%, 85%, 80%, or 75% identity to these sequences. Identity may be determined using any of the computer programs and parameters described herein, including BLAST2N with the default parameters or with any modified parameters. Homologous sequences also include RNA sequences in which uridines replace the thymines in the cDNA codes of SEQ ID NOs:1-169, 339-455, 561-784. The homologous sequences may be obtained using any of the procedures described herein or may result from the correction of a sequencing error as described above. It will be appreciated that the cDNA codes of SEQ ID NOs:1-169, 339-455, 561-784 can be represented in the traditional single character format (see, e.g. the inside back cover of Stryer, 1995) or in any other format which records the identity of the nucleotides in a sequence.

As used herein the term “polypeptide codes of SEQ ID NOs:170-338, 456-560, 785-918” encompasses the polypeptide sequences of SEQ ID NOs:170-338, 456-560, 785-918 which are encoded by the cDNAs of SEQ ID NOs:1-169, 339-455, 561-784, the polypeptide sequences encoded by the clone inserts of the deposited clone pool, polypeptide sequences homologous thereto, or fragments of any of the preceding sequences. Homologous polypeptide sequences refer to a polypeptide sequence having at least 99%, 98%, 97%, 96%, 95%, 90%, 85%, 80%, 75% identity to one of the polypeptide sequences of SEQ ID NOs:170-338, 456-560, 785-918. Identity may be determined using any of the computer programs and parameters described herein, including FASTA with the default parameters or with any modified parameters. The homologous sequences may be obtained using any of the procedures described herein or may result from the correction of a sequencing error as described above. The polypeptide fragments comprise at least 5, 6, 8, 10, 12, 15, 20, 25, 30, 35, 40, 50, 60, 75, 100, 150 or 200 consecutive amino acids of the polypeptides of SEQ ID NOs:170-338, 456-560, 785-918. Preferably, the fragments include polypeptides described herein as having a biological activity, or fragments comprising at least 5, 10, 15, 20, 25, 30, 35, 40, 50, 75, 100, or 150 consecutive amino acids of polypeptides described herein as having a biological activity. It will be appreciated that the polypeptide codes of the SEQ ID NOs: 170-338, 456-560, 785-918 can be represented in the traditional single character format or three letter format (see, the inside back cover of Stryer, 1995) or in any other format which relates the identity of the polypeptides in a sequence.

It will be appreciated by those skilled in the art that the nucleic acid codes of the invention and polypeptide codes of the invention can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of the invention, or one or more of the polypeptide codes of the invention. Another aspect of the present invention is a computer readable medium having recorded thereon at least 2, 5, 10, 15, 20, 25, 30, or 50 nucleic acid codes of the invention. Another aspect of the present invention is a computer readable medium having recorded thereon at least 2, 5, 10, 15, 20, 25, 30, or 50 polypeptide codes of the invention.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

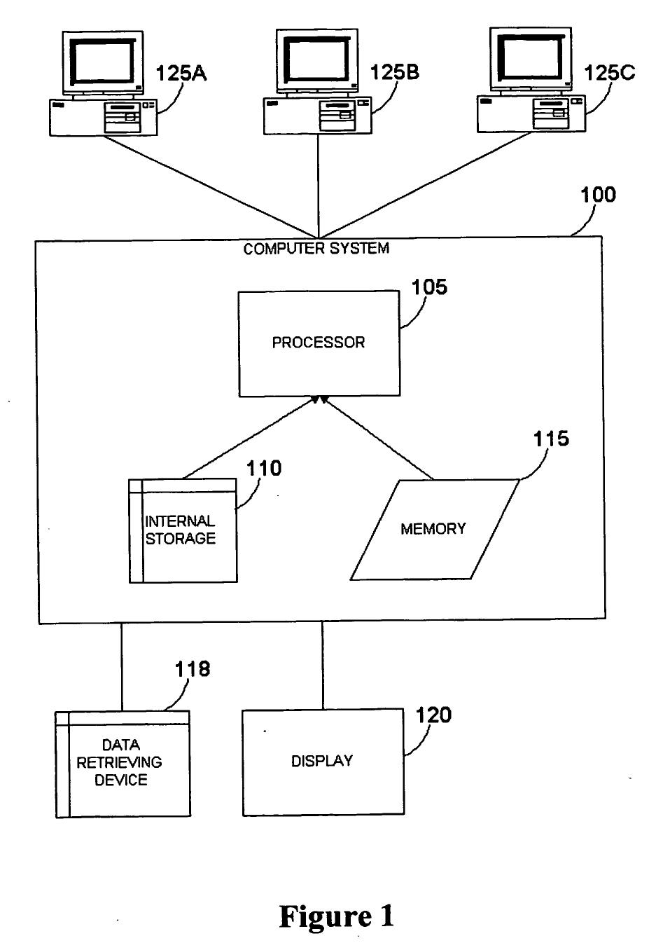

Embodiments of the present invention include systems, particularly computer systems which store and manipulate the sequence information described herein. One example of a computer system 100 is illustrated in block diagram form in FIG. 1. As used herein, “a computer system” refers to the hardware components, software components, and data storage components used to analyze the nucleotide sequences of the nucleic acid codes of the invention or the amino acid sequences of the polypeptide codes of the invention. In one embodiment, the computer system 100 is a Sun Enterprise 1000 server (Sun Microsystems, Palo Alto, Calif.). The computer system 100 preferably includes a processor for processing, accessing and manipulating the sequence data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data retrieving device 118 for reading the data stored on the internal data storage devices 110.

The data retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of the nucleic acid codes of the invention or the amino acid sequences of the polypeptide codes of the invention (such as search tools, compare tools, and modeling tools etc.) may reside in main memory 115 during execution.

In some embodiments, the computer system 100 may further comprise a sequence comparer for comparing the above-described nucleic acid codes of the invention or the polypeptide codes of the invention stored on a computer readable medium to reference nucleotide or polypeptide sequences stored on a computer readable medium. A “sequence comparer” refers to one or more programs which are implemented on the computer system 100 to compare a nucleotide or polypeptide sequence with other nucleotide or polypeptide sequences and/or compounds including but not limited to peptides, peptidomimetics, and chemicals stored within the data storage means. For example, the sequence comparer may compare the nucleotide sequences of nucleic acid codes of the invention or the amino acid sequences of the polypeptide codes of the invention stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention.

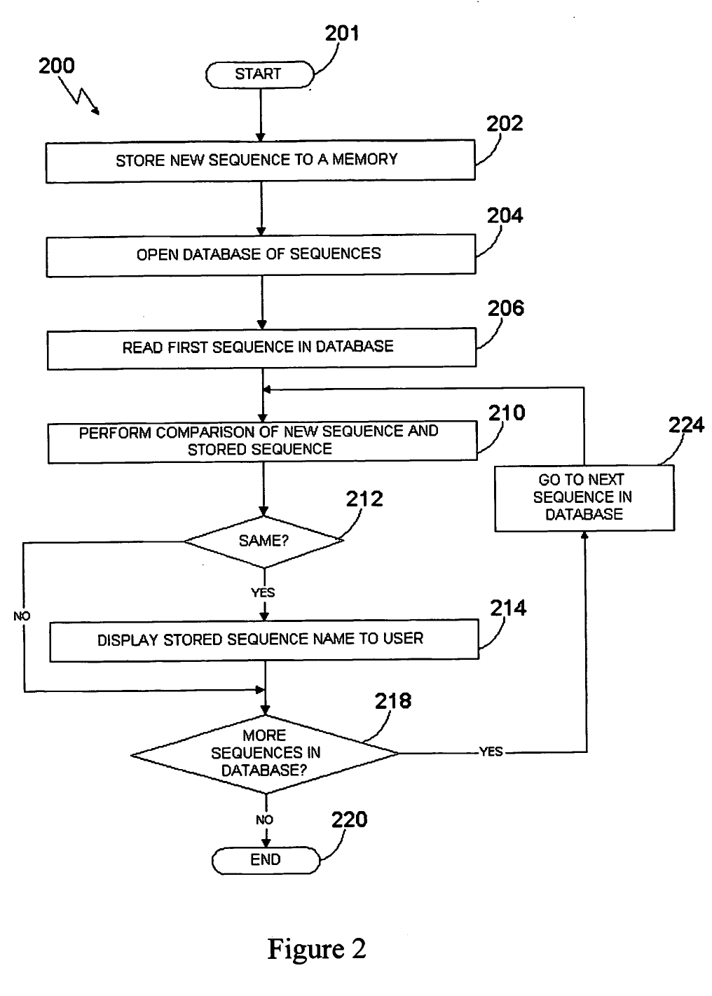

FIG. 2 is a flow diagram illustrating one embodiment of a process 200 for comparing a new nucleotide or protein sequence with a database of sequences in order to determine the homology levels between the new sequence and the sequences in the database. The database of sequences can be a private database stored within the computer system 100, or a public database such as GENBANK, PIR OR SWISSPROT that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of the invention or a polypeptide code of the invention, a data storage device having retrievably stored thereon reference nucleotide sequences or polypeptide sequences to be compared to the nucleic acid code of the invention or polypeptide code of the invention and a sequence comparer for conducting the comparison. The sequence comparer may indicate a homology level between the sequences compared or identify motifs implicated in biological function and structural motifs in the nucleic acid code of the invention and polypeptide codes of the invention or it may identify structural motifs in sequences which are compared to these nucleic acid codes and polypeptide codes. In some embodiments, the data storage device may have stored thereon the sequences of at least 2, 5, 10, 15, 20, 25, 30, or 50 of the nucleic acid codes of the invention or polypeptide codes of the invention.

Another aspect of the present invention is a method for determining the level of homology between a nucleic acid code of the invention and a reference nucleotide sequence, comprising the steps of reading the nucleic acid code and the reference nucleotide sequence through the use of a computer program which determines homology levels and determining homology between the nucleic acid code and the reference nucleotide sequence with the computer program. The computer program may be any of a number of computer programs for determining homology levels, including those specifically enumerated herein, including BLAST2N with the default parameters or with any modified parameters. The method may be implemented using the computer systems described above. The method may also be performed by reading 2, 5, 10, 15, 20, 25, 30, or 50 of the above described nucleic acid codes of the invention through the use of the computer program and determining homology between the nucleic acid codes and reference nucleotide sequences.

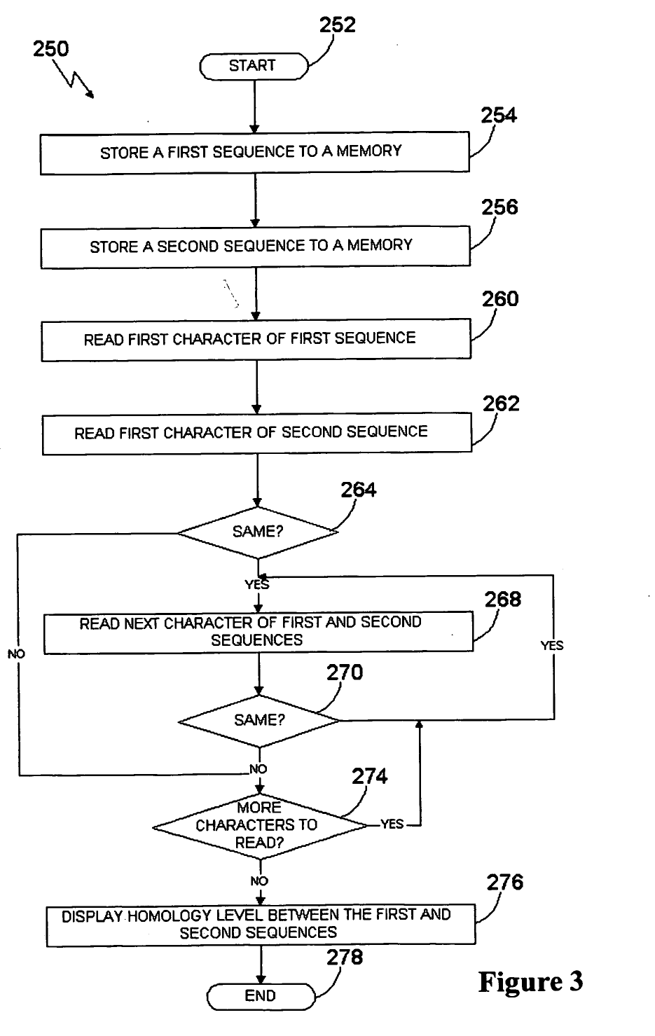

FIG. 3 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it should be in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there are no more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether the nucleotide sequences of the nucleic acid codes of the invention contain one or more single nucleotide polymorphisms (SNP) with respect to a reference nucleotide sequence. These single nucleotide polymorphisms may each comprise a single base substitution, insertion, or deletion.

Another aspect of the present invention is a method for determining the level of homology between a polypeptide code of the invention and a reference polypeptide sequence, comprising the steps of reading the polypeptide code of the invention and the reference polypeptide sequence through use of a computer program which determines homology levels and determining homology between the polypeptide code and the reference polypeptide sequence using the computer program.

Accordingly, another aspect of the present invention is a method for determining whether a nucleic acid code of the invention differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms. The method may be implemented by the computer systems described above and the method illustrated in FIG. 3. The method may also be performed by reading at least 2, 5, 10, 15, 20, 25, 30, or 50 of the nucleic acid codes of the invention and the reference nucleotide sequences through the use of the computer program and identifying differences between the nucleic acid codes and the reference nucleotide sequences with the computer program.

In other embodiments the computer based system may further comprise an identifier for identifying features within the nucleotide sequences of the nucleic acid codes of the invention or the amino acid sequences of the polypeptide codes of the invention. An “identifier” refers to one or more programs which identifies certain features within the above-described nucleotide sequences of the nucleic acid codes of the invention or the amino acid sequences of the polypeptide codes of the invention. In one embodiment, the identifier may comprise a program which identifies an open reading frame in the cDNAs codes of the invention.

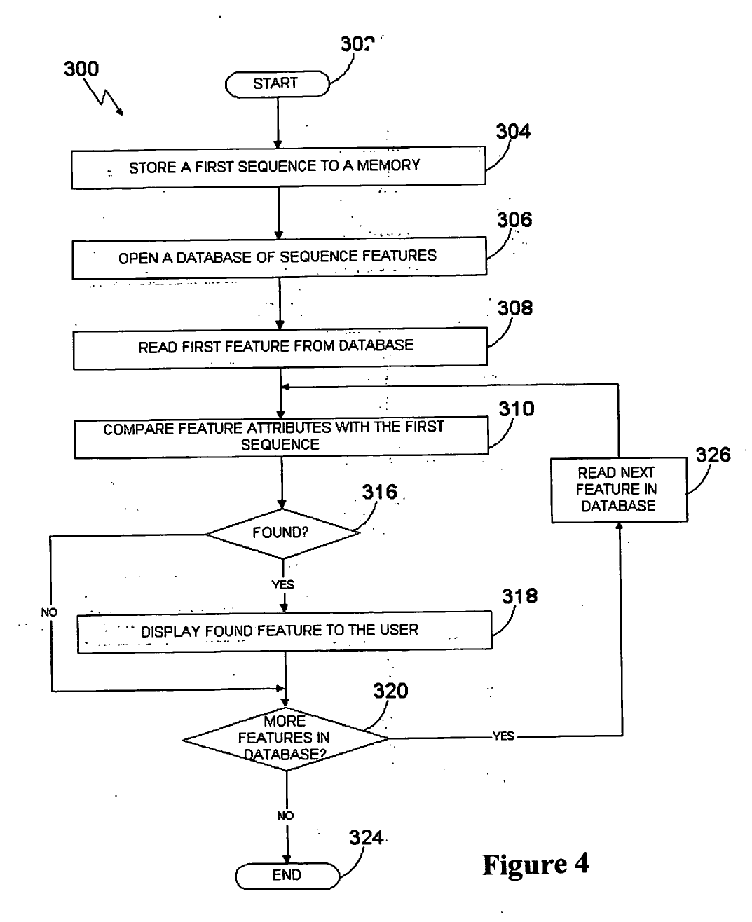

FIG. 4 is a flow diagram illustrating one embodiment of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group (www.gcg.com).

Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user.

The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence.

It should be noted, that if the feature attribute is not found in the first sequence at the decision state 316, the process 300 moves directly to the decision state 320 in order to determine if any more features exist in the database.

In another embodiment, the identifier may comprise a molecular modeling program which determines the 3-dimensional structure of the polypeptides codes of the invention. Such programs may use any methods known to those skilled in the art including methods based on homology-modeling, fold recognition and ab initio methods as described in Sternberg et al., 1999, which disclosure is hereby incorporated by reference in its entirety. In some embodiments, the molecular modeling program identifies target sequences that are most compatible with profiles representing the structural environments of the residues in known three-dimensional protein structures. (See, e.g., Eisenberg et al., U.S. Pat. No. 5,436,850 issued Jul. 25, 1995, which disclosure is hereby incorporated by reference in its entirety). In another technique, the known three-dimensional structures of proteins in a given family are superimposed to define the structurally conserved regions in that family. This protein modeling technique also uses the known three-dimensional structure of a homologous protein to approximate the structure of the polypeptide codes of the invention. (See e.g., Srinivasan, et al., U.S. Pat. No. 5,557,535 issued Sep. 17, 1996, which disclosure is hereby incorporated by reference in its entirety). Conventional homology modeling techniques have been used routinely to build models of proteases and antibodies. (Sowdhamini et al., (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods now enables the identification of likely folding patterns in a number of situations where the structural relatedness between target and template(s) is not detectable at the sequence level. Hybrid methods, in which fold recognition is performed using Multiple Sequence Threading (MST), structural equivalencies are deduced from the threading output using a distance geometry program DRAGON to construct a low resolution model, and a full-atom representation is constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalencies obtained from the MST output are converted into interresidue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Aszódi et al., (1997)).

The results of the molecular modeling analysis may then be used in rational drug design techniques to identify agents which modulate the activity of the polypeptide codes of the invention.

Accordingly, another aspect of the present invention is a method of identifying a feature within the nucleic acid codes of the invention or the polypeptide codes of the invention comprising reading the nucleic acid code(s) or the polypeptide code(s) through the use of a computer program which identifies features therein and identifying features within the nucleic acid code(s) or polypeptide code(s) with the computer program. In one embodiment, computer program comprises a computer program which identifies open reading frames. In a further embodiment, the computer program identifies linear or structural motifs in a polypeptide sequence. In another embodiment, the computer program comprises a molecular modeling program. The method may be performed by reading a single sequence or at least 2, 5, 10, 15, 20, 25, 30, or 50 of the nucleic acid codes of the invention or the polypeptide codes of the invention through the use of the computer program and identifying features within the nucleic acid codes or polypeptide codes with the computer program.

The nucleic acid codes of the invention or the polypeptide codes of the invention may be stored and manipulated in a variety of data processor programs in a variety of formats. For example, they may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the nucleic acid codes of the invention or the polypeptide codes of the invention. The following list is intended not to limit the invention but to provide guidance to programs and databases which are useful with the nucleic acid codes of the invention or the polypeptide codes of the invention. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, 1990), FASTA (Pearson and Lipman, 1988), FASTDB (Brutlag et al., 1990), Catalyst (Molecular Simulations Inc.), Catalyst/SRAPE (Molecular Simulations Inc.), Cerius2.DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### CONCLUSION

As discussed above, the GENSET polynucleotides and polypeptides of the present invention or fragments thereof can be used for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; as a reagent (including a labeled reagent) in assays designed to quantitatively determine levels of GENSET expression in biological samples; to compare with endogenous DNA sequences in patients to identify potential genetic disorders; as probes to hybridize and thus discover novel, related DNA sequences; as a source of information to derive PCR primers for genetic fingerprinting; for selecting and making oligomers for attachment to a “gene chip” or other support, including for examination for expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., (1993) to identify polynucleotides encoding the other protein with which binding occurs or to identify inhibitors of the binding interaction.

The proteins or polypeptides provided by the present invention can similarly be used in assays to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning; A Laboratory Manual”, 2d ed., Cole Spring Harbor Laboratory Press, Sambrook, J., E. F. Fritsch and T. Maniatis eds., 1989, and “Methods in Enzymology; Guide to Molecular Cloning Techniques”, Academic Press, Berger and Kimmel eds., 1987, which disclosures are hereby incorporated by reference in their entireties.

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims.

### EXAMPLES

Preparation of Antibody Compositions to the GENSET Protein

Substantially pure protein or polypeptide is isolated from transfected or transformed cells containing an expression vector encoding the GENSET protein or a portion thereof. The concentration of protein in the final preparation is adjusted, for example, by concentration on an Amicon filter device, to the level of a few micrograms/ml. Monoclonal or polyclonal antibody to the protein can then be prepared as follows:

A. Monoclonal Antibody Production by Hybridoma Fusion

Monoclonal antibody to epitopes in the GENSET protein or a portion thereof can be prepared from murine hybridomas according to the classical method of Kohler and Milstein, (1975) or derivative methods thereof. Also see Harlow and Lane. (1988).

Briefly, a mouse is repetitively inoculated with a few micrograms of the GENSET protein or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, (1980), which disclosure is hereby incorporated by reference in its entirety, and derivative methods thereof. Selected positive clones can be expanded and their monoclonal antibody product harvested for use. Detailed procedures for monoclonal antibody production are described in Davis, et al. (1986) Section 21-2.

B. Polyclonal Antibody Production by Immunization

Polyclonal antiserum containing antibodies to heterogeneous epitopes in the GENSET protein or a portion thereof can be prepared by immunizing suitable non-human animal with the GENSET protein or a portion thereof, which can be unmodified or modified to enhance immunogenicity. A suitable non-human animal is preferably a non-human mammal is selected, usually a mouse, rat, rabbit, goat, or horse. Alternatively, a crude preparation which has been enriched for GENSET concentration can be used to generate antibodies. Such proteins, fragments or preparations are introduced into the non-human mammal in the presence of an appropriate adjuvant (e.g. aluminum hydroxide, RIBI, etc.) which is known in the art. In addition the protein, fragment or preparation can be pretreated with an agent which will increase antigenicity, such agents are known in the art and include, for example, methylated bovine serum albumin (mBSA), bovine serum albumin (BSA), Hepatitis B surface antigen, and keyhole limpet hemocyanin (KLH). Serum from the immunized animal is collected, treated and tested according to known procedures. If the serum contains polyclonal antibodies to undesired epitopes, the polyclonal antibodies can be purified by immunoaffinity chromatography.

Effective polyclonal antibody production is affected by many factors related both to the antigen and the host species. Also, host animals vary in response to site of inoculations and dose, with both inadequate or excessive doses of antigen resulting in low titer antisera. Small doses (ng level) of antigen administered at multiple intradermal sites appears to be most reliable. Techniques for producing and processing polyclonal antisera are known in the art. An effective immunization protocol for rabbits can be found in Vaitukaitis et al. (1971), which disclosure is hereby incorporated by reference in its entirety.

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony et al., (1973), which disclosure is hereby incorporated by reference in its entirety. Plateau concentration of antibody is usually in the range of 0.1 to 0.2 mg/ml of serum (about 12 uM). Affinity of the antisera for the antigen is determined by preparing competitive binding curves, as described, for example, by Fisher (1980), which disclosure is hereby incorporated by reference in its entirety.

Antibody preparations prepared according to either the monoclonal or the polyclonal protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

### REFERENCES

Abbondanzo et al., (1993), Meth. Enzymol., Academic Press, New York, pp 803-823

Altschul et al., (1990), J. Mol. Biol. 215(3):403410

Altschul et al., (1993), Nature Genetics 3:266-272

Altschul et al., (1997), Nuc. Acids Res. 25:3389-3402

Ames et al., (1995), J. Immunol. Meth. 184:177-186.

Anton and Graham, (1995), J. Virol., 69: 4600-4606

Araki et al., (1995) Proc. Natl. Acad. Sci. USA. 92(1):1604.

Ashkenazi et al., (1991), Proc. Natl. Acad. Sci. USA 88:10535-10539.

Aszódi et al., (1997) Proteins: Structure, Function, and Genetics, Supplement 1:38-42

Attwood et al., (1996) Nucleic Acids Res. 24(1):182-8.

Attwood et al., (2000) Nucleic Acids Res. 28(1):225-7

Bartunek et al., (1996), Cytokine. 8(1):14-20.

Bateman et al., (2000) Nucleic Acids Res. 28(1):263-6

Baubonis (1993) Nucleic Acids Res. 21(9):2025-9.

Beaucage et al., (1981) Tetrahedron Lett, 22: 1859-1862

Benham et al. (1989) Genomics 4:509-517,

Better et al., (1988), Science. 240:1041-1043.

Bittle et al., (1985), Virol. 66:2347-2354.

Bowie et al, (1994), Science. 247:1306-1310.

Bradley (1987), Production and analysis of chimaeric mice. In: E. J. Robertson (Ed.), Teratocarcinomas and embryonic stem cells: A practical approach. IRL Press, Oxford, pp. 113.

Bram et al., (1993), Mol. Cell Biol., 13: 4760-4769

Brinkmnan et al., (1995) J. Immunol Methods. 182:41-50.

Brown et al., (1979) Meth. Enzymol. 68:109-151

Brutlag et al. (1990) Comp. App. Biosci. 6:237-245

Bucher and Bairoch (1994) Proceedings 2nd International Conference on Intelligent Systems for Molecular Biology. Altman et al, Eds., pp 53-61, AAAIPress, Menlo Park.

Burton et al. (1994), Adv. Immunol. 57:191-280

Bush et al., (1997), J. Chromatogr., 777 : 311-328.

Butt and Karathanasis (1995) Gene Expr. 4(6):3 19-36.

Carlson et al., (1997), J. Biol. Chem. 272(17):11295-11301.

Chai et al., (1993) Biotechnol. Appl. Biochem. 18:259-273.

Chang et al., (1993) Gene 127:95-8

Chee et al., (1996) Science. 274:610-614.

Chen et al. (1987) Mol. Cell. Biol. 7:2745-2752.

Chen et al., (1998), Cancer Res. 58(16):3668-3678.

Cherif et al., (1990) Proc. Natl. Acad. Sci. U.S.A., 87:6639-6643

Cho et al., (1998), Proc. Natl. Acad. Sci. USA, 95(7): 3752-3757.

Chou, (1989), Mol. Endocrinol. 3: 1511-1514.

Chow et al., ( 1985), Proc. Natl. Acad. Sci. USA. 82:910-914.

Cleland et al., (1993), Crit. Rev. Therapeutic Drug Carrier Systems. 10:307-377.

Coles et al., (1998) Hum Mol Genet 7:791-800

Compton (1991) Nature 350(6313):91-92.

Corpet et al. (2000) Nucleic Acids Res. 28(1):267-9

Cox et al., (1990) Science 250:245-250

Creighton (1983), Proteins: Structures and Molecular Principles, W. H. Freeman & Co. 2nd Ed., T. E., New York

Creighton, (1993), Posttranslational Covalent Modification of Proteins, W. H. Freeman and Company, New York B. C. Johnson, Ed., Academic Press, New York 1-12

Cunningham et al. (1989), Science 244:1081-1085.

Davis et al., (1986) Basic Methods in Molecular Biology, ed., Elsevier Press, NY, Decker and Parker, (1995) Curr. Opin. Cell. Biol. 7(3) :368-92

Dempsteret al., (1977) Stat. Soc., 39B:1-38.

Deng et al., (1998) Blood. 92(6):1981-1988.

Dent and Latchman (1993) The DNA mobility shift assay. In: Transcription Factors: A Practical Approach (Latchman D S, ed.) pp1-26. Oxford: IRL Press

Derrigo et al., (2000) Int. J. Mol. Med. 5(2) :111-23

Eckner et a., (1991) EMBO J. 10:3513-3522.

Edwards and Leatherbarrow, (1997) Analytical Biochemistry, 246, 1-6

Engvall, (1980) Meth. Enzymol. 70:419

Erlich, (1992) PCR Technology; Principles and Applications for DNA Amplification. W. H. Freeman and Co., New York

Feldman and Steg, (1996), Medecine/Sciences, 12:47-55

Feigner (1996) Hum Gene Ther. 7(15):1791-3.

Felici, (1991), J. Mol. Biol., 222:301-310

Fell et al, (1991), J. Immunol. 146:2446-2452.

Fields and Song, (1989), Nature, 340: 245-246

Fisher, (1980) Chap. 42 in: Manual of Clinical Immunology, 2d Ed. (Rose and Friedman, Eds.) Amer. Soc. For Microbiol., Washington, D.C.

Flotte et al, (1992) Am. J. Respir. Cell Mol. Biol. 7:349-356.

Fodor et al., (1991) Science 251:767-777.

Foster et al., (1996) Genomics 33:185-192

Fountoulakis et al, (1995) Biochem. 270:3958-3964.

Fraley et al, (1979) Proc. Natl. Acad. Sci. USA. 76:3348-3352.

Frazer et al., (1992) Genomics 14:574-584

Fried and Crothers, (1981) Nucleic Acids Res. 9:6505-6525

Fromont-Racine et al., (1997), Nature Genetics, 16(3): 277-282.

Fry et al., (1992) Biotechniques, 13: 124-131

Fudenberg, (1980) Chap. 26 in: Basic 503 Clinical Immunology, 3rd Ed. Lange, Los Altos, Calif.

Fuller S. A. et al. (1996) Immunology in Current Protocols in Molecular Biology,

Furth P. A. et al. (1994) Proc. Natl. Acad. Sci USA. 91:9302-9306.

Garner and Revzin, (1981) Nucleic Acids Res 9:3047-3060

Gentz et al, (1989) Proc Natl Acad Sci USA. 86(3):821-4.

Geysen et al., (1984), Proc. Natl. Acad. Sci. U.S.A. 81:3998-4002.

Ghosh and Bacchawat, (1991), Targeting of liposomes to hepatocytes, IN: Liver Diseases, Targeted diagnosis and therapy using specific rceptors and ligands. Eds., Marcel Dekeker, N.Y. pp. 87-104.

Gillies et al., (1989), J. Immunol Methods. 125:191-202.

Gillies et a., (1992), Proc Natl Acad Sci USA 89:1428-1432.

Gonnet et al., (1992), Science 256:1443-1445

Gopal (1985) Mol. Cell. Biol., 5:1188-1190.

Gossen et al., (1992) Proc. Natl. Acad. Sci. USA. 89:5547-5551.

Gossen et al., (1995) Science. 268:1766-1769.

Graham et al., (1973) Virol. 52:456-457.

Green et al., (1986) Ann. Rev. Biochem. 55:569-597

Greenspan and Bona (1989), FASEB J. 7(5):437-444.

Griffais et al., (1991) Nucleic Acids Res. 19: 3887-3891

Griffin et al., (1989) Science 245:967-971

Gu H. et al., (1993) Cell 73:1155-1164.

Gu H. et al., (1994) Science 265:103-106.

Guatelli et al., (1990) Proc. Natl. Acad. Sci. USA. 35:273-286.

Gyuris et al., (1993) Cell 75:791-803

Hames and Higgins (1985) Nucleic Acid Hybridization: A Practical Approach. Harnes and Higgins Ed., IRL Press, Oxford.

Hammerling (1981), Monoclonal Antibodies and T-Cell Hybridomas, Elsevier, N.Y. 563-681.

Hansson et al., (1999), J. Mol. Biol. 287:265-276.

Haravama (1998), Trends Biotechnol. 16(2): 76-82.

Harland et al., (1985) J. Cell. Biol. 101:1094-1095.

Harlow and Lane, (1988) Antibodies A Laboratory Manual. Cold Spring Harbor Laboratory. pp. 53-242

Harper et al., (1993), Cell, 75 : 805-816

Harrop et al., (1998), J. Immunol. 161(4):1786-1794.

Haynes et al., (1996) J Biotechnol. 44(1-3):37-42.

Henikoff and Henikoff, (1993), Proteins 17:49-61

Henikoff et al., (2000) Electrophoresis 21(9):1700-6

Henikoff et al., (2000) Nucleic Acids Res. 28(1):228-30

Higgins et al., (1996), Meth. Enzymol. 266:383-402

Hillier and Green (1991) PCR Methods Appl., 1: 124-8.

Hoess et al., (1986) Nucleic Acids Res. 14:2287-2300.

Hofmann et al., (1999) Nucl. Acids Res. 27:215-219.;

Holm and Sander (1996) Nucleic Acids Res. 24(1):206-9

Holm and Sander (1997) Nucleic Acids Res. 25(1):231-4

Holm and Sander (1999) Nucleic Acids Res. 27(1):244-7

Hoppe et al., (1994), FEBS Letters. 344:191.

Houghten (1985), Proc. Natl. Acad. Sci. USA 82:5131-5135.

Huang et al., (1996) Cancer Res 56(5): 1137-1141.

Hunkapiller et al., (1984) Nature. 310(5973): 105-11.

Huston et al., (1991), Meth. Enymol. 203:46_{—}88.

Huygen et al., (1996) Nature Medicine. 2(8):893-898.

Izant and Weintraub, (1984) Cell 36(4):1007-15

Jameson and Wolf, (1988), Comp. Appl. Biosci. 4:181-186

Julan et al., (1992) J. Gen. Virol. 73:3251-3255.

Kanegae et al., (1995) Nucl. Acids Res. 23:3816-3821.

Karlin and Altschul, (1990), Proc. Natl. Acad. Sci. USA 87:2267-2268

Kettleborough et al., (1994), Eur. L Immunol. 24:952-958.

Kim U-J. et al., (1996) Genomics 34:213-218.

Klein et al., (1987) Nature. 327:70-73.

Kohler and Milstein, (1975) Nature 256:495

Koller et al.; (1992) Annu. Rev. Immunol. 10:705-730.

Kostelny et al., (1992), J. Immunol. 148:1547-1553.

Landschulz et al., (1988), Science. 240:1759.

Ledbetter et al., (1990) Genomics 6:475-481

Lenhard et al., (1996) Gene. 169:187-190.

Levy et al., (1996) Gene Ther. 3(3):201-11.

Lewin, (1989), Proc. Natl. Acad. Sci. USA86:9832-8935.

Liautard et al., (1997), Cytokine. 9(4):233-241.

Linton et al., (1993) J. Clin. Invest. 92:3029-3037.

Liu et al., (1994) Proc. Natl. Acad. Sci. USA. 91: 4528-4262.

Lo Conte et al., (2000) Nucleic Acids Res. 28(1):257-9.

Lockhart et al., (1996) Nature Biotechnology 14: 1675-1680

Lorenzo and Blasco (1998) Biotechniques. 24(2):308-313.

Lucas (1994), In: Development and Clinical Uses of Haempophilus b Conjugate;

Makrides, (1999) Protein Expr. Purif. 17(2) :183-202

Malik et al., (1992), Exp. Hematol. 20:1028-1035.

Mansour et al., (1988) Nature. 336:348-352.

Marshall et al., (1994) PCR Methods and Applications. 4:80-84.

Maurer et al., (1999) Mol Membr Biol. 16(1):12940.

McCormick et al., (1994)Genet. Anal. Tech. Appl. 11:158-164.

McLaughlin et al., (1996) Am. J. Hum. Genet. 59:561-569.

Miller and Whelan, (1997) Hum Gene Ther. 8(7):803-15.

Muller et al., (1998), Structure. 6(9): 1153-1167.

Mullinax et al., (1992), BioTechniques. 12(6):864-869.

Murvai et al., (2000) Nucleic Acids Res. 28(1):260-2

Murzin et al., (1995) J Mol Biol. 247(4):536-40

Muzyczka et al., (1992) Curr. Topics in Micro. and Immunol. 158:97-129.

Nada et al., (1993) Cell 73:1125-1135.

Nagaraja et al, (1997. Genome Research 7:210-222

Nagy et al., (1993), Proc. Natl. Acad. Sci. USA 90: 8424-8428.

Nakai and Horton, (1999) Trends Biochem. Sci., 24:34-36

Nakai and Kanehisa (1992) Genomics 14, 897-911

Naramura et al., (1994), Immunol. Lett. 39:91-99.

Narang et al., (1979), Methods Enzymol 68:90-98

Neda et al., (1991) J. Biol. Chem. 266:14143-14146.

Nevill-Manning et al., (1998) Proc. Natl. Acad. Sci. USA. 95, 5865-5871

Nicolau et al., (1982) Biochim. Biophys. Acta. 721:185-190.

Nicolau et al., (1987), Meth. Enzymol., 149:157-76.

Nissinoff, (1991), J. Immunol. 147(8): 2429-2438.

O'Reilly et al., (1992) Baculovirus Expression Vectors: A Laboratory Manual. W. H. Freeman and Co., New York.

Obermayr et al., (1996) Eur. J. Hum. Genet. 4:242-245

Ohno et al., (1994) Science. 265:781-784.

Oi et al., (1986), BioTechniques 4:214.

Oldenburg et al., (1992), Proc. Natl. Acad. Sci. USA 89:5393-5397.

Orengo et al., (1997) Structure. 5(8):1093-108

Ouchterlony et al., (1973) Chap. 19 in: Handbook of Experimental Immunology D. Wier (ed) Blackwell

Padlan, (1991), Molec. Immunol. 28(4/5):489-498.

Parmley and, Smith, (1988) Gene 73:305-318

Patten, et al. (1997), Curr Opinion Biotechnol. 8:724-733.

Pearl et al., (2000) Biochem Soc Trans. 28(2):269-75

Pearson and Lipman, (1988), Proc. Natl. Acad. Sci. USA 85(8):2444-2448

Pease and William, (1990), Exp. Cell. Res. 190: 209-211.

Persic et al., (1997), Gene. 1879-81

Pesole et al., (2000) Nucleic Acids Res, 28(1): 193-196

Peterson et al., (1993), Proc. Natl. Acad. Sci. USA, 90: 7593-7597.

Pietu et al., (1996) Genome Research 6:492-503

Pinckard et al., (1967), Clin. Exp. Immunol 2:331-340.

Pitard et al., (1997), J. Immunol. Methods. 205(2):177-190.

Pongor et al. (1993) Protein Eng. 6(4):391-5

Potter et al., (1984) Proc. Natl. Acad. Sci. U.S.A. 81(22):7161-7165.

Prat et al., (1998), J. Cell. Sci. 111(Pt2):237-247.

Raeymaekers et al., (1995) Genomics 29:170-178

Ramunsen et al., (1997), Electrophoresis, 18: 588-598.

Rattan et al., (1992) Ann NY Acad Sci 663:48-62

Reid et al., (1990) Proc. Natl. Acad. Sci. U.S.A. 87:4299-4303.

Robbins et al., (1987), Diabetes. 36:838-845.

Robertson, (1987), Embryo-derived stem cell lines. In: E. J. Robertson Ed. Teratocarcinomas and embrionic stem cells: a practical approach. IRL Press, Oxford, pp. 71.

Roguska et al., (1994), Proc. Natl. Acad. Sci. U.S.A. 91:969-973.

Ron et al., (1993), Biol Chem., 268 2984-2988.

Rose et al., (1980) Chap. 12 in: Methods in Immunodiagnosis, 2d Ed. John Wiley 503 Sons, New York

Rossi et al., (1991) Pharmacol. Ther. 50:245-254,

Roth et al., (1996) Nature Medicine. 2(9):985-991.

Roux et al., (1989) Proc. Natl. Acad. Sci. U.S.A. 86:9079-9083.

Sambrook et al., (1989) Molecular Cloning: A Laboratory Manual. 2ed. Cold Spring Harbor Laboratory, Cold Spring Harbor, N.Y.

Samson et al., (1996) Nature, 382(6593):722-725.

Samulski et al., (1989) J. Virol. 63:3822-3828.

Sanchez-Pescador (1988) J. Clin. Microbiol. 26(10):1934-1938.

Sander and Schneider (1991) Proteins. 9(1):56-68.)

Sauer et al., (1988) Proc. Natl. Acad. Sci. U.S.A. 85:5166-5170.

Sawai et al., (1995), AJRI 34:26-34.

Schedl et al., (1993b), Nucleic Acids Res., 21: 4783-4787.

Schedl et al., (993a), Nature, 362: 258-261.

Schena et al. (1995) Science 270:467-470

Schena et al., (1996), Proc Natl Acad Sci USA,.93(20):10614-10619.

Schuler et al., (1996) Science 274:540-546

Schultz et al., (1998) Proc Natl Acad Sci USA 95, 5857-5864

Schwartz and Dayhoff, (1978), eds., Matrices for Detecting Distance Relationships: Atlas of Protein Sequence and Structure, Washington: National Biomedical Research Foundation

Sczakiel et al., (1995) Trends Microbiol. 3(6):213-217.

Seifter et al., (1990) Meth Enzymol 182:626-646

Shay et al., (1991), Biochem. Biophys. Acta, 1072: 1-7.

Shizuya et al., (1992) Proc. Natl. Acad. Sci. U.S.A. 89:8794-8797.

Shu et al., (1993), Proc. Natl. Acad. Sci. U.S.A. 90:7995-7999.

Skerra et al., (1988), Science 240:1038-1040.

Smith and Johnson (1988) Gene. 67(1):31-40.

Smith et al., (1983) Mol. Cell. Biol. 3:2156-2165.

Smith et al., (1996) Antiviral Res. 32(2):99-115.

Sonnhammer and Kahn D (1994) Protein Sci. 3(3):482-92

Sonnhammer et al., (1997) Proteins. 28(3):405-20

Sosnowski, et al., (1997) Proc Natl Acad Sci USA 94:1119-1123

Sowdhamini et al., (1997) Protein Engineering 10:207, 215

Sternberg (1994) Mamm. Genome. 5:397-404.

Sternberg (1992) Trends Genet. 8:1-16.

Sternberg et al, (1999) Curr Opin Struct Biol. 9(3):368-73.

Stone et al., (2000) J Endocrinol. 164(2): 103-18.

Stryer, (1995) Biochemistry, 4th edition

Studnicka et al, (1994), Protein Engineering. 7(6):805-814.

Sutcliffe et al, (1983), Science. 219:660-666.

Szabo et al., (1995) Curr Opin Struct Biol 5, 699-705

Tascon et al., (1996) Nature Medicine. 2(8):888-892.

Taryman et al., (1995), Neuron. 14(4):755-762.

Tatusov et al, (1997) Science, 278, 631 :637

Tatusov et al., (2000) Nucleic Acids Res. 28(1):33-6.)

Te Riele et al., (1990) Nature. 348:649-651.

Thomas et al., (1986) Cell. 44:419-428.

Thomas et al., (1987) Cell. 51:503-512.

Thompson et al, (1994), Nucleic Acids Res. 22(2):4673-4680

Traunecker et al., (1988), Nature. 331:84-86.

Tur-Kaspa et al., (1986) Mol. Cell. Biol. 6:716-718.

Tutt et al., (1991), J. Immunol. 147:60-69.

Urdea (1988) Nucleic Acids Research. 11:4937-4957.

Urdea et al., (1991) Nucleic Acids Symp. Ser. 24:197-200.

Vaitukaitis et al., (1971) J. Clin. Endocrinol. Metab. 33:988-991

Valadon et al, (1996), J. Mol. Biol., 261:11-22.

Van der Lugt et al., (1991) Gene. 105:263-267.

Vil et al., (1992) Proc Natl Acad Sci US 89:11337-11341.

Viasak et al., (1983) Eur. J. Biochem. 135:123-126.

Wabiko et al., (1986) DNA. 5(4):305-314.

Wagner et al., (1996) Nat Biotechnol. 14(7):840-4.

Walker et al., (1996) Clin. Chem. 42:9-13.

Wang et al., (1997), Chromatographia, 44:205-208.

Warrington et al., (1991) Genomics 11:701-708

Westerink, (1995), Proc. Natl. Acad. Sci USA., 92:4021-4025

White (1997) B. A. Ed. in Methods in Molecular Biology 67: Humana Press, Totowa

White et al. (1997) Genomics. 12:301-306.

Wilson et al., (1984) Cell. 37(3):767-78.

Wong et al., (1980) Gene. 10:87-94.

Wood et al., (1985) Proc. Natl. Acad. Sci. USA 82(6):1585-1588

Wood et al., (1993), Proc. Natl. Acad. Sci. USA,90:4582-4585.

Wu and Ataai, (2000) Curr Opin Biotechnol. 11(2):205-8.

Wu and Wu,(1987) J. Biol. Chem. 262:4429-4432.

Wu and Wu, (1988) Biochemistry. 27:887-892.

Yagi T. et al., (1990) Proc. Natl. Acad. Sci. U.S.A. 87:9918-9922.

Yona et al., (1999) Proteins. 37(3):360-78

Yoon et al, (1998), J. Immunol. 160(7):3170-3179.

Zheng, X. X. et al. (1995), J. Immunol. 154:5590-5600.

Zhu et al., (1998), Cancer Res. 58(15):3209-3214.

Zou et al., (1994)Curr. Biol. 4:1099-1103.

Throughout this application, various publications, patents and published patent applications are cited. The disclosures of these publications, patents and published patent specification referenced in this application are hereby incorporated by reference into the present disclosure to more fully describe the state of the art to which this invention pertains.

TABLE I

| SEQ ID NO | SEQ ID NO | SEQ ID NO | SEQ ID NO | |
|---|---|---|---|---|
| from priority | in present | in present | in present | |
| application | application | application | application | |
| (nucl) | (nucl.) | (prt) | (nucl.) | Clone ID |
| 37 | | | NUC561 | 486589; 523280 |
| 51 | | | NUC562 | 500707076; 609080; 642931 |
| 179 | | | NUC563 | 147941; 153834; 193100 |
| 180 | | | NUC564 | 147941 |
| 183 | | | NUC565 | 100038; 100419; 100523; 100546 |
| 201 | | | NUC566 | 528046 |
| 326 | | | NUC567 | 484503 |
| 362 | | | NUC568 | 211034; 211122 |
| 440 | | | NUC569 | 627628 |
| 452 | | | NUC570 | 500713596; 500733538; 500741977 |
| 483 | | | NUC571 | 500730326 |
| 500 | | | NUC572 | 482482 |
| 505 | | | NUC573 | 611492 |
| 528 | | | NUC574 | 221311 |
| 573 | | | NUC575 | 144150 |
| 574 | | | NUC576 | 626500 |
| 587 | | | NUC577 | 490129 |
| 588 | | | NUC578 | 129471 |
| 593 | NUC86 | PRT255 | NUC406 | 500762786 |
| 599 | | | NUC579 | 148991; 206343; 211039 |
| 603 | | | NUC580 | 212418 |
| 621 | | | NUC581 | 395370 |
| 628 | | | NUC582 | 116680 |
| 653 | | | NUC583 | 224425 |
| 670 | | | NUC584 | 225626 |
| 678 | NUC87 | PRT256 | NUC407 | 822794 |
| 678 | NUC88 | PRT257 | NUC407 | 337572 |
| 693 | | | NUC585 | 500760143 |
| 703 | | | NUC586 | 125325; 145574; 158295; 225872; |
| | | | | 334779 |
| 746 | | | NUC587 | 620699 |
| 770 | | | NUC588 | 500735221 |
| 775 | | | NUC589 | 131662; 131668; 177901; 200591 |
| 796 | | | NUC590 | 500756189 |
| 812 | | | NUC591 | 500694179 |
| 940 | | | NUC592 | 158339; 213121; 220652; 236981; |
| | | | | 239275; 239598; 244360; 582565 |
| 988 | | | NUC593 | 238123; 239495; 334569; 582920 |
| 996 | | | NUC594 | 334818 |
| 1036 | | | NUC595 | 483794; 519407; 633595; 633902 |
| 1064 | | | NUC596 | 608607 |
| 1151 | | | NUC597 | 500743552; 500744660 |
| 1190 | | | NUC598 | 101006; 509431 |
| 1458 | | | NUC599 | 313060; 313135; 313174 |
| 1590 | | | NUC600 | 178255 |
| 1853 | | | NUC601 | 114927 |
| 1904 | | | NUC602 | 107824 |
| 2028 | | | NUC603 | 170306 |
| 2173 | NUC89 | PRT258 | NUC408 | 642374 |
| 2368 | | | NUC604 | 210539; 331006 |
| 2553 | | | NUC605 | 106061 |
| 2556 | | | NUC606 | 106061 |
| 2658 | | | NUC607 | 654607 |
| 2690 | | | NUC608 | 172048; 172057 |
| 2755 | | | NUC609 | 101090 |
| 2800 | | | NUC610 | 119222; 119491; 151036 |
| 2843 | | | NUC611 | 619446; 619452; 633712 |
| 2852 | | | NUC612 | 145573 |
| 2932 | | | NUC613 | 650981 |
| 2955 | | | NUC614 | 130068 |
| 3078 | | | NUC615 | 100966; 99482 |
| 3280 | | | NUC616 | 147041 |
| 3326 | | | NUC617 | 132294; 132317 |
| 3387 | | | NUC618 | 126303; 134231; 135124 |
| 3439 | | | NUC619 | 502644 |
| 3501 | | | NUC620 | 625238; 632902; 635258 |
| 3633 | | | NUC621 | 120631 |
| 3678 | | | NUC622 | 200451 |
| 3714 | NUC90 | PRT259 | NUC409 | 231569 |
| 3714 | NUC91 | PRT260 | NUC409 | 145151 |
| 3714 | | | NUC409 | 153628 |
| 3796 | | | NUC623 | 131690; 588080 |
| 3801 | | | NUC624 | 199999 |
| 3804 | | | NUC625 | 483173; 510100; 650405 |
| 3892 | | | NUC626 | 626911; 627852; 631702; 633566 |
| 3985 | | | NUC627 | 229507; 229539; 236257 |
| 4005 | | | NUC628 | 237312; 335729; 490055 |
| 4063 | | | NUC629 | 521127 |
| 4088 | NUC92 | PRT261 | NUC410 | 128061 |
| 4088 | NUC93 | PRT262 | NUC410 | 118027 |
| 4088 | | | NUC410 | 166676 |
| 4111 | NUC94 | PRT263 | NUC411 | 627202 |
| 4111 | | | NUC411 | 538182; 620818; 625154; 628241; |
| | | | | 629431; 633031; 634788 |
| 4126 | | | NUC630 | 153486 |
| 4172 | | | NUC631 | 241664 |
| 4261 | NUC95 | PRT264 | NUC412 | 112311 |
| 4340 | | | NUC632 | 500703884; 633346; 634598 |
| 4436 | | | NUC633 | 204316 |
| 4609 | NUC1 | PRT170 | NUC339 | 502084 |
| 4647 | NUC2 | PRT171 | NUC340 | 589115 |
| 4660 | | | NUC634 | 643537 |
| 4664 | NUC3 | PRT172 | NUC341 | 1000902917 |
| 4678 | NUC4 | PRT173 | NUC342 | 602517 |
| 4678 | NUC5 | PRT174 | NUC342 | 478210 |
| 4678 | | | NUC342 | 763189 |
| 4682 | NUC6 | PRT175 | NUC343 | 500698315 |
| 4687 | NUC7 | PRT176 | NUC344 | 114180 |
| 4687 | | | NUC344 | 114106; 175654; 654896 |
| 4690 | | | NUC635 | 114106; 211516; 654896 |
| 4694 | NUC8 | PRT177 | NUC345 | 338112 |
| 4696 | NUC9 | PRT178 | NUC346 | 338100 |
| 4733 | NUC10 | PRT179 | NUC347 | 784093 |
| 4807 | NUC11 | PRT180 | NUC348 | 1000943975 |
| 4809 | NUC12 | PRT181 | NUC349 | 1000771934 |
| 4830 | NUC13 | PRT182 | NUC350 | 186661 |
| 4855 | NUC14 | PRT183 | NUC351 | 105855 |
| 4900 | NUC15 | PRT184 | NUC352 | 500742698 |
| 4908 | | | NUC636 | 538424 |
| 4943 | | | NUC637 | 144901; 170048; 206458; 240439; |
| | | | | 241215 |
| 4947 | NUC16 | PRT185 | NUC353 | 201980 |
| 4947 | NUC17 | PRT186 | NUC353 | 198002 |
| 4976 | | | NUC638 | 248323; 248866 |
| 5000 | NUC18 | PRT187 | NUC354 | 500739047 |
| 5002 | NUC19 | PRT188 | NUC355 | 1000904024 |
| 5005 | | | NUC639 | 155986; 222313; 237229 |
| 5011 | NUC20 | PRT189 | NUC356 | 125817 |
| 5040 | | | NUC640 | 646668 |
| 5058 | | | NUC641 | 224715 |
| 5071 | NUC21 | PRT190 | NUC357 | 147648 |
| 5089 | NUC22 | PRT191 | NUC358 | 1000839315 |
| 5117 | NUC23 | PRT192 | NUC359 | 122473 |
| 5141 | NUC24 | PRT193 | NUC360 | 585770 |
| 5141 | NUC25 | PRT194 | NUC360 | 123996 |
| 5162 | NUC26 | PRT195 | NUC361 | 1000904064 |
| 5167 | NUC27 | PRT196 | NUC362 | 482181 |
| 5178 | NUC28 | PRT197 | NUC363 | 500731597 |
| 5192 | NUC29 | PRT198 | NUC364 | 581232 |
| 5214 | | | NUC642 | 394359 |
| 5230 | NUC30 | PRT199 | NUC365 | 613647 |
| 5240 | NUC31 | PRT200 | NUC366 | 715437 |
| 5250 | NUC32 | PRT201 | NUC367 | 1000878517 |
| 5262 | NUC33 | PRT202 | NUC368 | 544474 |
| 5270 | NUC34 | PRT203 | NUC369 | 143880 |
| 5278 | NUC35 | PRT204 | NUC370 | 1000853793 |
| 5358 | NUC36 | PRT205 | NUC371 | 500732568 |
| 5453 | NUC37 | PRT206 | NUC372 | 427150 |
| 5453 | NUC38 | PRT207 | NUC372 | 593306 |
| 5453 | NUC39 | PRT208 | NUC372 | 593993 |
| 5453 | NUC40 | PRT209 | NUC372 | 590939 |
| 5453 | | | NUC372 | 432874; 435627 |
| 5494 | NUC41 | PRT210 | NUC373 | 155600 |
| 5494 | NUC42 | PRT211 | NUC373 | 641537 |
| 5499 | | | NUC643 | 500702809 |
| 5533 | NUC43 | PRT212 | NUC374 | 1000872335 |
| 5563 | NUC44 | PRT213 | NUC375 | 1000852500 |
| 5609 | NUC45 | PRT214 | NUC376 | 500720555 |
| 5657 | NUC46 | PRT215 | NUC377 | 500715373 |
| 5691 | NUC47 | PRT216 | NUC378 | 167435 |
| 5748 | NUC48 | PRT217 | NUC379 | 620429 |
| 5748 | NUC49 | PRT218 | NUC379 | 613335 |
| 5806 | NUC50 | PRT219 | NUC380 | 589848 |
| 5806 | NUC51 | PRT220 | NUC380 | 211883 |
| 5806 | NUC52 | PRT221 | NUC380 | 642603 |
| 5806 | NUC53 | PRT222 | NUC380 | 193316 |
| 5816 | NUC54 | PRT223 | NUC381 | 495917 |
| 5824 | NUC55 | PRT224 | NUC382 | 160935 |
| 5861 | NUC56 | PRT225 | NUC383 | 593736 |
| 5885 | NUC57 | PRT226 | NUC384 | 613887 |
| 5913 | NUC58 | PRT227 | NUC385 | 166601 |
| 5947 | NUC96 | PRT265 | NUC413 | 654627 |
| 5966 | NUC59 | PRT228 | NUC386 | 500762665 |
| 5966 | NUC60 | PRT229 | NUC386 | 500742089 |
| 5966 | NUC61 | PRT230 | NUC386 | 500759088 |
| 5970 | | | NUC644 | 193675; 423656 |
| 5974 | NUC62 | PRT231 | NUC387 | 650666 |
| 5983 | | | NUC645 | 626803 |
| 5985 | NUC63 | PRT232 | NUC388 | 594066 |
| 6011 | NUC97 | PRT266 | NUC414 | 1000886279 |
| 6080 | NUC64 | PRT233 | NUC389 | 642569 |
| 6081 | NUC65 | PRT234 | NUC390 | 519656 |
| 6108 | | | NUC646 | 145580 |
| 6159 | NUC66 | PRT235 | NUC391 | 1000903258 |
| 6231 | NUC67 | PRT236 | NUC392 | 715579 |
| 6238 | | | NUC647 | 500694849; 500699591; 500706028; |
| | | | | 500710562; 500724984; 625642; |
| | | | | 628058; 633030 |
| 6252 | NUC98 | PRT267 | NUC415 | 1000855876 |
| 6283 | NUC68 | PRT237 | NUC393 | 820495 |
| 6290 | NUC69 | PRT238 | NUC394 | 500709853 |
| 6290 | NUC70 | PRT239 | NUC394 | 500757399 |
| 6290 | NUC71 | PRT240 | NUC394 | 592868 |
| 6322 | NUC72 | PRT241 | NUC395 | 500739746 |
| 6329 | | | NUC648 | 615173 |
| 6334 | | | NUC649 | 237324 |
| 6345 | NUC73 | PRT242 | NUC396 | 500714172 |
| 6345 | NUC74 | PRT243 | NUC396 | 500716683 |
| 6350 | NUC75 | PRT244 | NUC397 | 1000869553 |
| 6358 | NUC76 | PRT245 | NUC398 | 608537 |
| 6384 | NUC77 | PRT246 | NUC399 | 1000906334 |
| 6400 | | | NUC650 | 149691 |
| 6418 | | | NUC651 | 237026 |
| 6431 | NUC78 | PRT247 | NUC400 | 614334 |
| 6453 | NUC99 | PRT268 | NUC416 | 211056 |
| 6636 | | | NUC652 | 608607 |
| 6660 | | | NUC653 | 129407 |
| 6688 | NUC100 | PRT269 | NUC417 | 646099 |
| 6727 | NUC79 | PRT248 | NUC401 | 199782 |
| 6727 | NUC80 | PRT249 | NUC401 | 821212 |
| 6727 | NUC81 | PRT250 | NUC401 | 202863 |
| 6835 | NUC101 | PRT270 | NUC418 | 158243 |
| 6865 | | | NUC654 | 612052 |
| 6892 | NUC102 | PRT271 | NUC419 | 153261 |
| 6892 | NUC103 | PRT272 | NUC419 | 650872 |
| 6892 | NUC104 | PRT273 | NUC419 | 599054 |
| 6892 | NUC105 | PRT274 | NUC419 | 152042 |
| 6892 | NUC106 | PRT275 | NUC419 | 493328 |
| 7000 | NUC107 | PRT276 | NUC420 | 538694 |
| 7041 | | | NUC655 | 142587; 145561; 146609; |
| | | | | 149065; 153394; 153773; |
| | | | | 205319; 206906; 215376; |
| | | | | 227424; 228016; 240538; |
| | | | | 242510; 530873; 588304 |
| 7533 | | | NUC656 | 500758154 |
| 7535 | | | NUC657 | 632835 |
| 7577 | | | NUC658 | 205411 |
| 7697 | NUC108 | PRT277 | NUC421 | 653966 |
| 7712 | NUC109 | PRT278 | NUC422 | 237552 |
| 7712 | | | NUC422 | 202997; 206456 |
| 8009 | NUC110 | PRT279 | NUC423 | 645452 |
| 8078 | NUC111 | PRT280 | NUC424 | 335367 |
| 8078 | NUC112 | PRT281 | NUC424 | 334488 |
| 8078 | NUC113 | PRT282 | NUC424 | 329736 |
| 8078 | NUC114 | PRT283 | NUC424 | 244355 |
| 8078 | NUC115 | PRT284 | NUC424 | 150197 |
| 8078 | NUC116 | PRT285 | NUC424 | 244242 |
| 8078 | NUC117 | PRT286 | NUC424 | 223147 |
| 8078 | NUC118 | PRT287 | NUC424 | 221735 |
| 8078 | NUC119 | PRT288 | NUC424 | 215414 |
| 8078 | NUC120 | PRT289 | NUC424 | 149875 |
| 8078 | NUC121 | PRT290 | NUC424 | 167198 |
| 8078 | NUC122 | PRT291 | NUC424 | 193511 |
| 8078 | NUC123 | PRT292 | NUC424 | 226917 |
| 8078 | NUC124 | PRT293 | NUC424 | 225461 |
| 8078 | NUC125 | PRT294 | NUC424 | 193742 |
| 8078 | | | NUC424 | 165071; 165245; 200864; 221825; |
| | | | | 243230; 581542 |
| 8079 | | | NUC659 | 628867 |
| 8097 | | | NUC660 | 486772; 511180 |
| 8166 | NUC126 | PRT295 | NUC425 | 642948 |
| 8166 | NUC127 | PRT296 | NUC425 | 638743 |
| 8262 | | | NUC661 | 151662 |
| 8341 | | | NUC662 | 101420 |
| 8534 | | | NUC663 | 131658; 196152; 243686 |
| 8666 | NUC128 | PRT297 | NUC426 | 763024 |
| 8666 | NUC129 | PRT298 | NUC426 | 500720430 |
| 8671 | | | NUC664 | 193411 |
| 8744 | | | NUC665 | 162906 |
| 8968 | NUC82 | PRT251 | NUC402 | 771827 |
| 8968 | NUC130 | PRT299 | NUC402 | 500695719 |
| 8968 | | | NUC402 | 620376; 635045 |
| 8994 | | | NUC666 | 199362; 227277; 242546 |
| 9297 | | | NUC667 | 651871 |
| 9327 | | | NUC668 | 247810 |
| 9332 | | | NUC669 | 199155; 200810; 336623 |
| 9406 | | | NUC670 | 106061 |
| 9407 | | | NUC671 | 106061 |
| 9668 | | | NUC672 | 168218; 197771; 205623; 228775; |
| | | | | 238794 |
| 9679 | NUC131 | PRT300 | NUC427 | 206381 |
| 9755 | | | NUC673 | 197091 |
| 9868 | | | NUC674 | 144783; 206407; 215714; 234057; |
| | | | | 336758; 582582 |
| 10044 | | | NUC675 | 107768; 111854; 500721812; 500723626; |
| | | | | 500723636; 500724389; |
| | | | | 500725580; 500729834; 500735442; |
| | | | | 500735787; 500758255; 500762395; |
| | | | | 586703; 589397; 612312; 635730; |
| | | | | 642849; 645812; 762987; 767609 |
| 10322 | NUC132 | PRT301 | NUC428 | 200895 |
| 10526 | NUC133 | PRT302 | NUC429 | 1000891255 |
| 10584 | | | NUC676 | 500745219 |
| 10650 | | | NUC677 | 187889; 242499 |
| 10739 | NUC134 | PRT303 | NUC430 | 637548 |
| 10743 | NUC135 | PRT304 | NUC431 | 767426 |
| 10744 | NUC136 | PRT305 | NUC432 | 500691428 |
| 10761 | | | NUC678 | 131060 |
| 10880 | NUC137 | PRT306 | NUC433 | 116153 |
| 10942 | NUC138 | PRT307 | NUC434 | 500699885 |
| 10942 | NUC139 | PRT308 | NUC434 | 746303 |
| 10942 | NUC140 | PRT309 | NUC434 | 500705937 |
| 10942 | | | NUC434 | 500705002; 500712632; 633931; |
| | | | | 634489; 813634; 816859 |
| 11019 | NUC141 | PRT310 | NUC435 | 150568 |
| 11278 | NUC142 | PRT311 | NUC436 | 495638 |
| 11342 | NUC143 | PRT312 | NUC437 | 143196 |
| 11562 | | | NUC679 | 187543 |
| 11688 | | | NUC680 | 165419; 165544; 166387; 181924; |
| | | | | 181930; 196904; 199001; 199269; |
| | | | | 224447; 238731; 243770 |
| 11735 | NUC144 | PRT313 | NUC438 | 633418 |
| 11735 | NUC145 | PRT314 | NUC438 | 422878 |
| 11735 | | | NUC438 | 500706283; 500711792; 500712711; |
| | | | | 500725618; 500738973; 651370 |
| 11813 | | | NUC681 | 633791 |
| 12039 | NUC146 | PRT315 | NUC439 | 546312 |
| 12043 | | | NUC682 | 632330 |
| 12048 | | | NUC683 | 101164 |
| 12098 | | | NUC684 | 135037 |
| 12202 | | | NUC685 | 168232; 243338 |
| 12220 | | | NUC686 | 433866 |
| 12243 | | | NUC687 | 659527 |
| 12263 | | | NUC688 | 139596 |
| 12276 | | | NUC689 | 624892; 628879; 631590; 633480 |
| 12490 | | | NUC690 | 500704734; 500744586; 500744972; |
| | | | | 611533; 634337 |
| 12604 | NUC147 | PRT316 | NUC440 | 614106 |
| 12604 | | | NUC440 | 178304 |
| 12657 | | | NUC691 | 238579; 248948; 397864; 521873; |
| | | | | 526295; 589951 |
| 12788 | NUC148 | PRT317 | NUC441 | 330777 |
| 12901 | NUC149 | PRT318 | NUC442 | 124608 |
| 12907 | NUC150 | PRT319 | NUC443 | 478617 |
| 12907 | NUC151 | PRT320 | NUC443 | 481184 |
| 13013 | | | NUC692 | 583731; 650307; 650848 |
| 13202 | | | NUC693 | 238886 |
| 13229 | NUC152 | PRT321 | NUC444 | 612301 |
| 13256 | NUC153 | PRT322 | NUC445 | 165123 |
| 13256 | NUC154 | PRT323 | NUC445 | 165643 |
| 13256 | | | NUC445 | 142964; 150214; 223536; 245008 |
| 13267 | NUC155 | PRT324 | NUC446 | 488818 |
| 13285 | | | NUC694 | 193487; 238191; 248387 |
| 26638 | | | NUC695 | 645819 |
| 26710 | | | NUC696 | 600909; 608784; 611758; 614721; |
| | | | | 619435; 620041; 620372; 625815; |
| | | | | 625933; 625983; 626308; 627299; |
| | | | | 627481; 628147; 628753; 631655; |
| | | | | 633039; 633371; 633760; 634553; |
| | | | | 642966 |
| 26726 | | | NUC697 | 421115 |
| 26786 | | | NUC698 | 500702480 |
| 26982 | | | NUC699 | 638872 |
| 27084 | NUC156 | PRT325 | NUC447 | 242080 |
| 27084 | | | NUC447 | 128161; 186671; 210505; 211578; |
| | | | | 214909; 221663; 222101; 223000; |
| | | | | 224361; 226849; 242326; 242424; |
| | | | | 243662; 244913; 247912 |
| 27273 | | | NUC700 | 525674; 601556 |
| 27301 | | | NUC701 | 135037 |
| 27336 | | | NUC702 | 500742735 |
| 27361 | | | NUC703 | 205346; 530902 |
| 27374 | | | NUC704 | 643006 |
| 27627 | | | NUC705 | 129706; 223196 |
| 27697 | | | NUC706 | 500701900 |
| 27877 | | | NUC707 | 99497 |
| 28413 | | | NUC708 | 135042 |
| 28517 | | | NUC709 | 150011; 201848 |
| 28518 | | | NUC710 | 500721700; 500729093; 500730152 |
| 29120 | NUC157 | PRT326 | NUC448 | 488444 |
| 29469 | | | NUC711 | 638852 |
| 29472 | | | NUC712 | 637812 |
| 29557 | | | NUC713 | 188208 |
| 29673 | | | NUC714 | 650606 |
| 29814 | | | NUC715 | 813496 |
| 30218 | | | NUC716 | 241681; 242553; 589203 |
| 30446 | | | NUC717 | 105288 |
| 30477 | | | NUC718 | 500724995; 500758517 |
| 30583 | | | NUC719 | 117932; 194613; 225013; 331614 |
| 30719 | | | NUC720 | 637363 |
| 31356 | | | NUC721 | 106998 |
| 31422 | NUC158 | PRT327 | NUC449 | 500732587 |
| 31554 | | | NUC722 | 222161 |
| 31627 | | | NUC723 | 173050 |
| 31726 | | | NUC724 | 393750 |
| 31744 | | | NUC725 | 176380 |
| 31790 | | | NUC726 | 625728 |
| 32102 | | | NUC727 | 500762549 |
| 32473 | NUC159 | PRT328 | NUC450 | 183902 |
| 32475 | NUC160 | PRT329 | NUC451 | 635993 |
| 32962 | | | NUC728 | 500740719 |
| 33130 | | | NUC729 | 165852; 165888 |
| 33712 | NUC161 | PRT330 | NUC452 | 398703 |
| 35005 | | | NUC730 | 124493 |
| 35185 | NUC83 | PRT252 | NUC403 | 589785 |
| 35258 | | | NUC731 | 224898 |
| 35326 | | | NUC732 | 145027 |
| 35597 | | | NUC733 | 237630 |
| 35912 | | | NUC734 | 637431 |
| 35984 | | | NUC735 | 117238 |
| 36122 | | | NUC736 | 226039 |
| 37337 | | | NUC737 | 143508; 196052; 221995 |
| 38112 | | | NUC738 | 129444 |
| 38220 | | | NUC739 | 608709 |
| 38311 | | | NUC740 | 600921 |
| 38631 | | | NUC741 | 194909 |
| 38749 | | | NUC742 | 163588 |
| 38890 | NUC162 | PRT331 | NUC453 | 500742815 |
| 38890 | NUC163 | PRT332 | NUC453 | 500735594 |
| 38890 | NUC164 | PRT333 | NUC453 | 500737569 |
| 38890 | NUC165 | PRT334 | NUC453 | 500730242 |
| 38890 | NUC166 | PRT335 | NUC453 | 500766374 |
| 38890 | NUC167 | PRT336 | NUC453 | 500711885 |
| 40163 | | | NUC743 | 244540 |
| 40975 | | | NUC744 | 106061 |
| 40991 | | | NUC745 | 106061 |
| 42896 | | | NUC746 | 137110 |
| 43190 | | | NUC747 | 244266 |
| 44053 | | | NUC748 | 236316; 249443; 392450; 449591; |
| | | | | 486460; 509697; 519210; 528872; |
| | | | | 585226; 589902; 601550 |
| 45091 | | | NUC749 | 238559 |
| 45179 | | | NUC750 | 132269 |
| 45274 | NUC84 | PRT253 | NUC404 | 1000867870 |
| 46679 | NUC85 | PRT254 | NUC405 | 140265 |
| 47171 | | | NUC751 | 420959 |
| 48024 | NUC168 | PRT337 | NUC454 | 113448 |
| 48548 | | | NUC752 | 227400 |
| 48603 | | | NUC753 | 200687; 244886 |
| 48670 | | | NUC754 | 164887 |
| 48671 | | | NUC755 | 221136 |
| 48823 | | | NUC756 | 525888 |
| 48901 | | | NUC757 | 525775 |
| 49018 | | | NUC758 | 229481 |
| 49034 | | | NUC759 | 237358 |
| 49133 | | | NUC760 | 224706; 582974 |
| 49140 | | | NUC761 | 636146 |
| 49261 | | | NUC762 | 186091 |
| 49387 | | | NUC763 | 212526 |
| 49416 | | | NUC764 | 530211 |
| 49426 | | | NUC765 | 313150; 313151 |
| 49493 | | | NUC766 | 213393 |
| 49640 | | | NUC767 | 626919 |
| 49863 | | | NUC768 | 181361; 382057; 631692 |
| 49871 | | | NUC769 | 181361; 382057; 631692 |
| 50015 | | | NUC770 | 203070; 331013 |
| 50049 | | | NUC771 | 196104 |
| 50112 | | | NUC772 | 118123; 335719 |
| 50185 | | | NUC773 | 489405; 496185 |
| 50241 | | | NUC774 | 145339; 156186; 244350 |
| 50353 | | | NUC775 | 119282 |
| 50763 | | | NUC776 | 500701668 |
| 50982 | | | NUC777 | 637372 |
| 51130 | | | NUC778 | 180402 |
| 51212 | | | NUC779 | 238923 |
| 51346 | | | NUC780 | 646118 |
| 51380 | NUC169 | PRT338 | NUC455 | 523002 |
| 51400 | | | NUC781 | 231102 |
| 51796 | | | NUC782 | 210281 |
| 51954 | | | NUC783 | 141971; 183117; 205855; 502241 |
| 52076 | | | NUC784 | 625028 |

TABLE II

| SEQ ID NO. | |
|---|---|
| in priority | |
| application | Chromosomal location |
| 51 | 3q26.2 |
| 452 | 2q31 |
| 483 | 11p11.2 |
| 505 | 20p12 |
| 573 | 5q21 |
| 796 | 15 |
| 1151 | 22q11.2 |
| 1590 | X |
| 2028 | X |
| 2932 | 8p23 |
| 3280 | 21 |
| 3326 | 15 |
| 3804 | 16p13.3 |
| 4172 | 1, 15q13-q14 |
| 4340 | 1 |
| 4609 | 4p13 |
| 4647 | 11q23, 11q23.3 |
| 4694 | 12p13.2 |
| 4733 | 14q32.1 |
| 4855 | 1q23-1q24 |
| 4908 | 12p13 |
| 5011 | 7q32, 7q32-36 |
| 5040 | 8p23 |
| 5089 | 4q11 |
| 5167 | 12 |
| 5278 | 19q13.2 |
| 5563 | 1, 2 |
| 5947 | 11q23 |
| 5974 | 4q28, 4q28-q31 |
| 6011 | 4 |
| 6290 | 2p13 |
| 6322 | 2p13 |
| 6345 | 16q24.3 |
| 6400 | 10q25-26 |
| 6892 | 4 |
| 7697 | 2p11, 4q28 |
| 8166 | 14q32 |
| 8666 | 16 |
| 8671 | 16 |
| 9755 | 3 |
| 10044 | 12q, 16 |
| 10322 | 1q11.1, 1q21 |
| 10584 | 3p21.3 |
| 10744 | 6 |
| 11019 | 11p11.2, 22q13 |
| 11342 | 7q21 |
| 12907 | 7q36.1 |
| 13202 | 15 |
| 13285 | 2q23-24, 11q23.2-24.2 |
| 27697 | 12p11.2 |
| 28517 | 2q31 |
| 28518 | 2q31 |
| 29673 | 16p13.3 |
| 29814 | 11 |
| 31422 | 11q13 |
| 31627 | 11 |
| 32962 | 9q32-33 |
| 33712 | 3p21.3, 3p21.31 |
| 35185 | 2 |
| 38631 | 15 |
| 48823 | 13q34-qter, 17 |
| 49018 | 7q22 |
| 49133 | 1q32 |
| 49387 | 17q21 |
| 50015 | 5q31 |
| 51380 | Xq28 |

TABLE III

| SEQ ID NO. in | Tissue Distribution |
|---|---|
| 37 | I: 11 |
| 51 | A: 55 B: 4 C: 1 E: 1 F: 30 G: 19 H: 9 |
| 179 | F: 16 K: 4 |
| 180 | F: 5 K: 1 |
| 183 | H: 6 |
| 201 | C: 1 |
| 326 | I: 3 |
| 362 | K: 2 |
| 440 | A: 7 |
| 452 | F: 1 G: 5 |
| 483 | G: 4 |
| 500 | I: 1 |
| 505 | A: 2 |
| 528 | K: 2 |
| 573 | F: 3 K: 1 |
| 574 | A: 1 |
| 587 | H: 1 I: 3 |
| 588 | F: 6 K: 2 |
| 593 | A: 2 C: 15 F: 2 G: 14 |
| 599 | A: 8 F: 7 K: 3 |
| 603 | K: 1 |
| 621 | C: 1 |
| 628 | F: 1 K: 3 |
| 653 | B: 6 F: 3 G: 1 I: 1 K: 7 |
| 670 | K: 11 |
| 678 | F: 1 K: 2 |
| 693 | B: 7 G: 7 |
| 703 | F: 9 K: 4 |
| 746 | A: 9 H: 1 K: 2 |
| 770 | G: 4 |
| 775 | F: 1 K: 13 |
| 796 | G: 1 |
| 812 | A: 13 F: 1 H: 1 |
| 940 | F: 8 |
| 988 | F: 8 K: 1 |
| 996 | K: 4 |
| 1036 | A: 13 I: 11 |
| 1064 | A: 12 B: 130 C: 16 D: 7 F: 1 G: 16 |
| 1151 | A: 9 |
| 1190 | B: 4 C: 1 H: 7 I: 6 |
| 1458 | J: 55 |
| 1590 | A: 1 |
| 1853 | A: 1 G: 1 H: 1 |
| 1904 | H: 1 |
| 2028 | A: 8 |
| 2173 | B: 1 C: 3 D: 1 G: 1 |
| 2368 | K: 4 |
| 2553 | B: 7 D: 5 H: 39 |
| 2556 | B: 7 D: 5 H: 39 |
| 2658 | D: 1 |
| 2690 | K: 2 |
| 2755 | H: 1 |
| 2800 | F: 8 K: 6 |
| 2843 | A: 8 G: 1 K: 3 |
| 2852 | F: 1 |
| 2932 | D: 1 |
| 2955 | K: 1 |
| 3078 | H: 2 |
| 3280 | F: 4 K: 3 |
| 3326 | H: 2 |
| 3387 | H: 7 |
| 3439 | I: 5 |
| 3501 | A: 10 C: 1 |
| 3633 | A: 6 H: 3 |
| 3678 | K: 2 |
| 3714 | F: 4 K: 1 |
| 3796 | F: 1 K: 1 |
| 3801 | K: 2 |
| 3804 | B: 1 C: 10 D: 1 G: 2 I: 11 |
| 3892 | A: 9 |
| 3985 | B: 1 C: 5 D: 1 H: 1 J: 6 |
| 4005 | F: 6 I: 2 K: 2 |
| 4063 | A: 2 B: 10 C: 4 |
| 4088 | K: 6 |
| 4111 | A: 11 |
| 4126 | F: 3 K: 1 |
| 4172 | F: 3 K: 2 |
| 4261 | H: 2 |
| 4340 | A: 3 G: 3 I: 1 |
| 4436 | F: 3 |
| 4609 | D: 10 |
| 4647 | D: 3 |
| 4660 | D: 1 |
| 4664 | B: 48 C: 2 H: 2 I: 3 |
| 4678 | C: 5 D: 17 G: 4 |
| 4682 | G: 1 |
| 4687 | D: 5 F: 2 H: 1 |
| 4690 | D: 3 F: 1 I: 1 K: 1 |
| 4694 | I: 3 |
| 4696 | I: 1 |
| 4733 | D: 1 |
| 4807 | B: 11 H: 1 |
| 4809 | I: 1 |
| 4830 | K: 1 |
| 4855 | H: 1 |
| 4900 | A: 1 |
| 4908 | A: 16 |
| 4943 | F: 6 K: 2 |
| 4947 | K: 3 |
| 4976 | J: 2 |
| 5000 | A: 2 B: 14 C: 17 D: 5 F: 1 G: 9 H: 3 I: 3 |
| 5002 | B: 2 |
| 5005 | C: 31 F: 1 J: 2 K: 2 |
| 5011 | H: 5 I: 2 |
| 5040 | B: 1 C: 2 D: 11 G: 1 J: 1 |
| 5058 | K: 1 |
| 5071 | F: 1 K: 1 |
| 5089 | I: 1 |
| 5117 | H: 7 |
| 5141 | F: 2 K: 1 |
| 5162 | A: 1 B: 8 |
| 5167 | I: 1 |
| 5178 | G: 1 |
| 5192 | F: 14 K: 3 |
| 5214 | A: 10 B: 19 C: 5 |
| 5230 | A: 3 B: 6 C: 9 D: 1 F: 1 G: 4 H: 3 I: 2 |
| 5240 | A: 1 |
| 5250 | A: 3 B: 39 C: 29 D: 3 H: 3 I: 1 K: 1 |
| 5262 | A: 3 B: 39 C: 34 D: 3 H: 3 I: 1 K: 1 |
| 5270 | B: 8 |
| 5278 | D: 1 |
| 5358 | G: 1 |
| 5453 | A: 2 C: 6 |
| 5494 | A: 1 D: 1 |
| 5499 | A: 3 |
| 5533 | A: 2 B: 7 C: 1 |
| 5563 | D: 1 F: 1 |
| 5609 | G: 3 |
| 5657 | G: 1 |
| 5691 | K: 1 |
| 5748 | A: 3 B: 1 C: 4 D: 1 G: 30 H: 1 |
| 5806 | A: 2 B: 7 C: 1 F: 1 I: 3 K: 3 |
| 5816 | B: 5 |
| 5824 | H: 3 |
| 5861 | B: 16 C: 27 D: 13 F: 7 H: 21 I: 12 K: 4 |
| 5885 | A: 1 |
| 5913 | K: 1 |
| 5947 | D: 2 |
| 5966 | G: 11 |
| 5970 | C: 4 |
| 5974 | D: 12 |
| 5983 | A: 5 |
| 5985 | B: 2 C: 5 H: 1 I: 1 K: 2 |
| 6011 | B: 1 |
| 6080 | B: 1 I: 1 |
| 6081 | I: 9 |
| 6108 | F: 5 |
| 6159 | B: 1 |
| 6231 | A: 1 |
| 6238 | A: 15 B: 5 G: 14 H: 3 I: 2 |
| 6252 | G: 7 |
| 6283 | F: 1 K: 1 |
| 6290 | B: 3 |
| 6322 | C: 1 |
| 6329 | A: 1 |
| 6334 | F: 51 K: 13 |
| 6345 | A: 9 G: 1 |
| 6350 | A: 1 B: 2 |
| 6358 | B: 9 H: 1 |
| 6384 | B: 1 C: 1 |
| 6400 | K: 1 |
| 6418 | K: 1 |
| 6431 | A: 1 B: 1 K: 1 |
| 6453 | F: 1 |
| 6636 | A: 6 B: 37 C: 11 D: 1 F: 1 G: 4 H: 1 J: 1 |
| 6660 | F: 1 K: 2 |
| 6688 | A: 1 B: 42 D: 1 G: 3 I: 3 K: 3 |
| 6727 | F: 16 G: 5 K: 103 |
| 6835 | K: 1 |
| 6865 | A: 1 I: 1 |
| 6892 | B: 23 C: 1 H: 2 I: 1 J: 12 |
| 7000 | B: 1 |
| 7041 | C: 1 F: 17 H: 1 |
| 7533 | G: 2 |
| 7535 | A: 5 |
| 7577 | F: 1 |
| 7697 | D: 10 F: 3 |
| 7712 | F: 1 K: 3 |
| 8009 | B: 2 C: 2 H: 1 |
| 8078 | F: 5 K: 20 |
| 8097 | I: 3 |
| 8166 | A: 7 B: 6 D: 8 G: 52 |
| 8262 | H: 1 |
| 8341 | A: 1 F: 2 G: 3 H: 2 I: 1 |
| 8534 | F: 1 K: 6 |
| 8666 | B: 4 D: 1 G: 16 K: 1 |
| 8671 | G: 1 K: 2 |
| 8744 | H: 1 |
| 8968 | A: 12 G: 3 H: 1 |
| 8994 | F: 1 K: 4 |
| 9297 | D: 1 |
| 9327 | K: 1 |
| 9332 | K: 4 |
| 9406 | B: 10 D: 3 H: 39 |
| 9407 | B: 10 D: 3 H: 39 |
| 9668 | F: 9 K: 41 |
| 9679 | B: 1 F: 1 K: 1 |
| 9755 | K: 5 |
| 9868 | F: 9 K: 1 |
| 10044 | A: 10 B: 10 D: 14 F: 2 G: 24 H: 5 K: 4 |
| 10322 | B: 1 F: 2 K: 3 |
| 10526 | B: 3 H: 3 |
| 10584 | A: 2 |
| 10650 | K: 3 |
| 10739 | A: 1 B: 1 G: 3 J: 1 |
| 10743 | C: 1 J: 1 |
| 10744 | A: 6 B: 3 G: 2 I: 3 |
| 10761 | H: 1 |
| 10880 | D: 1 G: 1 |
| 10942 | A: 30 |
| 11019 | F: 4 |
| 11278 | I: 1 |
| 11342 | F: 1 K: 2 |
| 11562 | F: 1 K: 4 |
| 11688 | F: 2 K: 12 |
| 11735 | A: 25 B: 57 C: 7 D: 1 F: 1 G: 1 K: 1 |
| 11813 | A: 8 |
| 12039 | B: 3 |
| 12043 | A: 6 |
| 12048 | F: 1 H: 1 |
| 12098 | A: 4 B: 8 H: 2 I: 1 |
| 12202 | K: 2 |
| 12220 | C: 2 |
| 12243 | A: 1 B: 132 C: 38 D: 31 F: 6 G: 11 |
| 12263 | H: 4 |
| 12276 | A: 18 |
| 12490 | A: 9 B: 11 C: 8 G: 4 |
| 12604 | A: 6 B: 14 C: 22 D: 1 F: 1 G: 4 H: 4 I: 1 |
| 12657 | B: 1 C: 6 J: 2 |
| 12788 | K: 2 |
| 12901 | G: 2 |
| 12907 | I: 6 |
| 13013 | D: 5 |
| 13202 | A: 2 B: 14 C: 9 D: 1 G: 3 H: 3 J: 3 K: 1 |
| 13229 | A: 3 |
| 13256 | F: 2 K: 5 |
| 13267 | I: 8 |
| 13285 | B: 1 C: 42 D: 5 I: 1 J: 10 |
| 26638 | D: 2 |
| 26710 | A: 90 B: 48 G: 27 |
| 26726 | C: 4 |
| 26786 | A: 24 |
| 26982 | A: 1 G: 1 |
| 27084 | F: 16 K: 83 |
| 27273 | C: 3 |
| 27301 | A: 3 B: 3 H: 2 |
| 27336 | B: 1 G: 1 |
| 27361 | F: 2 |
| 27374 | B: 1 D: 1 |
| 27627 | F: 1 K: 1 |
| 27697 | A: 1 C: 1 |
| 27877 | H: 1 I: 19 |
| 28413 | H: 1 |
| 28517 | K: 2 |
| 28518 | G: 3 |
| 29120 | I: 1 |
| 29469 | A: 2 G: 2 |
| 29472 | A: 1 |
| 29557 | C: 1 |
| 29673 | D: 1 |
| 29814 | A: 1 |
| 30218 | F: 7 K: 1 |
| 30446 | B: 1 C: 1 H: 10 |
| 30477 | G: 7 |
| 30583 | K: 5 |
| 30719 | A: 1 B: 9 I: 3 |
| 31356 | H: 2 |
| 31422 | G: 1 |
| 31554 | K: 1 |
| 31627 | C: 1 |
| 31726 | C: 1 |
| 31744 | F: 1 |
| 31790 | A: 25 |
| 32102 | G: 1 |
| 32473 | B: 1 |
| 32475 | B: 1 |
| 32962 | G: 1 |
| 33130 | K: 3 |
| 33712 | B: 1 C: 1 |
| 35005 | A: 1 |
| 35185 | C: 1 |
| 35258 | K: 1 |
| 35326 | B: 1 F: 3 G: 1 H: 1 I: 1 K: 3 |
| 35597 | F: 1 K: 1 |
| 35912 | A: 1 |
| 35984 | F: 1 |
| 36122 | K: 1 |
| 37337 | F: 1 K: 2 |
| 38112 | K: 1 |
| 38220 | A: 1 |
| 38311 | A: 1 |
| 38631 | K: 1 |
| 38749 | H: 1 |
| 38890 | G: 21 |
| 40163 | K: 2 |
| 40975 | B: 8 D: 1 H: 39 |
| 40991 | B: 8 D: 1 H: 39 |
| 42896 | H: 1 |
| 43190 | K: 1 |
| 44053 | C: 11 D: 1 H: 1 I: 2 J: 5 |
| 45091 | J: 1 |
| 45179 | H: 1 |
| 45274 | C: 4 H: 2 |
| 46679 | D: 3 F: 1 H: 1 |
| 47171 | C: 1 |
| 48024 | B: 4 C: 5 |
| 48548 | F: 1 |
| 48603 | F: 41 K: 19 |
| 48670 | K: 1 |
| 48671 | K: 2 |
| 48823 | C: 65 |
| 48901 | B: 1 G: 2 I: 2 |
| 49018 | B: 2 C: 2 F: 2 J: 2 |
| 49034 | D: 1 F: 2 H: 1 I: 3 K: 2 |
| 49133 | F: 4 K: 2 |
| 49140 | A: 1 C: 1 |
| 49261 | F: 3 K: 23 |
| 49387 | F: 2 K: 1 |
| 49416 | B: 18 C: 1 |
| 49426 | J: 51 |
| 49493 | C: 1 F: 1 I: 6 |
| 49640 | A: 5 F: 1 |
| 49863 | A: 6 C: 2 I: 1 |
| 49871 | A: 6 C: 2 I: 1 |
| 50015 | K: 2 |
| 50049 | K: 2 |
| 50112 | K: 3 |
| 50185 | I: 6 |
| 50241 | F: 3 H: 1 |
| 50353 | F: 1 K: 1 |
| 50763 | A: 14 |
| 50982 | A: 2 |
| 51130 | K: 2 |
| 51212 | J: 2 |
| 51346 | A: 2 B: 1 D: 1 |
| 51380 | B: 1 F: 1 |
| 51400 | F: 3 |
| 51796 | F: 1 K: 2 |
| 51954 | B: 13 C: 1 D: 1 F: 2 K: 1 |
| 52076 | A: 2 |

TABLE IV

| SEQ ID NO. in | |
|---|---|
| priority | |
| application | Tissue source |
| 37 | adenocarcinoma(2), carcinoid(2), testis(1), tonsil(1) |
| 51 | adipose tissue, white(4), cerebellum(3), cochlea(1), colon tumor rer+(2), dorsal root |
| | ganglion(1), hippocampus(1), kidney(1), liver(1), malignant melanoma, metastatic to lymph |
| | node(1), muscle(2), normal leg muscle(1), parathyroid tumor(1), pectoral muscle (after |
| | mastectomy)(1), placenta(1), substantia nigra(1), total brain(1) |
| 201 | pancreas(1) |
| 326 | ovarian tumor(1), uterus(1) |
| 440 | brain cortex(1), carcinoid tumor(1) |
| 483 | pbl(1), adenocarcinoma(1), astrocytoma(1), ovarian tumor(1), schizophrenic brain s-11 |
| | frontal lobe(1) |
| 500 | colon(4), colon tumor rer+(1), pooled germ cell tumors(1) |
| 505 | total brain(1) |
| 528 | brain(2), cerebellum(1), colon(1), ovarian tumor(6) |
| 573 | melanoma (mewo cell line)(1) |
| 587 | germinal center b cell(1), lymphoma(1), parathyroid tumor(1) |
| 593 | ovarian tumor(1), placenta(1) |
| 621 | placenta.(2) |
| 653 | 2 pooled tumors (clear cell type)(2), anaplastic oligodendroglioma(2), glioblastoma |
| | (pooled)(2) |
| 678 | ovarian tumor(1), prostate(1) |
| 693 | brain(1), placenta(1) |
| 703 | small cell carcinoma(2) |
| 746 | small cell carcinoma(1) |
| 770 | colon(1), frontal lobe(1), human pancreatic islets(1), normal leg muscle(1), ovarian tumor(1), |
| | pancreatic islet(2), senescent fibroblast(1), total brain(1) |
| 775 | anaplastic oligodendroglioma(1), frontal lobe(1) |
| 796 | adrenal adenoma(1), adrenal gland(1), breast tumor(3), placenta(2) |
| 812 | heart(1), brain(1), frontal lobe(3), neuroepithelial cells(1), retina(1), small cell carcinoma(1), |
| | total brain(1) |
| 988 | germinal center b cell(1) |
| 1064 | 2 pooled tumors (clear cell type)(1), ewing's sarcoma(2), adenocarcinoma(5), anaplastic |
| | oligodendroglioma(5), breast tumor(1), carcinoid(4), colon(3), colon tumor(1), colon tumor |
| | rer+(2), frontal lobe(4), germinal center b cell(7), kidney tumor(1), lung tumor(1), metastatic |
| | prostate bone lesion(2), ovarian tumor(6), parathyroid tumor(7), pectoral muscle (after |
| | mastectomy)(12), placenta(1), pooled germ cell tumors(4), senescent fibroblast(2), squamous |
| | cell carcinoma from base of tongue(1), tumor, 5 pooled (see description)(1) |
| 1151 | anaplastic oligodendroglioma(4), carcinoid(1), colon tumor rer+(1), medulloblastoma(1), |
| | normal prostate(1), parathyroid tumor(1), tumor(1) |
| 1190 | colon(1) |
| 1853 | 2 pooled high-grade transitional cell tumors(1), 2 pooled tumors (clear cell type)(1) |
| 2173 | 2 pooled tumors (clear cell type)(2), b-cell, chronic lymphotic leukemia(2), cd34+, cd38− |
| | from normal bone marrow donor(6), adenocarcinoma(1), alveolar rhabdomyosarcoma(1), |
| | anaplastic oligodendroglioma(3), breast(1), carcinoid(1), cerebellum(2), cochlea(3), |
| | colon(11), colon tumor rer+(2), dorsal root ganglion(1), early stage papillary serous |
| | carcinoma(1), epithelium (cell line)(1), follicular lymphoma(1), frontal lobe(15), germinal |
| | center b-cells(3), invasive adenocarcinoma(5), kidney(1), kidney tumor(2), larynx(1), |
| | liver(1), lung carcinoma(1), lymphoma(1), meningioma(1), moderately differentiated |
| | adenocarcinoma(2), moderately-differentiated adenocarcinoma(1), muscle(1), normal |
| | prostate(6), normal prostatic epithelial cells(1), oligodendroglioma(3), ovarian tumor(4), |
| | papillary serous carcinoma(1), pectoral muscle (after mastectomy)(23), pooled germ cell |
| | tumors(2), prostate(1), stem cell 34+/38+(2), thyroid(1), tumor, 5 pooled (see description)(4), |
| | two pooled squamous cell carcinomas(2) |
| 2553 | brain(2), tumor(1) |
| 2556 | brain(2), tumor(1) |
| 2755 | frontal lobe(1) |
| 2843 | b-cell, chronic lymphotic leukemia(2), anaplastic oligodendroglioma(5), carcinoid(1), |
| | placenta(1) |
| 2852 | frontal lobe(3) |
| 2932 | bone marrow(14), brain(1), hematopoietic from aml patient(1), liver(2), normal cortical |
| | stroma(1) |
| 3078 | frontal lobe(2) |
| 3280 | b-cell, chronic lymphotic leukemia(1), germinal center b cell(1), leiomyosarcoma(1), pooled |
| | germ cell tumors(1) |
| 3326 | muscle(3), normal leg muscle(1), parathyroid tumor(1) |
| 3387 | carcinoid(1), pooled germ cell tumors(4) |
| 3439 | 2 pooled tumors (clear cell type)(1), b-cell, chronic lymphotic leukemia(3), ewing's |
| | sarcoma(1), anaplastic oligodendroglioma(1), colon(2), glioblastoma (pooled)(1), mantle cell |
| | lymphoma(1), parathyroid tumor(1), senescent fibroblast(1), squamous cell carcinoma(1) |
| 3501 | anaplastic oligodendroglioma(1), breast(1), carcinoid(2), colon(1), colon tumor rer+(1), |
| | epithelium (cell line)(1), kidney tumor(1), ovarian tumor(1), parathyroid tumor(2), pooled |
| | germ cell tumors(4), senescent fibroblast(1), squamous cell carcinoma(1), testis(1), tumor(2) |
| 3633 | cerebellum(1), muscle(1), retina(4), small cell carcinoma(1), total brain(3) |
| 3714 | 2 pooled tumors (clear cell type)(1), colon(1) |
| 3804 | heart(1), anaplastic oligodendroglioma(1), carcinoid(1), colon tumor(2), germinal center b |
| | cell(2), kidney tumor(1), lung tumor(3), lymphoid(2), moderately differentiated |
| | adenocarcinoma(1), muscle(1), ovarian tumor(3), pectoral muscle (after mastectomy)(1), |
| | squamous cell carcinoma(3) |
| 3892 | blood(1), total brain(2) |
| 3985 | anaplastic oligodendroglioma(1), breast(1) |
| 4005 | 2 pooled tumors (clear cell type)(1), brain(2), germinal center b cell(1), muscle(1) |
| 4063 | cerebral cortex(1), brain(1), carcinoid(2), cerebellum(1), schizophrenic brain s-11 frontal |
| | lobe(1), senescent fibroblast(1), total brain(2) |
| 4111 | anaplastic oligodendroglioma(1), cerebellum(1), colon(1) |
| 4172 | retina(3) |
| 4340 | heart(1), blood(1), brain(2), colon(6), frontal lobe(9), invasive tumor (cell line)(1), |
| | melanocyte(1), neuroepithelial cells(1), senescent fibroblast(1), small cell carcinoma(2), total |
| | brain(3) |
| 4436 | b-cell, chronic lymphotic leukemia(1), bulk tumor(1), colon(24), early stage papillary serous |
| | carcinoma(3), lung carcinoma(1), pancreatic cancer(1), pooled germ cell tumors(1) |
| 4609 | blood(1) |
| 4647 | colon(1), liver(1) |
| 4660 | adenocarcinoma(1) |
| 4664 | 2 pooled tumors (clear cell type)(5), adenocarcinoma(3), brain(1), breast(1), colon(4), colon |
| | tumor rer+(1), frontal lobe(5), liver(1), neuroepithelial cells(1), normal prostate(1), ovarian |
| | tumor(1), ovary(1), total brain(1), tumor(1) |
| 4678 | colon tumor, rer+(2) |
| 4682 | 2 pooled tumors (clear cell type)(4), anaplastic oligodendroglioma(2), breast(3), carcinoid(1), |
| | glioblastoma (pooled)(1), pooled germ cell tumors(1) |
| 4687 | 2 pooled tumors (clear cell type)(1), carcinoid(3), colon(3), normal prostate(1), pooled germ |
| | cell tumors(1) |
| 4690 | carcinoid(1), colon(3), normal prostate(1), pooled germ cell tumors(1) |
| 4694 | colon tumor, rer+(1) |
| 4733 | colon(3), liver(14), pancreatic islet(1) |
| 4807 | 2 pooled tumors (clear cell type)(1), alveolar rhabdomyosarcoma(1), carcinoid(3), colon(2), |
| | normal prostate(2), normal prostatic epithelial cells(1), ovarian tumor(23), prostate(2), serous |
| | adenocarcinoma(1), total brain(2), tumor(1), tumor, 5 pooled (see description)(4) |
| 4809 | 2 pooled tumors (clear cell type)(1), alveolar rhabdomyosarcoma(1), carcinoid(3), colon(2), |
| | normal prostate(2), normal prostatic epithelial cells(1), ovarian tumor(28), prostate(2), serous |
| | adenocarcinoma(1), total brain(2), tumor(1), tumor, 5 pooled (see description)(5) |
| 4855 | b-cell, chronic lymphotic leukemia(1), carcinoid(1) |
| 4900 | 2 pooled tumors (clear cell type)(1), b-cell, chronic lymphotic leukemia(1), colon(1), liver(1), |
| | low-grade prostatic neoplasia(1), normal prostate(1), pooled germ cell tumors(1) |
| 4908 | alveolar rhabdomyosarcoma(1), colon(1), hemopoietic system(1), liver(1), malignant ascitic |
| | effusion(1), moderately-differentiated adenocarcinoma(1), neuroepithelial cells(1), |
| | parathyroid tumor(1), synovial membrane(1), uterus(1) |
| 4947 | parathyroid tumor(1), testis(2) |
| 5000 | 2 pooled tumors (clear cell type)(3), b-cell, chronic lymphotic leukemia(2), anaplastic |
| | oligodendroglioma(5), breast(4), carcinoid(2), colon tumor, rer+(1), germ cell tumor(1), |
| | germinal center b cell(6), invasive prostate tumor(1), lung tumor(1), metastatic prostate bone |
| | lesion(2), normal prostate(1), parathyroid tumor(2), pectoral muscle (after mastectomy)(3), |
| | pooled germ cell tumors(1), senescent fibroblast(4), stroma(1), thyroid(1), tumor, 5 pooled |
| | (see description)(2) |
| 5002 | pectoral muscle (after mastectomy)(1), senescent fibroblast(1) |
| 5005 | anaplastic oligodendroglioma(1), germinal center b cell(1), lobullar carcinoma in situ(1), |
| | lymphoma(1), oligodendroglioma(1), pooled germ cell tumors(8) |
| 5011 | breast cancer(1), normal prostate(5), seminal vesicles(1) |
| 5040 | bone marrow(18), brain(1), hematopoietic from aml patient(1), liver(2), normal cortical |
| | stroma(1) |
| 5089 | parotid gland(1) |
| 5117 | adenocarcinoma(2), breast(2), carcinoid(1), colon(3), colon tumor rer+(1), epithelium (cell |
| | line)(3), moderately differentiated adenocarcinoma(1), moderately-differentiated |
| | adenocarcinoma(2), normal prostate(3), normal prostatic epithelial cells(2), placenta(1), |
| | prostate(3), small cell carcinoma(1), squamous cell carcinoma(2), squamous cell carcinoma |
| | from base of tongue(1) |
| 5162 | hippocampus(1), schizophrenic brain s-11 frontal lobe(1) |
| 5167 | colon(3), colon tumor rer+(2), pooled germ cell tumors(1) |
| 5214 | kidney(1), ovarian tumor(1), pituitar gland(1), total brain(2) |
| 5230 | 2 pooled tumors (clear cell type)(5), placenta(1), breast(1), breast tumor(1), carcinoid(2), |
| | colon(1), colon carcinoma(1), colon mucosa(1), colon tumor rer+(4), germinal center b |
| | cell(1), kidney tumor(1), normal prostate(5), papillary serous carcinoma(1), parathyroid |
| | tumor(3), pectoral muscle (after mastectomy)(3), pooled germ cell tumors(2), senescent |
| | fibroblast(1), squamous cell carcinoma from base of tongue(1) |
| 5240 | bone(1), colon(2), frontal lobe(2), glioblastoma (pooled)(1), liver(1), ovarian tumor(1), |
| | parathyroid tumor(1), tumor, 5 pooled (see description)(1) |
| 5250 | 2 pooled tumors (clear cell type)(1), anaplastic oligodendroglioma(2), brain(5), carcinoid(1), |
| | colon(2), colon tumor rer+(1), pooled germ cell tumors(1) |
| 5262 | 2 pooled tumors (clear cell type)(1), anaplastic oligodendroglioma(2), brain(5), carcinoid(1), |
| | colon(2), colon tumor rer+(1), pooled germ cell tumors(1) |
| 5270 | ovarian tumor(1) |
| 5278 | 2 pooled tumors (clear cell type)(7), anaplastic oligodendroglioma(7), breast(1), breast |
| | tumor(4), carcinoid(3), colon(1), colon tumor rer+(2), glioblastoma (pooled)(1), liver(3), |
| | pooled germ cell tumors(6), thyroid(2) |
| 5358 | 2 pooled tumors (clear cell type)(1), anaplastic oligodendroglioma(2), brain(2), colon(2), |
| | germinal center b cell(4), melanocyte(1), normal prostate(3), parathyroid tumor(2), pectoral |
| | muscle (after mastectomy)(3), pooled germ cell tumors(4), senescent fibroblast(2) |
| 5494 | 2 pooled tumors (clear cell type)(2), cd34+, cd38− from normal bone marrow donor(2), |
| | anaplastic oligodendroglioma(2), brain(2), colon(2), colon tumor rer+(1), early stage |
| | papillary serous carcinoma(1), germinal center b cell(3), glioblastoma (pooled)(3), |
| | moderately-differentiated adenocarcinoma(1), normal prostate(1), normal prostatic epithelial |
| | cells(1), omentum(1), ovarian tumor(4), ovary(1), parathyroid tumor(3), pectoral muscle |
| | (after mastectomy)(1), pooled germ cell tumors(1), senescent fibroblast(2), stem cell |
| | 34+/38+(1), synovial membrane(1), synovial sarcoma(1), tumor(1) |
| 5499 | 2 pooled tumors (clear cell type)(1), brain(2), pancreatic islet(2) |
| 5533 | 2 pooled tumors (clear cell type)(2), anaplastic oligodendroglioma(3), breast(1), carcinoid(1), |
| | germinal center b cell(2), glioblastoma (pooled)(2), pooled germ cell tumors(2), senescent |
| | fibroblast(1), small cell carcinoma(2) |
| 5563 | colon(3), kidney(1), liver(1), neuroepithelial cells(1), normal prostatic epithelial cells(1), |
| | ovarian tumor(1), senescent fibroblast(1), total brain(1) |
| 5691 | 2 pooled high-grade transitional cell tumors(1), 2 pooled tumors (clear cell type)(3), |
| | adenocarcinoma(3), anaplastic oligodendroglioma(2), carcinoid(3), colon(2), germinal center |
| | b cell(1), glioblastoma (pooled)(3), medulloblastoma(1), ovarian tumor(1), parathyroid |
| | tumor(2), pectoral muscle (after mastectomy)(2), prostate(1), three pooled meningiomas(1), |
| | total brain(1) |
| 5748 | bone(2), anaplastic oligodendroglioma(5), breast(1), carcinoid(1), colon tumor rer+(1), |
| | frontal lobe(2), germinal center b cell(1), glioblastoma (pooled)(1), ovarian tumor(4), |
| | parathyroid tumor(2), pooled germ cell tumors(1), senescent fibroblast(1), tumor, 5 pooled |
| | (see description)(1) |
| 5806 | 2 pooled tumors (clear cell type)(4), female, 19 years old, normal leg muscle(2), alveolar |
| | rhabdomyosarcoma(1), anaplastic oligodendroglioma(4), breast(1), carcinoid(3), germinal |
| | center b cell(1), glioblastoma (pooled)(2), normal prostate(1) |
| 5816 | anaplastic oligodendroglioma(1), frontal lobe(4) |
| 5824 | 2 pooled tumors (clear cell type)(2), b-cell, chronic lymphotic leukemia(2), |
| | adenocarcinoma(1), anaplastic oligodendroglioma(3), blood(1), breast(1), carcinoid(9), |
| | cerebellum(1), colon(3), fibrotheoma(1), follicular lymphoma(1), germinal center b cell(2), |
| | glioblastoma (pooled)(2), kidney tumor(1), low-grade prostatic neoplasia(2), normal prostatic |
| | epithelial cells(1), ovarian tumor(2), parathyroid tumor(7), pooled germ cell tumors(1), |
| | senescent fibroblast(6), thyroid(1) |
| 5861 | adipose tissue, white(2), bone marrow from femur(1), carcinoid(1), epithelium(1), normal |
| | prostatic epithelial cells(1), pectoral muscle (after mastectomy)(1) |
| 5885 | 2 pooled tumors (clear cell type)(1), female, 19 years old, normal leg muscle(1), anaplastic |
| | oligodendroglioma(5), bone marrow stroma(1), colon tumor rer+(1), germinal center b |
| | cell(5), glioblastoma (pooled)(1), kidney tumor(1), melanocyte(2), moderately differentiated |
| | adenocarcinoma(1), moderately-differentiated adenocarcinoma(1), normal prostate(1), |
| | normal prostatic epithelial cells(2), ovarian tumor(2), parathyroid tumor(1), senescent |
| | fibroblast(1), three pooled meningiomas(1), tumor(1) |
| 5947 | 2 pooled tumors (clear cell type)(4), adenocarcinoma(1), colon tumor(3), intestine(1), liver(1) |
| 5970 | b-cell, chronic lymphotic leukemia(1), carcinoid(1), germinal center b cell(2), pooled germ |
| | cell tumors(3) |
| 5974 | blood(5), bone marrow(1), liver(4), reticulocyte(1) |
| 5983 | 2 pooled tumors (clear cell type)(1), adenocarcinoma(1), carcinoid(1), germinal center b |
| | cell(4), medulloblastoma(1), pooled germ cell tumors(1), tumor, 5 pooled (see description)(1) |
| 5985 | 2 pooled tumors (clear cell type)(3), b-cell, chronic lymphotic leukemia(2), anaplastic |
| | oligodendroglioma(5), breast(4), carcinoid(2), colon tumor, rer+(1), germ cell tumor(1), |
| | germinal center b cell(6), invasive prostate tumor(1), lung tumor(1), metastatic prostate bone |
| | lesion(2), normal prostate(1), parathyroid tumor(2), pectoral muscle (after mastectomy)(3), |
| | pooled germ cell tumors(1), senescent fibroblast(4), stroma(1), thyroid(1), tumor, 5 pooled |
| | (see description)(2) |
| 6011 | brain(5), carcinoid(1), frontal lobe(1), lung carcinoma(1), retina(1) |
| 6080 | 2 pooled tumors (clear cell type)(8), ewing's sarcoma(2), heart(1), adenocarcinoma(1), |
| | alveolar rhabdomyosarcoma(6), anaplastic oligodendroglioma(5), aorta(1), breast(4), bulk |
| | germ cell seminoma(2), colon(3), germinal center b cell(2), glioblastoma (pooled)(1), |
| | kidney(2), lung carcinoma(1), metastatic prostate bone lesion(4), normal prostate(1), normal |
| | prostatic epithelial cells(1), oligodendroglioma(1), ovary(2), parathyroid tumor(5), pectoral |
| | muscle (after mastectomy)(19), pooled germ cell tumors(1), prostate(1), senescent |
| | fibroblast(4), tumor, 5 pooled (see description)(1) |
| 6081 | brain(1), germinal center b cell(1), testis(1) |
| 6108 | 2 pooled tumors (clear cell type)(1), carcinoid(1), germinal center b cell(1), parathyroid |
| | tumor(1) |
| 6159 | 2 pooled tumors (clear cell type)(2), ewing's sarcoma(1), schwannoma tumor(1), adipose |
| | tissue, white(1), adrenal adenoma(3), amygdala(1), anaplastic oligodendroglioma(1), |
| | astrocytoma(2), bone marrow stroma(3), borderline ovarian carcinoma(1), cochlea(3), colon |
| | tumor(1), epithelium (cell line)(4), frontal lobe(3), germinal center b cell(1), human |
| | pancreatic islets(1), kidney tumor(3), larynx(1), liver(1), lung carcinoma(1), lung tumor(1), |
| | normal leg muscle(1), oligodendroglioma(1), ovarian tumor(8), parathyroid tumor(1), |
| | pectoral muscle (after mastectomy)(8), prostate tumor(1), senescent fibroblast(3), small cell |
| | carcinoma(4) |
| 6231 | adenocarcinoma(2), breast(3), colon(4), colon tumor(2), endometrioid ovarian metastasis(1), |
| | epithelium (cell line)(22), frontal lobe(1), germ cell tumor(3), invasive tumor (cell line)(13), |
| | moderately differentiated adenocarcinoma(1), normal prostatic epithelial cells(1), ovarian |
| | tumor(4), ovary(1), pancreatic islet(1), placenta(trophoblast)(1), pooled germ cell tumors(2), |
| | squamous cell carcinoma(1), synovial sarcoma(1), tumor(3), tumor, 5 pooled (see |
| | description)(2), two pooled squamous cell carcinomas(2) |
| 6238 | bone(1), colon(2), ovarian tumor(2), small cell carcinoma(2), total brain(1) |
| 6252 | 2 pooled tumors (clear cell type)(1), ewing's sarcoma(6), adenocarcinoma(1), alveolar |
| | rhabdomyosarcoma(3), anaplastic oligodendroglioma(2), brain(1), breast(2), breast tumor(1), |
| | carcinoid(2), cochlea(2), germ cell tumor(1), glioblastoma (pooled)(1), kidney(1), kidney |
| | tumor(6), liposarcoma(1), lung carcinoma(1), lymphoma(1), metastatic prostate bone |
| | lesion(6), muscle(1), normal prostate(1), normal prostatic epithelial cells(1), ovarian |
| | tumor(2), ovary(4), parathyroid tumor(1), pectoral muscle (after mastectomy)(5), pooled |
| | germ cell tumors(4), prostate(1), renal celll tumor(1), senescent fibroblast(3), stem cells(1), |
| | tumor, 5 pooled (see description)(4) |
| 6290 | 2 pooled tumors (clear cell type)(1), b-cell, chronic lymphotic leukemia(1), heart(3), |
| | lymphoma(1), adenocarcinoma(1), adipose tissue, white(1), brain(3), carcinoid(5), |
| | cerebellum(5), colon(8), epithelium (cell line)(1), frontal lobe(1), germinal center b-cells(1), |
| | kidney tumor(1), medulloblastoma(1), melanoma (mewo cell line)(1), normal prostate(1), |
| | omentum(1), ovarian tumor(1), pancreatic islet(1), placenta(3), pooled frontal lobe(1), |
| | retina(1), retinal fovaea(1), schizophrenic brain s-11 frontal lobe(1), senescent fibroblast(1), |
| | small cell carcinoma(3), synovial membrane(1), total brain(2) |
| 6322 | b-cell, chronic lymphotic leukemia(1), heart(4), lymphoma(1), adipose tissue, white(1), |
| | brain(3), carcinoid(4), cerebellum(5), colon(8), epithelium (cell line)(1), frontal lobe(1), |
| | germinal center b-cells(1), kidney tumor(1), melanoma (mewo cell line)(1), omentum(1), |
| | ovarian tumor(1), pancreatic islet(1), placenta(3), pooled frontal lobe(1), retinal fovaea(1), |
| | schizophrenic brain s-11 frontal lobe(1), senescent fibroblast(1), small cell carcinoma(3), |
| | synovial membrane(1), total brain(2) |
| 6329 | b-cell, chronic lymphotic leukemia(3), heart(3), lymphoma(2), adenocarcinoma(6), adipose |
| | tissue, white(1), adrenal adenoma(2), anaplastic oligodendroglioma(2), bone marrow |
| | stroma(1), brain(2), breast(1), carcinoid(7), cerebellum(5), colon(8), epithelium (cell line)(1), |
| | frontal lobe(1), germ cell tumor(2), germinal center b-cells(1), kidney tumor(1), larynx(1), |
| | lung tumor(1), medulloblastoma(1), melanoma (mewo cell line)(1), metastatic melanoma to |
| | bowel(1), moderately differentiated adenocarcinoma(1), normal prostate(2), omentum(1), |
| | ovarian tumor(1), pancreatic islet(2), papillary serous ovarian metastasis(1), parathyroid |
| | tumor(1), pectoral muscle (after mastectomy)(1), placenta(2), pooled frontal lobe(1), pooled |
| | germ cell tumors(3), prostate(1), retinal fovaea(1), schizophrenic brain s-11 frontal lobe(1), |
| | senescent fibroblast(3), small cell carcinoma(5), squamous cell carcinoma(1), synovial |
| | membrane(1), total brain(2), tumor(1), tumor, 5 pooled (see description)(1), two pooled |
| | squamous cell carcinomas(1) |
| 6334 | 2 pooled tumors (clear cell type)(3), b-cell, chronic lymphotic leukemia(2), anaplastic |
| | oligodendroglioma(9), glioblastoma (pooled)(3), kidney(1), normal prostate(3), ovarian |
| | tumor(1), senescent fibroblast(2) |
| 6345 | testis(3) |
| 6350 | 2 pooled tumors (clear cell type)(2), b-cell, chronic lymphotic leukemia(1), bone(1), |
| | adenocarcinoma(1), anaplastic oligodendroglioma(10), breast(1), breast tumor(1), colon(2), |
| | glioblastoma (pooled)(6), kidney(1), lung carcinoma(1), medulloblastoma(1), metastatic |
| | prostate bone lesion(1), normal prostate(1), pectoral muscle (after mastectomy)(4), pooled |
| | germ cell tumors(2), senescent fibroblast(2), squamous cell carcinoma(1), testis(1), tumor(1), |
| | tumor, 5 pooled (see description)(1) |
| 6358 | 2 pooled tumors (clear cell type)(2), anaplastic oligodendroglioma(4), brain(4), carcinoid(2), |
| | colon(3), colon tumor rer+(1), germinal center b cell(4), glioblastoma (pooled)(2), normal |
| | prostate(1), normal prostatic epithelial cells(1), parathyroid tumor(2), pectoral muscle (after |
| | mastectomy)(1), senescent fibroblast(3) |
| 6384 | epithelium(1), meningioma(1), parathyroid tumor(2), senescent fibroblast(1) |
| 6400 | blood(2), brain(2), colon(6), parathyroid tumor(1), tumor(1) |
| 6431 | adenocarcinoma(1) |
| 6453 | pooled germ cell tumors(3) |
| 6636 | 2 pooled tumors (clear cell type)(1), ewing's sarcoma(2), adenocarcinoma(3), anaplastic |
| | oligodendroglioma(3), breast tumor(1), carcinoid(4), colon(2), colon tumor(1), colon tumor |
| | rer+(2), frontal lobe(3), germinal center b cell(6), kidney tumor(1), ovarian tumor(4), |
| | parathyroid tumor(4), pectoral muscle (after mastectomy)(10), placenta(1), pooled germ cell |
| | tumors(2), senescent fibroblast(1), squamous cell carcinoma from base of tongue(1), tumor, 5 |
| | pooled (see description)(1) |
| 6688 | b-cell, chronic lymphotic leukemia(3), alveolar rhabdomyosarcoma(1), anaplastic |
| | oligodendroglioma(1), breast(2), carcinoid(1), colon(1), colon tumor(1), four pooled pituitary |
| | adenomas(1), germinal center b cell(2), glioblastoma (pooled)(1), kidney tumor(2), |
| | moderately-differentiated adenocarcinoma(1), muscle(2), pectoral muscle (after |
| | mastectomy)(1), pooled germ cell tumors(3), senescent fibroblast(3), synovial sarcoma(1), |
| | testis(1), tumor, 5 pooled (see description)(3) |
| 6727 | frontal lobe(1), schizophrenic brain s-11 frontal lobe(1) |
| 6865 | frontal lobe(4), germinal center b cell(1), muscle(1), ovarian tumor(2), pectoral muscle (after |
| | mastectomy)(3), senescent fibroblast(1), small cell carcinoma(1), thyroid(1) |
| 6892 | 2 pooled tumors (clear cell type)(5), adenocarcinoma(1), anaplastic oligodendroglioma(5), |
| | brain(5), breast(3), breast tumor(1), carcinoid(5), cerebellum(1), colon(3), colon tumor |
| | rer+(2), frontal lobe(5), germinal center b cell(3), glioblastoma (pooled)(2), moderately- |
| | differentiated adenocarcinoma(1), normal prostate(3), ovary(2), parathyroid tumor(3), |
| | pectoral muscle (after mastectomy)(1), placenta(1), pooled germ cell tumors(5), senescent |
| | fibroblast(3), tumor(1), tumor, 5 pooled (see description)(1) |
| 7000 | anaplastic oligodendroglioma(2), frontal lobe(2) |
| 7041 | germinal center b cell(1), total brain(2) |
| 7533 | 2 pooled tumors (clear cell type)(1), b-cell, chronic lymphotic leukemia(1), schwannoma |
| | tumor(1), anaplastic oligodendroglioma(1), astrocytoma(1), breast(1), cochlea(1), colon |
| | tumor rer+(2), germ cell tumor(1), germinal center b cell(2), glioblastoma (pooled)(3), |
| | hepatoma(4), medulloblastoma(1), metastatic prostate bone lesion(1), moderately- |
| | differentiated adenocarcinoma(1), ovarian tumor(1), prostate tumor(1), senescent |
| | fibroblast(1), squamous cell carcinoma(1), three pooled meningiomas(1) |
| 7535 | b-cell, chronic lymphotic leukemia(1), schwannoma tumor(1), anaplastic |
| | oligodendroglioma(1), astrocytoma(1), breast(1), cochlea(1), colon tumor rer+(2), germ cell |
| | tumor(1), germinal center b cell(2), glioblastoma (pooled)(3), hepatoma(4), |
| | medulloblastoma(1), metastatic prostate bone lesion(1), moderately-differentiated |
| | adenocarcinoma(1), ovarian tumor(1), senescent fibroblast(1), squamous cell carcinoma(1) |
| 7697 | colon(1), invasive adenocarcinoma(3), liver(1), moderately-differentiated adenocarcinoma(1) |
| 8009 | alveolar rhabdomyosarcoma(2), anaplastic oligodendroglioma(1), carcinoid(9), colon(1), |
| | colon tumor rer+(1), germinal center b cell(2), glioblastoma (pooled)(1), normal prostate(1), |
| | ovary(2), pooled germ cell tumors(2), thyroid(2), tumor(1) |
| 8078 | frontal lobe(2) |
| 8079 | b-cell, chronic lymphotic leukemia(2), bone(1), anaplastic oligodendroglioma(1), colon(1), |
| | pooled germ cell tumors(3) |
| 8097 | colon(1) |
| 8166 | 2 pooled tumors (clear cell type)(5), adenocarcinoma(6), anaplastic oligodendroglioma(3), |
| | breast tumor(1), carcinoid(1), colon(1), epithelium (cell line)(1), glioblastoma (pooled)(2), |
| | lung tumor(2), metastatic melanoma to bowel(1), moderately-differentiated |
| | adenocarcinoma(1), normal prostate(1), ovary(1), papillary serous ovarian metastasis(1), |
| | parathyroid tumor(1), pooled germ cell tumors(4), renal cell tumor(1), squamous cell |
| | carcinoma(5), synovial sarcoma(1), tumor(3), tumor, 5 pooled (see description)(3) |
| 8341 | 2 pooled tumors (clear cell type)(1), anaplastic oligodendroglioma(2), germinal center b |
| | cell(3), glioblastoma (pooled)(1), normal prostate(1), oligodendroglioma(1), pectoral muscle |
| | (after mastectomy)(2), tumor(1) |
| 8534 | germinal center b cell(4) |
| 8666 | 2 pooled high-grade transitional cell tumors(1), 2 pooled tumors (clear cell type)(5), b-cell, |
| | chronic lymphotic leukemia(2), adenocarcinoma(3), alveolar rhabdomyosarcoma(3), |
| | amygdala(1), anaplastic oligodendroglioma(3), blood(2), breast(1), carcinoid(3), colon(3), |
| | germinal center b cell(4), muscle(1), normal prostate(1), ovarian tumor(6), parathyroid |
| | tumor(2), pectoral muscle (after mastectomy)(4), pheochromocytoma(1), placenta(1), |
| | senescent fibroblast(11), two pooled squamous cell carcinomas(1) |
| 8671 | 2 pooled high-grade transitional cell tumors(1), 2 pooled tumors (clear cell type)(3), b-cell, |
| | chronic lymphotic leukemia(1), adenocarcinoma(6), alveolar rhabdomyosarcoma(3), |
| | amygdala(2), anaplastic oligodendroglioma(2), blood(2), breast(2), carcinoid(1), colon(2), |
| | germinal center b cell(5), liver cancer(1), metastatic prostate bone lesion(1), muscle(1), |
| | normal prostate(1), ovarian tumor(5), parathyroid tumor(2), pectoral muscle (after |
| | mastectomy)(5), placenta(2), pooled germ cell tumors(1), renal cell tumor(1), senescent |
| | fibroblast(8) |
| 8968 | b-cell, chronic lymphotic leukemia(1), neuroepithelial cells(1), total brain(1) |
| 9406 | brain(2), colon(1), tumor(1) |
| 9407 | brain(2), colon(1), tumor(1) |
| 9668 | 2 pooled tumors (clear cell type)(8), adenocarcinoma(1), anaplastic oligodendroglioma(1), |
| | breast carcinoma in situ(3), cerebellum(1), oligodendroglioma(1), papillary serous |
| | carcinoma(2), parathyroid tumor(26), pooled germ cell tumors(1) |
| 9679 | ovarian tumor(3), parathyroid tumor(1), uterus(1) |
| 9755 | 2 pooled tumors (clear cell type)(1), adenocarcinoma(1), carcinoid(1), ovarian tumor(1), |
| | pooled germ cell tumors(3), senescent fibroblast(2), total brain(2) |
| 9868 | brain(1), senescent fibroblast(1) |
| 10044 | 2 pooled high-grade transitional cell tumors(1), 2 pooled tumors (clear cell type)(3), b-cell, |
| | chronic lymphotic leukemia(1), adenocarcinoma(6), alveolar rhabdomyosarcoma(3), |
| | amygdala(2), anaplastic oligodendroglioma(2), blood(1), breast(2), carcinoid(1), colon(2), |
| | germinal center b cell(5), liver cancer(1), metastatic prostate bone lesion(1), normal |
| | prostate(1), osteosarcoma(1), ovarian tumor(5), parathyroid tumor(2), pectoral muscle (after |
| | mastectomy)(5), placenta(2), pooled germ cell tumors(1), renal cell tumor(1), senescent |
| | fibroblast(8) |
| 10322 | 2 pooled tumors (clear cell type)(9), b-cell, chronic lymphotic leukemia(2), anaplastic |
| | oligodendroglioma(4), breast(1), carcinoid(3), colon(3), colon tumor rer+(1), germinal center |
| | b cell(1), glioblastoma (pooled)(4), kidney tumor(1), low-grade prostatic neoplasia(1), |
| | metastatic melanoma to bowel(1), normal prostate(2), ovary bulk tumor(1), parathyroid |
| | tumor(3), pectoral muscle (after mastectomy)(3), pooled germ cell tumors(2), senescent |
| | fibroblast(2), small cell carcinoma(2), synovial sarcoma(2), two pooled squamous cell |
| | carcinomas(3) |
| 10526 | 2 pooled tumors (clear cell type)(2), ewing's sarcoma(4), adenocarcinoma(1), adipose tissue, |
| | white(1), alveolar rhabdomyosarcoma(3), anaplastic oligodendroglioma(5), brain(1), |
| | breast(4), breast tumor(1), carcinoid(2), colon(10), epithelium (cell line)(1), frontal lobe(7), |
| | germ cell tumor(1), glioblastoma (pooled)(1), invasive prostate tumor(1), kidney tumor(8), |
| | lymphoma(4), metastatic prostate bone lesion(2), muscle(3), normal prostate(2), normal |
| | prostatic epithelial cells(3), ovarian tumor(1), ovary(2), parathyroid tumor(4), pectoral |
| | muscle (after mastectomy)(26), placenta(1), pooled germ cell tumors(6), prostate(9), |
| | senescent fibroblast(6), small cell carcinoma(1), tumor, 5 pooled (see description)(2) |
| 10584 | 2 pooled tumors (clear cell type)(2), b-cell, chronic lymphotic leukemia(4), anaplastic |
| | oligodendroglioma(6), carcinoid(2), colon(3), colon tumor rer+(4), colon tumor, rer+(2), |
| | ovarian tumor(4) |
| 10650 | pooled germ cell tumors(2) |
| 10739 | 2 pooled tumors (clear cell type)(5), ewing's sarcoma(1), liver(1), alveolar |
| | rhabdomyosarcoma(1), anaplastic oligodendroglioma(7), breast(3), breast tumor(1), |
| | carcinoid(9), colon(5), colon tumor rer+(3), frontal lobe(8), germinal center b cell(3), |
| | glioblastoma (pooled)(11), invasive prostate tumor(1), kidney(1), metastatic prostate bone |
| | lesion(1), moderately-differentiated adenocarcinoma(1), normal prostate(2), normal prostatic |
| | epithelial cells(1), parathyroid tumor(4), pectoral muscle (after mastectomy)(6), placenta(1), |
| | pooled germ cell tumors(1), tumor(1), tumor, 5 pooled (see description)(1) |
| 10743 | 2 pooled tumors (clear cell type)(5), ewing's sarcoma(1), liver(1), schwannoma tumor(1), |
| | alveolar rhabdomyosarcoma(1), anaplastic oligodendroglioma(7), breast(3), breast tumor(1), |
| | carcinoid(10), colon(5), colon tumor rer+(3), germinal center b cell(3), glioblastoma |
| | (pooled)(11), invasive prostate tumor(1), kidney(1), liver(1), metastatic prostate bone |
| | lesion(1), moderately-differentiated adenocarcinoma(1), muscle(1), normal prostate(2), |
| | normal prostatic epithelial cells(1), ovarian tumor(4), parathyroid tumor(4), pectoral muscle |
| | (after mastectomy)(6), placenta(1), pooled germ cell tumors(2), thyroid(1), tumor(1), tumor, |
| | 5 pooled (see description)(1) |
| 10744 | heart(2), four pooled pituitary adenomas(1), frontal lobe(3), glioblastoma (pooled)(1), |
| | liver(1), moderately-differentiated adenocarcinoma(1), peripheral blood(1), retina(1) |
| 10880 | 2 pooled tumors (clear cell type)(3), ewing's sarcoma(1), adenocarcinoma(3), brain(1), |
| | carcinoid(8), germinal center b cell(1), glioblastoma (pooled)(5), kidney tumor(1), normal |
| | prostate(3), oligodendroglioma(1), parathyroid tumor(11), senescent fibroblast(4), three |
| | pooled meningiomas(1), total brain(3) |
| 10942 | 2 pooled tumors (clear cell type)(8), adenocarcinoma(1), anaplastic oligodendroglioma(1), |
| | breast carcinoma in situ(3), cerebellum(1), oligodendroglioma(1), papillary serous |
| | carcinoma(2), parathyroid tumor(26), pooled germ cell tumors(1) |
| 11019 | epidermis(1), ewing's sarcoma(2), heart(1), schwannoma tumor(1), adenocarcinoma(5), |
| | alveolar rhabdomyosarcoma(3), amygdala(1), brain(4), carcinoid(1), colon mucosa(1), |
| | endometrioid ovarian metastasis(1), epithelium (cell line)(2), frontal lobe(1), germ cell |
| | tumor(3), heart(1), invasive prostate tumor(2), kidney(2), kidney tumor(2), liposarcoma(1), |
| | liver(24), mantle cell lymphoma(3), medulloblastoma(1), metastatic prostate bone lesion(6), |
| | moderately-differentiated adenocarcinoma(1), muscle(1), normal prostatic epithelial cells(5), |
| | ovary(1), papillary serous ovarian metastasis(1), parathyroid tumor(1), placenta(2), pooled |
| | germ cell tumors(2), prostate(1), thyroid(1), tumor(1), tumor, 5 pooled (see description)(1), |
| | uterus(4) |
| 11278 | 2 pooled tumors (clear cell type)(4), bone(4), heart(2), anaplastic oligodendroglioma(4), |
| | carcinoid(3), colon tumor rer+(1), epithelium (cell line)(1), frontal lobe(12), kidney(2), |
| | liposarcoma(1), liver(6), lung carcinoma(1), muscle(6), normal prostate(2), ovary(2), |
| | parathyroid tumor(6), pectoral muscle (after mastectomy)(3), schizophrenic brain s-11 frontal |
| | lobe(1), senescent fibroblast(6), small cell carcinoma(4), synovial membrane(1) |
| 11342 | 2 pooled tumors (clear cell type)(3), blood(1), germinal center b cell(4), normal |
| | epithelium(1), pooled germ cell tumors(1), tumor, 5 pooled (see description)(1) |
| 11735 | adenocarcinoma(1), breast(2), colon(1), frontal lobe(2), placenta(1), pooled germ cell |
| | tumors(8) |
| 12039 | testis(1) |
| 12043 | spleen(1) |
| 12048 | 2 pooled tumors (clear cell type)(1), anaplastic oligodendroglioma(1), brain(2), colon tumor |
| | rer+(2), spleen(2) |
| 12098 | astrocytoma(1), ovarian tumor(1) |
| 12202 | 2 pooled tumors (clear cell type)(2), adenocarcinoma(1), cochlea(1), germinal center b |
| | cell(2), pituitary(1) |
| 12243 | alveolar rhabdomyosarcoma(2), anaplastic oligodendroglioma(1), carcinoid(9), colon(1), |
| | colon tumor rer+(1), germinal center b cell(2), glioblastoma (pooled)(2), metastatic prostate |
| | bone lesion(1), normal prostate(2), ovary(2), pooled germ cell tumors(2), thyroid(2), |
| | tumor(1) |
| 12263 | anaplastic oligodendroglioma(1), carcinoid(1), germinal center b cell(2), invasive |
| | adenocarcinoma(1), liver and spleen(2), papillary serous carcinoma(1) |
| 12490 | 2 pooled tumors (clear cell type)(3), b-cell, chronic lymphotic leukemia(1), ewing's |
| | sarcoma(1), adenocarcinoma(2), anaplastic oligodendroglioma(1), germinal center b cell(2), |
| | kidney tumor(1), larynx(1), mantle cell lymphoma(1), medulloblastoma(1), melanocyte(2), |
| | moderately-differentiated adenocarcinoma(1), ovarian tumor(12), parathyroid tumor(2), |
| | pooled germ cell tumors(4), prostate(2), tumor, 5 pooled (see description)(2) |
| 12604 | 2 pooled tumors (clear cell type)(12), b-cell, chronic lymphotic leukemia(4), bone(1), ewing's |
| | sarcoma(1), adenocarcinoma(1), anaplastic oligodendroglioma(10), carcinoid(3), colon(3), |
| | colon tumor rer+(1), germinal center b cell(1), glioblastoma (pooled)(3), invasive prostate |
| | tumor(1), kidney(1), larynx(1), muscle(1), normal leg muscle(1), normal prostate(3), ovarian |
| | tumor(1), parathyroid tumor(12), pectoral muscle (after mastectomy)(28), pooled germ cell |
| | tumors(3), prostate(3), renal cell tumor(1), senescent fibroblast(12), skeletal muscle(1) |
| 12657 | adenocarcinoma(1), carcinoid(2), colon tumor, rer+(2) |
| 12788 | 2 pooled tumors (clear cell type)(3), adenocarcinoma(1), adrenal adenoma(1), alveolar |
| | rhabdomyosarcoma(1), carcinoid(1), colon(1), colon tumor rer+(1), germinal center b cell(1), |
| | glioblastoma (pooled)(2), ovary(1), parathyroid tumor(1), senescent fibroblast(2), tumor, 5 |
| | pooled (see description)(1) |
| 12901 | anaplastic oligodendroglioma(2), carcinoid(1), germinal center b cell(6), invasive tumor (cell |
| | line)(1), parathyroid tumor(1) |
| 12907 | adipose tissue, white(1), carcinoid(1), parathyroid tumor(2), pooled frontal lobe(1), tumor, 5 |
| | pooled (see description)(1) |
| 13013 | invasive tumor (cell line)(1), parathyroid tumor(3) |
| 13202 | 2 pooled tumors (clear cell type)(1), b-cell, chronic lymphotic leukemia(3), breast(1), breast |
| | tumor(1), carcinoid(2), colon tumor rer+(1), colon tumor, rer+(1), germinal center b cell(1), |
| | kidney tumor(1), lung carcinoma(1), lung tumor(1), meningioma(2), moderately |
| | differentiated adenocarcinoma(1), normal prostate(3), ovarian tumor(1), squamous cell |
| | carcinoma(1), tumor, 5 pooled (see description)(1) |
| 13229 | cochlea(1), frontal cortex(1), pooled germ cell tumors(1) |
| 13256 | 2 pooled tumors (clear cell type)(2), adenocarcinoma(2), anaplastic oligodendroglioma(1), |
| | parathyroid tumor(1), pooled germ cell tumors(3) |
| 13285 | 2 pooled tumors (clear cell type)(6), carcinoid(1), colon tumor rer+(1), kidney(1), liver(1) |
| 26710 | 2 pooled tumors (clear cell type)(1), pooled germ cell tumors(1) |
| 27273 | anaplastic oligodendroglioma(1), cerebellum(1), germinal center b cell(2), moderately- |
| | differentiated adenocarcinoma(1), pectoral muscle (after mastectomy)(2) |
| 27301 | astrocytoma(2), ovarian tumor(1) |
| 27336 | 2 pooled tumors (clear cell type)(2), b-cell, chronic lymphotic leukemia(6), |
| | adenocarcinoma(1), anaplastic oligodendroglioma(9), carcinoid(6), colon(1), colon tumor |
| | rer+(1), colon tumor, rer+(6), frontal lobe(1), germinal center b cell(2), glioblastoma |
| | (pooled)(1), pooled germ cell tumors(4), senescent fibroblast(2) |
| 27361 | colon(1) |
| 27374 | b-cell, chronic lymphotic leukemia(1), pooled germ cell tumors(5) |
| 27627 | 2 pooled tumors (clear cell type)(1), anaplastic oligodendroglioma(1), frontal lobe(1), |
| | germinal center b cell(1), glioblastoma (pooled)(2), ovarian tumor(1) |
| 27697 | skeletal muscle(2), thyroid(1) |
| 27877 | carcinoid(1), colon(1), germinal center b cell(1) |
| 29469 | frontal lobe(4), germinal center b cell(1), ovarian tumor(2), pectoral muscle (after |
| | mastectomy)(3), senescent fibroblast(1), small cell carcinoma(1), thyroid(1) |
| 29557 | ewing's sarcoma(1), larynx(1), medulloblastoma(1), moderately-differentiated |
| | adenocarcinoma(1), ovarian tumor(7), parathyroid tumor(2), pooled germ cell tumors(1), |
| | prostate(1) |
| 29673 | heart(1), colon tumor(1), kidney tumor(1), lung tumor(3), lymphoid(2), moderately |
| | differentiated adenocarcinoma(1), muscle(1), ovarian tumor(3), squamous cell carcinoma(1) |
| 29814 | brain(2), colon(1), pancreatic islet(1) |
| 30218 | small cell carcinoma(2) |
| 30446 | adenocarcinoma(2), adrenal adenoma(3), colon tumor(1), glioblastoma (pooled)(2), kidney |
| | tumor(1), ovarian tumor(1), parathyroid tumor(1), prostate(1), small cell carcinoma(1) |
| 30583 | carcinoid(1), ovarian tumor(1), pooled germ cell tumors(2) |
| 30719 | 2 pooled tumors (clear cell type)(3), b-cell, chronic lymphotic leukemia(1), breast(2), |
| | colon(1), colon tumor, rer+(1), germinal center b cell(1), glioblastoma (pooled)(3), liver(2), |
| | normal prostate(2), pooled germ cell tumors(10) |
| 31356 | pooled germ cell tumors(1) |
| 31422 | 2 pooled tumors (clear cell type)(1), b-cell, chronic lymphotic leukemia(2), bone(1), |
| | anaplastic oligodendroglioma(11), bone marrow stroma(1), brain(2), breast(1), carcinoid(20), |
| | colon(6), colon tumor, rer+(1), germinal center b cell(2), glioblastoma (pooled)(3), kidney |
| | tumor(1), larynx(1), medulloblastoma(1), normal prostate(1), ovarian tumor(1), parathyroid |
| | tumor(1), pectoral muscle (after mastectomy)(2), pooled germ cell tumors(2), senescent |
| | fibroblast(5), tumor(1), tumor, 5 pooled (see description)(3) |
| 31554 | 2 pooled tumors (clear cell type)(1), carcinoid(1), germinal center b cell(4) |
| 31627 | 2 pooled tumors (clear cell type)(1), alveolar rhabdomyosarcoma(1), anaplastic |
| | oligodendroglioma(2), frontal lobe(7), normal prostate(1), oligodendroglioma(1), parathyroid |
| | tumor(1), pectoral muscle (after mastectomy)(3), pooled germ cell tumors(1) |
| 31744 | adenocarcinoma(1) |
| 31790 | ovarian tumor(1), synovial membrane(1) |
| 32102 | 2 pooled tumors (clear cell type)(1), b-cell, chronic lymphotic leukemia(2), heart(3), |
| | lymphoma(2), adenocarcinoma(6), adipose tissue, white(1), anaplastic oligodendroglioma(5), |
| | brain(2), carcinoid(9), cerebellum(5), colon(7), epithelium (cell line)(1), frontal lobe(4), germ |
| | cell tumor(1), glioblastoma (pooled)(3), juvenile granulosa tumor(1), kidney tumor(2), |
| | medulloblastoma(1), melanoma (mewo cell line)(1), metastatic melanoma to bowel(1), |
| | moderately-differentiated adenocarcinoma(1), normal epithelium(1), normal prostate(3), |
| | omentum(1), ovarian tumor(2), pancreatic islet(1), parathyroid tumor(4), pectoral muscle |
| | (after mastectomy)(2), placenta(1), pooled frontal lobe(1), pooled germ cell tumors(1), retinal |
| | fovaea(1), schizophrenic brain s-11 frontal lobe(1), senescent fibroblast(5), small cell |
| | carcinoma(4), synovial membrane(1), total brain(2), tumor, 5 pooled (see description)(1), two |
| | pooled squamous cell carcinomas(2) |
| 32473 | 2 pooled tumors (clear cell type)(3), heart(1), adipose tissue, white(1), alveolar |
| | rhabdomyosarcoma(3), anaplastic oligodendroglioma(3), breast(2), colon(2), colon tumor |
| | rer+(1), invasive prostate tumor(1), kidney(1), metastatic prostate bone lesion(1), normal |
| | prostate(1), pectoral muscle (after mastectomy)(22), pooled germ cell tumors(2), senescent |
| | fibroblast(1) |
| 32475 | 2 pooled tumors (clear cell type)(3), heart(1), adipose tissue, white(1), alveolar |
| | rhabdomyosarcoma(3), anaplastic oligodendroglioma(3), breast(2), colon(2), colon tumor |
| | rer+(1), kidney(1), metastatic prostate bone lesion(1), normal prostate(1), pectoral muscle |
| | (after mastectomy)(22), pooled germ cell tumors(2), senescent fibroblast(1) |
| 33712 | 2 pooled tumors (clear cell type)(1), adenocarcinoma(2), anaplastic oligodendroglioma(4), |
| | brain frontal cortex(2), breast(1), germinal center b cell(4), juvenile granulosa tumor(1), |
| | normal prostatic epithelial cells(2), ovarian tumor(1), three pooled meningiomas(2) |
| 35005 | bone(1), pooled germ cell tumors(1) |
| 35185 | spinal cord(1) |
| 35326 | brain(1), breast(2), carcinoid(1), colon(1), germinal center b cell(2), human pancreatic |
| | islets(1), liver(1), melanocyte(1), ovarian tumor(1), placenta(1), total brain(1) |
| 37337 | anaplastic oligodendroglioma(2), colon(2), glioblastoma (pooled)(3), juvenile granulosa |
| | tumor(1), pooled germ cell tumors(3), tumor, 5 pooled (see description)(1), uterus(2) |
| 38220 | 2 pooled tumors (clear cell type)(1), b-cell, chronic lymphotic leukemia(1), |
| | adenocarcinoma(2), breast(1), carcinoid(1), frontal lobe(1), germinal center b cell(2), normal |
| | prostate(1), normal prostatic epithelial cells(1), pooled germ cell tumors(3), total brain(2) |
| 38311 | bone(1), frontal lobe(1), germinal center b cell(1), moderately differentiated |
| | adenocarcinoma(1), placenta(1) |
| 38631 | brain(1) |
| 38749 | 2 pooled tumors (clear cell type)(4), breast(1), cochlea(1), colon tumor rer+(1), liver(1), |
| | moderately differentiated adenocarcinoma(1), normal prostate(1), pancreas (with no medical |
| | abnormalities)(1), parathyroid tumor(1), prostate(1) |
| 40975 | brain(2), tumor(1) |
| 40991 | brain(2), tumor(1) |
| 44053 | 2 pooled tumors (clear cell type)(1), bone(1), total brain(1) |
| 45179 | colon(1), metastatic prostate bone lesion(1) |
| 45274 | 2 pooled tumors (clear cell type)(6), cd34+, cd38− from normal bone marrow donor(1), |
| | ewing's sarcoma(1), heart(1), lung(1), adenocarcinoma(8), adrenal adenoma(1), alveolar |
| | rhabdomyosarcoma(3), anaplastic oligodendroglioma(5), borderline ovarian carcinoma(17), |
| | brain(3), breast(4), breast carcinoma in situ(20), bronchioalveolar carcinoma(4), carcinoid(4), |
| | cerebellum(1), colon(4), colon tumor rer+(3), early stage papillary serous carcinoma(3), |
| | glioblastoma (pooled)(2), invasive adenocarcinoma(8), invasive carcinoma(2), lobullar |
| | carcinoma in situ(2), low-grade prostatic neoplasia(1), lung carcinoma(1), lung tumor(1), |
| | normal prostate(2), normal prostatic epithelial cells(1), oligodendroglioma(3), ovarian |
| | tumor(2), papillary serous carcinoma(18), papillary serous ovarian metastasis(1), parathyroid |
| | tumor(5), pectoral muscle (after mastectomy)(7), pooled germ cell tumors(2), senescent |
| | fibroblast(3), stem cell 34+/38+(2), thymus(1) |
| 46679 | 2 pooled tumors (clear cell type)(4), anaplastic oligodendroglioma(1), pectoral muscle (after |
| | mastectomy)(1), pooled germ cell tumors(1) |
| 48024 | 2 pooled tumors (clear cell type)(3), heart(1), adipose tissue, white(1), alveolar |
| | rhabdomyosarcoma(3), anaplastic oligodendroglioma(3), breast(2), colon(2), colon tumor |
| | rer+(1), invasive prostate tumor(1), kidney(1), metastatic prostate bone lesion(1), normal |
| | prostate(1), pectoral muscle (after mastectomy)(22), pooled germ cell tumors(2), senescent |
| | fibroblast(1) |
| 48548 | liver(4) |
| 48823 | 2 pooled tumors (clear cell type)(1), bone(1), alveolar rhabdomyosarcoma(2), breast(1), |
| | epithelium (cell line)(1), glioblastoma (pooled)(1), lung(1), lung tumor(1), parathyroid |
| | tumor(1), pectoral muscle (after mastectomy)(1), renal cell tumor(1) |
| 48901 | germinal center b cell(1), pooled germ cell tumors(1) |
| 49018 | anaplastic oligodendroglioma(1), brain(1), breast(1), frontal lobe (see description)(1), |
| | testis(5) |
| 49034 | adipose tissue, white(1), pancreas (with no medical abnormalities)(1) |
| 49133 | retina(1) |
| 49140 | 2 pooled tumors (clear cell type)(1), adenocarcinoma(1), carcinoid(6), colon tumor rer+(1), |
| | glioblastoma (pooled)(2), moderately differentiated adenocarcinoma(1), normal prostate(1), |
| | pancreatic islet(2), parathyroid tumor(3) |
| 49387 | colon(1), pooled germ cell tumors(1) |
| 49416 | anaplastic oligodendroglioma(1), carcinoid(3), cerebellum(2), frontal lobe(2), germinal center |
| | b cell(2), parathyroid tumor(1), retina(1), tumor(1) |
| 49493 | neuroepithelial cells(1), ovarian tumor(4), thyroid(1) |
| 49640 | 2 pooled tumors (clear cell type)(1), anaplastic oligodendroglioma(1), brain(2), colon tumor |
| | rer+(1), spleen(2) |
| 49863 | b-cell, chronic lymphotic leukemia(1), carcinoid(1), germinal center b cell(2), pooled germ |
| | cell tumors(3) |
| 49871 | b-cell, chronic lymphotic leukemia(1), carcinoid(1), germinal center b cell(2), pooled germ |
| | cell tumors(3) |
| 50185 | total brain(1) |
| 50763 | cerebral cortex(3), brain(2), colon(1), colon tumor rer+(1), ovarian tumor(1), schizophrenic |
| | brain s-11 frontal lobe(1) |
| 50982 | 2 pooled tumors (clear cell type)(2), b-cell, chronic lymphotic leukemia(3), carcinoid(1), |
| | cochlea(1), germinal center b cell(2) |
| 51130 | adenocarcinoma(1) |
| 51212 | placenta.(2) |
| 51346 | bone(1), small cell carcinoma(2) |
| 51380 | placenta(4) |
| 51400 | liver(1) |
| 51954 | germinal center b cell(1), total brain(1) |
| 52076 | normal prostatic epithelial cells(1) |

TABLE V

| SEQ ID NO. in | | |
|---|---|---|
| priority | | High |
| application | Low frequency expression | frequency expression |
| 37 | salivary gland | |
| 51 | fetal brain, fetal kidney | brain, salivary gland, |
| | | liver |
| 179 | liver | |
| 180 | liver | |
| 183 | prostate | |
| 326 | salivary gland | |
| 362 | testis | |
| 440 | brain | |
| 452 | placenta | |
| 483 | placenta | |
| 500 | salivary gland | |
| 528 | testis | |
| 573 | liver | |
| 587 | salivary gland | |
| 588 | liver | |
| 593 | fetal kidney, placenta | |
| 599 | liver | |
| 628 | testis | |
| 653 | testis | |
| 670 | testis | |
| 693 | placenta | |
| 703 | liver | |
| 746 | brain | |
| 770 | placenta | |
| 775 | testis | |
| 812 | brain | |
| 940 | liver | |
| 988 | liver | |
| 996 | testis | |
| 1036 | salivary gland, brain | |
| 1064 | brain, liver, prostate | fetal brain |
| 1151 | brain | |
| 1190 | salivary gland, prostate | |
| 1458 | stomach/intestine | |
| 1904 | prostate | |
| 2028 | brain | |
| 2368 | testis | |
| 2553 | prostate | |
| 2556 | prostate | |
| 2690 | testis | |
| 2755 | prostate | |
| 2800 | liver, testis | |
| 2843 | brain | |
| 2932 | fetal liver | |
| 3078 | prostate | |
| 3280 | liver | |
| 3326 | prostate | |
| 3387 | prostate | |
| 3439 | salivary gland | |
| 3501 | brain | |
| 3633 | brain | |
| 3678 | testis | |
| 3714 | liver | |
| 3801 | testis | |
| 3804 | salivary gland, fetal kidney | |
| 3892 | brain | |
| 3985 | stomach/intestine | |
| 4005 | liver | |
| 4063 | fetal brain | |
| 4088 | testis | |
| 4111 | brain | |
| 4126 | liver | |
| 4172 | liver | |
| 4261 | prostate | |
| 4436 | liver | |
| 4609 | fetal liver | |
| 4647 | fetal liver | |
| 4660 | fetal liver | |
| 4664 | fetal brain | |
| 4678 | fetal liver | |
| 4687 | fetal liver | |
| 4690 | fetal liver | |
| 4694 | salivary gland | |
| 4696 | salivary gland | |
| 4733 | fetal liver | |
| 4807 | fetal brain | |
| 4809 | salivary gland | |
| 4855 | prostate | |
| 4908 | brain | |
| 4943 | liver | |
| 4947 | testis | |
| 4976 | stomach/intestine | |
| 5000 | fetal kidney | |
| 5005 | fetal kidney | |
| 5011 | prostate | |
| 5040 | fetal liver | |
| 5089 | salivary gland | |
| 5117 | prostate | |
| 5141 | liver | |
| 5162 | fetal brain | |
| 5167 | salivary gland | |
| 5192 | liver | |
| 5214 | fetal brain | |
| 5250 | fetal kidney, fetal brain | |
| 5262 | brain | fetal kidney, fetal |
| | | brain |
| 5270 | fetal brain | |
| 5278 | fetal liver | |
| 5453 | fetal kidney | |
| 5494 | fetal liver | |
| 5499 | brain | |
| 5533 | fetal brain | |
| 5563 | fetal liver | |
| 5609 | placenta | |
| 5748 | placenta | |
| 5816 | fetal brain | |
| 5824 | prostate | |
| 5861 | fetal liver, prostate, fetal kidney, | |
| | salivary gland | |
| 5947 | fetal liver | |
| 5966 | placenta | |
| 5970 | fetal kidney | |
| 5974 | fetal liver | |
| 5983 | brain | |
| 5985 | fetal kidney | |
| 6080 | salivary gland | |
| 6081 | salivary gland | |
| 6108 | liver | |
| 6238 | placenta, brain | |
| 6252 | placenta | |
| 6334 | liver | |
| 6345 | brain | |
| 6358 | fetal brain | |
| 6636 | fetal brain | |
| 6688 | fetal brain | |
| 6727 | testis | |
| 6865 | salivary gland | |
| 6892 | stomach/intestine, fetal brain | |
| 7041 | liver | |
| 7533 | placenta | |
| 7535 | brain | |
| 7697 | fetal liver | |
| 7712 | testis | |
| 8078 | testis | |
| 8097 | salivary gland | |
| 8166 | placenta, fetal liver | |
| 8262 | prostate | |
| 8534 | testis | |
| 8666 | placenta | |
| 8744 | prostate | |
| 8968 | brain | |
| 8994 | testis | |
| 9297 | fetal liver | |
| 9332 | testis | |
| 9406 | prostate | |
| 9407 | prostate | |
| 9668 | testis | |
| 9755 | testis | |
| 9868 | liver | |
| 10044 | fetal liver, placenta | |
| 10526 | prostate | |
| 10650 | testis | |
| 10743 | stomach/intestine | |
| 10744 | salivary gland | |
| 10761 | prostate | |
| 10880 | fetal liver | |
| 10942 | brain | |
| 11019 | liver | |
| 11278 | salivary gland | |
| 11562 | testis | |
| 11688 | testis | |
| 11735 | fetal brain | |
| 11813 | brain | |
| 12043 | brain | |
| 12202 | testis | |
| 12220 | fetal kidney | |
| 12243 | brain, testis, liver | fetal brain, fetal liver |
| 12263 | prostate | |
| 12276 | brain | |
| 12604 | fetal kidney | |
| 12657 | fetal kidney, stomach/intestine | |
| 12788 | testis | |
| 12901 | placenta | |
| 12907 | salivary gland | |
| 13013 | fetal liver | |
| 13229 | brain | |
| 13256 | testis | |
| 13267 | salivary gland | |
| 13285 | fetal brain | fetal kidney, |
| | | stomach/intestine |
| 26638 | fetal liver | |
| 26710 | brain | |
| 26726 | fetal kidney | |
| 26786 | brain | |
| 27084 | testis | |
| 27273 | fetal kidney | |
| 27361 | liver | |
| 27374 | fetal liver | |
| 27877 | salivary gland | |
| 28413 | prostate | |
| 28517 | testis | |
| 28518 | placenta | |
| 29120 | salivary gland | |
| 29673 | fetal liver | |
| 30218 | liver | |
| 30446 | prostate | |
| 30477 | placenta | |
| 30583 | testis | |
| 30719 | fetal brain, salivary gland | |
| 31356 | prostate | |
| 31790 | brain | |
| 33130 | testis | |
| 38749 | prostate | |
| 38890 | placenta | |
| 40163 | testis | |
| 40975 | prostate | |
| 40991 | prostate | |
| 42896 | prostate | |
| 44053 | stomach/intestine, fetal kidney | |
| 45091 | stomach/intestine | |
| 45179 | prostate | |
| 45274 | fetal kidney | |
| 46679 | fetal liver | |
| 48024 | fetal kidney | |
| 48603 | liver, testis | |
| 48671 | testis | |
| 48823 | fetal kidney | |
| 48901 | salivary gland | |
| 49018 | stomach/intestine | |
| 49034 | salivary gland | |
| 49133 | liver | |
| 49261 | testis | |
| 49387 | liver | |
| 49416 | fetal brain | |
| 49426 | stomach/intestine | |
| 49493 | salivary gland | |
| 49640 | brain | |
| 49863 | brain | |
| 49871 | brain | |
| 50015 | testis | |
| 50049 | testis | |
| 50112 | testis | |
| 50185 | salivary gland | |
| 50241 | liver | |
| 50763 | brain | |
| 51130 | testis | |
| 51212 | stomach/intestine | |
| 51400 | liver | |

What is claimed is:

1. An isolated polynucleotide, said polynucleotide comprising a nucleic acid sequence encoding:
i) A polypeptide comprising the amino acid sequence shown as SEQ ID NO:305;
ii) a polypeptide comprising any one of the amino acid sequences shown as SEQ ID NOs:170-304, 306-338,456-560, 785-918; or
iii) a biologically active fragment of any of said polypeptides.

2. The polynucleotide of claim 1, wherein said polypeptide comprises a signal peptide.

3. The polynucleotide of claim 1, wherein said polypeptide is a mature protein.

4. The polynucleotide of claim 1, wherein said polynucleotide comprises any one of the nucleic acid sequences shown as SEQ ID NOs:1-169, 339-455, or 561-784.

5. The polynucleotide of claim 1, wherein said polynucleotide is operably linked to a promoter.

6. An expression vector comprising the polynucleotide of claim 5.

7. A host cell recombinant for the polynucleotide of claim 1.

8. A non-human transgenic animal comprising the host cell of claim 7.

9. A pharmaceutical composition comprising the polynucleotide of claim 1, and a pharmaceutically acceptable carrier.

10. A method of making a GENSET polypeptide, said method comprising
a) providing a population of host cells comprising the polynucleotide of claim 5; and
b) culturing said population of host cells under conditions conducive to the production of said polypeptide within said host cells.

11. The method of claim 10, further comprising purifying said polypeptide from said population of host cells.

12. An isolated polynucleotide, said polynucleotide comprising any one of the nucleic acid sequences shown as SEQ ID NOs:1-169, 339-455, or 561-784.

13. A biologically active polypeptide encoded by the polynucleotide of claim 12.

14. An isolated polypeptide or biologically active fragment thereof, said polypeptide comprising any one of the amino acid sequences shown as SEQ ID NOs:170-338, 456-560, or 785-918.

15. The polypeptide of claim 14, wherein said polypeptide comprises a signal peptide.

16. The polypeptide of claim 14, wherein said polypeptide is a mature protein.

17. An antibody that specifically binds to the polypeptide of claim 14.

18. A pharmaceutical composition comprising the polypeptide of claim 14, and a pharmaceutically acceptable carrier.

19. A method of making a GENSET polypeptide, said method comprising
a) providing a population of cells comprising a polynucleotide encoding the polypeptide of claim 14, operably linked to a promoter;
b) culturing said population of cells under conditions conducive to the production of said polypeptide within said cells; and
c) purifying said polypeptide from said population of cells.

20. A method of determining whether a GENSET gene is expressed within a mammal, said method comprising the steps of:
a) providing a biological sample from said mammal
b) contacting said biological sample with either of:
i) a polynucleotide that hybridizes under stringent conditions to the polynucleotide of claim 1; or
ii) a polypeptide that specifically binds to the polypeptide of claim 14; and
c) detecting the presence or absence of hybridization between said polynucleotide and an RNA species within said sample, or the presence or absence of binding of said polypeptide to a protein within said sample;
wherein a detection of said hybridization or of said binding indicates that said GENSET gene is expressed within said mammal.

21. The method of claim 21, wherein said polynucleotide is a primer, and wherein said hybridization is detected by detecting the presence of an amplification product comprising the sequence of said primer.

22. The method of claim 21, wherein said polypeptide is an antibody.

23. A method of determining whether a mammal has an elevated or reduced level of GENSET gene expression, said method comprising the steps of:
a) providing a biological sample from said mammal; and
b) comparing the amount of the polypeptide of claim 14, or of an RNA species encoding said polypeptide, within said biological sample with a level detected in or expected from a control sample;
wherein an increased amount of said polypeptide or said RNA species within said biological sample compared to said level detected in or expected from said control sample indicates that said mammal has an elevated level of said GENSET gene expression, and wherein a decreased amount of said polypeptide or said RNA species within said biological sample compared to said level detected in or expected from said control sample indicates that said mammal has a reduced level of said GENSET gene expression.

24. A method of identifying a candidate modulator of a GENSET polypeptide, said method comprising:
a) contacting the polypeptide of claim 14 with a test compound; and
b) determining whether said compound specifically binds to said polypeptide;
wherein a detection that said compound specifically binds to said polypeptide indicates that said compound is a candidate modulator of said GENSET polypeptide.

25. The method of claim 24, further comprising testing the biological activity of said GENSET polypeptide in the presence of said candidate modulator, wherein an alteration in the biological activity of said GENSET polypeptide in the presence of said compound in comparison to the activity in the absence of said compound indicates that the compound is a modulator of said GENSET polypeptide.

26. A method for the production of a pharmaceutical composition comprising
a) identifying a modulator of a GENSET polypeptide using the method of claim 24; and
b) combining said modulator with a pharmaceutically acceptable carrier.
